(19) United States
(12) Patent Application Publication
(10) Pub. No.: US 2006/0286102 A1
(43) Pub. Date: Dec. 21, 2006

(54) Cell surface receptor isoforms and methods of identifying and using the same

(76) Inventors: Pei Jin, Palo Alto, CA (US); H. Michael Shepard, San Francisco, CA (US)

Correspondence Address: FISH & RICHARDSON, PC, P.O. BOX 1022, MINNEAPOLIS, MN, 55440-1022, US

(21) Appl. No.: 11/129,740

(22) Filed: May 13, 2005

Related U.S. Application Data
(60) Provisional application No. 60/666,825, filed on Mar. 30, 2005.
(60) Provisional application No. 60/571,289, filed on May 14, 2004.
(60) Provisional application No. 60/580,990, filed on Jun. 18, 2004.

(51) Int. Cl. A61K 39/395; C07H 21/04; C12P 21/06; C07K 14/705; C07K 16/28

(52) U.S. Cl. 424/143.1; 530/350; 530/388.22; 435/69.1; 435/320.1; 435/325; 536/23.5

(57) ABSTRACT

Isoforms of cell surface receptors, including isoforms of receptor tyrosine kinases, and pharmaceutical compositions containing the isoforms are provided. Chimeras of and conjugates containing the cell surface receptors that contain a portion, such as an extracellular domain, from one cell surface receptor, and a second portion, particularly an intron-encoded portion, from a second cell surface protein also are provided. The isoforms modulate the activity of a cell surface receptor. Methods for identifying and preparing isoforms of cell surface receptors including receptor tyrosine kinases are provided. Also provided are methods of treatment with the cell surface receptor isoforms.

### RELATED APPLICATIONS

Benefit of priority is claimed under 35 U.S.C. §119(e) to U.S. Provisional Application Ser. No. 60/666,825 to Pei Jin and H. Michael Shepard, filed Mar. 30, 2005, entitled “CELL SURFACE RECEPTOR ISOFORMS AND METHODS OF IDENTIFYING AND USING SAME;” to U.S. Provisional Application Ser. No. 60/571,289 to Pei Jin, filed May 14, 2004, entitled “CELL SURFACE RECEPTOR ISOFORMS AND METHODS OF IDENTIFYING AND USING SAME,”; and to U.S. Provisional Application Ser. No. 60/580,990 to Pei Jin, filed Jun. 18, 2004, entitled “CELL SURFACE RECEPTOR ISOFORMS AND METHODS OF IDENTIFYING AND USING SAME.”

This application also is related to U.S. application Ser. No. 10/846,113, filed May 14, 2004, and to corresponding published International PCT application No. WO 05/016966, published Feb. 24, 2005, entitled “INTRON FUSION PROTEINS, AND METHODS OF IDENTIFYING AND USING SAME.” This application also is related to International PCT application No. PCT US2005/17051 to Pei Jin and H. Michael Shepard, entitled “CELL SURFACE RECEPTOR ISOFORMS AND METHODS OF IDENTIFYING AND USING THE SAME,” filed the same day herewith.

The subject matter of each of these applications, provisional applications and international applications is incorporated herein by reference thereto.

### INCORPORATION BY REFERENCE OF SEQUENCE LISTING PROVIDED ON COMPACT DISCS

An electronic version on compact disc (CD-R) of a computer-readable form of the Sequence Listing is filed herewith in duplicate (labeled Copy #1 and Copy #2) along with a third CD-R labeled computer-readable form, the contents of which are incorporated by reference in their entirety. The computer-readable file on each of the aforementioned compact discs originally created on May 13, 2005, and created for resubmission on Aug. 1, 2005, is identical, 1,271 kilobytes in size, and is entitled 2817SEQ.001.txt.

### FIELD OF THE INVENTION

Isoforms of cell surface receptors, including isoforms of receptor tyrosine kinases and pharmaceutical compositions containing receptor tyrosine kinase isoforms are provided. The cell surface receptor isoforms and compositions containing them can be used in methods of treatment of diseases, such as cancer and inflammatory disease.

### BACKGROUND

Cell signaling pathways involve a network of molecules including polypeptides and small molecules that interact to relay extracellular, intercellular and intracellular signals. Such pathways interact like a relay, handing off signals from one member of the pathway to the next. Modulation of one member of the pathway can be relayed through the signal transduction pathway, resulting in modulation of activities of other pathway members and modulating outcomes of such signal transduction such as affecting phenotypes and responses of a cell or organism to a signal. Diseases and disorders can involve misregulated or changes in modulation of signal transduction pathways. A goal of drug development is to target such misregulated pathways to restore more normal regulation in the signal transduction pathway.

Receptor tyrosine kinases (RTKs) are among the polypeptides involved in many signal transduction pathways. RTKs play a role in a variety of cellular processes, including cell division, proliferation, differentiation, migration and metabolism. RTKs can be activated by ligands. Such activation in turn activates events in a signal transduction pathway, such as by triggering autocrine or paracrine cellular signaling pathways, for example, activation of second messengers, which results in specific biological effects. Ligands for RTKs specifically bind to the cognate receptors.

RTKs have been implicated in a number of diseases including cancers such as breast and colorectal cancers, gastric carcinoma, gliomas and mesodermal-derived tumors. Disregulation of RTKs has been noted in several cancers. For example, breast cancer can be associated with amplified expression of p185-HER2. RTKs also have been associated with diseases of the eye, including diabetic retinopathies and macular degeneration. RTKs also are associated with regulating pathways involved in angiogenesis, including physiologic and tumor blood vessel formation. RTKs also are implicated in the regulation of cell proliferation, migration and survival.

The human epidermal growth factor receptor 2 gene (HER-2; also referred to as ErbB2) encodes a receptor tyrosine kinase that has been implicated as an oncogene. HER-2 has a major mRNA transcript of 4.5 Kb that encodes a polypeptide of about 185 kD (P185HER2). P185HER2 contains an extracellular domain, a transmembrane domain and an intracellular domain with tyrosine kinase activity. Several polypeptide forms are produced from the HER-2 gene and include polypeptides generated by proteolytic processing and forms generated from alternatively spliced RNAs. Herstatins and fragments thereof are HER-2 binding proteins, encoded by the HER-2 gene. Herstatins (also referred to as p68HER-2) are encoded by an alternatively spliced variant of the gene encoding the p185-HER2 receptor. For example, one Herstatin occurs in fetal kidney and liver, and includes a 79 amino acid intron-encoded insert, relative to the membrane-localized receptor, at the C terminus (see U.S. Pat. No. 6,414,130 and U.S. Published Application No. 20040022785). Several Herstatin variants have been identified (see, e.g., U.S. Pat. No. 6,414,130; U.S. Published Application No. 20040022785, U.S. application Ser. No. 09/234,208; U.S. application Ser. No. 09/506,079; published international application Nos. WO0044403 and WO0161356). Herstatins lack an epidermal growth factor (EGF) homology domain and contains part of the extracellular domain, typically the first 340 amino acids, of p185-HER2. Herstatins contain subdomains I and II of the human epidermal growth factor receptor, the HER-2 extracellular domain and a C-terminal domain encoded by an intron. The resulting herstatin polypeptides typically contain 419 amino acids (340 amino acids from subdomains I and II, plus 79 amino acids from intron 8). The herstatin proteins lack extracellular domain IV, as well as the transmembrane domain and kinase domain.

In contrast, positive acting EGFR ligands, such as the epidermal growth factor and transforming growth factor-alpha, possess such domains. Additionally, binding of a herstatin does not activate the receptor. Herstatins can inhibit members of the EGF-family of receptor tyrosine kinases as well as the insulin-like growth factor-1 (IGF-1) receptor and other receptors. Herstatins prevent the formation of productive receptor dimers (homodimers and heterodimers) required for transphosphorylation and receptor activation. Alternatively or additionally, herstatin can compete with a ligand for binding to the receptor terminus (see, U.S. Pat. No. 6,414,130; U.S. Published Application No. 20040022785, U.S. application Ser. No. 09/234,208; U.S. application Ser. No. 09/506,079; published international application Nos. WO0044403 and WO0161356).

The tumor necrosis factor family of receptors (TNFRs) is another example of a family of receptors involved in signal transduction and regulation. The TNF ligand and receptor family regulate a variety of signal transduction pathways including those involved in cell differentiation, activation, and viability. TNFRs contain an extra-cellular domain, including a ligand binding domain, a transmembrane domain and an intracellular domain that participates in signal transduction. Additionally, TNFRs are typically trimeric proteins that trimerize at the cell surface. TNFRs play a role in inflammatory diseases, central nervous system diseases, autoimmune diseases, airway hyper-responsiveness conditions such as in asthma, rheumatoid arthritis and inflammatory bowel disease. TNFRs also play a role in infectious diseases, such as viral infection.

The TNF family of receptors (TNFR) exhibit homology among the extra-cellular domains. Some of these receptors initiate apoptosis, some initiate cell proliferation and some initiate both activities. Signaling by this family requires clustering of the receptors by trimeric ligand and subsequent association of proteins with the cytoplasmic region of the receptors. The TNFR family contains a sub family with homologous cytoplasmic 80-amino-acid domains. This domain is referred to as a death domain (DD), so named because proteins that contain this domain are involved in apoptosis. The distinction between members of the TNFR family is exemplified by two TNFRs coded by distinct genes. TNFR1 (55 kDa) signals the initiation of apoptosis and the activation of the transcription factor NFkB. TNFR2 (75 kDa) functions to signal activation of NFkB but not the initiation of apoptosis. TNFR1 contains a DD; TNFR2 does not.

Because of their involvement in a variety of diseases and conditions, cell surface receptors (CSRs) such as RTKs and TNFRs are targets for therapeutic intervention. Small molecule therapeutics that target RTKs have been designed. While it may be possible to design small molecules as therapeutics that target cell surface receptors and/or other receptors, there, however, are a number of limitations with such strategies. Small molecules can be limited to interactions with one receptor and thus unable to address conditions where multiple family members may be misregulated. Small molecules also can be promiscuous and affect receptors other than the intended target. Additionally, some small molecules bind irreversibly or substantially irreversibly to the receptors (i.e. subnanomolar binding affinity). The merits of such approaches have not been validated. Antibodies against receptor and/or receptor ligands can be used as therapeutics. Antibody treatments, however, can result in an immune response in a subject and thus, such treatments often need extensive tailoring to avoid complications in treatment. Thus, there exists an unmet need for therapeutics for treatment of diseases, including cancers and other diseases involving undesirable cell proliferation and inflammatory reactions, involving cell surface receptors that exhibit RTK activity and/or other cell surface proteins. Accordingly, among the objects herein, it is an object to provide such therapeutics and methods for identifying or discovering candidate therapeutics and methods of treatment.

### SUMMARY

Therapeutic molecules for treating diseases and disorders involving the signal transduction pathways and other cell surface receptor interactions are provided. The therapeutic molecules particularly target RTKs that participated in signal transduction pathways, including those involved in angiogenesis and neovascularization and cell proliferation, particular aberrant angiogenesis, neovascularization and/or cell proliferation. Also provided are compositions containing the molecules and methods for treating diseases and conditions, particularly diseases that include or exhibit or are manifested by aberrant angiogenesis, neovascularization and/or cell proliferation. Also provided are methods for identifying candidate therapeutics and methods of treatment by administering therapeutic molecules and compositions. The therapeutic molecules can be used for treating any such disease or disorder and exhibit activity, whereby such treatment is effective. Diseases and disorders include proliferative disorders, including tumors, immune disorders and inflammatory disorders. Targets include cells involved in angiogenesis and neovascularization and cells involved in inflammatory responses, cancers and other such disorders. Activity includes modulation of the activity of a cell surface receptor, including RTKs and TNFRs, such as by directly altering the activity by virtue of interaction with the receptor or indirectly by interacting with ligands.

Included among the therapeutic molecules are polypeptide CSR isoforms or peptidomimetic variants thereof or allelic variants of the CSR. Among these molecules are those that modulate an activity or function of a cell surface receptor (CSR), particularly a CSR that is a cognate receptor of the isoforms. Activities and functions modulated by isoforms include, for example, one or more of: dimerization, homodimerization, heterodimerization, trimerization, kinase activity, receptor-associated kinase activity, receptor-associated protease activity, autophosphorylation of the receptor tyrosine kinase, transphosphorylation of the receptor tyrosine kinase, phosphorylation of a signal transduction molecule, ligand binding, competition with the receptor tyrosine kinase for ligand binding, signal transduction, interaction with a signal transduction molecule, induction of apoptosis, membrane association and membrane localization.

Among the therapeutic molecules provided herein are those that modulate the activity of cellular receptors of angiogenic factors (positive and negative), which serve as points of intervention in a plurality of disease processes. Examples of situations in which ‘too much’ angiogenesis is bad include angiogenesis that supplies blood to tumor foci, or to other sites of disease (such as to the human eye in diabetes). In these cases, therapeutic molecules provided herein that inhibit the process are employed.

Isoforms of cell surface receptors, including isoforms of receptor tyrosine kinases, and pharmaceutical compositions containing the isoforms are provided. Chimeras of and conjugates containing the cell surface receptors that contain a portion, such as an extracellular domain, from one cell surface receptor, and a second portion, particularly an intron-encoded portion, from a second cell surface protein also are provided. The isoforms modulate the activity of a cell surface receptor, either directly, such as by interacting therewith to alter an activity, such as receptor dimerization, or indirectly, such as by interaction with a receptor ligand. Methods for identifying and preparing isoforms of cell surface receptors including receptor tyrosine kinases are provided. Also provided are methods of treatment with the cell surface receptor isoforms.

Activities of the receptor tyrosine kinase (RTK) or TNFR (or other cell surface receptors) modulated by the therapeutic molecules provided herein include, but are not limited to, for example, one or more of dimerization, homodimerization, hetero-dimerization, trimerization, kinase activity, autophosphorylation of the receptor tyrosine kinase, transphosphorylation of the receptor tyrosine kinase, phosphorylation of a signal transduction molecule, ligand binding, competition with the receptor tyrosine kinase for ligand binding, signal transduction, interaction with a signal transduction molecule, induction of apoptosis, receptor-associated kinase activity, receptor-associated protease activity, membrane association and membrane localization. Modulation includes, for example, inhibition (such as activity as an antagonist) of an activity and also enhancement (such as activity as an agonist) of an activity. By virtue of modulation of such activity the effects of such receptors are modulated or otherwise modified.

The therapeutic molecules provided herein typically are polypeptides or peptidomimetics (including polypeptides with modified bonds) or other modified forms of polypeptides designed, for example, for improved bioavailability, delivery, stability, resistance to proteases and other properties. Contemplated are modifications of the molecules with changes that alter properties, such as bioavailability, protein stability and other such properties, for their use as therapeutics.

Exemplary of the molecules are polypeptides. Also included are allelic variants of any of the polypeptides. The allelic variants include any of the variants of the receptor, particularly variants in an extracellular domain, present in a population of the mammal in which a particular receptor occurs. Chimeric molecules, conjugates and conjugates of intron portions of the intron fusion proteins also are provided. The chimeric molecules and conjugates can include portions from molecules with different ligand binding and/or receptor interacting specificities. For example, conjugates or chimeras that contain an extracellular domain or portion thereof linked directly or indirectly to an intron region, such as an intron of a herstatin, are provided. The chimeras and conjugates include portions from CSR isoforms provided herein and known to those of skill in the art including any described in U.S. Provisional Application Ser. No. 60/571,289, U.S. Provisional Application Ser. No. 60/580,990, U.S. application Ser. No. 10/846,113, published International PCT application No. WO 05/016966, U.S. Pat. No. 6,414,130; U.S. Published Application No. 20040022785, U.S. application Ser. No. 09/234,208; U.S. application Ser. No. 09/506,079; published international application Nos. WO0044403 and WO0161356.

Isolated polypeptides and variants thereof are provided. The polypeptides are isoforms of cell surface receptors (CSR isoforms) and chimeras and conjugates thereof. Some CSR isoforms, such as intron fusion proteins, are missing all or part of a functional domain or other structural feature such that the activity of the domain is reduced or eliminated and/or a structure is altered compared to the full-length cognate receptor. Other examples include intron fusion proteins in which the rearrangements that occur during alternative splicing can result in either positive or negatively acting molecules. In particular, among the polypeptides provided herein are soluble or non-membrane bound forms of receptors. The polypeptides include a sequence of amino acids that has at least 80%, 85%, 90% or 95% sequence identity with a sequence of amino acids set forth in any of SEQ ID NOS: 91, 93, 95, 115, 117, 119, 121, 123, 125, 127, 129, 131, 133, 135, 137, 139, 141, 143, 145, 147, 149, 151, 153, 155, 168, 170, 172, 174, 176, 178, 180, 182, 184, 186, 188, 190, 192, 194, 196, 198, 200, 202, 204, 206, 208, 210, 212, 214, 216, 218, 220, 222, 224, and 226 and allelic variations thereof. Such homology is exhibited along at least 70%, 80%, 85%, 90%, 95%, 97% or 100% of the full-length of the polypeptide. Sequence identity is compared along the full length of the polypeptide represented by each SEQ ID to the full length sequence of the isolated polypeptide, and each of SEQ ID NOS: 91, 93, 95, 115, 117, 119, 121, 123, 125, 127, 129, 131, 133, 135, 137, 139, 141, 143, 145, 147, 149, 151, 153, 155, 168, 170, 172, 174, 176, 178, 180, 182, 184, 186, 188, 190, 192, 194, 196, 198, 200, 202, 204, 206, 208, 210, 212, 214, 216, 218, 220, 222, 224, and 226 is a cell surface receptor isoform. Exemplary of such polypeptides are isolated polypeptides containing the sequence of amino acids set forth in any of SEQ ID NOS: 91, 93, 95, 115, 117, 119, 121, 123, 125, 127, 129, 131, 133, 135, 137, 139, 141, 143, 145, 147, 149, 151, 153 and 155 as are isolated polypeptides that have the sequence of amino acids set forth in any of SEQ ID NOS: 91, 93, 95, 115, 117, 119, 121, 123, 125, 127, 129, 131, 133, 135, 137, 139, 141, 143, 145, 147, 149, 151, 153 155, 168, 170, 172, 174, 176, 178, 180, 182, 184, 186, 188, 190, 192, 194, 196, 198, 200, 202, 204, 206, 208, 210, 212, 214, 216, 218, 220, 222, 224, and 226. Also provided are chimeras of these molecules and also chimeras of these molecules and herstatins.

Provided are isolated polypeptides that are receptor isoforms and that contain at least one domain of a cell surface receptor linked to at least one amino acid encoded by an intron of a gene encoding a cognate cell surface receptor. The cell surface receptor is selected from among DDR1 (discoidin domain receptor), KIT (receptor for c-kit), FGFR-1, FGFR-2, FGFR-4, (fibroblast growth factor receptors 1, 2 and 4) TNFR1B (tumor necrosis factor receptor1B; also referred to as TNFRSF1B), VEGFR-1, VEGFR-2, VEGFR-3, (vascular endothelial growth factor receptors 1, 2, and 3), RON (recepteur d'origine nantais; also known as macrophage stimulating 1 receptor), MET (also known as hepatocyte growth factor receptor), TEK (endothelial-specific receptor tyrosine kinase), Tie-1 (tyrosine kinase with immunoglobulin and epidermal growth factor homology domains receptor), CSF1R (colony stimulating factor 1 receptor), PDGFR-B (platelet-derived growth factor receptor B), EphA1, EphA2, and EphB1 (erythropoietin-producing hepatocellular receptor A1, A2 and B1, respectively). Exemplary of such polypeptides are those that contain the sequence of amino acids selected from among the sequences of amino acids set forth in SEQ ID NOS: 91, 93, 95, 115, 117, 119, 121, 123, 125, 127, 129, 131, 133, 135, 137, 139, 141, 143, 145, 147, 149, 151, 153, 155, 168, 170, 172, 174, 176, 178, 180, 182, 184, 186, 188, 190, 192, 194, 196, 198, 200, 202, 204, 206, 208, 210, 212, 214, 216, 218, 220, 222, 224, and 226.

Also provided are isolated polypeptide that are cell surface receptors that lack at least part of a transmembrane domain such that the resulting polypeptide is not membrane localized or bound and it modulates an activity, including a biological activity, of the cell surface receptor. The polypeptides can include exon insertions. Among these are cell surface receptor isoforms selected from among isoforms of FGFR-4, KIT and TNFR. Exemplary of the isolated polypeptides are those that have at least 80%, 85%, 90%, 95%, 97%, or 100% sequence identity with a sequence of amino acids set forth in any of SEQ ID NOS: 91, 93, 95, 115, 117, 119, 121, 123, 125, 127, 129, 131, 133, 135, 137, 139, 141, 143, 145, 147, 149, 151, 153, 155, 168, 170, 172, 174, 176, 178, 180, 182, 184, 186, 188, 190, 192, 194, 196, 198, 200, 202, 204, 206, 208, 210, 212, 214, 216, 218, 220, 222, 224, and 226. Sequence identity is compared along the full length of each SEQ ID to the sequence of the full length of the isolated polypeptide. The isolated polypeptides can further lack a cell surface receptor cytoplasmic domain.

Also provided are isolated polypeptides that contain an intron-encoded sequence of amino acids and lack a cell surface receptor cytoplasmic domain. The intron is an intron and is selected from among nucleic acids KIT, FGFR-4, TNFR2, VEGFR-1, RON, TEK, Tie-1, and EphA1, or is an intron from any of SEQ ID NOS: 91, 93, 95, 121, 123, 129, 131, 133, 135, 137, 139, 141, 149, 151, or 153. Also provided are polypeptides that further lack a transmembrane domain. Among these are isolated polypeptides that modulate an activity or function of a cell surface receptor. These polypeptides include TNFR isoforms, such as, but not limited to, TNFR1 (also referred to as TNFRSF1A), TNFR2 and TNFRrp, the low-affinity nerve growth factor receptor, Fas antigen, CD40, CD27, CD30, 4-1BB, OX40, DR3, DR4, DR5, and herpes virus entry mediator (HVEM).

Also provided are chimeric intron fusion protein isoforms that contain an N-terminal portion that effects binding to a CSR linked to an intron, such as the intron or a portion thereof whereby the resulting chimera modulates, particularly, inhibits, an activity of one or more CSRs. The chimeras include N-terminal and/or intron portions of any of the isoforms provided herein and also a herstatin, linked to an intron from a different intron fusion protein isoform. The portions of the chimeras can be linked via a linker or via 2 or more amino acids. Alternatively, the chimera can be a chemical conjugate.

Also provided are CSR isoforms conjugates and chimeras in which the N-terminal portion and intron-encoded portion are linked directly or via a linker and are from the same or a different CSR isoforms, including any provided herein, a herstatin or any other CSR. The two portions can be linked via a linker, such as a polypeptide or chemical linker. The isoform conjugates modulate, typically inhibit, the activity of one or more CSRs. The CSRs include those that participate in signal transduction, particularly CSRs involved in pathways that participate in angiogenesis, inflammatory responses and cell proliferation (see, e.g., FIG. 1).

Provided herein are CSR isoforms that contain at least one domain of a CSR receptor and lack one or more amino acids of another domain of the CSR receptor such as the transmembrane domain and/or protein kinase domain, whereby an activity is reduced or abolished compared to the CSR. CSR isoforms include polypeptides that contain an intron-encoded sequence of amino acids, wherein the intron is from a gene encoding the CSR. For example, a CSR isoform can contain at least one domain of the CSR receptor operatively linked to at least one amino acid encoded by an intron of a gene encoding the CSR. Among the CSR isoforms provided herein are polypeptides that contain one or more domains of an Ephrin (Eph) receptor, a fibroblast growth factor (FGF) receptor, a DDR receptor, a MET receptor, a RON receptor, a TEK/TIE receptor, a VEGF receptor, PDGF receptor, CSF1 receptor, a KIT receptor and a TNFR receptor.

Provided herein are EphA isoforms. The isoforms are isolated polypeptides that contain at least one domain of an EphA receptor. The polypeptides contain an ephrin ligand binding domain and lack one or more amino acids corresponding to the transmembrane domain of the EphA receptor, whereby the membrane localization of the polypeptide is reduced or abolished compared to the EphA receptor. Included are polypeptides where the EphA receptor is selected from among EphA1, EphA2, EphA3, EphA4, EphA5, EphA6, EphA7, and EphA8. In one example, such polypeptides include a sequence as set forth in any one of SEQ ID NO: 253-260 or an allelic variant thereof. The allelic variant can be an allelic variation present in any one of SEQ ID NOS: 289-293. EphA isoforms include polypeptides that lack all or part of a protein kinase domain compared to the EphA receptor and/or that lack all or part of a Sterile Alpha Motif domain (SAM) compared to the EphA receptor.

In one example, an EphA isoform has at least one domain of an EphA1 receptor as set forth in SEQ ID NO:253. Such isoforms include EphA1 isoforms where the polypeptide lacks one or more amino acids of a protein kinase domain of the EphA1 receptor, whereby the kinase activity of the polypeptide is reduced or abolished compared to the EphA1 receptor. EphA1 isoforms also include polypeptides that have at least 80% sequence identity with a sequence of amino acids set forth in any of SEQ ID NOS: 149, 151 and 153 or that contain the amino acid sequence set forth in any of SEQ ID NOS: 149, 151 and 153 or an allelic variant thereof. Allelic variants include the allelic variations as set forth in SEQ ID NO: 289. EphA1 isoforms include polypeptides that contain the same number of amino acids as set forth in any of SEQ ID NOS: 149, 151 and 153.

Provided herein are EphA2 isoforms. EphA2 isoforms include at least one domain of an EphA2 receptor as set forth in SEQ ID NO:254, where the polypeptide lacks one or more amino acids of a transmembrane domain and protein kinase domain compared to the EphA2 receptor, whereby the membrane localization and the protein kinase activity of the polypeptide are reduced or abolished compared to the EphA2 receptor. EphA2 isoforms include polypeptides that contain one or more amino acids of a fibronectin domain compared to the EphA2 receptor. Examples of EphA2 isoforms also include polypeptides that have at least 80% sequence identity with a sequence of amino acids as set forth in SEQ ID NO: 168 or contains the sequence of amino acids set forth in SEQ ID NO: 168 or an allelic variant thereof. Allelic variants include, but are not limited to, allelic variations as set forth in SEQ ID NO: 290. EphA2 isoforms include isoforms that contain the same number of amino acids as set forth in the SEQ ID NO:168.

Also provided herein are EphB isoforms that include polypeptides lacking one or more amino acids of a transmembrane domain compared to the EphB receptor, whereby the membrane localization of the polypeptide is reduced or abolished compared to the EphB receptor. Among the EphB isoforms provided are those where the EphB receptor is selected from EphB1, EphB2, EphB3, EphB4, EphB5, and EphB6 and where the EphB receptor comprises a sequence as set forth in any one of SEQ ID NOS: 261-265 or an allelic variant thereof. Allelic variants include, but are not limited to, allelic variations set forth in SEQ ID NOS: 294-298. Exemplary EphB isoforms include isoforms that lack one or more amino acids of a protein kinase domain of the EphB receptor, whereby the protein kinase activity of the polypeptide is reduced or abolished compared to the EphB receptor and isoforms that lacks one or more amino acids of a Sterile Alpha Motif domain (SAM) of the EphB receptor. In one example, an EphB1 isoform includes an ephrin binding domain. EphB isoforms also include isoforms that lack one or more amino acids of a fibronectin domain of the EphB receptor. Among the EphB isoforms provided herein are isoforms that have at least 80% sequence identity with a sequence of amino acids as set forth in any of SEQ ID NOS: 155, 170, 172 and 174 and isoforms that contain the sequence of amino acids set forth in any of SEQ ID NOS: 155, 170, 172 and 174 or an allelic variant thereof. Allelic variants include, but are not limited to, allelic variations set forth in SEQ ID NOS: 294 and 297. EphB isoforms include isoforms that contain the same number of amino acids as set forth in any of SEQ ID NOS: 155, 170, 172 and 174.

FGFR isoforms are provided herein. Included are FGFR isoforms that contain at least one domain of an FGFR-1, wherein the polypeptide comprises an immunoglobulin domain corresponding to amino acids 253-357 of FGFR-1 set forth in SEQ ID NO:268 and lacks all of a transmembrane domain corresponding to amino acids 375-397 of the FGFR-1. FGFR isoforms also include isoforms that lack one or more amino acids of a protein kinase domain of FGFR-1, whereby the protein kinase activity of the polypeptide is reduced or abolished compared to the FGFR-1 and/or that contain one or more amino acids of an immunoglobulin domain corresponding to amino acids 156-246 of FGFR-1. FGFR isoforms provided include isoforms that have at least 80% sequence identity with a sequence of amino acids set forth in SEQ ID NOS: 119 or 176 and isoforms that contain any of SEQ ID NOS: 119 and 176 or an allelic variant thereof. Allelic variants include, but are not limited to, allelic variations set forth in SEQ ID NO: 300. FGFR-1 isoforms include isoforms that have the same number of amino acids as set forth in any of SEQ ID NOS: 119 and 176.

Also provided are FGFR-2 isoforms that have at least one domain of an FGFR-2 as set forth in SEQ ID NO: 269, where the polypeptide lacks a transmembrane domain and a protein kinase domain compared to FGFR-2, whereby the membrane localization and protein kinase activity of the polypeptide is reduced or abolished compared to FGFR-2. Such isoforms include polypeptides that have at least 80% sequence identity with a sequence of amino acids set forth in SEQ ID NOS: 178, 180, 182 and 184 and isoforms that contain the sequence of amino acids set forth in SEQ ID NOS: 178, 180, 182 or 184 or an allelic variant thereof. Allelic variants include, but are not limited to, allelic variations set forth in SEQ ID NO: 301. FGFR-2 isoforms include isoforms that have the same number of amino acids as set forth in any of SEQ ID NOS: 178, 180, 182 or 184. Exemplary FGFR-2 isoforms also include isoforms that lack an immunoglobulin domain corresponding to amino acids 41-125 of the FGFR-2.

FGFR-4 isoforms are provided herein that contain at least one domain of an FGFR-4, such as an immunoglobulin domain corresponding to amino acids 249-351 of the FGFR-4 set forth in SEQ ID NO:271 and lack a transmembrane domain and protein kinase domain of the FGFR-4, whereby the membrane localization and protein kinase activity of the polypeptide is reduced or abolished compared to FGFR-4. FGFR isoforms include isoforms that have at least 80% sequence identity with a sequence of amino acids set forth in SEQ ID NO: 121 and isoforms that contain the sequence of amino acids set forth in SEQ ID NO: 121 or an allelic variant thereof. Allelic variants include, but are not limited to, allelic variations set forth in SEQ ID NO:303. FGFR-4 isoforms include isoforms that have the same number of amino acids as set forth in SEQ ID NO: 121.

Provided herein are DDR1 isoforms, that are polypeptides that contain at least one domain of a DDR1 as set forth in SEQ ID NO: 250, where the polypeptide lacks a transmembrane domain and a protein kinase domain compared to the DDR1, whereby the membrane localization and protein kinase activity of the polypeptide is reduced or abolished compared to DDR1, and the polypeptide has at least 80% sequence identity with a sequence of amino acids set forth in SEQ ID NOS: 115 or 117. DDR1 isoforms include isoforms that contain the sequence of amino acids set forth in SEQ ID NOS: 115 or 117 or an allelic variant thereof, such as but not limited to the allelic variations as set forth in SEQ ID NO: 286. DDR1 isoforms include isoforms that have the same number of amino acids as set forth in SEQ ID NOS: 115 or 117.

Also provided herein are MET receptor isoforms that are polypeptides which contain at least one domain of a MET receptor operatively linked to at least one amino acid encoded by an intron of a gene encoding MET, where the polypeptide lacks a transmembrane domain, protein kinase domain and at least one additional domain compared to a MET receptor set forth in SEQ ID NO:274, whereby the membrane localization and protein kinase activity of the polypeptide is reduced or abolished compared to the MET receptor. MET receptor isoforms include isoforms where the additional domain lacking as compared to the MET receptor is a Sema domain, a plexin domain or an IPT/TIG domain. MET receptor isoforms include isoforms that have at least 80% sequence identity with a sequence of amino acids as set forth in any of SEQ ID NOS: 186, 188, 190, 192, 196, 198, 200, 202, 204, 206, 208 and 214 and isoforms that contain the sequence of amino acids set forth in any of SEQ ID NOS: 186, 188, 190, 192, 196, 198, 200, 202, 204, 206, 208 and 214 or an allelic variant thereof. Allelic variants include, but are not limited to, allelic variations set forth in SEQ ID NO:306. MET isoforms include isoforms that have the same number of amino acids as set forth in any of SEQ ID NOS: 186, 188, 190, 192, 196, 198, 200, 202, 204, 206, 208 and 214.

RON receptor isoforms are provided herein. RON receptor isoforms include polypeptides that have a plexin domain of the RON receptor as set forth in SEQ ID NO: 277; and lack a transmembrane domain of the RON receptor, whereby the membrane localization of the polypeptide is reduced or abolished compared to the RON receptor. RON receptor isoforms include isoforms that lack one or more amino acids of a protein kinase domain compared to the RON receptor as set forth in SEQ ID NO: 277, whereby the protein kinase activity of the polypeptide is reduced or abolished compared to the RON receptor and/or contain one or more amino acids of at least one IPT/TIG domain of the RON receptor. RON receptor isoforms include isoforms that have at least 80% sequence identity with a sequence of amino acids as set forth in any of SEQ ID NOS: 216, 218 and 220 and isoforms that contain the sequence of amino acids set forth in any of SEQ ID NOS: 216, 218 and 220 or an allelic variant thereof, such as but not limited to allelic variations set forth in SEQ ID NO: 308. RON receptor isoforms also include isoforms that have the same number of amino acids as set forth in any of SEQ ID NOS: 216, 218 and 220.

Provided herein are TEK isoforms that contain at least one domain of a TEK receptor as set forth in SEQ ID NO: 278, where the isoform lacks a transmembrane domain, and a protein kinase domain, whereby the membrane localization and protein kinase activity of the polypeptide are reduced or abolished compared to the TEK receptor; and lacks one or more amino acids of at least one fibronectin domain compared to the TEK receptor. TEK isoforms include isoforms where the fibronectin domain lacking corresponds to amino acids 444-529, 543-626, or 639-724 of SEQ ID NO: 278 and where one or more amino acids of the three fibronectin domains of the TEK receptor corresponding to amino acids 444-529, 543-626, and 639-724 of SEQ ID NO: 278 is lacking. TEK isoforms include isoforms that have at least 80% sequence identity with a sequence of amino acids as set forth in any of SEQ ID NOS: 131 and 133 and isoforms that contain the sequence of amino acids set forth in any of SEQ ID NOS: 131 and 133 or an allelic variant thereof, such as but not limited to allelic variations as set forth in SEQ ID NO: 309. TEK isoforms also include isoforms that contain the same number of amino acids as set forth in any of SEQ ID NOS: 131 and 133.

Tie-1 receptor isoforms are provided herein that contain all or part of at least one domain of a Tie-1 receptor as set forth in SEQ ID NO: 279, where the isoform lacks a transmembrane domain and a protein kinase domain compared to the Tie-1 receptor, whereby the membrane localization and protein kinase activity of the polypeptide are reduced or abolished compared to the Tie-1 receptor; and the isoform contains a sequence of amino acids set forth in any of SEQ ID NOS: 135, 137, 139, 141, 143 and 222 or an allelic variant thereof. Allelic variants include, but are not limited to, allelic variations set forth in SEQ ID NO: 310. Tie-1 receptor isoforms include isoforms that have the same number of amino acids as set forth in any of SEQ ID NOS: 135, 137, 139, 141, 143 and 222.

Provided herein are VEGFR isoforms. VEGFR isoforms include VEGFR-1 isoforms that contain a sequence of amino acids that has at least 80% sequence identity with the sequence of amino acids as set forth in SEQ ID NO: 123 and that lack a transmembrane domain and a protein kinase domain compared to the VEGFR-1 receptor set forth in SEQ ID NO: 282. Such isoforms include polypeptides that contain the sequence of amino acids set forth in SEQ ID NO: 123 or an allelic variant thereof and isoforms that contain the same number of amino acids as set forth in any of SEQ ID NO: 123. VEGFR isoforms include VEGFR-2 and VEGFR-3 isoforms that contain at least one domain of a VEGFR set forth in any of SEQ ID NOS:283 and 284, where the polypeptide lacks one or more amino acids of a transmembrane domain of the VEGFR, whereby the membrane localization of the polypeptide is reduced or abolished compared to the VEGFR. VEGFR-2 and VEGFR-3 isoforms also include isoforms that lack one or more amino acids of a protein kinase domain, whereby the protein kinase activity of the polypeptide is reduced or abolished compared to the VEGFR and isoforms that lack one or more amino acids of an immunoglobulin domain compared to the VEGFR. VEGFR-2 and VEGFR-3 isoforms include polypeptides that have at least 80% sequence identity with a sequence of amino acids as set forth in any of SEQ ID NOS: 125, 127, 224 and 226 and polypeptides that contain the sequence of amino acids set forth in any of SEQ ID NOS: 125, 127, 224 and 226 or an allelic variant thereof. Allelic variants can include, but are not limited to the allelic variations as set forth in SEQ ID NOS: 313 and 314. VEGFR-2 and VEGFR-3 isoforms also include isoforms that have the same number of amino acids as set forth in any of SEQ ID NOS: 125, 127, 224 and 226.

PDGFR isoforms are provided herein. Included are PDGFR isoforms that contain at least one domain of a PDGFR-B as set forth in SEQ ID NO: 276, wherein the polypeptide lacks one or more amino acids of a transmembrane domain of the PDGFR-B, whereby the membrane localization of the polypeptide is reduced or abolished compared to the PDGFR-B. PDGFR isoforms also include isoforms that lack one or more amino acids of a protein kinase domain of the PDGFR-B, whereby the protein kinase activity of the polypeptide is reduced or abolished compared to the PDGFR-B and isoforms that contain one or more amino acids of an immunoglobulin domain of the PDGFR-B. Also included are PDGFR isoforms that have at least 80% sequence identity with a sequence of amino acids set forth in SEQ ID NO: 147 and isoforms that contain the sequence of amino acids set forth in SEQ ID NO: 147 or an allelic variant thereof. Allelic variants can include, but are not limited to the allelic variations as set forth in SEQ ID NO: 307. PDGFR isoforms also include isoforms that have the same number of amino acids as set forth in SEQ ID NO: 147.

Also provided herein are CSF1R isoforms that contain at least one domain of a CSF1R as set forth in SEQ ID NO: 249, where the polypeptide lacks one or more amino acids of a transmembrane domain of the CSF1R, whereby the membrane localization of the polypeptide is reduced or abolished compared to the CSF1R. CSF1R isoforms also include isoforms that lack one or more amino acids of a protein kinase domain of the CSF1R, whereby the protein kinase activity of the polypeptide is reduced or abolished compared to the CSF1R and isoforms that contain one or more amino acids of an immunoglobulin domain of the CSF1R. Included are CSF1R isoforms that have at least 80% sequence identity with a sequence of amino acids set forth in SEQ ID NOS: 145 and isoforms that contain the sequence of amino acids set forth in SEQ ID NOS: 145 or an allelic variant thereof, such as but not limited to allelic variations as set forth in SEQ ID NO: 285. Exemplary CSF1R isoforms also include isoforms that contain the same number of amino acids as set forth in SEQ ID NO: 145.

KIT receptor isoforms are provided herein. Included are KIT receptor isoforms that contain at least one domain of a KIT receptor as set forth in SEQ ID NO:273 and lack one or more amino acids of a transmembrane domain and a protein kinase domain of the KIT receptor, whereby the membrane localization and protein kinase activity of the polypeptide are reduced or abolished compared to the KIT receptor and isoforms that contain at least one immunoglobulin domain of the KIT receptor. KIT isoforms include isoforms that have at least 80% sequence identity with a sequence of amino acids set forth in SEQ ID NOS: 93 and isoforms that contain the sequence of amino acids set forth in SEQ ID NO: 93 or an allelic variant thereof, such as but not limited to the allelic variations as set forth in SEQ ID NO: 305. KIT receptor isoforms include isoforms that have the same number of amino acids as set forth in SEQ ID NO: 93.

Provided herein are TNFR isoforms that contain at least one cysteine rich c6 domain of a TNFR as set forth in SEQ ID NOS:280 or 281 and lack all of the transmembrane domain of the TNFR, whereby the membrane localization of the polypeptide is reduced or abolished compared to the TNFR. TNFR isoforms include isoforms that contain at least two cysteine rich c6 domains of the TNFR. TNFR isoforms also include isoforms that have at least 80% sequence identity with a sequence of amino acids set forth in SEQ ID NO: 95 and isoforms that contain the sequence set forth in SEQ ID NO: 95 or an allelic variant thereof. Allelic variation includes but is not limited to allelic variations as set forth in SEQ ID NO: 312. TNFR isoforms also include isoforms that have the same number of amino acids as set forth in SEQ ID NO: 95.

The isolated polypeptides (e.g, CSR isoforms) can be encoded by a gene in a mammal, particularly a human, and can be isolated from a mammalian cell or prepared from nucleic acid cloned from such cell or can be synthesized from nucleic acid prepared by any means or can be synthesized as polypeptides. Exemplary mammals include humans and other primates, horses, cattle, dogs, cats and other domesticated animals, and rodents, such as rats and mice. The isolated polypeptides can be identified by the methods provided herein, known to those of skill in the art and/or also in, for example, copending application U.S. application Ser. No. 10/846,113 and published PCT application No. WO 2005/016966.

Also provided are pharmaceutical compositions that contain any of the isolated polypeptides provided herein and combinations thereof and combinations with other receptor isoforms, including a herstatin. Included among the compositions are those that contain a polypeptide that complexes with a receptor tyrosine kinase or a tumor necrosis factor receptor. The pharmaceutical compositions can be used to treat diseases that include inflammatory diseases, immune diseases, cancers, and other diseases that manifest aberrant angiogenesis or neovascularization or cell proliferation. Cancers include breast, lung, colon, gastric cancers, pancreatic cancers and others. Inflammatory diseases, include, for example, diabetic retinopathies and/or neuropathies and other inflammatory vascular complications of diabetes, autoimmune diseases, including autoimmune diabetes, atherosclerosis, Crohn's disease, diabetic kidney disease, cystic fibrosis, endometriosis, diabetes-induced vascular injury, inflammatory bowel disease, Alzheimer's disease and other neurodegenerative diseases, and other diseases known to those of skill in the art that involve proliferative responses, immune responses and inflammatory responses and others in which RTKs, particularly those noted in FIG. 1 and throughout the disclosure herein are implicated, involved or in which they participate.

Also provided are nucleic acid molecules encoding any of the polypeptides. Vectors containing the nucleic acid molecules are provided as are cells containing the vectors or nucleic acid molecules. Among the nucleic acid molecules provided are those that contain an intron and an exon, where the intron contains a stop codon; the nucleic acid molecule encodes an open reading frame that spans an exon intron junction; and the open reading frame terminates at the stop codon in the intron. The intron can encode one or more amino acids of the encoded polypeptide or the codon can be a first codon (and possibly the only codon) in the intron.

Also provided are chimeric polypeptides that contain a portion, typically one domain, of one cell surface receptor (CSR) isoform and a portion of a second, different CSR isoform, typically at least one domain. The chimeric isoform modulates the activity of one or more tyrosine kinase receptor. Each portion contains at least 4, 5, 6, 7, 8, 10, 12, 15, 20, 25, 30, 50, 100, 150 or more amino acid residues. At least one of the isoforms is an isoform provided herein.

Also provided are nucleic acid molecules that contain a sequence of nucleotides that has at least 90% sequence identity with a sequence of nucleotides set forth in any of SEQ ID NOS: 90, 92, 94, 114, 116, 118, 120, 122, 124, 126, 128, 130, 132, 134, 136, 138, 140, 142, 144, 146, 148, 150, 152, 154, 167, 169, 171, 173, 175, 177, 179, 181, 183, 185, 187, 189, 191, 193, 195, 197, 199, 201, 203, 205, 207, 209, 211, 213, 215, 217, 219, 221, 223, and 225 or an allelic variant thereof. Sequence identity is compared along the full length of each SEQ ID to the full length sequence of the isolated nucleic acid molecule, and each of SEQ ID NOS: 90, 92, 94, 114, 116, 118, 120, 122, 124, 126, 128, 130, 132, 134, 136, 138, 140, 142, 144, 146, 148, 150, 152, 154, 167, 169, 171, 173, 175, 177, 179, 181, 183, 185, 187, 189, 191, 193, 195, 197, 199, 201, 203, 205, 207, 209, 211, 213, 215, 217, 219, 221, 223, and 225 is a cell surface receptor isoform. In particular, nucleic acid molecules containing the sequence of nucleotides set forth in any of SEQ ID NOS: 90, 92, 94, 114, 116, 118, 120, 122, 124, 126, 128, 130, 132, 134, 136, 138, 140, 142, 144, 146, 148, 150, 152, 154, 167, 169, 171, 173, 175, 177, 179, 181, 183, 185, 187, 189, 191, 193, 195, 197, 199, 201, 203, 205, 207, 209, 211, 213, 215, 217, 219, 221, 223, and 225 are provided. Also provided are vectors containing any of the nucleic acid molecules and cells containing the nucleic acid molecules or vectors.

Pharmaceutical compositions containing the nucleic acid molecules and/or vectors are provided. Such compositions can be used in methods of gene therapy, including in vivo methods and ex vivo methods.

Methods of treating a disease or condition by administering any of the pharmaceutical compositions are provided. Diseases or conditions include, but are not limited to, for example, cancers, inflammatory diseases, infectious diseases, angiogenesis-related diseases or diseases involving aberrant angiogenesis or neovascularization, cell proliferative conditions, immune disorders and neurodegenerative diseases. Also included are methods of treatment where the pharmaceutical compositions contain one or more polypeptides that inhibit(s) angiogenesis, cell proliferation, cell migration, viral entry, viral infection, tumor cell growth or tumor cell metastasis.

Exemplary of diseases and disorders are any of rheumatoid arthritis, multiple sclerosis and posterior intraocular inflammation, uveitic disorders, ocular surface inflammatory disorders, neovascular disease, proliferative vitreoretinopathy, atherosclerosis, endometriosis, rheumatoid arthritis, hemangioma, diabetes mellitus, diabetic retinopathies, inflammatory bowel disease, Crohn's disease, psoriasis, Alzheimer's disease, lupus, vascular stenosis, restenosis, inflammatory joint disease, atherosclerosis, urinary obstructive syndromes, asthma, carcinoma, lymphoma, blastoma, sarcoma, and leukemia, lymphoid malignancies, squamous cell cancer, small-cell lung cancer, non-small cell lung cancer, adenocarcinoma of the lung, squamous carcinoma of the lung, cancer of the peritoneum, hepatocellular cancer, gastric cancer, stomach cancer, gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney/renal cancer, prostate cancer, vulval cancer, thyroid cancer, hepatic carcinoma, anal carcinoma, penile carcinoma, and head and neck cancer and other cancers. Other diseases or conditions include those caused by or mediated by or involving a virus or a parasite, such as, but not limited to, Myxoma virus, Vaccinia virus, Tanapox virus, Epstein-Barr virus, Herpes simplex virus, Cytomegalovirus, Herpesvirus saimiri, Hepatitis B virus, African swine fever virus, Parovirus, Human Immune deficiency virus (HIV), Hepatitis C virus, Influenza virus, Respiratory syncytial virus, Measles virus, Vesicular stomatitis virus, Dengue virus and Ebola virus.

Also provided are combinations and kits containing the combinations, with optional instructions and/or reagents. These combinations contain compositions that contain two and one or more different cell surface receptor isoforms and/or a therapeutic drug or a cell surface receptor isoform and a therapeutic drug. The isoforms and/or drugs can be in separate compositions or in a single composition or one composition containing two or more of the agents and the other containing the other agents or other such formal. Methods of treatment by administering the components of the combination are provided. Each component can be administered separately, simultaneously, intermittently, in a single composition or combinations thereof.

### BRIEF DESCRIPTION OF THE FIGURE

FIG. 1 depicts angiogenic and endothelial cell maintenance pathways. Target points for CSR isoform modulation of one or more pathway steps are indicated. In particular, the figure depicts steps in the formation, maintenance and remodeling of the vasculature. These include the role(s) of VEGF's in recruitment of circulating endothelial precursors (CEPs), the roles of angioipoietin-2 in vessel destabilization.

### DETAILED DESCRIPTION

### A. Definitions

Unless defined otherwise, all technical and scientific terms used herein have the same meaning as is commonly understood by one of skill in the art to which the invention(s) belong. All patents, patent applications, published applications and publications, GENBANK sequences, websites and other published materials referred to throughout the entire disclosure herein, unless noted otherwise, are incorporated by reference in their entirety. In the event that there is a plurality of definitions for terms herein, those in this section prevail. Where reference is made to a URL or other such identifier or address, it is understood that such identifiers can change and particular information on the internet can come and go, but equivalent information is known and can be readily accessed, such as by searching the internet and/or appropriate databases. Reference thereto evidences the availability and public dissemination of such information.

As used herein, a cell surface receptor (CSR) is a protein that is expressed on the surface of a cell and typically includes a transmembrane domain or other moiety that anchors it to the surface of a cell. As a receptor it binds to ligands that mediate or participate in an activity of the cell surface receptor, such as signal transduction or ligand internalization. Cell surface receptors include, but are not limited to, single transmembrane receptors and G-protein coupled receptors. Receptor tyrosine kinases, such as growth factor receptors, also are among such cell surface receptors.

As used herein, a receptor tyrosine kinase (RTK) refers to a protein, typically a glycoprotein, that is a member of the growth factor receptor family of proteins. Growth factor receptors are typically involved in cellular processes including cell growth, cell division, differentiation, metabolism and cell migration. RTKs also are known to be involved in cell proliferation, differentiation and determination of cell fate as well as tumor growth. RTKs have a conserved domain structure including an extracellular domain, a membrane-spanning (transmembrane) domain and an intracellular tyrosine kinase domain. Typically, the extracellular domain binds to a polypeptide growth factor or a cell membrane-associated molecule or other ligand. The tyrosine kinase domain is involved in positive and negative regulation of the receptor.

Receptor tyrosine kinases are grouped into families based on, for example, structural arrangements of sequence motifs in their extracellular domains. Structural motifs include, but are not limited to repeats of regions of: immunoglobulin, fibronectin, cadherin, epidermal growth factor and kringle repeats. Classification by structural motifs has identified greater than 16 families of RTKs, each with a conserved tyrosine kinase domain. Examples of RTKs include, but are not limited to, erythropoietin-producing hepatocellular (EPH) receptors, epidermal growth factor (EGF) receptors, fibroblast growth factor (FGF) receptors, platelet-derived growth factor (PDGF) receptors, vascular endothelial growth factor (VEGF) receptors, cell adhesion RTKs (CAKs), Tie/Tek receptors, insulin-like growth factor (IGF) receptors, and insulin receptor related (IRR) receptors. Exemplary genes encoding RTKs include, but are not limited to, ErbB2, ErbB3, DDR1, DDR2, EGFR, EphA1, EphA8, FGFR-2, FGFR-4, FLT1 (fms-related tyrosine kinase 1 receptor; also known as VEGFR-1), FLK1 (also known as VEGFR-2), MET, PDGFR-A, PDGFR-B, and TEK (also known as TIE-2).

Dimerization of RTKs activates the catalytic tyrosine kinase domain of the receptor and tyrosine autophosphorylation. Autophosphorylation in the kinase domain maintains the tyrosine kinase domain in an activated state. Autophosphorylation in other regions of the protein influences interactions of the receptor with other cellular proteins. In some RTKs, ligand binding to the extracellular domain leads to dimerization of the receptor. In some RTKs, the receptor can dimerize in the absence of ligand. Dimerization also can be increased by receptor overexpression.

As used herein, a tumor necrosis factor receptor (TNFR) refers to a member of a family of receptors that have a characteristic repeating extracellular cysteine-rich motif such as found in TNFR1 and TNFR2. TNFRs also have a variable intracellular domain that differs between members of the TNFR family. The TNFR family of receptors includes, but is not limited to, TNFR1, TNFR2, TNFRrp, the low-affinity nerve growth factor receptor, Fas antigen, CD40, CD27, CD30, 4-1BB, OX40, DR3, DR4, DR5, and herpesvirus entry mediator (HVEM). Ligands for TNFRs include TNF-α, lymphotoxin, nerve growth factor, Fas ligand, CD40 ligand, CD27 ligand, CD30 ligand, 4-1BB ligand, OX40 ligand, APO3 ligand, TRAIL and LIGHT. TNFRs include an extracellular domain, including a ligand binding domain, a transmembrane domain and an intracellular domain that participates in signal transduction. TNFRs are typically trimeric proteins that trimerize at the cell surface.

As used herein, an isoform of a cell surface receptor (also referred to herein as a CSR isoform), such as an isoform of a receptor tyrosine kinase, refers to a receptor that lacks a domain or portion thereof sufficient to alter an activity of the receptor or modulate an activity compared to a wildtype and/or predominant form of the receptor or lacks a structural feature, such as a domain. Thus, a CSR isoform refers to a receptor that lacks a domain or portion of a domain sufficient to alter an activity, typically a biological activity, of the receptor. A CSR isoform lacks a domain or portion of a domain sufficient to alter or modulate an activity of the receptor. A CSR isoform can include an isoform that has one or more biological activities that are altered from the receptor; for example, an isoform can include the alteration of the extracellular domain of p185-HER2, altering the isoform from a positively acting regulatory polypeptide of the receptor to a negatively acting regulatory polypeptide of the isoform, e.g. from a receptor domain into a ligand. Generally, an activity is altered in an isoform at least 0.1, 0.5, 1, 2, 3, 4, 5, or 10 fold compared to a wildtype and/or predominant form of the receptor. Typically, an activity is altered by at least 2, 5, 10, 20, 50, 100 or 1000 fold or more. In one embodiment, alteration of an activity is a reduction in the activity. With reference to an isoform, alteration of activity refers to difference in activity between the particular isoform, which is shortened, compared to the unshortened form of the receptor. Alteration of an activity includes an enhancement or a reduction of activity. In one embodiment, an alteration of an activity is a reduction in biological activity; the reduction can be at least 0.1 0.5 1, 2, 3, 4, 5, or 10 fold compared to a wildtype and/or predominant form of the receptor. Typically, a biological activity is reduced 5, 10, 20, 50, 100 or 1000 fold or more.

As used herein, reference to modulating the activity of a cell surface receptor means that a CSR interacts in some manner with the receptor and activity, such as ligand binding or dimerization or other signal-transduction-related activity is altered.

As used herein, reference to a CSR isoform with altered activity refers to an alteration in an activity by virtue of the different structure or sequence of the CSR isoform compared to a cognate receptor.

As used herein, an intron fusion protein refers to an isoform that lacks one or more domain(s) or portion of one or more domain(s) resulting in an alteration of an activity of a receptor. The activity can be altered by the intron fusion protein directly, such as by interaction with the receptor, or indirectly by interacting with a receptor ligand or co-factor or other modulator of receptor activity. Intron fusion proteins isolated from cells or tissues or that have the sequence of such polypeptides isolated from cells or tissues, are “natural.” Those that do not occur naturally but that are synthesized or prepared by linking a molecule to an intron such that the resulting construct modulates the activity of a CSR are “synthetic.” Included among intron fusion proteins are cell surface receptor isoforms that lack one or more domain(s) or portion of one or more domain(s) resulting in an alteration of an activity of a receptor. In addition, an intron fusion protein contains one or more amino acids not encoded by an exon (with reference to the predominant or wildtype form of a receptor), operatively linked to exon-encoded amino acids. Generally such isoforms are shortened compared to a wildtype or predominant form encoded by a CSR gene. They, however, can include insertions or other modifications in the exon portion and, thus, be of the same size or larger than the predominant form. Each, however, includes an intron-encoded portion (at least one amino acid, generally at least, 2, 3, 4, 5, 8, 10, 15, 20, 25, 30, 35, 40, 45, 50, 55, 60, 65, 70, 75 and more amino acids). An intron fusion protein can be encoded by an alternatively spliced RNA and/or RNA molecules identified in silico by identifying potential splice sites and then producing such molecules by recombinant methods. Typically, an intron fusion protein is shortened by the presence of one or more stop codons in an intron fusion protein-encoding RNA that are not present in the corresponding sequence of an RNA encoding a wildtype or predominant form of a CSR polypeptide. Addition of amino acids and/or a stop codon can result in an intron fusion protein that differs in size and sequence from a wildtype or predominant form of a polypeptide.

Intron fusion proteins for purposes herein include natural combinatorial and synthetic intron fusion proteins. A natural intron fusion protein refers to a polypeptide that is encoded by an alternatively spliced RNA molecule that contains one or more amino acids encoded by an intron linked to one or more portions of the polypeptide encoded by one or more exons of a gene. Alternatively spliced mRNA is isolated or can be prepared synthetically by joining splice donor and acceptor sites in a gene. A natural intron fusion protein contains one or more amino acids and/or a stop codon encoded by an intron sequence and generally occurs in cells and/or tissues, but can be identified from a gene by identifying splice donor and acceptor sites and identifying possible encoded spliced variants. A combinatorial intron fusion protein refers to a polypeptide that is shortened compared to a wildtype or predominant form of a polypeptide. Typically, the shortening removes one or more domains or a portion thereof from a polypeptide such that an activity is altered. Combinatorial intron fusion proteins often mimic a natural intron fusion protein in that one or more domains or a portion thereof is/are deleted in a natural intron fusion protein derived from the same gene or derived from a gene in a related gene family. Those that do not occur naturally but that are synthesized or prepared by linking a molecule to an intron such that the resulting construct modulates the activity of a CSR are “synthetic.”

As used herein, natural with reference to intron fusion proteins, refers to any protein, polypeptide or peptide or fragment thereof (by virtue of the presence of the appropriate splice acceptor/donor sites) that is encoded within the genome of an animal and/or is produced or generated in an animal or that could be produced from a gene. Natural intron fusion proteins include allelic variants. Intron fusion proteins can be modified post-translationally.

As used herein, an exon refers to a nucleic acid molecule containing sequence of nucleotides that is transcribed into RNA and is represented in a mature form of RNA, such as mRNA (messenger RNA), after splicing and other RNA processing. An mRNA contains one or more exons operatively linked. Exons can encode polypeptides or a portion of a polypeptide. Exons also can contain non-translated sequences for example, translational regulatory sequences. Exon sequences are often conserved and exhibit homology among gene family members.

As used herein, an intron refers to a sequence of nucleotides that is transcribed into RNA and is then typically removed from the RNA by splicing to create a mature form of an RNA, for example, an mRNA. Typically, nucleotide sequences of introns are not incorporated into mature RNAs, nor are intron sequences or a portion thereof typically translated and incorporated into a polypeptide. Splice signal sequences such as splice donors and acceptors are used by the splicing machinery of a cell to remove introns from RNA. It is noteworthy that an intron in one splice variant can be an exon (i.e., present in the spliced transcript) in another variant. Hence, spliced mRNA encoding an intron fusion protein can include an exon(s) and introns.

As used herein, splicing refers to a process of RNA maturation where introns in the mRNA are removed and exons are operatively linked to create a messenger RNA (mRNA).

As used herein, alternative splicing refers to the process of producing multiple mRNAs from a gene. Alternate splicing can include operatively linking less than all the exons of a gene, and/or operatively linking one or more alternate exons that are not present in all transcripts derived from a gene.

As used herein, exon deletion refers to an event of alternative RNA splicing that produces a nucleic acid molecule that lacks at least one exon compared to an RNA molecule encoding a wildtype or predominant form of a polypeptide. An RNA molecule that has a deleted exon can be produced by such alternative splicing or by any other method, such as an in vitro method to delete the exon.

As used herein, exon insertion, refers to an event of alternative RNA splicing that produces a nucleic acid molecule that contains at least one exon not typically present in an RNA molecule encoding a wildtype or predominant form of a polypeptide. An RNA molecule that has an inserted exon can be produced by such alternative splicing or by any other method, such as an in vitro method to add or insert the exon.

As used herein, exon extension refers to an event of alternative RNA splicing that produces a nucleic acid molecule that contains at least one exon that is greater in length (number of nucleotides contained in the exon) than the corresponding exon in an RNA encoding a wildtype or predominant form of a polypeptide. An RNA molecule that has an extended exon can be produced by such alternative splicing or by any other method, such as an in vitro method to extend the exon. In some instances, as described herein, an mRNA produced by exon extension encodes an intron fusion protein.

As used herein, exon truncation refers to an event of alternative RNA splicing that produces a nucleic acid molecule that contains a truncation or shortening of one or more exons such that the one or more exons are shorter in length (number of nucleotides) compared to a corresponding exon in an RNA molecule encoding a wildtype or predominant form of a polypeptide. An RNA molecule that has a truncated exon can be produced by such alternative splicing or by any other method, such as an in vitro method to truncate the exon.

As used herein intron retention refers to an event of alternative RNA splicing that produces a nucleic acid molecule that contains an intron or a portion thereof operatively linked to one or more exons. An RNA molecule that retains an intron or portion thereof can be produced by such alternative splicing or by any other method, such as in vitro method to produce an RNA molecule with a retained exon. In some cases, as described herein, an mRNA molecule produced by intron retention encodes an intron fusion protein.

As used herein, a gene, also referred to as a gene sequence, refers to a sequence of nucleotides transcribed into RNA (introns and exons), including nucleotide sequence that encodes at least one polypeptide. A gene includes sequences of nucleotides that regulate transcription and processing of RNA. A gene also includes regulatory sequences of nucleotides such as promoters and enhancers, and translation regulation sequences.

As used herein, a splice site refers to one or more nucleotides within the gene that participate in the removal of an intron and/or the joining of an exon. Splice sites include splice acceptor sites and splice donor sites.

As used herein, cognate receptor with reference to the isoforms provided herein refers to the receptor that is encoded by the same gene as the particular isoform. Generally, the cognate receptor also is a predominant form in a particular cell or tissue. For example, herstatin is encoded by a splice variant of the pre-mRNA which encodes p185-HER2 (ErbB2 receptor). Thus, p185-HER2 is the cognate receptor for herstatin.

As used herein, a wildtype form, for example, a wildtype form of a polypeptide, refers to a polypeptide that is encoded by a gene. Typically a wildtype form refers to a gene (or RNA or protein derived therefrom) without mutations or other modifications that alter function or structure; wildtype forms include allelic variation among and between species.

As used herein, a predominant form, for example, a predominant form of a polypeptide, refers to a polypeptide that is the major polypeptide produced from a gene. A “predominant form” varies from source to source. For example, different cells or tissue types can produce different forms of polypeptides, for example, by alternative splicing and/or by alternative protein processing. In each cell or tissue type, a different polypeptide can be a “predominant form.”

As used herein, a domain refers to a portion (typically a sequence of three or more, generally 5 or 7 or more amino acids) of a polypeptide chain that can form an independently folded structure within a protein made up of one or more structural motifs (e.g. combinations of alpha helices and/or beta strands connected by loop regions) and/or that is recognized by virtue of a functional activity, such as kinase activity. A protein can have one, or more than one, distinct domain. For example, a domain can be identified, defined or distinguished by homology of the sequence therein to related family members, such as homology and motifs that define an extracellular domain. In another example, a domain can be distinguished by its function, such as by enzymatic activity, e.g. kinase activity, or an ability to interact with a biomolecule, such as DNA binding, ligand binding, and dimerization. A domain independently can exhibit a biological function or activity such that the domain independently or fused to another molecule can perform an activity, such as, for example proteolytic activity or ligand binding. A domain can be a linear sequence of amino acids or a non-linear sequence of amino acids from the polypeptide. Many polypeptides contain a plurality of domains. For example, receptor tyrosine kinases typically include, an extracellular domain, a membrane-spanning (transmembrane) domain and an intracellular tyrosine kinase domain.

As used herein, a polypeptide lacking all or a portion of a domain refers a polypeptide that has a deletion of one or more amino acids or all of the amino acids of a domain compared to a cognate polypeptide. Amino acids deleted in a polypeptide lacking all or part of a domain need not be contiguous amino acids within the domain of the cognate polypeptide. Polypeptides that lack all or a part of a domain can include the loss or reduction of an activity of the polypeptide compared to the activity of a cognate polypeptide or loss of a structure in the polypeptide.

For example, if a cognate receptor has a transmembrane domain, then a receptor isoform polypeptide lacking all or a part of the transmembrane domain can have a deletion of 1, 2, 3, 4, 5, 6, 7, 8, 9, 10, 11, 12, 13, 14, 15, 16, 17, 18, 19, 20 or more amino acids between amino acids corresponding to the same amino acid positions in the cognate receptor.

As used herein, a polypeptide that contains a domain refers to a polypeptide that contains a complete domain with reference to the corresponding domain of a cognate receptor. A complete domain is determined with reference to the definition of that particular domain within a cognate polypeptide. For example, a receptor isoform comprising a domain refers to an isoform that contains a domain corresponding to the complete domain as found in the cognate receptor. If a cognate receptor, for example, contains a transmembrane domain of 21 amino acids between amino acid positions 400-420, then a receptor isoform that comprises such transmembrane domain, contains a 21 amino acid domain that has substantial identity with the 21 amino acid domain of the cognate receptor. Substantial identity refers to a domain that can contain allelic variation and conservative substitutions as compared to the domain of the cognate receptor. Domains that are substantially identical do not have deletions, non-conservative substitutions or insertions of amino acids compared to the domain of the cognate receptor. Domains (i.e., a furin domain, an Ig-like domain) often are identified by virtue of structural and/or sequence homology to domains in particular proteins.

Such domains are known to those of skill in the art who can identify such. For exemplification herein, definitions are provided, but it is understood that it is well within the skill in the art to recognize particular domains by name. If needed appropriate software can be employed to identify domains.

As used herein, an extracellular domain is a portion of the cell surface receptor that occurs on the surface of the receptor and includes the ligand binding site(s). In one example, an ephrin receptor ligand binding domain (EPH_{—}1bd) is the portion of the polypeptide that mediates binding of a protein receptor to an ephrin ligand. Typically, EphA receptors bind to GPI-anchored ephrin-A ligands, while EphB receptors bind to ephrin-B proteins that have a transmembrane and cytoplasmic domain.

A Receptor L domain (RLD), such as for example in ErbB2, is another example of a domain that includes a ligand binding site. Each L domain contains a single-stranded right hand beta-helix that can associate with a second L domain to form a three-dimensional bilobal structure surrounding a central space of sufficient size to accommodate a ligand molecule.

As used herein, a furin domain is a domain recognized as such by those of skill in the art and is a cysteine rich region. Furin is a type 1 transmembrane serine protease. A furin domain functions as a cleavage site for furin protease.

As used herein a Sema domain is a domain recognized as such by those of skill in the art and is a receptor recognition and binding module. The Sema domain is characterized by a conserved set of cysteine residues, which form four disulfide bonds to stabilize the structure. The Sema domain fold is a variation of a β propeller topology, with seven blades radially arranged around a central axis. Each blade contains a four-stranded antiparallel β sheet. The Sema domain uses a ‘loop and hook’ system to close the circle between the first and the last blades. The blades are constructed sequentially with an N-terminal β-strand closing the circle by providing the outermost strand of the seventh (C-terminal) blade. The β-propeller is further stabilized by an extension of the N-terminus, providing an additional, fifth β-strand on the outer edge of blade 6.

As used herein, a plexin domain is a domain recognized as such by those of skill in the art and contains a cysteine rich repeat. Plexins are receptors that as a complex interact with membrane-bound semaphorins. The plexins contain three domains with homology to c-met, the receptor for scatter factor-induced motility, but they lack the intrinsic tyrosine kinase activity of c-met. Intracellullarly, invariant arginines identify a plexin domain with homology to guanosine triphosphatase-activating proteins. A protein can contain one, or more than one, plexin domain. As described herein, the MET receptor contains a single plexin domain.

As used herein, the F 5/8 type C domain is a domain recognized as such by those of skill in the art and is a domain that exhibits a distorted jelly-roll β-barrel motif, containing eight antiparallel strands arranged in two β-sheets. The lower part of the β-barrel is characterized by a preponderance of basic residues and three adjacent protruding loops. The portion of the polypeptide that forms the F 5/8 type C domain contains two conserved cysteines, which link the extremities of the domain by a disulfide bond.

As used herein an Ig-like domain is a domain recognized as such by those of skill in the art and is a domain containing folds of beta strands forming a compact folded structure of two beta sheets stabilized by hydrophobic interactions and sandwiched together by an intra-chain disulfide bond. In one example, an Ig-like C-type domain contains seven beta strands arranged as four-strand plus three-strand so that four beta strands form one beta sheet and three beta strands form the second beta sheet. In another example, an Ig-like V-type domain contains nine beta strands arranged as four beta strands plus five beta strands (Janeway C. A. et al. (eds): Immunobiology—the immune system in health and disease, 5th edn. New York, Garland Publishing, 2001.).

As used herein, a fibronectin type-III (FN3) domain is a domain recognized as such by those of skill in the art and contains a conserved β sandwich fold with one β sheet containing four strands and the other sheet containing three strands. The folded structure of an FN3 domain and an Ig-like domain are topologically very similar except the FN3 domain lacks a conserved disulfide bond. The portion of the polypeptide encoding an FN3 domain also is characterized by a short stretch of amino acids containing an Arg-Gly-Asp (RGD) that mediates interactions with cell adhesion molecules to modulate thrombosis, inflammation, and tumor metastasis. In one example, EphA1 contains two FN3 domains.

As used herein, an IPT/TIG domain is a domain recognized as such by those of skill in the art and has an immunoglobulin fold-like domain. Proteins contain one, or more than one, IPT/TIG domain. IPT/TIG domains are found in plexins, transcription factors, and extracellular regions of receptor proteins, such as for example the cell surface receptors MET and RON as described herein, that appear to regulate cell proliferation and cellular adhesion (Johnson C A et al, Journal of Medical Genetics, 40:311-319, (2003)).

As used herein, an EGF domain is a domain recognized as such by those of skill in the art and contains a repeat pattern involving a number of conserved cysteine residues which are important to the three-dimensional structure of the protein, and hence its recognition by receptors and other molecules. The EGF domain as described herein contains six cysteine residues which are involved in forming disulfide bonds. An EGF domain forms a two-stranded β sheet followed by a loop to a C-terminal short two-stranded sheet. Subdomains between the conserved cysteines vary in length. Repeats of EGF domains are typically found in the extracellular domain of membrane-bound proteins, such as for example in Tie-1 as described herein. A variation of the EGF domain is the laminin (Lam) EGF domain which, as described herein, has eight instead of six conserved cysteines and therefore is longer than the average EGF module and contains a further disulfide bond C-terminal of the EGF-like region.

As used herein, a C6 domain is a cysteine rich domain of typically about 110 to 160 amino acids in the N-terminal region of the polypeptide. It can be subdivided into four, or in some cases three or more, modules of about 40 residues containing 6 conserved cysteines that participate in intrachain disulfide bonds. A protein can have one, or more than one, C6 domain. As described herein, for example, TNFR2 contains three C6 domains.

As used herein, a transmembrane domain spans the plasma membrane anchoring the receptor and generally includes hydrophobic residues.

As used herein, a cytoplasmic domain is a domain that participates in signal transduction and occurs in the cytoplasmic portion of a transmembrane cell surface receptor. In one example, the cytoplasmic domain can include a protein kinase (PK) domain. A PK domain is recognized as such by those of skill in the art and is a domain that contains a conserved catalytic core. The conserved catalytic core is recognized to have a glycine-rich stretch of residues in the vicinity of a lysine residue in the N-terminal extremity of the domain, which has been shown to be involved in ATP binding, and an aspartic acid residue in the central part of the catalytic domain, which is important for the catalytic activity of the enzyme. Typically, the PK domain can be a serine/threonine protein kinase or a tyrosine protein kinase domain depending on the substrate specificity of the receptor domain such that, for example, a protein containing a tyrosine kinase domain phosphorylates substrate proteins on tyrosine residues whereas, for example, a protein containing a serine/threonine protein kinase domain phosphorylates substrate proteins on serine or threonine residues.

As used herein, sterile α motif (SAM) domain is considered a protein-protein interaction module. A SAM domain is recognized as such by those of skill in the art and is a domain that spreads over typically about 70 residues to form an independently folded structure arranged in a small five-helix bundle with two large interfaces. In one example, such as for example in the SAM domain of EphB2, each of the interfaces is able to form dimers. The ability of the SAM domain to form homo- or hetero-oligomers creates a binding surface that mediates protein protein interactions.

As used herein, an allelic variant or allelic variation references to a polypeptide encoded by a gene that differs from a reference form of a gene (i.e. is encoded by an allele). Typically the reference form of the gene encodes a wildtype form and/or predominant form of a polypeptide from a population or single reference member of a species. Typically, allelic variants, which include variants between and among species typically have at least 80%, 90% or greater amino acid identity with a wildtype and/or predominant form from the same species; the degree of identity depends upon the gene and whether comparison is interspecies or intraspecies. Generally, intraspecies allelic variants have at least about 80%, 85%, 90% or 95% identity or greater with a wildtype and/or predominant form, including 96%, 97%, 98%, 99% or greater identity with a wildtype and/or predominant form of a polypeptide.

As used herein, modification in reference to modification of a sequence of amino acids of a polypeptide or a sequence of nucleotides in a nucleic acid molecule and includes deletions, insertions, and replacements of amino acids and nucleotides, respectively.

As used herein, an open reading frame refers to a sequence of nucleotides that encodes a functional polypeptide or a portion thereof, typically at least about fifty amino acids. An open reading frame can encode a full-length polypeptide or a portion thereof. An open reading frame can be generated by operatively linking one or more exons or an exon and intron, when the stop codon is in the intron and all or a portion of the intron is in a transcribed mRNA.

As used herein, a polypeptide refers to two or more amino acids covalently joined. The terms “polypeptide” and “protein” are used interchangeably herein.

As used herein, truncation or shortening with reference to the shortening of a nucleic acid molecule or protein, refers to a sequence of nucleotides or amino acids that is less than full-length compared to a wildtype or predominant form of the protein or nucleic acid molecule.

As used herein, a reference gene refers to a gene that can be used to map introns and exons within a gene. A reference gene can be genomic DNA or portion thereof, that can be compared with, for example, an expressed gene sequence, to map introns and exons in the gene. A reference gene also can be a gene encoding a wildtype or predominant form of a polypeptide.

As used herein, a family or related family of proteins or genes refers to a group of proteins or genes, respectively that have homology and/or structural similarity and/or functional similarity with each other.

As used herein, a premature stop codon is a stop codon occurring in the open reading frame of a sequence before the stop codon used to produce or create a full-length form of a protein, such as a wildtype or predominant form of a polypeptide. The occurrence of a premature stop codon can be the result of, for example, alternative splicing and mutation.

As used herein, an expressed gene sequence refers to any sequence of nucleotides transcribed or predicted to be transcribed from a gene. Expressed gene sequences include, but are not limited to, cDNAs, ESTs, and in silico predictions of expressed sequences, for example, based on splice site predictions and in silico generation of spliced sequences.

As used herein, an expressed sequence tag (EST) is a sequence of nucleotides generated from an expressed gene sequence. ESTs are generated by using a population of mRNA to produce cDNA. The cDNA molecules can be produced for example, by priming from the polyA tail present on mRNAs. cDNA molecules also can be produced by random priming using one or more oligonucleotides which prime cDNA synthesis internally in mRNAs. The generated cDNA molecules are sequenced and the sequences are typically stored in a database. An example of an EST database is dbEST found online at ncbi.nlm.nih.gov/dbEST. Each EST sequence is typically assigned a unique identifier and information such as the nucleotide sequence, length, tissue type where expressed, and other associated data is associated with the identifier.

As used herein, a kinase is a protein that is able to phosphorylate a molecule, typically a biomolecule, including macromolecules and small molecules. For example, the molecule can be a small molecule, or a protein. Phosphorylation includes auto-phosphorylation. Some kinases have constitutive kinase activity. Other kinases require activation. For example, many kinases that participate in signal transduction are phosphorylated. Phosphorylation activates their kinase activity on another biomolecule in a pathway. Some kinases are modulated by a change in protein structure and/or interaction with another molecule. For example, complexation of a protein or binding of a molecule to a kinase can activate or inhibit kinase activity.

As used herein, designated refers to the selection of a molecule or portion thereof as a point of reference or comparison. For example, a domain can be selected as a designated domain for the purpose of constructing polypeptides that are modified within the selected domain. In another example, an intron can be selected as a designated intron for the purpose of identifying RNA transcripts that include or exclude the selected intron.

As used herein, modulate and modulation refer to a change of an activity of a molecule, such as a protein. Exemplary activities include, but are not limited to, biological activities, such as signal transduction and protein phosphorylation. Modulation can include an increase in the activity (i.e., up-regulation agonist activity) a decrease in activity (i.e., down-regulation or inhibition) or any other alteration in an activity (such as periodicity, frequency, duration, kinetics). Modulation can be context dependent and typically modulation is compared to a designated state, for example, the wildtype protein, the protein in a constitutive state, or the protein as expressed in a designated cell type or condition.

As used herein, inhibit and inhibition refer to a reduction in an activity, such as a biological activity, relative to the uninhibited activity.

As used herein, a composition refers to any mixture. It can be a solution, a suspension, liquid, powder, a paste, aqueous, non-aqueous or any combination thereof.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related. A kit is a packaged combination that optionally includes instructions for use of the combination or elements thereof and/or optionally include other reagents and vessels and tools and devices employed in the methods for which the kits are intended.

As used herein, a pharmaceutical effect refers to an effect observed upon administration of an agent intended for treatment of a disease or disorder or for amelioration of the symptoms thereof.

As used herein, treatment means any manner in which the symptoms of a condition, disorder or disease or other indication, are ameliorated or otherwise beneficially altered.

As used herein therapeutic effect means an effect resulting from treatment of a subject that alters, typically improves or ameliorates the symptoms of a disease or condition or that cures a disease or condition. A therapeutically effective amount refers to the amount of a composition, molecule or compound which results in a therapeutic effect following administration to a subject.

As used herein, the term “subject” refers to animals, including mammals, such as human beings. As used herein, a patient refers to a human subject.

As used herein, an activity refers to a function or functioning or changes in or interactions of a biomolecule, such as polypeptide. Exemplary, but not limiting of such activities are: complexation, dimerization, multimerization, receptor-associated kinase activity or other enzymatic or catalytic activity, receptor-associated protease activity, phosphorylation, dephosphorylation, autophosphorylation, ability to form complexes with other molecules, ligand binding, catalytic or enzymatic activity, activation including auto-activation and activation of other polypeptides, inhibition or modulation of another molecule's function, stimulation or inhibition of signal transduction and/or cellular responses such as cell proliferation, migration, differentiation, and growth, degradation, membrane localization, membrane binding, and oncogenesis. An activity can be assessed by assays described herein and by any suitable assays known to those of skill in the art, including, but not limited to in vitro assays, including cell-based assays, in vivo assays, including assays in animal models for particular diseases. Biological activities refer to activities exhibited in vivo. For purposes herein, biological activity refers to any of the activities exhibited by a polypeptide provided herein.

As used herein, angiogenic diseases (or angiogenesis-related diseases) are diseases in which the balance of angiogenesis is altered or the timing thereof is altered. Angiogenic diseases include those in which an alteration of angiogenesis, such as undesirable vascularization, occurs. Such diseases include, but are not limited to cell proliferative disorders, including cancers, diabetic retinopathies and other diabetic complications, inflammatory diseases, endometriosis and other diseases in which excessive vascularization is part of the disease process, including those noted above.

As used herein, complexation refers to the interaction of two or more molecules such as two molecules of a protein to form a complex. The interaction can be by noncovalent and/or covalent bonds and includes, but is not limited to, hydrophobic and electrostatic interactions, Van der Waals forces and hydrogen bonds. Generally, protein-protein interactions involve hydrophobic interactions and hydrogen bonds. Complexation can be influenced by environmental conditions such as temperature, pH, ionic strength and pressure, as well as protein concentrations.

As used herein, dimerization refers to the interaction of two molecules of the same type, such as two molecules of a receptor. Dimerization includes homodimerization where two identical molecules interact. Dimerization also includes heterodimerization of two different molecules, such as two subunits of a receptor and dimerization of two different receptor molecules. Typically, dimerization involves two molecules that interact with each other through interaction of a dimerization domain contained in each molecule.

As used herein, a ligand antagonist refers to the activity of a CSR isoform that antagonizes an activity that results from ligand interaction with a CSR.

As used herein, in silico refers to research and experiments performed using a computer. In silico methods include, but are not limited to, molecular modeling studies, biomolecular docking experiments, and virtual representations of molecular structures and/or processes, such as molecular interactions.

As used herein, biological sample refers to any sample obtained from a living or viral source or other source of macromolecules and biomolecules, and includes any cell type or tissue of a subject from which nucleic acid or protein or other macromolecule can be obtained. The biological sample can be a sample obtained directly from a biological source or to sample that is processed For example, isolated nucleic acids that are amplified constitute a biological sample. Biological samples include, but are not limited to, body fluids, such as blood, plasma, serum, cerebrospinal fluid, synovial fluid, urine and sweat, tissue and organ samples from animals and plants and processed samples derived therefrom. Also included are soil and water samples and other environmental samples, viruses, bacteria, fungi algae, protozoa and components thereof.

As used herein, macromolecule refers to any molecule having a molecular weight from the hundreds up to the millions. Macromolecules include peptides, proteins, nucleotides, nucleic acids, and other such molecules that are generally synthesized by biological organisms, but can be prepared synthetically or using recombinant molecular biology methods.

As used herein, a biomolecule is any compound found in nature, or derivatives thereof. Exemplary biomolecules include but are not limited to: oligonucleotides, oligonucleosides, proteins, peptides, amino acids, peptide nucleic acids (PNAs), oligosaccharides and monosaccharides.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Also included in the term “nucleic acid” are analogs of nucleic acids such as peptide nucleic acid (PNA), phosphorothioate DNA, and other such analogs and derivatives or combinations thereof. Nucleic acid can refer to polynucleotides such as deoxyribonucleic acid (DNA) and ribonucleic acid (RNA). The term also includes, as equivalents, derivatives, variants and analogs of either RNA or DNA made from nucleotide analogs, single (sense or antisense) and double-stranded polynucleotides. Deoxyribonucleotides include deoxyadenosine, deoxycytidine, deoxyguanosine and deoxythymidine. For RNA, the uracil base is uridine.

As used herein, the term “polynucleotide” refers to an oligomer or polymer containing at least two linked nucleotides or nucleotide derivatives, including a deoxyribonucleic acid (DNA), a ribonucleic acid (RNA), and a DNA or RNA derivative containing, for example, a nucleotide analog or a “backbone” bond other than a phosphodiester bond, for example, a phosphotriester bond, a phosphoramidate bond, a phophorothioate bond, a thioester bond, or a peptide bond (peptide nucleic acid). The term “oligonucleotide” also is used herein essentially synonymously with “polynucleotide,” although those in the art recognize that oligonucleotides, for example, PCR primers, generally are less than about fifty to one hundred nucleotides in length.

Polynucleotides can include nucleotide analogs, for example, mass modified nucleotides, which allow for mass differentiation of polynucleotides; nucleotides containing a detectable label such as a fluorescent, radioactive, luminescent or chemiluminescent label, which allow for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well-known methods (see, for example, Weiler et al. Nucleic acids Res. 25: 2792-2799 (1997)).

As used herein, synthetic, in the context of a synthetic sequence and synthetic gene refers to a nucleic acid molecule that is produced by recombinant methods and/or by chemical synthesis methods.

As used herein, oligonucleotides refer to polymers that include DNA, RNA, nucleic acid analogues, such as PNA, and combinations thereof. For purposes herein, primers and probes are single-stranded oligonucleotides or are partially single-stranded oligonucleotides.

As used herein, primer refers to an oligonucleotide containing two or more deoxyribonucleotides or ribonucleotides, generally more than three, from which synthesis of a primer extension product can be initiated. Experimental conditions conducive to synthesis include the presence of nucleoside triphosphates and an agent for polymerization and extension, such as DNA polymerase, and a suitable buffer, temperature and pH.

As used herein, production by recombinant means by using recombinant DNA methods means the use of the well-known methods of molecular biology for expressing proteins encoded by cloned DNA.

As used herein, “isolated,” with reference to a molecule, such as a nucleic acid molecule, oligonucleotide, polypeptide or antibody, indicates that the molecule has been altered by the hand of man from how it is found in its natural environment. For example, a molecule produced by and/or contained within a recombinant host cell is considered “isolated.” Likewise, a molecule that has been purified, partially or substantially, from a native source or recombinant host cell, or produced by synthetic methods, is considered “isolated.” Depending on the intended application, an isolated molecule can be present in any form, such as in an animal, cell or extract thereof; dehydrated, in vapor, solution or suspension; or immobilized on a solid support.

As used herein, the term “vector” refers to a nucleic acid molecule capable of transporting another nucleic acid to which it has been linked. One type of vector is an episome, i.e., a nucleic acid capable of extra chromosomal replication. Vectors include those capable of autonomous replication and/or expression of nucleic acids to which they are linked. Vectors capable of directing the expression of genes to which they are operatively linked are referred to herein as “expression vectors.” In general, expression vectors are often in the form of “plasmids,” which are generally circular double stranded DNA loops that, in their vector form are not bound to the chromosome. “Plasmid” and “vector” are used interchangeably as the plasmid is the most commonly used form of vector. Other such other forms of expression vectors that serve equivalent functions and that become known in the art subsequently hereto.

As used herein, “transgenic animal” refers to any animal, generally a non-human animal, e.g., a mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. This molecule can be stably integrated within a chromosome, i.e., replicate as part of the chromosome, or it can be extrachromosomally replicating DNA. In the typical transgenic animals, the transgene causes cells to express a recombinant form of a protein.

As used herein, a reporter gene construct is a nucleic acid molecule that includes a nucleic acid encoding a reporter operatively linked to a transcriptional control sequences. Transcription of the reporter gene is controlled by these sequences. The activity of at least one or more of these control sequences is directly or indirectly regulated by another molecule such as a cell surface protein, a protein or small molecule involved in signal transduction within the cell. The transcriptional control sequences include the promoter and other regulatory regions, such as enhancer sequences, that modulate the activity of the promoter, or control sequences that modulate the activity or efficiency of the RNA polymerase. Such sequences are herein collectively referred to as transcriptional control elements or sequences. In addition, the construct can include sequences of nucleotides that alter translation of the resulting mRNA, thereby altering the amount of reporter gene product.

As used herein, “reporter” or “reporter moiety” refers to any moiety that allows for the detection of a molecule of interest, such as a protein expressed by a cell, or a biological particle. Typical reporter moieties include, for example, fluorescent proteins, such as red, blue and green fluorescent proteins (see, e.g., U.S. Pat. No. 6,232,107, which provides GFPs from Renilla species and other species), the lacZ gene from E. coli, alkaline phosphatase, chloramphenicol acetyl transferase (CAT) and other such well-known genes. For expression in cells, nucleic acid encoding the reporter moiety, referred to herein as a “reporter gene,” can be expressed as a fusion protein with a protein of interest or under to the control of a promoter of interest.

As used herein, the phrase “operatively linked” with reference to sequences of nucleic acids means the nucleic acid molecules or segments thereof are covalently joined into one piece of nucleic acid such as DNA or RNA, whether in single or double stranded form. The segments are not necessarily contiguous, rather two or more components are juxtaposed so that the components are in a relationship permitting them to function in their intended manner. For example, segments of RNA (exons) can be operatively linked such as by splicing, to form a single RNA molecule. In another example, DNA segments can be operatively linked, whereby control or regulatory sequences on one segment control permit expression or replication or other such control of other segments. Thus, in the case of a regulatory region operatively linked to a reporter or any other polynucleotide, or a reporter or any polynucleotide operatively linked to a regulatory region, expression of the polynucleotide/reporter is influenced or controlled (e.g., modulated or altered, such as increased or decreased) by the regulatory region. For gene expression, a sequence of nucleotides and a regulatory sequence(s) are connected in such a way to control or permit gene expression when the appropriate molecular signal, such as transcriptional activator proteins, are bound to the regulatory sequence(s). Operative linkage of heterologous nucleic acid, such as DNA, to regulatory and effector sequences of nucleotides, such as promoters, enhancers, transcriptional and translational stop sites, and other signal sequences, refers to the relationship between such DNA and such sequences of nucleotides. For example, operative linkage of heterologous DNA to a promoter refers to the physical relationship between the DNA and the promoter such that the transcription of such DNA is initiated from the promoter by an RNA polymerase that specifically recognizes, binds to and transcribes the DNA in reading frame.

As used herein, the term “operatively linked” with reference to amino acids in polypeptides refers to covalent linkage (direct or indirect) of the amino acids. For example, when used in the context of the phrase “at least one domain of a cell surface receptor operatively linked to at least one amino acid encoded by an intron of a gene encoding a cell surface receptor,” means that the amino acids of a domain from a cell surface receptor are covalently joined to amino acids encoded by an intron from a cell surface receptor gene such as by linkage, typically direct linkage via peptide bonds, or the linkage also can be effected indirectly, such as via a linker or via non-peptidic linkage. Hence, a polypeptide that contains at least one domain of a cell surface receptor operatively linked to at least one amino acid encoded by an intron of a gene encoding a cell surface receptor can be an intron fusion protein. It contains one or more amino acids that are not found in a predominant form of the receptor but rather contains a portion that is encoded by an intron of the gene that encodes the predominant form. These one or more amino acids are encoded by an intron sequence of the gene encoding the cell surface receptor. Nucleic acids encoding such polypeptides can be produced when an intron sequence is spliced or otherwise covalently joined in-frame to an exon sequence that encodes a domain of a cell surface receptor. Translation of the nucleic acid molecule produces a polypeptide where the amino acid(s) of the intron sequence are covalently joined to a domain of the cell surface receptor. They also can be produced synthetically by linking a portion containing an exon to a portion containing an intron, including chimeric intron fusion proteins in which the exon is encoded by a gene for a different cell surface receptor isoform from the intron portion.

As used herein, the phrase “generated from a nucleic acid” in reference to the generating of a polypeptide, such as an isoform and intron fusion protein, includes the literal generation of a polypeptide molecule and the generation of an amino acid sequence of a polypeptide from translation of the nucleic acid sequence into a sequence of amino acids.

As used herein, a promoter region refers to the portion of DNA of a gene that controls transcription of the DNA to which it is operatively linked. The promoter region includes specific sequences of DNA that are sufficient for RNA polymerase recognition, binding and transcription initiation. This portion of the promoter region is referred to as the promoter. In addition, the promoter region includes sequences that modulate this recognition, binding and transcription initiation activity of the RNA polymerase. These sequences can be cis acting or can be responsive to trans acting factors. Promoters, depending upon the nature of the regulation, can be constitutive or regulated.

As used herein, regulatory region means a cis-acting nucleotide sequence that influences expression, positively or negatively, of an operatively linked gene. Regulatory regions include sequences of nucleotides that confer inducible (i.e., require a substance or stimulus for increased transcription) expression of a gene. When an inducer is present or at increased concentration, gene expression can be increased. Regulatory regions also include sequences that confer repression of gene expression (i.e., a substance or stimulus decreases transcription). When a repressor is present or at increased concentration gene expression can be decreased. Regulatory regions are known to influence, modulate or control many in vivo biological activities including cell proliferation, cell growth and death, cell differentiation and immune modulation. Regulatory regions typically bind to one or more trans-acting proteins, which results in either increased or decreased transcription of the gene.

Particular examples of gene regulatory regions are promoters and enhancers. Promoters are sequences located around the transcription or translation start site, typically positioned 5′ of the translation start site. Promoters usually are located within 1 Kb of the translation start site, but can be located further away, for example, 2 Kb, 3 Kb, 4 Kb, 5 Kb or more, up to and including 10 Kb. Enhancers are known to influence gene expression when positioned 5′ or 3′ of the gene, or when positioned in or a part of an exon or an intron. Enhancers also can function at a significant distance from the gene, for example, at a distance from about 3 Kb, 5 Kb, 7 Kb, 10 Kb, 15 Kb or more.

Regulatory regions also include, in addition to promoter regions, sequences that facilitate translation, splicing signals for introns, maintenance of the correct reading frame of the gene to permit in-frame translation of mRNA, stop codons, leader sequences and fusion partner sequences, internal ribosome binding sites (IRES), elements for the creation of multigene or polycistronic messages, and polyadenylation signals to provide proper polyadenylation of the transcript of a gene of interest and can be optionally included in an expression vector.

As used herein, the “amino acids,” which occur in the various amino acid sequences appearing herein, are identified according to their well-known, three-letter or one-letter abbreviations (see Table 1). The nucleotides, which occur in the various DNA fragments, are designated with the standard single-letter designations used routinely in the art.

As used herein, “amino acid residue” refers to an amino acid formed upon chemical digestion (hydrolysis) of a polypeptide at its peptide linkages. The amino acid residues described herein are generally in the “L” isomeric form. Residues in the “D” isomeric form can be substituted for any L-amino acid residue, as long as the desired functional property is retained by the polypeptide. NH2 refers to the free amino group present at the amino terminus of a polypeptide. COOH refers to the free carboxy group present at the carboxyl terminus of a polypeptide. In keeping with standard polypeptide nomenclature described in J. Biol. Chem., 243:3552-59 (1969) and adopted at 37 C.F.R. §§ 1.821-1.822, abbreviations for amino acid residues are shown in Table 1:

TABLE 1
Table of Correspondence

SYMBOL

| 1-Letter | 3-Letter | AMINO ACID |
|---|---|---|
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | Histidine |
| Q | Gln | Glutamine |
| E | Glu | glutamic acid |
| Z | Glx | Glu and/or Gln |
| W | Trp | Tryptophan |
| R | Arg | Arginine |
| D | Asp | aspartic acid |
| N | Asn | Asparagines |
| B | Asx | Asn and/or Asp |
| C | Cys | Cysteine |
| X | Xaa | Unknown or other |

All sequences of amino acid residues represented herein by a formula have a left to right orientation in the conventional direction of amino-terminus to carboxyl-terminus. In addition, the phrase “amino acid residue” is defined to include the amino acids listed in the Table of Correspondence modified, non-natural and unusual amino acids. Furthermore, it should be noted that a dash at the beginning or end of an amino acid residue sequence indicates a peptide bond to a further sequence of one or more amino acid residues or to an amino-terminal group such as NH₂ or to a carboxyl-terminal group such as COOH.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. co., p. 224).

Such substitutions may be made in accordance with those set forth in TABLE 2 as follows:

TABLE 2

| Original | Conservative |
|---|---|
| residue | substitution |
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions also are permissible and can be determined empirically or in accord with other known conservative or non-conservative substitutions.

As used herein, a peptidomimetic is a compound that mimics the conformation and certain stereochemical features of a biologically active form of a particular peptide. In general, peptidomimetics are designed to mimic certain desirable properties of a compound, but not the undesirable properties, such as flexibility, that lead to a loss of a biologically active conformation and bond breakdown. Peptidomimetics can be prepared from biologically active compounds by replacing certain groups or bonds that contribute to the undesirable properties with bioisosteres. Bioisosteres are known to those of skill in the art. For example the methylene bioisostere CH2S has been used as an amide replacement in enkephalin analogs (see, e.g., Spatola (1983) pp. 267-357 in Chemistry and Biochemistry of Amino Acids, Peptides, and Proteins, Weinstein, Ed. volume 7, Marcel Dekker, New York). Morphine, which can be administered orally, is a compound that is a peptidomimetic of the peptide endorphin. For purposes herein, polypeptides in which one or more peptidic bonds that form the backbone of a polypeptide are replaced with bioisoteres are peptidomimetics.

As used herein, “similarity” between two proteins or nucleic acids refers to the relatedness between the sequence of amino acids of the proteins or the nucleotide sequences of the nucleic acids. Similarity can be based on the degree of identity and/or homology of sequences of residues and the residues contained therein. Methods for assessing the degree of similarity between proteins or nucleic acids are known to those of skill in the art. For example, in one method of assessing sequence similarity, two amino acid or nucleotide sequences are aligned in a manner that yields a maximal level of identity between the sequences. “Identity” refers to the extent to which the amino acid or nucleotide sequences are invariant. Alignment of amino acid sequences, and to some extent nucleotide sequences, also can take into account conservative differences and/or frequent substitutions in amino acids (or nucleotides). Conservative differences are those that preserve the physico-chemical properties of the residues involved. Alignments can be global (alignment of the compared sequences over the entire length of the sequences and including all residues) or local (the alignment of a portion of the sequences that includes only the most similar region or regions).

“Identity” per se has an art-recognized meaning and can be calculated using published techniques. (See, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). While there exist a number of methods to measure identity between two polynucleotide or polypeptides, the term “identity” is well known to skilled artisans (Carillo, H. & Lipton, D., SIAM J Applied Math 48:1073 (1988)).

As used herein, sequence identity compared along the full length of a polypeptide compared to another polypeptide refers to the percentage of identity of an amino acid in a polypeptide along its full-length. For example, if a polypeptide A has 100 amino acids and polypeptide B has 95 amino acids, identical to amino acids 1-95 of polypeptide A, then polypeptide B has 95% identity when sequence identity is compared along the full length of a polypeptide A compared to full length of polypeptide B. As discussed below, and known to those of skill in the art, various programs and methods for assessing identity are known to those of skill in the art. High levels of identity, such as 90% or 95% identity, readily can be determined without software.

As used herein, by homologous (with respect to nucleic acid and/or amino acid sequences) means about greater than or equal to 25% sequence homology, typically greater than or equal to 25%, 40%, 60%, 70%, 80%, 85%, 90% or 95% sequence homology; the precise percentage can be specified if necessary. For purposes herein the terms “homology” and “identity” are often used interchangeably, unless otherwise indicated. In general, for determination of the percentage homology or identity, sequences are aligned so that the highest order match is obtained (see, e.g.: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; Carillo et al. (1988) SIAM J Applied Math 48:1073). By sequence homology, the number of conserved amino acids is determined by standard alignment algorithms programs, and can be used with default gap penalties established by each supplier. Substantially homologous nucleic acid molecules would hybridize typically at moderate stringency or at high stringency all along the length of the nucleic acid of interest. Also contemplated are nucleic acid molecules that contain degenerate codons in place of codons in the hybridizing nucleic acid molecule.

Whether any two nucleic acid molecules have nucleotide sequences that are at least 60%, 70%, 80%, 85%, 90%, 95%, 96%, 97%, 98% or 99% “identical” or “homologous” can be determined using known computer algorithms such as the “FAST A” program, using for example, the default parameters as in Pearson et al. (1988) Proc. Natl. Acad. Sci. USA 85:2444 (other programs include the GCG program package (Devereux, J., et al., Nucleic Acids Research 12(I):387 (1984)), BLASTP, BLASTN, FASTA (Atschul, S. F., et al., J Molec Biol 215:403 (1990); Guide to Huge Computers, Martin J. Bishop, ed., Academic Press, San Diego, 1994, and Carillo et al. (1988) SIAM J Applied Math 48:1073). For example, the BLAST function of the National Center for Biotechnology Information database can be used to determine identity. Other commercially or publicly available programs include, DNAStar “MegAlign” program (Madison, Wis.) and the University of Wisconsin Genetics Computer Group (UWG) “Gap” program (Madison Wis.)). Percent homology or identity of proteins and/or nucleic acid molecules can be determined, for example, by comparing sequence information using a GAP computer program (e.g., Needleman et al. (1970) J. Mol. Biol. 48:443, as revised by Smith and Waterman ((1981) Adv. Appl. Math. 2:482). Briefly, the GAP program defines similarity as the number of aligned symbols (i.e., nucleotides or amino acids), which are similar, divided by the total number of symbols in the shorter of the two sequences. Default parameters for the GAP program can include: (1) a unary comparison matrix (containing a value of 1 for identities and 0 for non-identities) and the weighted comparison matrix of Gribskov et al. (1986) Nucl. Acids Res. 14:6745, as described by Schwartz and Dayhoff, eds., ATLAS OF PROTEIN SEQUENCE AND STRUCTURE, National Biomedical Research Foundation, pp. 353-358 (1979); (2) a penalty of 3.0 for each gap and an additional 0.10 penalty for each symbol in each gap; and (3) no penalty for end gaps.

Therefore, as used herein, the term “identity” or “homology” represents a comparison between a test and a reference polypeptide or polynucleotide. As used herein, the term at least “90% identical to” refers to percent identities from 90 to 99.99 relative to the reference nucleic acid or amino acid sequence of the polypeptide. Identity at a level of 90% or more is indicative of the fact that, assuming for exemplification purposes a test and reference polypeptide length of 100 amino acids are are compared, no more than 10% (i.e., 10 out of 100) of the amino acids in the test polypeptide differs from that of the reference polypeptide. Similar comparisons can be made between test and reference polynucleotides. Such differences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleic acid or amino acid substitutions, insertions or deletions. At the level of homologies or identities above about 85-90%, the result should be independent of the program and gap parameters set; such high levels of identity can be assessed readily, often by manual alignment without relying on software.

As used herein, an aligned sequence refers to the use of homology (similarity and/or identity) to align corresponding positions in a sequence of nucleotides or amino acids. Typically, two or more sequences that are related by 50% or more identity are aligned. An aligned set of sequences refers to 2 or more sequences that are aligned at corresponding positions and can include aligning sequences derived from RNAs, such as ESTs and other cDNAs, aligned with genomic DNA sequence.

As used herein, “primer” refers to a nucleic acid molecule that can act as a point of initiation of template-directed DNA synthesis under appropriate conditions (e.g., in the presence of four different nucleoside triphosphates and a polymerization agent, such as DNA polymerase, RNA polymerase or reverse transcriptase) in an appropriate buffer and at a suitable temperature. It will be appreciated that certain nucleic acid molecules can serve as a “probe” and as a “primer.” A primer, however, has a 3′ hydroxyl group for extension. A primer can be used in a variety of methods, including, for example, polymerase chain reaction (PCR), reverse-transcriptase (RT)-PCR, RNA PCR, LCR, multiplex PCR, panhandle PCR, capture PCR, expression PCR, 3′ and 5′ RACE, in situ PCR, ligation-mediated PCR and other amplification protocols.

As used herein, “primer pair” refers to a set of primers that includes a 5′ (upstream) primer that hybridizes with the 5′ end of a sequence to be amplified (e.g. by PCR) and a 3′ (downstream) primer that hybridizes with the complement of the 3′ end of the sequence to be amplified.

As used herein, “specifically hybridizes” refers to annealing, by complementary base-pairing, of a nucleic acid molecule (e.g. an oligonucleotide) to a target nucleic acid molecule. Those of skill in the art are familiar with in vitro and in vivo parameters that affect specific hybridization, such as length and composition of the particular molecule. Parameters particularly relevant to in vitro hybridization further include annealing and washing temperature, buffer composition and salt concentration. Exemplary washing conditions for removing non-specifically bound nucleic acid molecules at high stringency are 0.1×SSPE, 0.1% SDS, 65° C., and at medium stringency are 0.2×SSPE, 0.1% SDS, 50° C. Equivalent stringency conditions are known in the art. The skilled person can readily adjust these parameters to achieve specific hybridization of a nucleic acid molecule to a target nucleic acid molecule appropriate for a particular application.

As used herein, an effective amount is the quantity of a therapeutic agent necessary for preventing, curing, ameliorating, arresting or partially arresting a symptom of a disease or disorder.

As used herein, unit dose form refers to physically discrete units suitable for human and animal subjects and packaged individually as is known in the art.

### B. Cell Surface Receptor (CSR) Isoforms

Provided herein are cell surface receptor (CSR) isoforms, families of CSR isoforms and methods of preparing CSR isoforms. The CSR isoforms differ from the cognate receptors in that there are insertions and/or deletions and the resulting CSR isoforms exhibit a difference in one or more activities or functions compared to the cognate receptor. Such changes include a change in a biological activity, such as elimination of kinase activity, and/or elimination of all or part of a transmembrane domain. The CSR isoforms provided herein can be used for modulating the activity of a cell surface receptor. They also can be used as targeting agents for delivery of molecules, such as drugs or toxins or nucleic acids, to targeted cells or tissues.

CSR isoforms can contain a new domain and/or exhibit a new or different biological function compared to a wildtype and/or predominant form of the receptor. For example, intron-encoded amino acids can introduce a new domain or portion thereof into an isoform. Biological activities that can be altered include, but are not limited to, protein-protein interactions such as dimerization, multimerization and complex formation, specificity and/or affinity for ligand, cellular localization and relocalization, membrane anchoring, enzymatic activity such as kinase activity, response to regulatory molecules including regulatory proteins, cofactors, and other signaling molecules, such as in a signal transduction pathway. Generally, a biological activity is altered in an isoform at least 0.1, 0.5, 1, 2, 3, 4, 5, or 10 fold compared to a wildtype and/or predominant form of the receptor. Typically, a biological activity is altered 10, 20, 50, 100 or 1000 fold or more. For example, an isoform can be reduced in a biological activity.

CSR isoforms also can modulate an activity of a wildtype and/or predominant form of the receptor. For example, a CSR isoform can interact directly or indirectly with a CSR isoform and modulate a biological activity of the receptor. Biological activities that can be altered include, but are not limited to, protein-protein interactions such as dimerization, multimerization and complex formation, specificity and/or affinity for ligand, cellular localization and relocalization, membrane anchoring, enzymatic activity such as kinase activity, response to regulatory molecules including regulatory proteins, cofactors, and other signaling molecules, such as in a signal transduction pathway.

A CSR isoform can interact directly or indirectly with a cell surface receptor to cause or participate in a biological effect, such as by modulating a biological activity of the cell surface receptor. A CSR isoform also can interact independently of a cell surface receptor to cause a biological effect, such as by initiating or inhibiting a signal transduction pathway. For example, a CSR isoform can initiate a signal transduction pathway and enhance or promote cell growth. In another example, a CSR isoform can interact with the cell surface receptor as a ligand causing a biological effect for example by inhibiting a signal transduction pathway that can impede or inhibit cell growth. Hence, the isoforms provided herein can function as cell surface receptor ligands in that they interact with the targeted receptor in the same manner that a cognate ligand interacts with and alters receptor activity. The isoforms can bind as a ligand, but not necessarily, to a ligand binding site and serve to block receptor dimerization. They act as ligands in that they interact with the receptor. The CSR isoforms also can act by binding to ligands for the receptor and/or by preventing receptor activities, such as dimerization.

For example, a CSR isoform can compete with a CSR for ligand binding. A CSR isoform, when it binds to receptor, can be a negative effector ligand, which results in inhibition of receptor function. It also is possible that some CSR isoforms bind a cognate receptor, resulting in activation of the receptor. A CSR isoform can act as a competitive inhibitor of a CSR, for example, by complexing with a CSR isoform and altering the ability of the CSR to multimerize (e.g. dimerize or trimerize) with other CSRs. A CSR isoform can compete with a CSR for interactions with other polypeptides and cofactors in a signal transduction pathway. The cell surface isoforms and families of isoforms provided herein include, but are not limited to, isoforms of receptor tyrosine kinases (also referred to herein as RTK isoforms) and isoforms of other families of CSRs, such as TNFs and other G-protein-coupled receptors. In one example, a CSR isoform is a soluble polypeptide. For example, a CSR isoform lacks at least part or all of a transmembrane domain. Soluble isoforms can and/or modulate a biological activity of a wildtype or predominant form of a receptor (see for example, Kendall et al. (1993) PNAS 90: 10705, Werner et al. (1992) Molec. Cell Biol. 12: 82, Heaney et al. (1995) PNAS 92: 2365, Fukunaga et al. (1990) PNAS 87:8702, Wypych et al. (1995) Blood 85: 66-73, Barron et al. (1994) Gene 147:263, Cheng et al. (1994) Science 263: 1759, Dastot et al. (1996) PNAS 93:10723, Abramovich et al. (1994) FEBS Lett 338:295, Diamant et al. (1997) FEBS Lett 412:379, Ku et al. (1996) Blood 88:4124, Heaney M L and Golde D W (1998), J Leukocyte Biol. 64:135-146).

A cell surface receptor isoform can be produced by any method known in the art including isolation of isoforms expressed in cells, tissues and organisms, and by recombinant methods and by methods including in silico steps, synthetic methods and any methods known to those of skill in the art. Isoforms of cell surface receptors, including isoforms of receptor tyrosine kinases, can be encoded by alternatively spliced RNA molecules transcribed from a receptor tyrosine kinase gene. Such isoforms include exon deletion, exon extension, exon truncation and intron retention alternatively spliced RNAs. CSR isoforms, include receptor isoforms that contain sequences encoded by introns (or alternative exons); also referred to as intron fusion proteins.

Pharmaceutical compositions containing one or more different CSR isoforms are provided. Also provided are methods of treatment of diseases and conditions by administering the pharmaceutical compositions or delivering a CSR isoform, such by administering a vector that encodes the isoform. Administration can be effected in vivo or ex vivo.

Methods of identifying and producing CSR isoforms and nucleic acid molecules encoding CSR isoforms are provided herein. Also provided are methods for expressing, isolating and formulating CSR isoforms.

Classes of CSR Isoforms

As noted, CSR isoforms are polypeptides that lack a domain or portion of a domain sufficient to remove or reduce or otherwise alter, including having a positive or negative effect, on biological activity compared to the cognate unbound form of the receptor. Some CSR isoforms also have completely novel functions as a result of the gain or loss of domains, or even single amino acid replacements. CSR isoforms represent splice variants of a gene (or recombinant shortened variants) and can be generated by alternate splicing or by recombinant or synthetic methods. CSR isoforms can be encoded by alternatively spliced RNAs. CSR isoforms also can be generated by recombinant methods and by use of in silico and synthetic methods.

Typically, a CSR isoform produced from an alternatively spliced RNA is not a predominant form of a polypeptide encoded by a gene. In some instances, a CSR isoform can be a tissue-specific or developmental stage-specific polypeptide or disease specific (i.e., can be expressed at a different level from tissue-to-tissue or stage-to-stage or in a disease state compared to a non-diseased state or only may be expressed in the tissue, at the stage or during the disease process or progress). Alternatively spliced RNA forms that can encode CSR isoforms include, but are not limited to, exon deletion, exon retention, exon extension, exon truncation, and intron retention alternatively spliced RNAs. Included among CSR isoforms are intron fusion proteins.

(a) Alternative Splicing and Generation of CSR Isoforms

Genes in eukaryotes include introns and exons that are transcribed by RNA polymerase into RNA products generally referred to as pre-mRNA. Pre-mRNAs are typically intermediate products that are further processed through RNA splicing and processing to generate a final messenger RNA (mRNA). Typically, a final mRNA contains exons sequences and is obtained by splicing out the introns. Boundaries of introns and exons are marked by splice junctions, sequences of nucleotides that are used by the splicing machinery of the cell as signals and substrates for removing introns and joining together exon sequences. Exons are operatively linked together to form a mature RNA molecule. Typically, one or more exons in an mRNA contains an open reading frame encoding a polypeptide. In many cases, an open reading frame can be generated by operatively linking two or more exons; for example, a coding sequence can span exon junctions and an open reading frame is maintained across the junctions.

RNA also can undergo alternative splicing to produce a variety of different mRNA transcripts from a single gene. Alternatively spliced mRNAs can contain different numbers of and/or arrangements of exons. For example, a gene that has 10 exons can generate a variety of alternatively spliced mRNAs. Some mRNAs can contain all 10 exons, some with only 9, 8, 7, 6, 5 etc. In addition, products, for example, with 9 of the 10 exons, can be among a variety of mRNAs, each with a different exon missing. Alternatively spliced mRNAs can contain additional exons, not typically present in an RNA encoding a predominant or wild type form. Addition and deletion of exons includes addition and deletion, respectively of a 5′ exon, 3′exon and an exon internal in an RNA. Alternatively spliced RNA molecules also include addition of an intron or a portion of an intron operatively linked to or within an RNA. For example, an intron normally removed by splicing in an RNA encoding a wildtype or predominant form can be present in an alternatively spliced RNA. An intron or intron portion can be operatively linked within an RNA, such as between two exons. An intron or intron portion can be operatively linked at one end of an RNA, such as at the 3′ end of a transcript. In some examples, the presence of intron sequence within an RNA terminates transcription based on poly-adenylation sequences within an intron.

Alternative RNA splicing patterns can vary depending upon the cell and tissue type. Alternative RNA splicing also can be regulated by developmental stage of an organism, cell or tissue type. For example, RNA splicing enzymes and polypeptides that regulate RNA splicing can be present at different concentrations in particular cell and tissue types and at particular stages of development. In some cases, a particular enzyme or regulatory polypeptide can be absent from a particular cell or tissue type or at particular stage of development. These differences can produce different splicing patterns for an RNA within a cell or tissue type or stage, thus giving rise to different populations of mRNAs. Such complexity can generate a number of protein products appropriate for particular cell types or developmental stages.

Alternatively spliced mRNAs can generate a variety of different polypeptides, also referred to herein as isoforms. Such isoforms can include polypeptides with deletions, additions and shortenings. For example, a portion of an open reading frame normally encoded by an exon can be removed in an alternatively spliced mRNA, thus resulting in a shorter polypeptide. An isoform can have amino acids removed at the N or C terminus or the deletion can be internal. An isoform can be missing a domain or a portion of a domain as a result of a deleted exon. Alternatively spliced mRNAs also can generate polypeptides with additional sequences. For example, a stop codon can be contained in an exon; when this exon is not included in an mRNA, the stop codon is not present and the open reading frame continues into the sequences contained in downstream exons. In such examples, additional open reading frame sequences add additional amino acid residues to a polypeptide and can include addition of a new domain or a portion thereof.

(b) Intron Fusion Proteins

One class of isoforms is intron fusion proteins. An intron fusion protein is an isoform that lacks a domain or portion of a domain sufficient to remove or reduce a biological activity of a receptor. In addition, an intron fusion protein contains one or more amino acids not encoded by an exon, operatively linked to exon-encoded amino acids and/or is shortened compared to a wildtype or predominant form encoded by a CSR gene. Typically, an intron fusion protein is shortened by the presence of one or more stop codons in an intron fusion protein-encoding RNA that are not present in the corresponding sequence of an RNA encoding a wildtype or predominant form of a CSR polypeptide. Addition of amino acids and/or a stop codon can result in an intron fusion protein that differs in size and sequence from a wildtype or predominant form of a polypeptide.

An intron fusion protein is modified in one or more biological activities. For example, addition of amino acids in an intron fusion protein can add, extend or modify a biological activity compared to a wildtype or predominant form of a polypeptide. For example, fusion of an intron encoded amino acid sequence to a protein can result in the addition of a domain with new functionality. Fusion of an intron encoded polypeptide to a protein also can modulate an existing biological activity of a protein, such as by inhibiting a biological activity, for example, inhibition of dimerization or inhibition of kinase activity.

Intron fusion proteins include natural and combinatorial intron fusion proteins. A natural intron fusion protein is encoded by an alternatively spliced RNA that contains one or more introns or a portion thereof operatively linked to one or more exons of a gene. A natural intron fusion protein contains one or more amino acids encoded by an intron sequence and/or an intron fusion protein can be shortened as a result of one or more stop codons encoded by an intron sequence operatively linked to one or more exons. A combinatorial intron fusion protein is a polypeptide that is shortened compared to a wildtype or predominant form of a polypeptide. Typically, the shortening removes one or more domains or a portion thereof from a polypeptide. Combinatorial intron fusion proteins often mimic a natural intron fusion protein in that one or more domains or a portion thereof is/are deleted as in a natural intron fusion protein derived from the same gene sequence or derived from a gene sequence in a related gene family.

i. Natural Intron Fusion Proteins

Natural intron fusion proteins are generated from a class of alternatively spliced mRNAs that includes mRNAs that have incorporated intron sequences into mRNA as well as exon sequences, such as intron retention RNA molecules and some exon extension RNAs. They include all such variants that occur and can be isolated from a cell or tissue, identified in a database or synthesized based upon the sequence and structure of a gene. Any splice variant that is possible and that includes one or more codons (including only a stop codon) from an intron is considered a natural intron fusion protein.

The incorporated intron sequences can include one or more introns or a portion thereof. Such mRNAs can arise by a mechanism of intron retention. For example, a pre-mRNA is exported from the nucleus to the cytoplasm of the cell before the splicing machinery has removed one or more introns. In some cases, splice sites can be actively blocked, for example by cellular proteins, preventing splicing of one or more introns.

Retention of one or more introns or a portion thereof also can lead to the generation of isoforms referred to herein as natural intron fusion proteins. For example, an intron sequence can contain an open reading frame that is operatively linked to the exon sequences by RNA splicing. Intron-encoded sequences can add amino acids to a polypeptide, for example, at either the N or C terminus of a polypeptide, or internally within a polypeptide. In some examples, an intron sequence also can contain one or more stop codons. An intron encoded stop codon that is operatively linked with an open reading frame in one or more exons can terminate the encoded polypeptide. Thus, an isoform can be produced that is shortened as a result of the stop codon. In some examples, an intron retained in an mRNA can result in the addition of one or more amino acids and a stop codon to an open reading frame, thereby producing an isoform that terminates with an intron encoded sequence.

Provided herein are natural intron fusion proteins, that can be generated by intron retention, including intron fusion proteins with addition of domains or portion of domains encoded by an intron and intron fusion proteins with one or more domains or portion of domain deleted. For example, an intron sequence can be operatively linked in place of an exon sequence that is typically within an mRNA for a gene. A domain or portion thereof encoded by the exon is thus deleted from and intron encoded amino acids are included in the encoded polypeptide.

In another example, an intron sequence is operatively linked in addition to the typically present exons in an mRNA. In one example, an operatively linked intron sequence can introduce a stop codon in-frame with exon sequences encoding a polypeptide. In another example, an operatively linked intron sequence can introduce one or more amino acids into a polypeptide. In some embodiments, a stop codon in-frame also is operatively linked with exon sequences encoding a polypeptide, thereby generating an mRNA encoding a polypeptide with intron-encoded amino acids at the C terminus.

In one example of a natural intron fusion protein, one or more amino acids encoded by an intron sequence are operatively linked at the C terminus of a polypeptide. For example, an intron fusion protein is generated from a nucleic acid sequence that contains one or more exon sequences at the 5′ end of an RNA followed by one or more intron sequences or a portion of an intron sequence retained at the 3′ end of an RNA. An intron fusion protein produced from such nucleic acid contains exon-encoded amino acids at the N-terminus and one or more amino acids encoded by an intron sequence at the C-terminus. In another example, an intron fusion protein is generated from a nucleic acid by operatively linking a stop codon encoded within an intron sequence to one or more exon sequences, thereby generating a nucleic acid sequence encoding shortened polypeptide.

ii. Combinatorial Intron Fusion Proteins

Intron fusion proteins also can be generated by recombinant methods and/or in silico and synthetic methods to produce polypeptides that are modified compared to a wildtype or predominant form of a polypeptide. Typically, combinatorial intron fusion proteins are shortened polypeptides compared to a wildtype or predominant form. Shortening can remove one or more domains or a portion thereof.

Combinatorial intron fusion proteins are mimics of so-called natural intron fusion proteins in that one or more domains or a portion thereof that are deleted in a natural intron fusion protein derived from the same gene sequence or derived from a gene sequence in a related gene family is/are deleted. For example, as is described further herein, by aligning sequences of gene family members, intron and exons, structures and encoded protein domains can be identified in the nucleic acid. Recombinant nucleic acid molecules encoding polypeptides can be synthesized that contain one or more exons and an intron or portion thereof. Such recombinant molecules can contain one or more amino acids and/or a stop codon encoded by an intron, operatively linked to an exon, producing an intron fusion protein. Recombinant polypeptides also can be produced that contain a combinatorial intron fusion protein. As part of this method, potential immunogenic epitopes can be recognized using motif scanning, and modified with conservative amino acid substitutions or by other modifications well known in the art, such as PEGylation. Generally, any therapeutic intron fusion protein can be modified in this same way to achieve optimized pharmacokinetics or avoid immunogenicity.

(c) Intron-Encoded Isoforms

Another CSR isoform is an intron-encoded isoform. An intron-encoded isoform contains an intron sequences or portions thereof from an isoform, such as a natural intron fusion protein. An intron-encoded isoform can interact with a wildtype form or predominant form of a polypeptide produced from the same gene as the intron-encoded isoform. An intron-encoded isoforms can interact with a molecule in a signal transduction pathway that interact with a wildtype form or predominant form of a polypeptide produced from the same gene as the intron-encoded isoform. An intron-encoded isoform can be expressed or produced as a fusion with exon-encoded sequences. An intron-encoded isoform can be expressed or produced as a fusion with heterologous sequences such as a starting methionine. Stop codons can be engineered in the encoding nucleic acid molecule to terminate an intron-encoded isoform within or at the end of the intron sequence.

(d) Isoforms Generated by Exon Modifications

CSR isoforms can be generated by modification of an exon relative to a corresponding exon of an RNA encoding a wildtype or predominant form of a CSR polypeptide. Exon modifications include alternatively spliced RNA forms such as exon truncations, exon extensions, exon deletions and exon insertions. These alternatively spliced RNA molecules can encode CSR isoforms which differ from a wildtype or predominant form of a CSR polypeptide by including additional amino acids and/or by lacking amino acid residues present in a wildtype or predominant form of a CSR polypeptide.

Exon insertions are alternative spliced RNA molecules that contains at least one exon not typically present in an RNA encoding a wildtype or predominant form of a polypeptide. An inserted exon can operatively link additional amino acids encoded by the inserted exon to the other exons present in an RNA. An inserted exon also can contain one or more stop codons such that the RNA encoded polypeptide terminates as a result of such stop codons. If an exon containing such stop codons is inserted upstream of an exon that contains the stop codon used for polypeptide termination of a wildtype or predominant form of a polypeptide, a shortened polypeptide can be produced.

An inserted exon can maintain an open reading frame, such that when the exon is inserted, the RNA encodes an isoform containing an amino acid sequence of a wildtype or predominant form of a polypeptide with additional amino acids encoded by the inserted exon. An inserted exon can be inserted 5′, 3′ or internally in an RNA, such that additional amino acids encoded by the inserted exon are linked at the N terminus, C-terminus or internally, respectively in an isoform. An inserted exon also can change the reading frame of an RNA in which it is inserted, such that an isoform is produced that contains only a portion of the sequence of amino acids in a wildtype or predominant form of a polypeptide. Such isoforms can additionally contain amino acid sequence encoded by the inserted exon and also can terminate as a result of a stop codon contained in the inserted exon.

CSR isoforms also can be produced from exon deletion events. An exon deletion refers to an event of alternative RNA splicing that produces a nucleic acid molecule that lacks at least one exon compared to an RNA encoding a wildtype or predominant form of a polypeptide. Deletion of an exon can produce a polypeptide of alternate size such as by removing sequences that encode amino acids as well as by changing the reading frame of an RNA encoding a polypeptide. An exon deletion can remove one or more amino acids from an encoded polypeptide; such amino acids can be N-terminal, C-terminal or internal to a polypeptide depending upon the location of the exon in an RNA sequence. Deletion of an exon in an RNA also can cause a shift in reading frame such that an isoform is produced containing one or more amino acids not present in a wildtype or predominant form of a polypeptide. A shift in reading frame also can result in a stop codon in the reading frame producing an isoform that terminates at a sequence different from that of a wildtype or predominant form of a polypeptide. In one example, a shift of reading frame produces an isoform that is shortened compared to a wildtype or predominant form of a polypeptide. Such shortened isoforms also can contain sequences of amino acids not present in a wildtype or predominant form of a polypeptide.

CSR isoforms also can be produced by exon extension in an RNA. Exon extension is an event of alternative RNA splicing that produces a nucleic acid molecule that contains at least one exon that is greater in length (number of nucleotides contained in the exon) than the corresponding exon in an RNA encoding a wildtype or predominant form of a polypeptide. Additional sequence contained in an exon extension can encode additional amino acids and/or can contain a stop codon that terminates a polypeptide. An exon insertion containing an in-frame stop codon can produce a shortened isoform, that terminates in the sequence of the exon extension. An exon insertion also can shift the reading frame of an RNA, resulting in an isoform containing one or more amino acids not present in a wildtype or predominant form of a polypeptide and/or an isoform that terminates at a sequence different from that of a wildtype or predominant form of a polypeptide. An exon extension can include sequences contained in an intron of an RNA encoding a wildtype or predominant form of a polypeptide and thereby produce an intron fusion protein.

CSR isoforms also can be produced by exon truncation. Exon truncations are RNA molecules that contain a shortening of one or more exons such that the one or more exons are shorter in length (number of nucleotides) compared to a corresponding exon in an RNA encoding a wildtype or predominant form of a polypeptide. An RNA molecule with an exon truncation can produce a polypeptide that is shortened compared to a wildtype or predominant form of a polypeptide. An exon truncation also can result in a shift in reading frame such that an isoform is produced containing one or more amino acids not present in a wildtype or predominant form of a polypeptide. A shift in reading frame also can result in a stop codon in the reading frame producing an isoform that terminates at a sequence different from that of a wildtype or predominant form of a polypeptide.

Alternatively spliced RNA molecules including exon modifications can produce CSR isoforms that a lack a domain or a portion thereof sufficient to reduce or remove a biological activity. For example, exon modified RNA molecules can encode shortened CSR polypeptides that lack a domain or portion thereof. Exon modified RNA molecules also can encode polypeptides where a domain is interrupted by inserted amino acids and/or by a shift in reading frame that interrupts a domain with one or more amino acids not present in a wildtype or predominant form of a polypeptide.

### C. Receptor Tyrosine Kinase Isoforms

CSR isoforms provided herein include isoforms of receptor tyrosine kinases (RTKs), including receptor tyrosine kinase intron fusion proteins. The receptor tyrosine kinases (RTKs) are a large family of structurally related growth factor receptors. RTKs are involved in cellular processes including cell growth, differentiation, metabolism and cell migration. RTKs also are known to be involved in cell proliferation, differentiation and determination of cell fate. Members of the family include, but are not limited to, epidermal growth factor (EGF) receptors, platelet-derived growth factor (PDGF) receptors, fibroblast growth factor (FGF) receptors, insulin-like growth factor (IGF) receptors, nerve growth factor (NGF) receptors, vascular endothelial growth factor (VEGF) receptors, receptors to ephrin (termed Eph), hepatocyte growth factor (HGF) receptors (termed MET), TEK/Tie-2 (the receptor for angiopoietin-1), discoidin domain receptors (DDR) and others, such as Tyro3/Ax1.

Provided herein are RTK isoforms that are modified in one more domains of an RTK such that they lack a domain of an RTK or a portion of a domain sufficient to remove or reduce a biological activity of an RTK. Also provided are RTK isoforms modified at one or more amino acids of an RTK sequence such as by shortening and/or addition of one more amino acids. Additional amino acids can add a new domain or a portion thereof. RTK isoforms can be modified in a biological activity including, but not limited to, dimerization, kinase activity, signal transduction, ligand binding, membrane association and membrane localization. RTK isoforms also can modulate a biological activity of an RTK.

1. RTK Domains and Biological Activities

RTKs have a conserved domain structure including an extracellular domain, a membrane-spanning (transmembrane) domain and an intracellular tyrosine kinase domain. The extracellular domain can bind to a ligand, such as a polypeptide growth factor or a cell membrane-associated molecule. Some RTKs have been classified as orphan receptors, having no identified ligand. Some RTKs are classified as constitutive RTKs, active without ligand binding.

Typically, dimerization of RTKs activates the catalytic tyrosine kinase domain of the receptor and subsequent activities in signal transduction. RTKs can be homodimers or heterodimers. For example, PDGF is a heterodimer composed of α and β subunits. VEGF receptors are homodimers. EGF receptors can be either heterodimers or homodimers. In another example, ErbB3, in the presence of the ligand heregulin, heterodimerizes with other members of the ErbB family (EGFR family) such as ErbB2 and ErbB3. Many RTKs are capable of autophosphorylation when dimerized, such as by transphosphorylation between subunits. Autophosphorylation in the kinase domain maintains the tyrosine kinase domain in an activated state. Autophosphorylation in other regions of the protein can influence interaction of the receptor with other cellular proteins.

RTKs interact in signal transduction pathways. For example, RTKs, when activated can phosphorylate other signaling molecules. For example, EGFR interacts in signal transduction pathways involved in processes including proliferation, dedifferentiation, apoptosis, cell migration and angiogenesis. EGFR family members can recruit signaling molecules through protein:protein interactions; some interactions involve specific binding of signaling molecules to tyrosine phosphorylated sites on the receptor. For example, the Grb2/Sos complex can bind to phosphotyrosine sites on EGFR, in turn activating the Ras/Raf/MAPK signaling cascade, which influences cell proliferation, migration and differentiation. Other exemplary signaling molecules include other RTKs, G-coupled receptors, integrins, phospholipase C, Ca²⁺/calmodulin-dependent kinases, transcriptional activators, cytokines and other kinases.

2. Receptor Tyrosine Kinase Isoforms

RTK isoforms lack a domain or a portion of a domain of a receptor tyrosine kinase. Thus, an RTK isoforms differs from its cognate RTK in one or more biological activities. In addition, an RTK isoform can modulate a biological activity of an RTK, such as by interacting with an RTK directly or indirectly. Biological activities include, but are not limited to, protein-protein interactions such as dimerization, multimerization and complex formation, specificity and/or affinity for ligand, cellular localization and relocalization, membrane anchoring, enzymatic activity such as kinase activity, response to regulatory molecules including regulatory proteins, cofactors, and other signaling molecules, such as in a signal transduction pathway.

3. RTK Isoform Structure and Activity

In one embodiment, an RTK isoform is modified in a kinase domain. For example, an RTK isoform contains a deletion of a kinase domain or a portion thereof. The deletion need not be a deletion of the entire domain, one or more amino acids can be deleted within the domain. The deletion can be at the N-terminus of the kinase domain, the C-terminus or internally within the domain. In another example, an RTK isoform contains addition of amino acids in a kinase domain. The addition of amino acids can be at the N-terminus of the domain, the C-terminus or anywhere internally within a kinase domain.

In one aspect of the embodiment, kinase activity of an RTK isoform is altered. For example, kinase activity of an RTK isoform is reduced or eliminated. In one example, substrate specificity of the kinase activity of an RTK isoform is altered. For example, an RTK isoform is capable of autophosphorylation but not phosphorylation of other polypeptides, such as polypeptides in a signal transduction pathway. In another example, an RTK isoform phosphorylates other polypeptides but is not capable of autophosphorylation. Kinase activity of an RTK isoform can be enhanced in activity. Kinase activity of an RTK isoform can be altered in regulation. For example, the kinase activity can be constitutively active or constitutively inactive, for example, unregulated by the addition of ligand, by receptor dimerization, by complexation such as through protein:protein interactions, and/or by autophosphorylation.

In one embodiment, an RTK isoform is modified in a transmembrane domain. For example, an RTK isoform contains a deletion of a transmembrane domain or a portion thereof. The deletion can be at the N-terminus of a transmembrane domain, the C-terminus or internally within the domain. In another example, an RTK isoform contains addition of amino acids in a transmembrane domain. The addition of amino acids can be at the N-terminus of the domain, the C-terminus or anywhere internally within the transmembrane domain.

In one aspect of the embodiments, membrane association and/or localization of an RTK isoform is altered. For example, an RTK isoform can be a soluble protein (e.g. not membrane localized), where a wildtype or a predominant form of the RTK is membrane localized. For example, an RTK isoform can be secreted extracellularly or localized in the cytoplasm or internally within a cellular organelle. An RTK isoform can be altered in its membrane localization. For example, an RTK isoform can associate with internal membranes, such as membranes of cellular organelles, but not the cytoplasmic membrane. An RTK isoform can be reduced in its association with a membrane, such that the proportion of membrane associated protein is altered; for example, some of the protein is soluble and some is membrane associated. An RTK isoform also can be altered in the orientation with or within a membrane compared to the orientation of a wildtype or predominant form of an RTK. For example, more or less of the polypeptide can be embedded within the membrane. More or less of the polypeptide can be associated with either side of the cellular membrane. For example, orientation can be altered such that more of the RTK isoform is found in the cytoplasm or extracellularly compared to a wildtype or predominant form of an RTK.

In one embodiment, an RTK isoform is altered in its dimerization activity. For example, an RTK-isoform homodimerizes (i.e. an RTK isoform: RTK isoform complex) but does not heterodimerize or is reduced in heterodimerization with a wildtype or predominant form of an RTK derived from the same gene. In another example, an RTK-isoform does not homodimerize with itself, or is reduced in homodimerization activity but can heterodimerize with a wildtype or predominant form of an RTK from the same gene or a different gene. In another example, an RTK isoform is reduced in heterodimerization with RTKs from other genes but heterodimerizes with RTKs from the same gene.

In one embodiment, an RTK isoform is altered in its signal transduction activity. For example, an RTK isoform is altered in its association with other cellular proteins or cofactors in a signal transduction pathway. For example, an RTK isoform is altered in an interaction such as, but not limited to, an interaction with another RTK, a G-coupled receptor, an integrin, phospholipase C, a Ca²⁺/calmodulin-dependent kinase, a transcriptional activator or regulator, a cytokine and another kinase. In another example, an RTK isoform alters signal transduction of an RTK. For example, an RTK isoform interacts with an RTK and alters its activity in signal transduction, such as by inhibiting or by stimulating signal transduction by the RTK.

In one embodiment, an RTK isoform is altered in two or more biological activities. For example, an RTK isoform is altered in kinase activity and membrane association. In another example, an RTK isoform is altered in kinase activity and dimerization. In yet another example, an RTK isoform is altered in kinase activity, dimerization and membrane association. For example, an RTK isoform is modified in a kinase domain and a transmembrane domain. In another example, insertion of addition of amino acids interrupts the kinase domain and transmembrane domains. In another embodiment, an RTK isoform is modified at a domain junction, or outside the linear sequence of amino acids for a domain and the modification alters a structure, such as the 3-dimensional structure of a domain such as a kinase domain, or a transmembrane domain.

4. Modulation of RTKs by RTK Isoforms

RTK isoforms can modulate or alter a biological activity of an RTK, such as by interacting directly or indirectly with an RTK. Biological activities include, but are not limited to, protein-protein interactions such as dimerization, multimerization and complex formation, specificity and/or affinity for ligand, cellular localization and relocalization, membrane anchoring, enzymatic activity such as kinase activity, response to regulatory molecules including regulatory proteins, cofactors, and other signaling molecules, such as in a signal transduction pathway. In one embodiment, interaction of an RTK isoform with an RTK, inhibits an RTK biological activity. In another embodiment, interaction of an RTK isoform with an RTK, stimulates a biological activity of an RTK.

For example, an RTK isoform competes with an RTK for ligand binding. An RTK isoform can be employed as a “ligand sponge” to remove free ligand and thereby regulate or modulate the activity of an RTK. In another example, an RTK isoform acts as a negatively acting ligand when heterodimerized or complexed with an RTK, for example, by preventing trans-autophosphorylation. An RTK isoform that lack the protein kinase domain, or a portion thereof sufficient to alter kinase activity, can inhibit activation of an RTK in a trans dominant manner.

In one embodiment, an RTK isoform acts as a competitive inhibitor of RTK dimerization. For example, an RTK isoform interacts with an RTK and prevents that RTK from homodimerizing or from heterodimerizing. An isoform that inhibits receptor dimerization can modulate downstream signal transduction pathways, such as by complexing with the receptor and inhibiting receptor activation as downstream signaling. An RTK isoform also acts as a competitive inhibitor of an RTK by competing directly with an RTK for interactions with other polypeptides and cofactors in a signal transduction pathway.

### D. TNFR Isoforms

CSR isoforms provided herein include isoforms of tumor necrosis factor receptors (TNFRs). TNFR isoforms lack a domain or a portion of a domain of a TNFR receptor. Thus, a TNFR isoform differs from its cognate TNFR in one or more biological activities. In addition, a TNFR isoform can modulate a biological activity of a TNFR, such as by interacting with a TNFR directly or indirectly. Biological activities include, but are not limited to, protein-protein interactions such as trimerization, multimerization and complex formation, specificity and/or affinity for ligand, cellular localization and relocalization, membrane anchoring, response to regulatory molecules including regulatory proteins, cofactors, and other signaling molecules, such as in a signal transduction pathway.

1. TNFR Domains and Biological Activities

The TNF ligand and receptor family regulate a variety of signal transduction pathways including those involved in cell differentiation, activation, and viability. TNFRs have a characteristic repeating extracellular cysteine-rich motif and a variable intracellular domain that differs between members of the TNFR family. The TNFR family of receptors includes, but is not limited to, TNFR1, TNFR2, TNFRrp, the low-affinity nerve growth factor receptor, Fas antigen, CD40, CD27, CD30, 4-1BB, OX40, DR3, DR4, DR5, and herpesvirus entry mediator (HVEM). Ligands for TNFRs include TNF-α, lymphotoxin, nerve growth factor, Fas ligand, CD40 ligand, CD27 ligand, CD30 ligand, 4-1BB ligand, OX40 ligand, APO3 ligand, TRAIL and LIGHT. TNFRs include an extracellular domain, including a ligand binding domain, a transmembrane domain and an intracellular domain that participates in signal transduction. These receptors have names. For example, TNFR1 also is referred to as p55 or p60; and TNFR2 also is referred to as p75 or p80. TNFRs are typically trimeric proteins that trimerize at the cell surface. Trimerization is important for biological activity of TNFRs.

TNFRs have a characteristic extracellular domain with a cysteine-rich motif. The extracellular domain includes a ligand binding domain. Typically, each TNFR member binds a unique ligand. A few receptors such as TNFR1 and TNFR2 and DR4 and DR5 have overlapping ligand specificity. TNFRs also trimerize. Trimerization can be induced by ligand interaction. TNFR ligands also can be trimers. Some TNFRs can be proteolytically processed to produce a secreted form of the receptor. The secreted form also trimerizes and retains certain biological activities such as ligand binding, interaction with the membrane bound form of the receptor, and inhibition of the membrane-bound form of the receptor.

TNFRs can trigger signal transduction. For example, TNFR1 activates intracellular pathways involved in apoptosis. TNFR1 trimerizes upon binding TNF ligand. Trimerization induces association of the receptor's death domains. Adapter proteins such as TRADD, TRAF-2, FADD and RIP also associate with the receptor. TRAF-2 and RIP associations activate NF-κB and JNK/AP-1 pathways, including a cascade of kinases. FADD association activates a caspase cascade and subsequent apoptosis.

2. TNFR Isoform Structure and Activity

In one embodiment, a TNFR isoform is modified in a transmembrane domain. For example, a TNFR isoform contains a deletion of a transmembrane domain or a portion thereof. The deletion can be at the N-terminus of a transmembrane domain, the C-terminus or internally within the domain. In another example, a TNFR isoform contains addition of amino acids in a transmembrane domain. The addition of amino acids can be at the N-terminus of the domain, the C-terminus or anywhere internally within the transmembrane domain.

In one aspect of the embodiments, membrane association and/or localization of a TNFR isoform is altered. For example, a TNFR isoform can be a soluble protein (e.g. not membrane localized), where a wildtype or a predominant form of the TNFR is membrane localized. For example, a TNFR isoform can be secreted extracellularly or localized in the cytoplasm or internally within a cellular organelle. A TNFR isoform can be altered in its membrane localization. For example, a TNFR isoform can associate with internal membranes, such as membranes of cellular organelles, but not the cytoplasmic membrane. A TNFR isoform can be reduced in its association with a membrane, such that the proportion of membrane associated protein is altered; for example, some of the protein is soluble and some is membrane associated. A TNFR isoform also can be altered in the orientation with or within a membrane compared to the orientation of a wildtype or predominant form of a TNFR. For example, more or less of the polypeptide can be embedded within the membrane. More or less of the polypeptide can be associated with either side of the cellular membrane. For example, orientation can be altered such that more of a TNFR isoform is found in the cytoplasm or extracellularly compared to a wildtype or predominant form of a TNFR.

In one embodiment, a TNFR isoform is modified in an intracellular domain. For example, a TNFR isoform contains a deletion of an intracellular domain or a portion thereof. The deletion can be at the N-terminus of an intracellular domain, the C-terminus or internally within the domain. In another example, a TNFR isoform contains addition of amino acids in an intracellular domain. The addition of amino acids can be at the N-terminus of the domain, the C-terminus or anywhere internally within the intracellular domain.

In one embodiment, an TNFR isoform is altered in its trimerization activity. For example, a TNFR isoform homotrimerizes (i.e. a TNFR isoform: TNFR isoform complex) but does not heterotrimerize or is reduced in heterotrimerization with a wildtype or predominant form of a TNFR derived from the same gene. In another example, a TNFR isoform does not homotrimerize with itself, or is reduced in homotrimerization activity but can heterotrimerize with a wildtype or predominant form of a TNFR from the same gene or a different gene. In one embodiment, a TNFR isoform acts as a competitive inhibitor of TNFR trimerization. For example, a TNFR interacts with a TNFR and prevents that TNFR from trimerizing.

In one embodiment, an TNFR isoform is altered in its signal transduction activity. For example, a TNFR isoform is altered in its association with other cellular proteins or cofactors in a signal transduction pathway. For example, a TNFR isoform is altered in an interaction such as, but not limited to, an interaction with a ligand and an adapter protein such as TRADD (TNFR-associated death domain), TRAF-2, FADD (Fas-associated death domain) and RIP (receptor interacting protein). In another example, a TNFR isoform alters signal transduction of a TNFR. For example, a TNFR isoform interacts with a TNFR and alters its activity in signal transduction, such as by inhibiting or by stimulating signal transduction by the TNFR.

In an exemplary embodiment, a TNFR isoform is altered in two or more biological activities. For example, a TNFR isoform is altered in signal transduction and membrane association. In another example, a TNFR isoform is altered in signal transduction and trimerization. In yet another example, a TNFR isoform is altered in kinase activity, trimerization and membrane association. In another embodiment, an TNFR isoform is modified in an intracellular domain and a transmembrane domain. For example, the two domains, or a portion of the domains are deleted. In another example, insertion or addition of amino acids interrupts the intracellular domain and transmembrane domains. In another embodiment, a TNFR isoform is modified at a domain junction, or outside the linear sequence of amino acids for a domain and the modification alters a structure, such as the 3-dimensional structure of a domain such as an intracellular domain, or a transmembrane domain.

3. Modulation of TNFRs by TNFR Isoforms

TNFR isoforms can modulate or alter a biological activity of a TNFR, such as by interacting directly or indirectly with a TNFR. Biological activities include, but are not limited to, protein-protein interactions such as trimerization, multimerization and complex formation, specificity and/or affinity for ligand, cellular localization and relocalization, membrane anchoring, response to regulatory molecules including regulatory proteins, cofactors, and other signaling molecules, such as in a signal transduction pathway. In one embodiment, interaction of a TNFR isoform with a TNFR, inhibits a TNFR biological activity. In another embodiment, interaction of a TNFR isoform with a TNFR, stimulates a biological activity of a TNFR.

For example, a TNFR isoform competes with a TNFR for ligand binding. A TNFR isoform can be employed as a “ligand sponge” to remove free ligand and thereby regulate or modulate the activity of a TNFR. In another example, a TNFR isoform acts as a negatively acting ligand when trimerized or complexed with a TNFR, for example, by preventing signal transduction and/or by inhibiting interaction with a member of a signal transduction pathway, such as adapter proteins. In one embodiment, a TNFR isoform acts as a competitive inhibitor of TNFR trimerization. For example, a TNFR isoform interacts with a TNFR and prevents that TNFR from trimerizing. An isoform that inhibits receptor trimerization can modulate downstream signal transduction pathways, such as by complexing with the receptor and inhibiting receptor activation as downstream signaling.

### E. Methods for Identifying and Generating CSR Isoforms

CSR isoforms can be generated by analysis and identification of naturally occurring genes and expression products (RNAs) using cloning methods in combination with bioinformatics methods such as sequence alignments and domain mapping and selections.

Provided herein are methods herein for identifying and isolating CSR isoforms that utilize cloning of expressed gene sequences and alignment with a gene sequence such as a genomic DNA sequence. For example, one or more isoforms can be isolated by selecting a candidate gene, such as a receptor tyrosine kinase. Expressed sequences, such as cDNA molecules or regions of cDNAs, are isolated. Primers can be designed to amplify a cDNA or a region of a cDNA. In one example, primers are designed which overlap or flank the start codon of the open reading frame of a candidate gene and primers are designed which overlap or flank the stop codon of the open reading frame. Primers can be used in PCR, such as in reverse transcriptase PCR (RT-PCR) with mRNA, to amplify nucleic acid molecules encoding open reading frames. Such nucleic acid molecules can be sequenced to identify those that encode an isoform. In one example, nucleic acid molecules of different sizes (e.g. molecular masses) from a predicted size (such as a size predicted for encoding a wildtype or predominant form) are chosen as candidate isoforms. Such nucleic acid molecules then can be analyzed, such by a method described herein, to further select isoform-encoding molecules having specified properties.

Computational analysis is performed using the obtained nucleic acid sequences to further select candidate isoforms. For example, cDNA sequences are aligned with a genomic sequence of a selected candidate gene. Such alignments can be performed manually or by using bioinformatics programs such as SIM4, a computer program for analysis of splice variants. Sequences with canonical donor-acceptor splicing sites (e.g. GT-AG) are selected. Molecules can be chosen which represent alternatively spliced products such as exon deletion, exon retention, exon extension and intron retention can be selected.

Sequence analysis of isolated nucleic acid molecules also can be used to further select isoforms that retain or lack a domain and/or biological function compared to a wildtype or predominant form. For example, isoforms encoded by isolated nucleic acid molecules can be analyzed using bioinformatics programs such as described herein to identify protein domains. Isoforms then can be selected which retain or lack a domain or a portion thereof.

In one embodiment of the method, isoforms are selected that lack a transmembrane domain or portion thereof sufficient to lack or significantly reduce membrane localization. For example, isoforms are selected that are shortened before a transmembrane domain or that are shortened within a transmembrane domain. Isoforms also can be selected that lack a transmembrane domain or portion thereof and have one or more amino acids operatively linked in place of the missing domain or portion of a domain. Such isoforms can be the result of alternative splicing events such as exon extension, intron retention, exon deletion and exon insertion. In some case, such alternatively spliced RNA molecules alter the reading frame of an RNA and/or operatively link sequences not found in an RNA encoding a wildtype or predominant form. Isoforms also can be selected that lack a kinase domain or portion thereof. Isoforms can be selected that lack a kinase domain or portion thereof and also lack a transmembrane domain or portion thereof. Isoforms also can be selected that lack a multimerization domain, such as a dimerization or trimerization domain, and/or an intracellular domain that interacts with and participates in signal transduction activity.

For example, nucleic acid molecules encoding candidate RTK isoforms can be further selected for isoforms that lack a kinase domain, a transmembrane domain, an extracellular domain or a portion thereof. Nucleic acid molecules can be selected which encode an RTK isoform and have a biological activity that differs from a wildtype or predominant form of an RTK. In one example, RTK isoforms are selected that lack a transmembrane domain such that the isoforms are not membrane localized and are secreted from a cell. In another example, TNFR isoforms are identified and selected that lack a transmembrane domain, a portion thereof. TNFR isoforms also can be selected that lack an intracellular domain or that lack an intracellular domain and a transmembrane domain.

Allelic Variants of Isoforms

Allelic variants of CSR isoform sequences can be generated or identified that differ in one or more amino acids from a particular CSR isoform. Allelic variation occurs among members of a population or species and also between species. For example, isoforms can be derived from different alleles of a gene; each allele can have one or more amino acid differences from the other. Such alleles can have conservative and/or non-conservative amino acid differences. Allelic variants also include isoforms produced or identified from different subjects, such as individual subjects or animal models or other animals. Amino acid changes can result in modulation of an isoform biological activity. In some cases, an amino acid difference can be “silent,” having no or virtually no detectable effect on a biological activity. Allelic variants of isoforms also can be generated by mutagenesis. Such mutagenesis can be random or directed. For example, allelic variant isoforms can be generated that alter amino acid sequences or a potential glycosylation site to effect a change in glycosylation of an isoform, including alternate glycosylation, increased or inhibition of glycosylation at a site in an isoform. Allelic variant isoforms can be at least 90% identical in sequence to an isoform. Generally, an allelic variant isoform from the same species is at least 95%, 96%, 97%, 98%, 99% identical to an isoform, typically an allelic variant is 98%, 99%, 99.5% identical to an isoform.

### F. Exemplary CSR Isoforms

The methods herein can be used to generate CSR isoforms from a variety of genes. One exemplary group of genes is receptor tyrosine kinases. Receptor tyrosine kinases (RTKs) are a large collection of genes and encoded polypeptides that can be grouped into families based on, for example, structural arrangements of sequence motifs in the polypeptides. For example, structural motifs in the extracellular domains such as, immunoglobulin, fibronectin, cadherin, epidermal growth factor and kringle repeats can be used to group RTKs. Such classification by structural motifs has identified greater then 16 families of RTKs, each with a conserved tyrosine kinase domain. Examples of RTKs include, but are not limited to, erythropoietin-producing hepatocellular (EPH) receptors (also referred to as ephrin receptors), epidermal growth factor (EGF) receptors, fibroblast growth factor (FGF) receptors, platelet-derived growth factor (PDGF) receptors, vascular endothelial growth factor (VEGF) receptors, cell adhesion RTKs (CAKs), Tie/Tek receptors, hepatocyte growth factor (HGF) receptors (termed MET), TEK/Tie-2 (the receptor for angiopoietin-1), discoidin domain receptors (DDR), insulin growth factor (IGF) receptors, insulin receptor-related (IRR) receptors and others, such as Tyro3/Ax1. Exemplary genes encoding RTKs include, but are not limited to, ErbB2, ErbB3, DDR1, DDR2, EGFR, EphA1, EphA2, EphA3, EphA4, EphA5, EphA6, EphA7, EphA8, EphB1, EphB2, EphB3, EphB4, EphB5, EphB6, FGFR-1, FGFR-2, FGFR-3, FGFR-4, FLT1 (also known as VEGFR-1), VEGFR-2, VEGFR-3 (also known as VEGFRC), MET, RON, PDGFR-A, PDGFR-B, CSF1R, Flt3, KIT, TIE-1 and TEK (also known as TIE-2) and genes encoding the RTKs noted above and not set forth.

RTKs participate in a variety of signal transduction pathways. RTKs regulate critical cellular processes including cell proliferation, dedifferentiation, apoptosis, cell migration and angiogenesis. RTK activation and thus subsequent activation of a signal transduction pathway is generally dependent on receptor activation, such as by activation of the receptor by ligand binding and autophosphorylation. RTKs can be subject to misregulation leading to misregulation of signal transduction. Such misregulation is associated with a number of diseases and conditions. Alternatively, certain RTKs are expressed on cells and lead to or participate in alteration in cellular activities, such as oncogenic transformation. Such expression and/or misregulation is associated with a number of diseases and conditions, including but not limited to diseases involving abnormal cell proliferation, such as neoplastic diseases, restenosis, disease of the anterior eye, cardiovascular diseases, obesity and a variety of others.

RTK isoforms provided herein and generated by methods provided herein can be used to modulate a biological activity of an RTK, such as an RTK endogenous to a particular cell type or tissue. The ability to modulate a biological activity of an RTK allows re-regulation of misregulated RTKs as well as directed regulation of cellular pathways in which RTKs participate. Modulating a biological activity of an RTK includes direct modulation, whereby an RTK isoform interacts with an RTK, such as by complexation with an RTK, modulation of homodimerization and/or heterodimerization of an RTK and/or modulation of trans-phosphorylation of an RTK, including inhibition of phosphorylation of an RTK. Modulation of an RTK also includes indirect modulation whereby an RTK isoform indirectly affects a biological activity of an RTK. Indirect modulation includes isoforms that act as a “ligand sponge,” competing for ligand binding with an RTK. Indirect modulation also includes interactions of an isoform with signaling molecules in a signaling pathway, thus modulating the activity such as by competition with interactions of such signaling molecules with an RTK. Exemplary RTK isoforms and uses of such RTK isoforms in targeting and regulating RTK activity are described below.

1. EGFR

EGFR (epidermal growth factor receptor) is a 170 kDa protein that binds to EGF, a small, 53 amino acid protein-ligand that stimulates the proliferation of epidermal cells and a variety of other cell types. EGF receptors are widely expressed in epithelial, mesenchymal and neuronal tissues and play important roles in proliferation and differentiation. EGF Receptor is characterized by several functional domains. The EGFR protein (GenBank No. NP_{—}005219 set forth as SEQ ID NO:252 is characterized by two Receptor L Domains between amino acids 57-168 and amino acids 361-481. Receptor L Domains make up the bilobal ligand binding site. A Furin-like cysteine rich region, typically involved in the signal transduction mechanism of receptor tyrosine kinases and receptor aggregation, can be found in EGFR between amino acids 184-338. The transmembrane domain of EGFR lies between amino acids 646-668 and protein kinase domain lies between amino acids 712-968.

EGFR polypeptides include allelic variants of EGFR. For example, an allelic variant contains one or more amino acid changes compared to SEQ ID NO:252. For example, one or more amino acid variations can occur in the protein kinase domain of EGFR. An allelic variant can include amino acid changes at position 719 where, for example, G is replaced by C, or at position 858 where, for example, L is replaced by R, or at position 861 where, for example, L is replaced by Q. An allelic variation also can include one or more amino acid changes, such as at position 521 (SNP NO: 11543848) where, for example, R can be replaced by K. In one example, an allelic variant includes one or more amino acid changes compared to SEQ ID NO:252 and the variant exhibits a change in biological activity. Amino acid changes occurring in the protein kinase domain, such as at position 719, 858, or 861, can be associated with a response to Gefitinib in patients with non-small-cell lung cancer indicating an essential role of the EGFR signaling pathway in the tumor, or, such as at position 858, can be associated with enhanced activity of the EGFR receptor in response to EGF as assessed by autophosphorylation of EGFR. An exemplary EGFR allelic variant containing one or more amino acid changes described above is set forth as SEQ ID NO: 288.

EGF receptors are encoded by a family of related genes known as also erbB genes (e.g. ErbB2, ErbB3, ErbB4) and HER genes (e.g. Her-2). The EGF receptor family includes four members, EGF-receptor (HER-1; erbB-1), human epidermal growth factor receptor-2 (HER-2; ErbB2), HER-3 (ErbB3) and HER-4 (ErbB4). The ligand for EGFR/HER-1 is EGF, while the ligand for HER-2, HER-3 and HER-4 is neuregulin-1 (NRG-1). NRG-1 preferentially binds to either HER-3 or HER-4 after which the bound receptor subunit heterodimerizes with HER-2. HER-4 also is capable of homodimerization to form an active receptor.

Misregulation of the ErbB family has been implicated in a number of different types of cancer. For example, overexpression of EGFR is associated with a number of human tumors including, but not limited to, esophageal, stomach, bladder and colon cancers, gliomas and meningiomas, squamous carcinoma of the lungs, and ovarian, cervical and renal carcinomas. Using the methods provided herein, RTK isoforms and pharmaceutical compositions containing RTK isoforms can be generated for use as therapeutic agents which target and re-regulate misregulation of EGF receptors.

a. ErbB2

ErbB2 is a member of the EGF receptor family. The ErbB2 protein (GenBank No. NP_{—}004439 set forth as SEQ ID NO:266) is characterized by two Receptor L Domains between amino acids 52-173 and amino acids 366-486; a Furin-like cysteine rich region between amino acids 189-343; the transmembrane domain between amino acids 653-675; and protein kinase domain between amino acids 720-976. A ligand that binds with high affinity has not been identified for ErbB2. Instead, ErbB3 or ErbB4 when bound by ligand (NRG-1) heterodimerize with ErbB2 to form an active receptor dimer. In addition, ErbB2 exhibits constitutive activity (homodimerization and kinase activity) in the absence of ligand. In addition, overexpression of ErbB2 is capable of cell transformation. ErbB2 overexpression has been identified in a variety of cancers, including breast, ovarian, gastric and endometrial carcinomas. Thus, targeting ErbB2 homodimers can regulate ErbB2 homodimerization. For example, an ErbB2 RTK isoform can target and down-regulate ErbB2 overexpression. Additionally, an ErbB2 RTK-isoform can target ErbB3 and/or ErbB4 through heterodimerization.

ErbB2 proteins include allelic variants of ErbB2. In one example, an allelic variant contains one or more amino acid changes compared to SEQ ID NO:266. For example, one or more amino acid variations can occur in the transmembrane domain of ErbB2. An allelic variant can include amino acid changes at position 655 where, for example, I is replaced by V. In one example, an allelic variant includes one or more amino acid changes compared to SEQ ID NO:266 and the variant exhibits a change in a biological activity. Amino acid changes occurring in the transmembrane domain of ErbB2, such as at position 655, can be associated with increased risk of prostate cancer, gastric cancer, or breast cancer. An exemplary ErbB2 allelic variant containing one or more amino acid changes described above is set forth as SEQ ID NO: 299.

Provided herein are exemplary ErbB2 isoforms that lack one or more domains or a part thereof compared to a cognate ErbB2 such as set forth in SEQ ID NO:266. Included are exemplary ErbB2 isoforms that lack a transmembrane domain and lack a kinase domain, such as the polypeptides set forth in SEQ ID NOS: 96-98 and 108. Such isoforms can contain other domains of ErbB2. For example, the exemplary ErbB2 isoform set forth as SEQ ID NO: 96 is characterized by two Receptor L Domains between amino acids 54-175 and amino acids 368-488, and a Furin-like cysteine rich region between amino acids 191-345. The exemplary ErbB2 isoform set forth as SEQ ID NOS: 97 and 98 are characterized by two Receptor L Domains between amino acids 52-173 and amino acids 366-486, and a furin-like cysteine rich region between amino acids 189-343. The exemplary ErbB2 isoform set forth as SEQ ID NO: 108 is characterized by a portion of a Receptor L Domain between amino acids 52-75.

ErbB2 isoforms can be used to modulate RTKs such as in the treatment of cancers characterized by the overexpression of EGFR receptors such as those characterized by overexpression of ErbB2 and/or ErbB3. ErbB-2 isoforms can be used as a treatment for autoimmune diseases which involve EGFR family members in the maintenance of inflammation and hyperproliferation, including asthma. ErbB2 isoforms also can be used to target RTKs in conditions including Menetrier's disease, Alzheimer's disease and as modulators, for example as an antagonist for bone resorption.

b. ErbB3

ErbB3 also is a member of the EGF receptor family involved in regulating development of neuronal survival and synaptogenesis, astrocytic differentiation and microglial activation. The ErbB3 protein (GenBank No. NP_{—}001973 set forth as SEQ ID NO:267) is characterized by two Receptor L Domains between amino acids 55-167 and between amino acids 353-474; a Furin-like cysteine rich region between amino acids 180-332; transmembrane domain between amino acids 644-666; and protein kinase domain between amino acids 709-965. The ligand for ErbB3 is NRG-1. Although NRG-1 can bind to ErbB3 and ErbB4, ErbB3 binds NRG-1 with an affinity an order of magnitude lower than ErbB4. ErbB3 has lower tyrosine kinase activity compared to other members of the EGFR family. It is capable of recruiting alternative signaling molecules, for example, phosphatidylinositol-3 kinase. ErbB3 overexpression has been implicated in a number of human cancers such as breast, lung and bladder cancers and adenocarcinomas.

ErbB3 isoforms can be used to target RTKs such as in the treatment of cancers characterized by the overexpression of EGFR receptors such as those characterized by overexpression of ErbB2 and/or ErbB3. ErbB3 isoforms can target ErbB3 homodimers. ErbB3 isoforms can target ErbB2 through heterodimerization of an ErbB3 isoform with ErbB2. ErbB3 isoforms can be used for treatment of diseases and conditions in which EGFR receptors are involved. For example, ErbB3 isoforms can be used as a treatment for autoimmune diseases which involve EGFR family members in the maintenance of inflammation and hyperproliferation, including asthma. ErbB3 isoforms also can be used to target RTKs in conditions including Menetrier's disease, Alzheimer's disease and as modulators, for example as an antagonist for bone resorption.

2. Discoidin Domain Receptors—DDR1

Discoidin domain receptors (e.g. DDR1) are a family of RTKs that are thought to play a role in cell adhesion. The DDR1 protein (GenBank No. NP_{—}054699 set forth as SEQ ID NO: 250) is characterized by a F5/8 type C domain, also known as the discoidin (DS) domain, between amino acids 46-182; the transmembrane domain between amino acids 417-439; and protein kinase domain between amino acids 610-913. The discoidin domain is a unique structural motif in the extracellular domain that is homologous to the Dictyostelium discoideum (slime mold) protein discoidin-1, a carbohydrate-binding protein involved in cell aggregation. The discoidin-like domain, although not found in other RTKs, is found in other extracellular molecules that are known to interact with cellular membrane proteins (e.g., coagulation factors V and VIII).

DDR1 proteins include allelic variants of DDR1. In one example, an allelic variant contains one or more amino acid changes compared to SEQ ID NO:250. For example, one or more amino acid variations can occur in the F5/8 type C or discoidin domain of DDR1. An allelic variant can include amino acid changes at position 53 where, for example, W can be replaced by A, or at position 55 where, for example, D can be replaced by A, or at position 66 where, for example, S can be replaced by A, or at position 68 where, for example, D can be replaced by A, or at position 105 where, for example, R can be replaced by A, or at position 106 where, for example, H can be replaced by A, or at position 110 where, for example, L can be replaced by A, or at position 112 where, for example, K can be replaced by A, or at position 173 where, for example, V can be replaced by A, or at position 174 where, for example, M can be replaced by A, or at position 175 where, for example, S can be replaced by A. In one example, an allelic variant includes one or more amino acid changes compared to SEQ ID NO:250 and the variant exhibits a change in a biological activity. Amino acid changes occurring in the discoidin domain of DDR1, such as those at position 105 and 175, can result in reduced activation and phosphorylation of DDR1 due to an inability to bind to collagen. Other amino acid changes in the discoidin domain of DDR1, such as those at positions 106, 173, and 174, can result in a marked reduction in the ability of DDR1 to bind to collagen. An exemplary DDR1 allelic variant containing one or more amino acid changes described above is set forth as SEQ ID NO: 286.

DDRs are widely expressed in fetal and adult organs and tissues. DDR1 is expressed primarily in epithelial cells in brain, lung, kidney and gastrointestinal tract, whereas DDR2 is expressed in brain, heart, and muscle. DDR also may play an important role in brain development. DDR tyrosine kinases have been linked to human cancers. For example, DDR1 can bind to collagen (e.g. types I through VI) and mediate collagen-induced activation of matrix metalloproteinase-1. Matrix metalloproteinase-1 is involved in the degradation of extracellular matrix, which allows neoplastic cells to metastasize. Overexpression of DDR1 has been linked to cancers such as breast, ovarian and esophageal cancers and a variety of central nervous system neoplasms, such as pediatric brain cancers. Activation of DDR1 also has been implicated in inflammatory responses.

Exemplary DDR isoforms include DDR1 isoforms set forth in SEQ ID NO: 106, 115 and 117. These exemplary DDR1 isoforms lack one or more domains or a part thereof compared to a cognate DDR1 such as set forth in SEQ ID NO:250. The exemplary DDR1 isoforms set forth as SEQ ID NOS: 106, 115, and 117 contain an F5/8 type C domain between amino acids 46-182, and lack the transmembrane and protein kinase domains.

DDR1 isoforms, including DDR1 isoforms herein, can include allelic variation in the DDR1 polypeptide. For example, a DDR1 isoform can include one or more amino acid differences present in an allelic variant. In one example, a DDR1 isoform includes one or more allelic variation as set forth in SEQ ID NO:286. Examples of allelic variation include variants in the F5/8 type C and discoidin domains, including, but not limited to amino acid variation at positions corresponding to amino acids 53, 55, 66, 68, 105, 106, 110, 113, 173, 174, or 175 of SEQ ID NO:286.

DDR1 isoforms can be used to modulate DDR1 RTK. For example, a DDR1 isoform can be used to down regulate DDR1 overexpression and or activation in diseases and conditions in which DDR1 is involved.

3. Eph Receptors

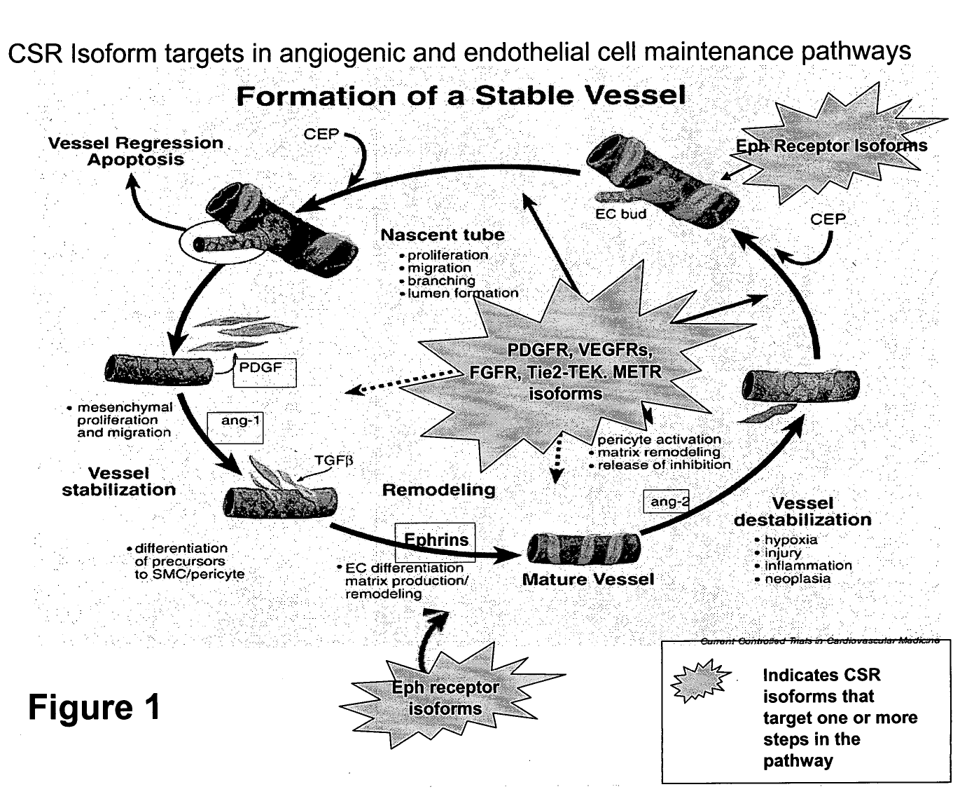

Eph receptors (erythropoietin-producing hepatocellular receptors; also referred to as ephrin receptors) are the largest known family of RTKs. The ligands for Eph receptors are ephrins (Eph receptor interacting protein). The Eph and Ephrin system includes at least fourteen Eph receptor tyrosine kinase proteins and nine ephrin membrane ligands. The Eph receptors and Ephrin membrane proteins play important roles in disease and development (see, e.g., FIG. 1). For example, binding of cell surface Eph and ephrin proteins results in bi-directional signals that regulate the cytoskeletal, adhesive and motile properties of the interacting cells. Through these signals Eph and Ephrin proteins are involved in early embryonic cell movements, which establish the germ layers, and in cell movements involved in formation of tissue boundaries and the pathfinding of axons. Ligand and receptor are membrane-bound molecules and signaling can occur through either protein. The ephrins have been separated into two classes based on the manner in which they are anchored to the cell membrane; type A ligands are linked to the cell membrane by a glycosylpho-phatidylinositol (GPI) linkage and type B ligands encode for a transmembrane domain. Eph receptors include, but are not limited to, EphA1, EphA2, EphA3, EphA4, EphA5, EphA6, EphA7, EphA8, EphB1, EphB2, EphB3, EphB4, EphB5, EphB6.

Ephrin receptors are characterized by a cytoplasmic tyrosine kinase domain, a conserved cysteine-rich domain, two fibronectin type III domains and an immunoglobulin-like N-terminal ligand binding domain. Further, two tyrosine residues near the transmembrane domain are highly conserved and phosphorylated in response to ligand binding and appear to be critical for enzymatic function. Other sites of protein-protein interaction also are mediated by sterile alpha motifs and postsynaptic density protein, disc large, zona occludens binding motifs located near the C-terminal end of some Eph receptors. Sterile alpha motifs (SAM) mediate cell-cell initiated signal transduction via the binding of SH2-containing proteins to a conserved tyrosine that is phosphorylated and in many cases mediates homodimerization.

The Eph family of RTKs is involved in a variety of cellular processes, including embryonic patterning, neuronal targeting, vascular development and angiogenesis. Particularly due to a role in angiogenesis, Eph receptors have been implicated in human cancers, such as breast cancer. Misregulation of EphA receptors also are involved in pathological conditions. For example, upregulation of the EphA receptor tyrosine kinase stimulates vascular endothelial cell growth factor (VEGF)-induced angiogenesis, common in certain eye diseases, rheumatoid arthritis and cancer. An EphA isoform, such as an isoform acting as an EphA receptor antagonist can be used to block or inhibit inappropriate angiogenesis. EphB receptors have been implicated in cancers such as colorectal cancers. EphB receptors also play a role in dendritic spine development (post-synaptic targets for excitatory synapses) and may be implicated in neurodegenerative disorders. Exemplary EphA and EphB isoforms are set forth in SEQ ID NOS: 107, 149, 151, 153, 155, 168, 170, 172, and 174.

a. EphA1

EphA1 is a type A Eph receptor. The EphA1 protein (GenBank No. NP_{—}005223 set forth as SEQ ID NO:253) is characterized by an Ephrin ligand binding domain between amino acids 27-204, two fibronectin type III domains between amino acids 333-431 and between amino acids 448-528; a transmembrane domain between amino acids 548-570; protein kinase domain between amino acids 624-880, and two SAM domains (SAM-1 between amino acids 911-975, and SAM-2 between amino acids 910-976) at the carboxy terminus.

EphA1 proteins include allelic variants of EphA1. In one example, an allelic variant contains one or more amino acid changes compared to SEQ ID NO:253, such as the allelic variations set forth in SEQ ID NO:289. One or more amino acid variations can occur, for example, in the ephrin ligand binding domain of EphA1, such as an amino acid change at position 160 where, for example, A can be replaced by V.

Type A Eph receptors bind to type A ephrins, which are linked to cell membranes via a GPI anchor. EphA1 is expressed widely in differentiated epithelial cells, including skin, adult thymus, kidney and adrenal cortex. Overexpression of EphA1 has been implicated in a variety of human cancers, including head and neck cancer. EphA1 isoforms can be used to target such diseases and other conditions in which Eph receptors have been implicated.

Exemplary EphA1 isoforms include EphA1 isoforms set forth in SEQ ID NOS: 107, 149, 151, and 153. These exemplary EphA1 isoforms lack one or more domains or a part thereof compared to a cognate EphA1 such as set forth in SEQ ID NO:253. The exemplary EphA1 isoforms set forth as SEQ ID NOS:149 and 153 contain an ephrin ligand binding domain between amino acids 27-204 and one of two fibronectin type III domains between amino acids 333-431. The isoform set forth as SEQ ID NO: 149 lacks a fibronectin type III domain, a transmembrane domain, protein kinase domain, and two SAM domains compared to the cognate receptor. The exemplary EphA1 isoform set forth as SEQ ID NO: 151 contains the ephrin ligand binding domain between amino acids 27-204, but does not contain fibronectin type III domains, transmembrane domain, protein kinase domain and SAM domains. The exemplary EphA1 isoform set forth as SEQ ID NO: 107 contains the ephrin ligand binding domain between amino acids 1-114, but does not contain fibronectin type III domains, transmembrane domain, protein kinase domain and SAM domains.

EphA1 isoforms, including EphA1 isoforms herein, can include allelic variation in the EphA1 polypeptide. For example, an EphA1 isoform can include one or more amino acid differences present in an allelic variant. In one example, an EphA1 isoform includes one or more allelic variations as set forth in SEQ ID NO:289. An allelic variation can include one or more amino acid changes in the ephrin ligand binding domain, such as at position 160.

b. EphA2

EphA2 binds ephrin-A3, ephrin-A1, ephrin-A4, an ephrin-A2. EphA2 expression is frequently elevated in cancer and is highly expressed in tumor tissues including breast, prostate, non-small cell lung cancers, colon, kidney, lung, ovary, stomach, uterus, and aggressive melanomas. EphA2 has also been found in Schwann cells, the primitive streak and hindbrain in restricted expression pattern. It has been suggested that EphA2 does not simply function as a marker, but as an active participant in malignant progression. The normal cellular functions of EphA2 are not well understood, but tumor-based models suggests potential roles for EphA2 in the regulation of cell growth, survival, migration, and angiogenesis.

The EphA2 receptor set forth as SEQ ID NO:254 (GenBank No. NP_{—}004422) is characterized by an ephrin ligand binding domain between amino acids 28-201, two fibronectin type III domains between amino acids 329-424 and between amino acids 436-519, a transmembrane domain between amino acids 536-558, protein kinase domain between amino acids 613-871; and two SAM domains (SAM-1 between amino acids 902-966, and SAM-2 between amino acids 901-968) at the carboxy terminus.

EphA2 proteins include allelic variants of EphA2. In one example, an allelic variant contains one or more amino acid changes compared to positions corresponding to the amino acid sequence set forth as SEQ ID NO:254. For example, one or more amino acid variations can occur in the ephrin ligand binding domain of EphA2. An allelic variant can include amino acid changes at position 94 (SNP NO: 1058370) where, for example, I can be replaced by N, or at position 96 (SNP NO: 1058371) where, for example, I can be replaced by F, or at position 99 (SNP NO: 1058372) where, for example, K can be replaced by N. Additional examples of allelic variation can occur in the fibronectin type III domain. An allelic variant can include amino acid changes at position 350 (SNP NO: 11543934) where, for example, P is replaced by T. One or more amino acid variations also can occur in the protein kinase domain. An allelic variant can include amino acid changes at position 825 where, for example, E can be replaced by K. An exemplary EphA2 allelic variant containing one or more amino acid changes described above is set forth as SEQ ID NO: 290.

Exemplary EphA2 isoforms lack one or more domains or a part thereof compared to a cognate EphA2 such as set forth in SEQ ID NO:254. The exemplary EphA2 isoform set forth as SEQ ID NO: 168 contains an ephrin ligand binding domain between amino acids 28-201, a fibronectin type III domain between amino acids 329-424 and a portion of another fibronectin type III domain between amino acids 436-497. SEQ ID NO: 168 does not contain the transmembrane, protein kinase, and SAM domains. EphA2 isoforms, including EphA2 isoforms herein, can include allelic variation in the EphA2 polypeptide. For example, an EphA2 isoform can include one or more amino acid difference present in an allelic variant. In one example, an EphA2 isoform includes one or more allelic variations as set forth in SEQ ID NO:290. An allelic variation can include a position corresponding to amino acid positions 94, 96, or 99 in SEQ ID NO:254, or for example, in the fibronectin type III domain, such as at a position corresponding to amino acid 350 in SEQ ID NO:254.

C. EphA8

EphA8 is a type A Eph receptor. Type A Eph receptors bind to type A ephrins, which are linked to cell membranes via a GPI anchor. EphA8 has been implicated in cell migration and cell adhesion as well as nervous system development, including axon guidance. EphA8 isoforms can be used to target such diseases and other conditions in which Eph receptors have been implicated.

The EphA8 receptor (GenBank No. NP_{—}065387 set forth as SEQ ID NO:260) is characterized by an Ephrin ligand binding domain between amino acids 31-204, two fibronectin type III domains between amino acids 329-425 and amino acids 437-524, a transmembrane domain between amino acids 541-563, protein kinase domain between 635-892 and two SAM domains (SAM-1 between amino acids 931-992 and SAM-2 between amino acids 927-994).

EphA8 proteins include allelic variants of EphA8. In one example, an allelic variant contains one or more amino acid changes compared to positions corresponding to the amino acid sequence set forth as SEQ ID NO:260. For example, one or more amino acid variations can occur in the fibronectin type III domain of EphA8. An allelic variant can include amino acid changes at position 444 (SNP NO: 2295021) where, for example, V can be replaced by M. Allelic variations also can occur at position 301 (SNP NO: 638524) where, for example, A can be replaced by V, or at position 612 (SNP NO:999765) where, for example, E can be replaced by Q. An exemplary EphA8 allelic variant containing one or more amino acid changes described above is set forth as SEQ ID NO: 293.

d. EphB1

EphB1 has been shown to bind to ephrin-B2, ephrin-B1, ephrin-A3, ephrin-A1 and ephrin-B3. EphB1 is expressed in developing and adult neural tissue. EphB1 signaling pathways impact responses relevant to vascular development, including cell attachment, migration and capillary-like assembly responses.

The EphB1 protein (GenBank No. NP_{—}004432 set forth as SEQ ID NO:261) is characterized by an Ephrin ligand binding domain between amino acids 19-196, two fibronectin type III domains between amino acids 323-414 and between amino acids 434-518, transmembrane domain between amino acids 541-563, protein kinase domain between amino acids 619-878, and two SAM domains (SAM-1 between amino acids 909-973, and SAM-2 between amino acids 908-975) at the carboxy terminus.

EphB1 proteins include allelic variants of EphB1. In one example, an allelic variant contains one or more amino acid changes compared to positions corresponding to the amino acid sequence set forth as SEQ ID NO:261. For example, one or more amino acid variations can occur in the ephrin ligand binding domain of EphB1. An allelic variant can include amino acid changes at position 87 (SNP NO:1042794) where, for example, T can be replaced by S, or at position 152 (SNP NO: 1042793 where, for example, G can be replaced by R. Additional examples of amino acid changes can occur in the fibronectin type III domain. An allelic variant can include amino acid changes at position 367 (SNP NO: 1042789) where, for example, R is replaced by G, or at position 485 (SNP NO: 1042788) where, for example, R is replaced by S. One or more amino acid changes also can occur in the protein kinase domain. An allelic variant can include amino acid changes at position 813 (SNP NO:1042786) where, for example, V can be replaced by I, or at position 847 (SNP NO:1042785) where, for example, M can be replaced by T. Another example of amino acid changes can occur in the SAM domain. An allelic variant can include amino acid changes at position 973 (SNP NO:1042784) where, for example, R is replaced by W. Allelic variations also can occur at position 274 (SNP NO: 1126906) where, for example, T is replaced by R. An exemplary EphB1 allelic variant containing one or more amino acid changes described above is set forth as SEQ ID NO: 294.

Exemplary EphB1 isoforms lack one or more domains or a part thereof compared to a cognate EphB1 such as set forth in SEQ ID NO:261. The exemplary EphB1 isoform set forth as SEQ ID NO: 155 contains a portion of an ephrin ligand binding domain between amino acids 19-167 and lacks fibronectin type III domains, transmembrane domain, protein kinase domain, and SAM domains compared with a cognate EphB1 receptor (e.g. SEQ ID NO:261).

EphB1 isoforms, including EphB1 isoforms herein, can include allelic variation in the EphB1 polypeptide. For example an EphB1 isoform can include one or more amino acid differences present in an allelic variant. In one example, an EphB1 isoform includes one or more allelic variation as set forth in SEQ ID NO:294. An allelic variation can include one or more amino acid changes in the ephrin ligand binding domain, such as positions corresponding to amino acid positions 87 and 152 of SEQ ID NO:261.

e. EphB4

EphB4 receptors bind to ephrin-B2 and ephrin-B1 proteins. Ephrin-B proteins transduce signals, such that bidirectional signaling can occur upon interaction with Eph receptor.

The EphB4 receptor polypeptide (GenBank No. NP_{—}004435 set forth as SEQ ID NO:264) is characterized by an ephrin ligand binding domain between amino acids 17-197, two fibronectin type III domains between amino acids 324-414 and between amino acids 434-519, transmembrane domain between amino acids 541-563, cytoplasmic protein kinase domain between 615-874, and two SAM domains (SAM-1 between amino acids 905-969, and SAM-2 between amino acids 904-971) at the carboxy terminus.

EphB4 proteins can include allelic variants of EphB4. In one example, an allelic variant contains one or more amino acid changes compared to SEQ ID NO:264. For example, one or more amino acid variations can occur in the fibronectin type III domain of EphB4. An allelic variant can include amino acid changes at position 463 (SNP NO:7457245) where, for example, A can be replaced by D, or at position 471 (SNP NO:3891495) where, for example, Y can be replaced by D. Additional amino acid changes can occur in the SAM domain. An allelic variant can include amino acid changes at position 926 (SNP NO: 1056997) where, for example, E can be replaced by D. An exemplary EphB4 allelic variant containing one or more amino acid changes described above is set forth as SEQ ID NO: 297.

Exemplary EphB4 isoforms include the EphB4 isoforms set forth in SEQ ID NO: 170, 172 and 174. These exemplary EphB4 isoforms lack one or more domains or a part thereof compared to a cognate EphB4 such as set forth in SEQ ID NO:264. The exemplary EphB4 isoform set forth as SEQ ID NO: 170 contains an ephrin ligand binding domain between amino acids 17-197. SEQ ID NO: 170 does not contain fibronectin type III domains, transmembrane domain, protein kinase domain, and SAM domains. The exemplary EphB4 isoform set forth as SEQ ID NO: 172 contains an ephrin ligand binding domain between amino acids 17-197, a fibronectin type III domain between amino acids 324-414 and a portion of another fibronectin type III domain between amino acids 434-514. SEQ ID NO: 172 does not contain the transmembrane domain, protein kinase domain, and SAM domains. The exemplary EphB4 isoform set forth as SEQ ID NO: 174 contains an ephrin ligand binding domain between amino acids 17-197 and a portion of a fibronectin type III domain between amino acids 324-413. SEQ ID NO: 174 does not contain the second fibronectin type III domain, transmembrane domain, protein kinase domain, and SAM domains.

EphB4 isoforms, including EphB4 isoforms herein, can include allelic variation in the EphB4 polypeptide. For example an EphB4 isoform can include one or more amino acid differences present in an allelic variant. In one example, an EphB4 isoform includes one or more allelic variation as set forth in SEQ ID NO:297. An allelic variation can include one or more amino acid changes in the fibronectin type III domain, such as at positions corresponding to amino acid positions 463 or 471 of SEQ ID NO:264.

4. Fibroblast Growth Factor Receptors

The fibroblast growth factor receptor (FGFR) family includes FGFR-1, FGFR-2, FGFR-3, FGFR-4 and FGFR-5. There are at least 23 known FGF proteins that are capable of binding to one or more FGF receptors. FGF receptors are structurally characterized by three N-terminal Ig-like domains (extracellular), a transmembrane domain and the split tyrosine-kinase domain at the C-terminus (cytoplasmic). FGFs and their receptors are involved in stimulation of cellular proliferation, promoting angiogenesis and wound healing, and modulating cell motility and differentiation. FGFRs have been implicated in a variety of human cancers as well as diseases of the eye.

a. FGFR-1

FGFR-1 has specificity for FGF-1, -2, and -4 and is expressed in a number of cell types including fibroblasts, endothelial cells, certain epithelial cells, vascular smooth muscle cells, lymphocytes, macrophages, and numerous tumor cells.

The FGFR-1 polypeptide (GenBank No. AAA35835 set forth as SEQ ID NO:268) is characterized by three immunoglobulin-like domains; domain 1 between amino acids 35-119, domain 2 between amino acids 156-246, and domain 3 between amino acids 253-357. FGFR-1 also has a transmembrane domain between amino acids 375-397 and protein kinase domain between amino acids 476-752.

FGFR-1 proteins include allelic variants of FGFR-1. In one example, an allelic variant contains one or more amino acid changes compared to positions corresponding to the amino acid sequence set forth as SEQ ID NO:268. For example, one or more amino acid variations can occur in the immunoglobulin domain of FGFR-1. An allelic variant can include amino acid changes at position 97 where, for example, G can be replaced by D, or at position 99 where, for example, Y can be replaced by C, or at position 165 where, for example, A can be replaced by S, or at position 190 where, for example, K can be replaced by E, or at position 192 where, for example, S can be replaced by G, or at position 198 where, for example, D can be replaced by G, or at position 275 where, for example, C can be replaced by Y. Additional amino acid changes can occur in the protein kinase domain. An allelic variant can include amino acid changes at position 605 where, for example, V can be replaced by M, or at position 664 where, for example, W can be replaced by R, or at position 717 where, for example, M can be replaced by R. One or more amino acid change also can occur at position 22 where, for example, R can be replaced by S, or at position 250 where, for example P can be replaced by R, or at position 770 where, for example, P can be replaced by S, or at position 816 where, for example G can be replaced by R, or at position 820 where, for example, R can be replaced by C. In one example, an allelic variant includes one or more amino acid change compared to SEQ ID NO:268 and the variant exhibits a change in a biological activity. Polypeptides containing amino acid changes in either the immunoglobulin or protein kinase domain of FGFR-1, such as those at positions 97, 99, 165, 275, 605, 664, or 717, can be characterized as loss-of function mutations. In the context of a cognate receptor (such as SEQ ID NO: 268) such changes cause autosomal dominant Kallmann syndrome. Amino acid changes occurring in the protein kinase domain, such as at position 717, can impair PLC gamma association with the receptor and inhibit FGF-mediated phosphotidylinositol and Ca2+ mobilization; these changes, however, do not affect FGF-mediated mitogenesis. Additional allelic variants, such at position 250, can be associated with autosomal dominant skeletal disorders such as Pfeiffer syndrome. An exemplary FGFR-1 allelic variant containing one or more amino acid changes described above is set forth as SEQ ID NO:300.

Exemplary FGFR-1 isoforms include FGFR-1 isoforms set forth in SEQ ID NOS: 119 and 176. These exemplary FGFR-1 isoforms lack one or more domains or a part thereof compared to a cognate FGFR-1 such as set forth in SEQ ID NO:268. The exemplary FGFR-1 isoform set forth as SEQ ID NO: 119 contains immunoglobulin-like domain 2 between amino acids 67-157 and a portion of immunoglobulin-like domain 3 between amino acids 164-220. The exemplary FGFR-1 isoform set forth as SEQ ID NO: 176 contains immunoglobulin-like domain 2 between amino acids 70-159 and immunoglobulin-like domain 3 between amino acids 166-268. These exemplary isoforms each lack the transmembrane and protein kinase domains compared to a cognate FGFR-1 polypeptide (e.g. SEQ ID NO:268).

FGFR-1 isoforms, including FGFR-1 isoforms herein, can include allelic variation in the FGFR-1 polypeptide. For example, a FGFR-1 isoform can include one or more amino acid differences present in an allelic variant. In one example, a FGFR-1 isoform includes one or more allelic variation as set forth in SEQ ID NO:300. An allelic variant can include one or more amino acid change in the immunoglobulin domain, such as at positions corresponding to amino acid positions 97, 99, 165, 190, 192, and 198 of SEQ ID NO:268. An additional allelic variant can include one or more amino acid changes at a position corresponding to amino acid position 22 of SEQ ID NO:268.

b. FGFR-2

FGFR-2 is a member of the fibroblast growth factor receptor family. Ligands to FGFR-2 include a number of FGF proteins, such as, but not limited to, FGF-1 (basic FGF), FGF-2 (acidic FGF), FGF-4 and FGF-7. FGF receptors are involved in cell-cell communication of tissue remodeling during development as well as cellular homeostasis in adult tissues. Overexpression of, or mutations in, FGFR-2 have been associated with hyperproliferative diseases, including a variety of human cancers, including breast, pancreatic, colorectal, bladder and cervical malignancies. FGFR-2 isoforms such as FGFR-2 intron fusion proteins can be used to treat conditions in which FGFR-2 is upregulated, including cancers.

The FGFR-2 protein (GenBank No. NP_{—}000132 set forth as SEQ ID NO:269) is characterized by three immunoglobulin-like domains; domain 1 between amino acids 41-125, domain 2 between amino acids 159-249, and domain 3 between amino acids 256-360. FGFR-2 also contains a transmembrane domain between amino acids 378-400 and protein kinase domain between amino acids 481-757.

FGFR-2 proteins include allelic variants of FGFR-2. In one example, an allelic variant contains one or more amino acid changes compared to SEQ ID NO:269. For example, one or more amino acid variations can occur in the immunoglobulin domain of FGFR-2. An allelic variant can include amino acid changes at position 105 where, for example Y can be replaced by C, or at position 162 where, for example, M can be replaced by T, or at position 172 where, for example, A can be replaced by F, or at position 186 (SNP NO: 755793) where, for example, M can be replaced by T, or at position 267 where, for example, S can be replaced by P, or at position 276 where, for example, F can be replaced by V, or at position 278 where, for example, C can be replaced by F, or at position 281 where, for example, Y can be replaced by C, or at position 289 where, for example, Q can be replaced by P, or at position 290 where, for example, W can be replaced by C, or at position 315 where, for example, A can be replaced by S, or at position 338 where, for example, G can be replaced by R, or at position 340 where, for example, Y can be replaced by H, or at position 341 where, for example, T can be replaced by P, or at position 342 where, for example, C can be replaced by R, Y, S, F, or W, or at position 344 where, for example, A can be replaced by P or G, or at position 347 where, for example, S can be replaced by C, or at position 351 where, for example, S can be replaced by C, or at position 354 where, for example, S can be replaced by C. Further examples of amino acid changes can occur in the transmembrane domain. An allelic variant can include amino acid changes at position 384 where, for example, G can be replaced by R. Additional amino acid changes also can occur in the protein kinase domain. An allelic variant can include amino acid changes at position 549 where, for example, N can be replaced by H, or at position 565 where, for example, E can be replaced by G, or at position 641 where, for example, K can be replaced by R, or at position 659 where, for example, K can be replaced by N, or at position 663 where, for example, G can be replaced by E, or at position 678 where, for example, R can be replaced by G. Allelic variations also can occur at position 6 where, for example, R can be replaced by P, or at position 31 where, for example, T can be replaced by I, or at position 152 where, for example, R can be replaced by G, or at position 252 where, for example, S can be replaced by W or L, or at position 253 where, for example, P can be replaced by S or R, or at position 372 where, for example, S can be replaced by C, or at position 375 where, for example, Y can be replaced by C. In one example, an allelic variant includes one or more amino acid change compared to SEQ ID NO:269 and the variant exhibits a change in a biological activity. Amino acid changes occurring in the immunoglobulin domain, such as at positions 105, 172, 267, 276, 278, 281, 289, 290, 315, 338, 340, 341, 342, 344, 347, 351, 354, or the protein kinase domain, such as at positions 549, 565, 641, 659, 663, or 678, or other amino acid changes, such as at positions 252, 253, or 375, are associated with syndromic craniosynostosis including Apert, Crouzon, or Pfeiffer syndromes when such amino acid changes are present in a cognate FGFR-2 such as set forth in SEQ ID NO: 269. An exemplary FGFR-2 allelic variant containing one or more amino acid changes described above is set forth as SEQ ID NO: 301.

Exemplary FGFR-2 isoforms include FGFR-2 isoforms set forth in SEQ ID NOS: 178, 180, 182 and 184. These exemplary FGFR-2 isoforms lack one or more domains or a part thereof compared to a cognate FGFR-2 such as set forth in SEQ ID NO:269. The exemplary FGFR-2 isoform set forth as SEQ ID NO: 184 contains three immunoglobulin-like domains; domain 1 between amino acids 41-125, domain 2 between amino acids 159-249 and domain 3 between amino acids 256-360, but lacks transmembrane and protein kinase domains. The exemplary FGFR-2 isoform set forth as SEQ ID NO: 180 contains the immunoglobulin-like domains 1, 2 and a portion of domain 3 (between amino acids 41-125, 159-249 and 256-313, respectively), but is missing transmembrane and protein kinase domains. The exemplary FGFR-2 isoform set forth as SEQ ID NO: 178 contains immunoglobulin-like domain 1 between amino acids 41-125 and domain 2 between amino acids 159-249, but lacks immunoglobulin-like domain 3, and transmembrane and protein kinase domains. The exemplary FGFR-2 isoform set forth as SEQ ID NO: 182 contains immunoglobulin-like domains 2 between amino acids 44-134 and domain 3 between amino acids 141-245, but does not contain an immunoglobulin-like domain 1, a transmembrane domain and protein kinase domain.

FGFR-2 isoforms, including FGFR-2 isoforms herein, can include allelic variation in the FGFR-2 polypeptide. For example, a FGFR-2 isoform can include one or more amino acid differences present in an allelic variant. In one example, a FGFR-2 isoform includes one or more allelic variation as set forth in SEQ ID NO:301. An allelic variation can include one or more amino acid changes in the immunoglobulin domain, such as at positions 105, 162, 172, 186, 267, 276, 278, 281, 289, 290, 315, 338, 340, 341, 342, 344, 347, 351, or 354. Additional allelic variations can include one or more amino acid changes, such as at positions 6, 31, 152, 252, or 253.

C. FGFR-4

FGFR-4 is a member of the FGF receptor tyrosine kinase family. FGFR-4 regulation is modified in some cancer cells. For example, in some adenocarcinomas FGFR-4 is down-regulated compared with expression in normal fibroblast cells. Alternate forms of FGFR-4, are expressed in some tumor cells. For example, ptd-FGFR-4 lacks a portion of the FGFR-4 extracellular domain but contains the third Ig-like domain, a transmembrane domain and a kinase domain. This isoform is found in pituitary gland tumors and is tumorigenic. FGFR-4 isoforms can be used to treat diseases and conditions in which FGFR4 is misregulated. For example, an FGFR-4 isoform can be used to down regulate tumorigenic FGFR-4 isoforms such as ptd-FGFR-4.

The FGFR-4 protein (GenBank No. NP_{—}002002 set forth as SEQ ID NO: 271) is characterized by three immunoglobulin-like domains; domain 1 between amino acids 35-113, domain 2 between amino acids 152-242, and domain 3 between amino acids 249-351. FGFR-4 also contains a transmembrane domain between amino acids 370-386 and protein kinase domain between amino acids 467-743.

FGFR-4 proteins include allelic variants of FGFR-4. In one example, an allelic variant contains one or more amino acid changes compared to SEQ ID NO:271. For example, one or more amino acid variations can occur in the immunoglobulin domain of FGFR-4. An allelic variant can include amino acid changes at position 275 (SNP NO: 11954456) where, for example, S is replaced by R, or at position 297 (SNP NO:1057633) where, for example, D is replaced by V. Additional amino acid changes can occur in the protein kinase domain. An allelic variant can include an amino acid change at position 616 (SNP NO:2301344) where, for example, R can be replaced by L. Allelic variations also can occur at position 10 (SNP NO: 1966265) where, for example, V can be replaced by I, or at position 136 (SNP NO: 376618) where, for example, P can be replaced by L, or at position 388 (SNP NO: 351855) where, for example, G can be replaced by R. An exemplary FGFR-4 allelic variant containing one or more amino acid changes described above is set forth as SEQ ID NO: 303.

Exemplary FGFR-4 isoforms lack one or more domains or a part thereof compared to a cognate FGFR-4 such as set forth in SEQ ID NO:271. Exemplary FGFR-4 isoforms include FGFR-4-isoforms set forth in SEQ ID NOS: 91, 109 and 121. The exemplary FGFR-4 isoform set forth as SEQ ID NO: 121 contains immunoglobulin-like domain 1 between amino acids 35-113, domain 2 between amino acids 152-242, and domain 3 between amino acids 249-351, but lacks a transmembrane and protein kinase domains. The exemplary FGFR-4 isoform set forth as SEQ ID NO: 109 contains immunoglobulin-like domain 2 between amino acids 62-154 and a portion of domain 3 between amino acids 161-209, but does not contain an immunoglobulin-like domain 1, a transmembrane and protein kinase domains. The exemplary FGFR-4 isoform set forth as SEQ ID NO: 91 lacks the immunoglobulin-like domains, the transmembrane domain and the protein kinase domain present in the cognate receptor (e.g. SEQ ID NO:271).

FGFR-4 isoforms, including FGFR-4 isoforms herein, can include allelic variation in the FGFR-4 polypeptide. For example, a FGFR-4 isoform can include one or more amino acid differences present in an allelic variant. In one example, a FGFR-4 isoform includes one or more allelic variation as set forth in SEQ ID NO:303. An allelic variation can include one or more amino acid changes in the immunoglobulin domain, such as at amino acids corresponding to positions 275 or 297 of SEQ ID NO:271. Additional allelic variants can include one or more amino acid changes, such as at amino acids corresponding to amino acid positions 10 or 136 of SEQ ID NO:271.

5. Platelet-Derived Growth Factor Receptors

Platelet-derived growth factor receptors (PDGFRs) are homo or heterodimers that contain two subunits, α and β. Receptor subunits are comprised of five Ig-like domains at the N-terminus, a transmembrane domain, and a split kinase domain at the C-terminus.

The PDGFR-A protein (GenBank No. NP_{—}006197 set forth as SEQ ID NO: 275) is characterized by three immunoglobulin-like domains; domain 1 between amino acids 42-102, domain 2 between amino acids 228-292, and domain 3 between amino acids 319-412. PDGFR-A also contains a transmembrane domain between amino acids 527-549 and protein kinase domain between amino acids 593-953. The PDGFR-B protein (GenBank No. NP_{—}002600 set forth as SEQ ID NO: 276) is characterized by two immunoglobulin-like domains between amino acids 32-119 and amino acids 213-311, a transmembrane domain between amino acids 534-556, and protein kinase domain between amino acids 600-958.

PDGF receptors can include allelic variation, for example, PDGFR-B and PDGFR-A allelic variants. In one example, an allelic variant contains one or more amino acid changes compared to SEQ ID NOS:275 or 276. For example, with respect to PDGFR-B, allelic variations can include one or more amino acid change at position 29 (SNP NO:17110944) where, for example, I is replaced by F, or at position 194 (SNP NO:2229560) where, for example, I is replaced by T, or at position 345 (SNP NO:2229558) where, for example, P is replaced by S. An exemplary PDGFR-B allelic variant containing one or more amino acid changes described above is set forth as SEQ ID NO: 307.

PDGF receptors and ligands are involved in a variety of cellular processes, including clot formation, extracellular matrix synthesis, chemotaxis of immune cells apoptosis and embryonic development. Overexpression of PDGF receptors has been linked to a number of human carcinomas, including stomach, pancreas, lung and prostate. Activation of the platelet derived growth factor receptor (PDGFR) is associated with benign prostatic hypertrophy and prostate cancer as well as other cancer types. Activation of PDGFR also is associated with smooth muscle proliferation in development of atherosclerosis. PDGFR also has been implicated in modulating proliferative vitreoretinopathy, a common medical problem caused by the proliferation of fibroblastic cells behind the retina, resulting in retinal detachment. Similar to its receptor, PDGF ligand is a homo or heterodimer of A and/or B chains. The α-PDGF receptor can be activated by either PDGF-A or PDGF-B. A β-PDGF receptor only can be activated by the PDGF-B chain. Two additional members of the PDGF family also have been isolated, PDGF-C and PDGF-D.

Exemplary PDGFR isoforms include the isoforms set forth in SEQ ID NO:111 and 147. These exemplary PDGFR isoforms lack one or more domains or a part thereof compared to a cognate PDGFR such as set forth in SEQ ID NO:276. The exemplary PDGFR-A isoform set forth as SEQ ID NO: 111 is characterized by one immunoglobulin-like domains between amino acids 41-102, but does not contain a transmembrane domain or protein kinase domain. The exemplary PDGFR-B isoform set forth as SEQ ID NO: 147 is characterized by two immunoglobulin-like domains between amino acids 32-119 and amino acids 213-310, but does not contain transmembrane domain or protein kinase domain.

PDGFR isoforms, including PDGFR isoforms herein, can include allelic variation in the PDGFR polypeptide. For example, a PDGFR isoform can include one or more amino acid differences present in an allelic variant. In one example, a PDGFR isoform includes one or more allelic variation as set forth in SEQ ID NO:307. An allelic variation can include one or more amino acid changes, such as at amino acids corresponding to positions 29 or 194 of SEQ ID NO:276.

PDGFR isoforms can be used to target diseases and conditions in which PDGFR is involved, including hyperproliferative diseases, such as proliferative vitreoretinopathy and smooth muscle hyperproliferative conditions including atherosclerosis.

Flt3 (fins-related tyrosine kinase 3), CSF1R (colony stimulating factor 1 receptor) and KIT (receptor for c-kit) also are members of the PDGFR RTK subfamily. The CSF1R protein (GenBank No. NP_{—}005202 set forth as SEQ ID NO: 249) is characterized by three immunoglobulin-like domains; domain 1 between amino acids 19-102, domain 2 between amino acids 202-324, and domain 3 between amino acids 412-487. CSF1R also is characterized by a transmembrane domain between amino acids 515-537 and protein kinase domain between amino acids 582-910. CSF1R proteins include allelic variants of CSF1R. In one example, an allelic variant contains one or more amino acid changes compared to a cognate CFS1R receptor such as set forth in SEQ ID NO:249. For example, one or more amino acid variations can occur in the immunoglobulin-like domain 2 of CSF1R. An allelic variant can include one or more amino acid changes as position 279 (SNP NO: 3829986) where, for example, V can be replaced by M. Allelic variants also can include amino acid changes at position 362 (SNP NO:10079250) where, for example, H can be replaced by R, or position 969 (SNP NO: 1801271 where, for example, Y can be replaced by C. An exemplary CSF1R allelic variant containing one or more amino acid changes described above is set forth as SEQ ID NO: 285.

The exemplary CSF1R isoform set forth as SEQ ID NO: 145 contains an immunoglobulin-like domain 1 between amino acids 19-102, a partial immunoglobulin-like domain 2 between amino acids 202-296. SEQ ID NO: 145 does not contain Ig-like domain 3, a transmembrane or protein kinase domain. CSF1R isoforms, including CSF1R isoforms herein, can include allelic variation in the CSF1R polypeptide. For example, a CSF1R isoform can include one or more amino acid differences present in an allelic variant. In one example, a CSF1R isoform includes one or more allelic variation as set forth in SEQ ID NO:285. An allelic variation can include one or more amino acid changes in the immunoglobulin-like domain 2, such as at positions 279. Allelic variations also can include one or more amino acid changes, such as at position 362.

The KIT receptor (GenBank No. NP_{—}000213 set forth as SEQ ID NO:273) is characterized by an immunoglobulin-like domain between amino acids 210-336, a transmembrane domain between amino acids 521-543, and protein kinase domain between amino acids 589-924. KIT receptor include allelic variants of KIT. In one example, an allelic variant contains one or more amino acid changes compared to SEQ ID NO:273, such as set forth in SEQ ID NO:305. For example, one or more amino acid variations can occur in the transmembrane domain of KIT. An allelic variant can include one or more amino acid changes at position 541 (SNP NO: 3822214) where, for example, M can be replaced by L or V. Additional examples of amino acid changes can occur in the protein kinase domain. An allelic variant can include one or more amino acid changes at position 664 where, for example, G can be replaced by R, or at position 788 where, for example, C can be replaced by R, or at position 801 where, for example, T can be replaced by I, or at position 816 where, for example, D can be replaced by V, H, or Y, or at position 820 where, for example, D is replaced by V, or at position 822 where, for example, N can be replaced by K or Y, or at position 823 where, for example, Y can be replaced by D or C, or at position 835 where, for example, W can be replaced by R, or at position 869 where, for example, P can be replaced by S, or at position 900 where, for example, Y can be replaced by F. Allelic variants also can include one or more amino acid change at position 52, where, for example, D is replaced by N, or at position 136 where, for example, C is replaced by R, or at position 178 where, for example, A is replaced by T, or at position 557 where, for example, W is replaced by R.

In one example, an allelic variant includes one or more amino acid changes compared to SEQ ID NO:273 and the variant exhibits a change in a biological activity. For example, an allelic variant contains one or more amino acid changes occurring in the protein kinase domain of KIT, such as at positions 816, 823, 822, or 801. In another example, one or more amino acid changes occur in the protein kinase domain, such as at position 900, and are associated with diminished receptor phosphorylation, association with adaptor proteins such as CrkII, and activation. In the context of a wildtype or predominant form of the receptor such allelic variation can be associated with a disease or condition, for example, testicular seminomas, intracranial germinomas, chronic myelogenous leukemia, human peibaldism and idiopathic myelofibrosis.

The exemplary KIT isoform set forth as SEQ ID NO: 93 contains an immunoglobulin-like domain between amino acids 210-336, but does not contain a transmembrane domain or protein kinase domain. KIT isoforms, including KIT isoforms herein, can include allelic variation in the KIT polypeptide. For example, a KIT isoform can include one or more amino acid differences present in an allelic variant. In one example, a KIT isoform includes one or more allelic variations as set forth in SEQ ID NO:305. An allelic variation can include one or more amino acid changes, such as at amino acids corresponding to positions 136 or 178 of SEQ ID NO:273.

The Flt3 receptor (GenBank No. NP_{—}004110 set forth as SEQ ID NO:272) is characterized by an immunoglobulin-like domain between amino acids 78-161 and between amino acids 257-345, a transmembrane domain between amino acids 542-564, and a tyrosine kinase domain between amino acids 610-943. Flt3 proteins include allelic variants of Flt3. In one example, an allelic variant contains one or more amino acid changes compared to SEQ ID NO:272, such as those set forth in SEQ ID NO:304. For example, one or more amino acid variations can occur in the tyrosine kinase domain of Flt3. An allelic variant can include amino acid changes at position 835 where, for example, D can be replaced by Y, H, or F, or at position 836 where, for example, I can be replaced by S, or at position 841 where, for example, N can be replaced by I or Y, or at position 842 where, for example Y can be replaced by H. In one example, an allelic variant includes one or more amino acid changes compared to SEQ ID NO:272 and the variant exhibits a change in a biological activity. One or more amino acid changes occurring in the tyrosine kinase domain of Flt3 receptor, such as at positions 835 or 841, can result in the constitutive activation of downstream targets of Flt3, such as signal transducer and activator of transcription STAT5, in the absence of Flt3 ligand stimulation. One or more amino acid changes can be present in the tyrosine kinase domain of Flt3, such as at positions 835, 836, and 842, also can be associated with a disease or condition, for example the progression from myelodysplastic syndrome to acute myeloid leukemia in infants and adults.

Flt3 is expressed in placenta and various adult tissues such as gonads, brain and in hematopoietic cells. Flt3 is associated with biological regulation in gonads, brain and nervous systems. Flt3 has been implicated as a target for pediatric cancers such as pediatric AML. KIT is involved in regulation in a broad variety of cell types including erythroid cells, interstitial cells, mast cells and germ cells. KIT is associated with a variety of cancers including gastrointestinal stromal tumors. RTK isoforms of Flt3, CSF1R and KIT can be used in the treatment of diseases and conditions in which the RTK are involved.

6. MET (Receptor for Hepatocye Growth Factor)

MET is a RTK for hepatocyte growth factor (HGF), a multifunctional cytokine controlling cell growth, morphogenesis and motility. HGF, a paracrine factor produced primarily by mesenchymal cells, induces mitogenic and morphogenic changes, including rapid membrane ruffling, formation of microspikes, and increased cellular motility. Signaling through MET can increase tumorigenicity, induce cell motility and enhance invasiveness in vitro and metastasis in vivo. MET signaling also can increase the production of protease and urokinase, leading to extracellular matrix/basal membrane degradation, which are important for promoting tumor metastasis.

MET is a RTK that is highly expressed in hepatocytes. MET is comprised of two disulfide-linked subunit, a 50-kD α subunit and a 145-kD β subunit. In the fully processed MET protein, the α subunit is extracellular, and the β subunit has extracellular, transmembrane, and tyrosine kinase domains. The ligand for MET is hepatocyte growth factor (HGF). Signaling through FGF and MET stimulates mitogenic activity in hepatocytes and epithelial cells, including cell growth, motility and invasion. As with other RTKs, these properties link MET to oncogenic activities. In addition to a role in cancer, MET also has been shown to be a critical factor in the development of malaria infection. Activation of MET is required to make hepatocytes susceptible to infection by malaria, thus MET is a prime target for prevention of the disease.

The MET receptor (GenBank No. NP_{—}000236 set forth as SEQ ID NO:274) is characterized by a Sema domain between amino acids 55-500. In addition to hepatocyte growth factor receptor, the Sema domain occurs in semaphorins, which are a large family of secreted and transmembrane proteins, some of which function as repellent signals during axon guidance. In MET, the Sema domain has been shown to be involved in receptor dimerization in addition to ligand binding. The MET protein also is characterized by a plexin cysteine rich repeat between amino acids 519-562, three IPT/TIG domains between amino acids 563-655, amino acids 657-739 and amino acids 742-836. IPT stands for Immunoglobulin-like fold shared by Plexins and Transcription factors. TIG stands for the Immunoglobulin-like domain in transcription factors (Transcription factor IG). TIG domains in MET likely play a role in mediating some of the interactions between extracellular matrix and receptor signaling. The MET protein also is characterized by a transmembrane domain between amino acids 951-973 and cytoplasmic protein kinase domain between amino acids 1078-1337.

MET receptors include allelic variants of MET. In one example, an allelic variant contains one or more amino acid changes compared to SEQ ID NO:274. For example, one or more amino acid variations can occur in the Sema domain of MET. An allelic variant can include amino acid changes at position 113 where, for example, K is replaced by R, or at position 114 where, for example, D is replaced by N, or at position 145 where, for example, V is replaced by A, or at position 148 where, for example, H is replaced by R, or at position 151 where, for example, T is replaced by P, or at position 158 where, for example, V is replaced by A, or at position 168 where, for example, E is replaced by D, or at position 193 where, for example, I is replaced by T, or at position 216 where, for example, V is replaced by L, or at position 237 where, for example, V is replaced by A, or at position 276 where, for example, T is replaced by A, or at position 314 where, for example, F is replaced by L, or at position 337 where, for example, L is replaced by P, or at position 340 where, for example, D is replaced by V, or at position 382 where, for example, N is replaced by D, or at position 400 where, for example, R is replaced by G, or at position 476 where, for example, H is replaced by R, or at position 481 where, for example, L is replaced by M, or at position 500 where, for example, D is replaced by G. In a further example, one or more amino acid variation can occur in the plexin cysteine rich repeat domain of MET. An allelic variant can include amino acid changes at position 542 where, for example, H can be replaced by Y. In other examples, one or more amino acid variation can occur in the IPT/TIG domains of MET. An allelic variant can include amino acid changes at position 622 where, for example, L is replaced by S, or at position 720 where, for example, F is replaced by S, or at position 729 where, for example, A is replaced by T. In an additional example, one or more amino acid variations can occur in the protein kinase domain of MET. An allelic variant can include amino acid changes at position 1094 where, for example, H is replaced by R or at position 1100 where, for example, N is replaced by Y or at position 1230 where, for example, Y is replaced by C, or at position 1235 where, for example, Y is replaced with D, or at position 1250 where, for example, M is replaced by T. Allelic variants also can include one or more amino acid changes, such as at position 37 where, for example, V is replaced by A, or at position 39 where, for example M is replaced by T, or at position 42 where, for example, Q is replaced by R, or at position 501 where, for example, Y can be replaced by H, or at position 511 where, for example, T can be replaced by A. In one example, an allelic variant includes one or more amino acid changes compared to SEQ ID NO:274 and the variant exhibits a change in a biological activity. An exemplary MET allelic variant containing one or more amino acid changes described above is set forth as SEQ ID NO: 306. Amino acid changes occurring in the tyrosine kinase domain of MET receptor, such as those described above, can be associated with dysregulated function of MET. For example, in the context of a wildtype or predominant form of the receptor, allelic changes in MET receptor are implicated in the development of human cancer including the promotion of tumor invasion, angiogenesis, and metastasis.

Exemplary isoforms of MET provided herein lack one or more domains or a part thereof compared to a cognate MET receptor such as set forth in SEQ ID NO:274. Exemplary MET receptor isoforms provided herein (e.g. SEQ ID NOS: 103, 186, 188, 190, 192, 194, 196, 198, 200, 202, 204, 206, 208, 210, 212, and 214) lack a transmembrane domain and/or a protein kinase domain. In addition, exemplary MET isoforms provided herein contain one or more domains of a wildtype or predominant form of MET receptor (e.g. set forth as SEQ ID NO:274). For example, MET receptor isoforms set forth as SEQ ID NOS: 103, 190, 192, 196, 198, 200, 202, 204, 206, 208, 210, 212, and 214 all contain complete Sema domains. MET isoforms set forth as SEQ ID NOS: 103, 192, 196, 198, 200, 202, 206, 208, 210, 212, and 214 contain complete plexin cysteine rich repeat domains. Met receptor isoforms can include one or more IPT/TIG domains. For example, MET receptor isoforms set forth as SEQ ID NOS: 103, 198, 200, 202, 204, 206, 208, 210, 212, and 214 contain at least one complete IPT/TIG domain. MET receptor isoforms set forth as SEQ ID NOS: 103, 208, 210, 212, and 214 all contain at least two complete IPT/TIG domains. MET receptor isoforms set forth as SEQ ID NOS: 103 and 212 contain three complete IPT/TIG domains. Among the MET receptor isoforms provided herein are isoforms that contain a portion of a domain compared to a wildtype or predominant form of MET receptor (e.g. set forth as SEQ ID NO:274). For example, MET receptor isoforms set forth as SEQ ID NOS: 186, 188, and 194 contain portions of the Sema domain between amino acids 55-412, 55-468, and 55-400, respectively. The MET receptor isoform set forth as SEQ ID NO: 196 contains a portion of an IPT/TIG domain between amino acids 563-621. MET receptor isoforms set forth as SEQ ID NOS: 198, 200 and 204, in addition to the one full IPT/TIG domain, contain a portion of a second IPT/TIG domain (between amino acids 657-664, 657-719, and 629-672, respectively). The MET receptor isoform set forth as SEQ ID NO: 210, in addition to the two full IPT/TIG domains, contains a portion of a third IPT/TIG domain between amino acids 742-823.

MET isoforms, including MET isoforms herein, can include allelic variation in the MET polypeptide. For example, a MET isoform can include one or more amino acid differences present in an allelic variant. In one example, a MET isoform includes one or more allelic variations as set forth in SEQ ID NO:306. An allelic variation can include one or more amino acid change in the Sema domain, such as at positions 113, 114, 145, 148, 151, 158, 168, 193, 216, 237, 276, 314, 337, 340, 382, 400, 476, 481, or 500. Allelic variations also can occur in the plexin cysteine rich repeat domain, such as at position 542. Further allelic variations also can occur in the IPT/TIG domain, such as at positions 622, 720, or 729. Allelic variations also can include other amino acid changes, such as at positions 37, 39, 42, 501, or 511.

MET isoforms can be used in treating or preventing metastatic cancer, and in inhibiting angiogenesis, such as angiogenesis necessary for tumor growth. Therapeutic applications of MET isoforms include lung cancer, malignant peripheral nerve sheath tumors (MPNST), colon cancer, gastric cancer, and cutaneous malignant melanoma.

MET isoforms also can be used in combination with other anti-angiogenesis drugs to prevent tumor cell invasiveness. Anti-angiogenesis drugs produce a state of hypoxia in tumors which can promote tumor cell invasion by sensitizing cells to HGF stimulation. MET isoforms can target and modulate biological activity of MET, such as by inhibiting or down-regulating MET when, anti-angiogenesis drugs are given, thus preventing or inhibiting tumor cell invasiveness.

Therapeutic applications of MET isoforms also include prevention of malaria. Plasmodium, the causative agent of malaria, must first infect hepatocytes to initiate a mammalian infection. Sporozoites migrate through several hepatocytes, by breaching their plasma membranes, before infection is finally established in one of them. Wounding of hepatocytes by sporozoite migration induces the secretion of hepatocyte growth factor (HGF), which renders hepatocytes susceptible to infection. Infection depends on activation of the HGF receptor, MET, by secreted HGF. The malaria parasite exploits MET as a mediator of signals that make the host cell susceptible to infection. HGF/MET signaling induces rearrangements of the host-cell actin cytoskeleton that are required for the early development of the parasites within hepatocytes. MET-isoforms can be administered as a therapeutic to down-regulate MET, thus inhibiting or preventing induction of MET signaling by malaria parasite and therefore inhibiting or preventing malaria infection.

RON (recepteur d'origine nantais; also known as macrophage stimulating 1 receptor) is another member of the MET subfamily of RTKs. A ligand for RON is macrophage-stimulating protein (MSP). RON is expressed in cells of epithelial origin. RON plays a role in epithelial cancers including lung cancer and colon cancers. RON and MET are expressed in ovarian cancers and are suggested to confer a selective advantage to cancer cells, thus promoting cancer progression. RON also is overexpressed in certain colorectal cancers. Germline mutations in the RON gene have been linked to human tumorigenesis. RON isoforms can be used to modulate RON, such as by modulating RON activity in diseases and conditions where RON is overexpressed.

The RON protein (GenBank No. NP_{—}002438 set forth as SEQ ID NO:277) is characterized by a Sema domain between amino acids 58-507, a plexin cysteine rich domain between amino acids 526-568, three IPT/TIG domains (between amino acids 569-671, amino acids 684-767, and amino acids 770-860), a transmembrane domain between amino acids 960-982 and cytoplasmic protein kinase domain between amino acids 1082-1341.

RON receptors include allelic variants of RON. In one example, an allelic variant contains one or more amino acids changes compared to SEQ ID NO:277, such as those set forth in SEQ ID NO:308. For example, one or more amino acid variations can occur in the Sema domain of RON. An allelic variant can include single nucleotide polymorphisms (SNP) at position 113 (SNP No. 3733136) where, for example, G is replaced by S, or at position 209 where, for example, G is replaced by A, or at position 322 (SNP No. 2230593) where, for example, Q is replaced by R, or at position 440 (SNP No. 2230592) where, for example, N is replaced by S. An amino acid variation also can occur at position 523 (SNP No. 2230590) where, for example, R is replaced by Q, or at position 946 (SNP No. 13078735) where, for example V is replaced by M. Additionally, one or more amino acid variations can occur in the protein kinase domain of RON. An allelic variant can include amino acid changes at position 1195 (SNP No. 7433231) where, for example, G is replaced by S, or at position 1335 (SNP No. 1062633) where, for example, R is replaced by G, or at position 1232 where, for example, D is replaced by V, or at position 1254 where, for example, M is replaced by T. In one example, an allelic variant includes one or more amino acid changes compared to SEQ ID NO:277 and the variant exhibits a change in a biological activity. Allelic variants, for example in the context of a wildtype or predominant form of the receptor, can be associated with a disease or condition. For example, amino acid changes occurring in the tyrosine kinase domain of RON, such as at positions corresponding to 1232 and 1254 of SEQ ID NO:277, can be associated with oncogenic cell transformation and tumor development by causing cellular accumulation of b-catenin whereby increases in the levels of b-catenin are associated with cancer.

SEQ ID NOS: 129, 216, 218 and 220 set forth exemplary RON isoforms. Exemplary RON isoforms lack one or more domains or a part thereof compared to a cognate RON such as set forth in SEQ ID NO:277. For example, exemplary RON isoforms set forth as SEQ ID NOS: 129, 216, 218 and 220 lack a transmembrane domain and protein kinase domain. The exemplary RON isoform set forth as SEQ ID NO:129 is characterized by a truncated Sema domain between amino acids 58-495. SEQ ID NO: 129 does not contain the plexin cysteine rich domain and IPT/TIG domains. The exemplary RON isoform set forth as SEQ ID NO: 216 also is characterized by a truncated Sema domain between amino acids 58-410, a complete plexin cysteine rich domain between amino acids 420-462, and a portion of an IPT/TIG domain between amino acids 463-521. The exemplary RON isoform set forth as SEQ ID NO: 220 contains complete Sema and plexin cysteine rich domains as well as a portion of an IPT/TIG domain between amino acids 569-627. SEQ ID NO: 218 sets forth an exemplary RON isoform that contains a complete Sema domain, plexin cysteine rich domain, and three IPT/TIG domains.

RON isoforms, including RON isoforms herein, also can include allelic variation in the RON polypeptide. For example a RON isoform can include one or more amino acid differences present in an allelic variant. In one example, a RON isoform includes one or more allelic variations as set forth in SEQ ID NO:308. An allelic variant can include one or more amino acid changes in the Sema domain, such as at positions 113, 209, 322. or 440. An allelic variant also can include one or more amino acid change, such as at position 523.

7. Vascular Endothelial Growth Factor (VEGF)

The vascular endothelial growth factor (VEGF) is a family of closely related growth factors with a conserved pattern of eight cysteine residues and sharing common VEGF receptors. VEGF receptors include VEGFR-1 (Flt-1) VEGFR-2 (Flk-1/KDR), and VEGFR-3 (Flt-4). Ligands for VEGF receptors include vascular endothelial growth factor-A (also known as vasculotropin (VAS) or vascular permeability factor (VPF)), VEGF-B, VEGF-C, VEGF-D and placental growth factor (P1GF). The VEGF proteins and receptors play an important role in many aspects of angiogenesis, including cell migration, proliferation and tube formation, thus linking these proteins to the pathogenesis of many types of cancer. Flt-1, Flk, and Flt-4/KDR are genes encoding VEGFR family members.

Exemplary RTK-isoforms for targeting VEGFR-related diseases and conditions include VEGFR isoforms set forth in SEQ ID NOS: 99-102, 110, 123, 125, 127, 224 and 226. Such isoforms can be used in the treatment of acute inflammatory disease, such as Kawasaki disease, rheumatoid arthritis, diabetic retinopathy, retinopathy and psoriasis, as well as re-regulation of abnormal angiogenesis. Additionally VEGFR-isoforms can be used for treatment of cancers including breast carcinoma.

a. VEGFR-1 (Flt-1)

Flt-1 (fms-like tyrosine kinase-1) is a member of the VEGF receptor family of tyrosine kinases. Ligands for Flt-1 include VEGF-A and P1GF (placental growth factor). Since Flt-1 and its ligands are important for angiogenesis, disregulation of these proteins have significant impacts on a variety of diseases stemming from abnormal angiogenesis, such as proliferation or metastasis of solid tumors, rheumatoid arthritis, diabetic retinopathy, retinopathy and psoriasis. Flt-1 also has been implicated in Kawasaki disease, a systemic vasculitis with microvascular hyperpermeability.

The VEGFR-1 polypeptide set forth as SEQ ID NO:282 (GenBank No. NP_{—}002010) is characterized by four immunoglobulin-like domains; domain 1 between amino acids 231-337, domain 2 between 332-427, domain 3 between amino acids 558-656, and domain 4 between amino acids 661-749. VEGR-1 also contains a transmembrane domain between amino acids 764-780 and protein kinase domain between amino acids 827-1154.

SEQ ID NOS: 99-102, 110 and 123 set forth exemplary VEGFR-1 isoforms. The exemplary VEGFR-1 isoforms lack one or more domains or a part thereof compared to a cognate VEGFR-1 such as set forth in SEQ ID NO:282. For example, the exemplary VEGFR-1 isoforms lack a transmembrane domain and protein kinase domain compared to a cognate VEGFR-1 (e.g. SEQ ID NO:282). Such isoforms also can lack additional domains or portions of domains of a cognate VEGFR-1. The exemplary VEGFR-1 isoforms set forth as SEQ ID NOS: 99, 100 and 110 contain two immunoglobulin-like domains between amino acids 231-337 and between amino acids 332-427, but do not contain immunoglobulin-like domains 2 and 3. The exemplary VEGFR-1 isoform set forth as SEQ ID NO: 101 contains immunoglobulin-like domain 1 between amino acids 231-337 and a portion of immunoglobulin-like domain 2 between amino acids 332-394. The exemplary VEGFR-1 isoform set forth as SEQ ID NO: 102 contains a portion of one immunoglobulin-like domain between amino acids 231-331. VEGFR-1 isoforms, including VEGFR-1 isoforms herein, can include allelic variation in the VEGFR-1 polypeptide, such as one or more amino acid changes compared to a cognate VEGFR-1 polypeptide (e.g., SEQ ID NO: 282).

b. VEGFR-2 (KDR/Flk-1)

VEGFR-2 (KDR/Flk-1) is a member of the VEGF receptor family of tyrosine kinases. Ligands for VEGFR-2 includes VEGF. VEGF interacts with its receptors, VEGFR-2 and VEGFR-1, expressed on endothelial and hematopoietic stem cells, and thereby promotes recruitment of these cells to neo-angiogenic sites, accelerating the revascularization process. As such, VEGF is found in several types of tumors and has a tumoral angiogenic activity in vitro and in vivo. The interaction of VEGF with VEGFR-1 mediates cell migration whereas the interaction of VEGF with VEGFR-2 mediates cell proliferation. The VEGFR-2 receptor is the main human receptor responsible for the VEGF activity in physiological and pathological vascular development, and VEGF-KDR signaling pathway is a potential target for the development of anti- and pro-angiogenic agents.

The VEGFR-2 protein (GenBank No. NP_{—}002244 set forth as SEQ ID NO:283) is characterized by three immunoglobulin-like domains; domain 1 between amino acids 224-325, domain 2 between amino acids 333-418, and domain 3 between amino acids 666-766. VEGFR-2 also contains a transmembrane domain between amino acids 763-785 and protein kinase domain between amino acids 834-1160.

VEGFR-2 proteins include allelic variants of VEGFR-2. In one example, an allelic variant contains one or more amino acids changes compared to SEQ ID NO: 283. For example, one or more amino acid variations can occur in the immunoglobulin-like domain of VEGFR-2. An allelic variant can include single nucleotide polymorphisms (SNP) at position 297 (SNP No: 2305948) where, for example, V can be replaced by I, or at position 349 (SNP No: 1824302) where, for example, R can be replaced by K, or at position 392 (SNP No: 2034964) where, for example, D can be replaced by N. Additionally, one or more amino acid variations can occur in the protein kinase domain of VEGFR-2. An allelic variant can include amino acid changes at position 835 (SNP No: 1139775) where, for example, K is replaced by N, or at position 848 (SNP No: 1139776) where, for example, V is replaced by E, or at position 952 (SNP No: 13129474) where, for example, V is replaced by I. One or more amino acid changes also can occur in the transmembrane domain. An allelic variant can include amino acid changes at position 772 (SNP No: 1062832) where, for example A is replaced by T. An amino acid variation also can occur at position 472 (SNP No: 1870377) where, for example, Q is replaced by H, or at position 787 (SNP No: 1139774) where, for example, R is replaced by G, or at position 1147 where, for example, P is replaced by S, or at position 1210 (SNP No: 11540507) where, for example, P is replaced by I, or at position 1347 (SNP No: 1139777) where, for example, S is replaced by T. In one example, an allelic variant includes one or more amino acid changes compared to SEQ ID NO:283 and the variant exhibits a change in biological activity. Allelic variants, for example in the context of a wildtype or predominant form of the receptor, can be associated with a disease or condition. For example, amino acid changes occurring in the kinase domain of VEGFR-2, such as at position 1147 described herein, can be associated with tumors such as those found in Juvenile hemangiomas. An exemplary VEGFR-2 allelic variant containing one or more amino acid changes described above is set forth as SEQ ID NO: 313.

Exemplary isoforms of VEGFR-2 include isoforms lacking one or more domains or a part thereof compared to a cognate VEGFR-2 such as set forth in SEQ ID NO:283. Such isoforms include the isoform set forth in SEQ ID NO: 224 that does not contain transmembrane or protein kinase domains. The exemplary VEGFR-2 isoform set forth as SEQ ID NO:224 is characterized by immunoglobulin-like domains between amino acids 224-325, amino acids 333-418, and a portion of a third immunoglobulin-like domain between amino acids 666-691.

VEGFR-2 isoforms, including VEGFR-2 isoforms herein, can include allelic variation in the VEGFR-2 polypeptide. For example a VEGFR-2 isoform can include one or more amino acid differences present in an allelic variant. In one example, a VEGFR-2 isoform includes one or more allelic variations as set forth in SEQ ID NO:313. An allelic variant can include one or more amino acid changes in the immunoglobulin-like domain, such as at positions 297, 349, or 392. Allelic variants also can include one or more amino acid change such as at position 472.

c. VEGFR-3

VEGFR-3 is expressed predominantly in lymphatic endothelial cells. VEGFR-3 signaling is crucial for development and maintenance of lymphatic vessels. Mouse models expressing VEGFR-3 can be used to assess effects on lymphatic tissue development and maintenance in the presence of VEGFR-3 isoforms. VEGFR-3 also can have effects on blood vascular endothelium.

The VEGFR-3 polypeptide (GenBank No. NP_{—}002011 set forth as SEQ ID NO:284) is characterized by four immunoglobulin-like domains; domain 1 between amino acids 231-328, domain 2 between amino acids 349-398, domain 3 between amino acids 571-655 and domain 4 between amino acids 677-766. VEGFR-3 also contains a transmembrane domain between amino acids 776-798 and protein kinase domain between amino acids 845-1169.

VEGFR-3 polypeptides include allelic variants of VEGFR-3. In one example, an allelic variant contains one or more amino acids changes compared to SEQ ID NO: 284. For example, one or more amino acid variations can occur in the protein kinase domain of VEGFR-3. An allelic variant can include single nucleotide polymorphisms (SNP) at position 854 where, for example, G can be replaced by S, or at position 890 (SNP No: 448012) where, for example, Q can be replaced by H, or at position 915 where, for example, A can be replaced by P, or at position 916 where, for example, C and be replaced by W, or at position 933 where, for example, G can be replaced by R, or at position 954 where, for example, P can be replaced by S, or at position 1008 where, for example, P can be replaced by L, or at position 1041 where, for example, R can be replaced by W or Q, or at position 1137 where, for example, P can be replaced by L, or at position 1164 (SNP No: 1049080) where, for example, D can be replaced by E. An amino acid variation also can occur at position 24 where, for example, D is replaced by G, or at position 134 where, for example, D is replaced by G, or at position 149 where, for example, N can be replaced by D, or at position 494 (SNP No: 307826) where, for example T can be replaced by A, or at position 1189 (SNP No: 744282) where, for example, R can be replaced by C. In one example, an allelic variant includes one or more amino acid changes compared to SEQ ID NO:284 and the variant exhibits a change in a biological activity. Amino acid changes occurring in the tyrosine kinase domain can interfere with VEGFR-3 signaling, such as those described herein at positions 854, 915, 916, 933, 1041, and 1137. Allelic variants, for example in the context of a wildtype or predominant form of the receptor, can be associated with a disease or condition. For example, amino acid changes occurring in the tyrosine kinase domain can be associated with primary congenital lymphoedema; amino acid changes at position 954 can be associated with tumors such as juvenile hemangiomas. An exemplary VEGFR-3 allelic variant containing one or more amino acid changes described above is set forth as SEQ ID NO: 314.

Exemplary VEGFR-3 isoforms lack one or more domains or a part thereof compared to a cognate VEGFR-3 such as set forth in SEQ ID NO:284. SEQ ID NOS: 125, 127 and 226 set forth exemplary VEGFR-3 isoforms that lack a transmembrane and protein kinase domains. Such isoforms contain other domains of VEGFR-3. The exemplary VEGFR-3 isoform set forth as SEQ ID NO:226 is characterized by immunoglobulin-like domain 1 between amino acids 231-328, domain 2 between amino acids 349-398, domain 3 between amino acids 571-655, and a portion of a domain 4 between amino acids 677-723. SEQ ID NO: 127 is characterized by one immunoglobulin-like domain between amino acids 231-272.

VEGFR-3 isoforms, including VEGFR-3 isoforms herein, also can include allelic variation in the VEGFR-3 polypeptide, compared to a cognate VEGFR-3 receptor such as set forth in SEQ ID NO:284. For example a VEGFR-3 isoform can include one or more amino acid differences present in an allelic variant such as set forth in SEQ ID NO:314, for example at positions corresponding to amino acid position 24, 134, 149 or 494 of SEQ ID NO:284.

8. TIE

Tie-1 and Tie-2/TEK (tyrosine kinase with immunoglobulin-like and EGF-like domains) receptors are endothelial RTKs with immunoglobulin and epidermal growth factor homology domains. Exemplary RTK-isoforms for targeting Tie/TEK receptors include RTK isoforms set forth in SEQ ID NO: 104, 105, 112, 113, 131, 133, 135, 137, 139, 141, 143 and 222. Such RTK isoforms can be used for treatment of diseases and conditions in which the Tie/Tek receptor is implicated, including anti-angiogenesis therapy in diseases such as cancer, eye diseases, and rheumatoid arthritis. Other diseases and conditions that can be treated with TIE/TEK isoforms include inflammatory diseases such as arthritis, rheumatism, and psoriasis, benign tumors and preneoplastic conditions, myocardial angiogenesis, hemophilic joints, scleroderma, vascular adhesions, atherosclerotic plaque neovascularization, telangiectasia, and wound granulation. Additional targets for TEK receptor isoforms include diseases in which TEK is overexpressed, for example, chronic myeloid leukemia.

a. Tie-1

Tie-1 is a receptor tyrosine kinase that plays an essential role in vascular development and angiogenesis where it is thought to be required for vessel maturation and stabilization. Tie-1 also acts as an antiapoptotic survival signal. Tie-1 expression is associated with endothelial cells and neovascularization and physically associates with the related receptor TEK. Tie-1 also is expressed in a variety of tumors and metastases including lung and breast and also is involved in thyroid tumorigenesis. Tie-1 is strongly induced during wound healing. The ligands responsible for activating Tie-1 remain unidentified.

The Tie-1 receptor set forth as SEQ ID NO:279 (GenBank No. NP_{—}005415 set forth as SEQ ID NO: 279) is characterized by two immunoglobulin domains between amino acids 139-197 and amino acids 365-428, an EGF domain between amino acids 224-255, a laminin EGF-like domain between amino acids 231-272, three fibronectin type III domains (between amino acids 446-533, amino acids 546-632, and amino acids 644-729), transmembrane domain between amino acids 764-786, and cytoplasmic protein kinase domain between 839-1107.

Tie-1 proteins include allelic variants of Tie-1. In one example, an allelic variant contains one or more amino acids changes compared to SEQ ID NO: 279. For example, one or more amino acid variations can occur in the immunoglobulin domain of Tie-1. An allelic variant can include single nucleotide polymorphisms (SNP) at position 142 (SNP No: 11545380) where, for example, A can be replaced by T. An amino acid variation also can occur at position 1109 (SNP No: 6698998) where, for example, R is replaced by C. An exemplary Tie-1 allelic variant containing one or more amino acid changes described above is set forth as SEQ ID NO: 310.

Exemplary Tie-1 isoforms lack one or more domains or a part thereof compared to a cognate Tie-1 such as set forth in SEQ ID NO:279. For example, the exemplary Tie-1 isoforms provided herein lack transmembrane and protein kinase domains. Such exemplary Tie-1 isoforms include the Tie-1 isoforms set forth in SEQ ID NOS: 113, 135, 137, 139, 141, 143 and 222. These isoforms contain other domains of the Tie-1 receptor. The exemplary Tie-1 isoform set forth as SEQ ID NOS: 113 and 222 are characterized by two immunoglobulin domains between amino acids 139-197 and amino acids 365-428, an EGF domain between amino acids 224-255, a laminin EGF-like domain between amino acids 231-272, and three fibronectin type III domains (between amino acids 446-533, amino acids 546-632, and amino acids 644-729). The exemplary Tie-1 isoforms set forth as SEQ ID NOS: 137, 141 and 143 contain an immunoglobulin domain between amino acids 139-197, an EGF domain between amino acids 224-255 and a laminin EGF-like domain between amino acids 231-272. The exemplary Tie-1 isoforms set forth as SEQ ID NOS: 135 and 139 contain at least a portion of the immunoglobulin domain.

Tie-1 isoforms, including Tie-1 isoforms herein, can include allelic variation in the Tie-1 polypeptide. For example, a Tie-1 isoform can include one or more amino acid differences compared to a cognate Tie-1 receptor (e.g. SEQ ID NO:279). In one example, a Tie-1 isoform includes one or more allelic variations as set forth in SEQ ID NO:310. For example, an allelic variant of a Tie-1 isoform can include an amino acid change in the immunoglobulin domain, such as at position 142.

b. Tie-2 (TEK)

The known ligands for Tie-2/TEK include angiopoietin (Ang)-1 and Ang-2. These RTKs play an important role in the development of the embryonic vasculature and continue to be expressed in adult endothelial cells. Tie-2/TEK is a RTK that is expressed almost exclusively by vascular endothelium. Expression of Tie-2/TEK is important for the development of the embryonic vasculature. Overexpression and/or mutation of Tie-2/TEK has been linked to pathogenic angiogenesis, and thus tumor growth, as well as myeloid leukemia.

The Tie-2/TEK protein (GenBank No. NP_{—}000450 set forth as SEQ ID NO:278) is characterized by a laminin EGF-like domain between amino acids 219-268, three fibronectin type III domains (between amino acids 444-529, amino acids 543-626, and amino acids 639-724), a transmembrane domain between amino acids 748-770, and cytoplasmic protein kinase domain between amino acids 824-1092.

TEK proteins include allelic variants of TEK. In one example, an allelic variant contains one or more amino acids changes compared to SEQ ID NO: 278. For example, one or more amino acid variations can occur in fibronectin type III domain of TEK. An allelic variant can include single nucleotide polymorphisms (SNP) at position 486 (SNP No: 1334811) where, for example, V can be replaced by I, or at position 695 where, for example, I can be replaced by T, or at position 724 (SNP No. 4631561) where, for example, A can be replaced by T. An allelic variant also can occur in the protein kinase domain of TEK. An allelic variant can include amino acid changes at position 849 where, for example, R can be replaced by W. An amino acid variation also can occur at position 346 where, for example, P can be replaced by Q. In one example, an allelic variant includes one or more amino acid changes compared to SEQ ID NO:278 and the variant exhibits a change in a biological activity. Allelic variants, for example in the context of a wildtype or predominant form of the receptor, can be associated with a disease or condition. For example, amino acid changes occurring in the kinase domain of TEK receptor, such as at position 849, can be associated with vascular dysmorphogenesis due to increased activity of TEK. An exemplary TEK allelic variant containing one or more amino acid changes described above is set forth as SEQ ID NO: 309.

Exemplary Tie-2/TEK isoforms lack one or more domains or a part thereof compared to a cognate TEK such as set forth in SEQ ID NO:278. For example, exemplary TEK isoforms set forth in SEQ ID NOS: 104, 105, 112, 131 and 133 lack a transmembrane domain and kinase domain. Tie-2/TEK isoforms can contain other domains of a Tie-2/TEK cognate receptor. The exemplary TEK isoforms set forth as SEQ ID NO: 104 contains a laminin EGF-like domain between amino acids 219-268 and three fibronectin type III domains between amino acids 401-486, amino acids 500-580, and amino acids 593-678. The exemplary TEK isoforms set forth as SEQ ID NO: 105 contains a laminin EGF-like domain between amino acids 219-268 and three fibronectin type III domains between amino acids 444-529, amino acids 543-623, and amino acids 636-721. The exemplary TEK isoforms set forth as SEQ ID NO: 112 contains a laminin EGF-like domain between amino acids 196-245 and three fibronectin type III domains between amino acids 378-463, amino acids 477-557, and amino acids 570-655. The exemplary TEK isoform set forth as SEQ ID NO: 131 contains a laminin EGF-like domain between amino acids 219-268, but is missing the three fibronectin type III domains. The exemplary TEK isoform set forth as SEQ ID NO: 133 contains a laminin EGF-like domain between amino acids 219-268 and a portion of a fibronectin type III domain between amino acids 444-497.

TEK isoforms, including TEK isoforms herein, can include allelic variation in the TEK polypeptide. For example, a TEK isoform can include one or more amino acid differences present in an allelic variant. In one example, a TEK isoform includes one or more allelic variations as set forth in SEQ ID NO:309. An allelic variant can include one or more amino acid change in the fibronectin type III domain, such as at position 486 or 695. An allelic variant also can include one or more amino acid change, such as at position 346.

9. Tumor Necrosis Factor Receptors (TNFRs)

The TNF (tumor necrosis factor) ligand and receptor family regulate a variety of signal transduction pathways including those involved in cell differentiation, activation, and viability. TNFRs have a characteristic repeating extracellular cysteine-rich motif and a variable intracellular domain that differs between members of the TNFR family. The TNFR family of receptors includes, but is not limited to, TNFR1, TNFR2, TNFRrp, the low-affinity nerve growth factor receptor, Fas antigen, CD40, CD27, CD30, 4-1BB, OX40, DR3, DR4, DR5, and herpesvirus entry mediator (HVEM). Ligands for TNFRs include TNF-α, lymphotoxin, nerve growth factor, Fas ligand, CD40 ligand, CD27 ligand, CD30 ligand, 4-1BB ligand, OX40 ligand, APO3 ligand, TRAIL and LIGHT. TNFRs include an extracellular domain, including a ligand binding domain, a transmembrane domain and an intracellular domain that participates in signal transduction. Additionally, TNFRs are typically trimeric proteins that trimerize at the cell surface. Trimerization is important for biological activity of TNFRs.

TNF plays a key role in inflammatory and infectious diseases. TNF binds two receptors, TNFR1 and TNFR2 that can transduce intracellular signals when expressed on the cell surface. TNFR1 is a major mediator of biological signaling involved in cell apoptosis, cytotoxicity, fibroblast proliferation, synthesis of prostaglandin E2 and resistance to Chlamydia. TNFR2 is involved in proliferation of thermocytes, TNF-dependent proliferative response to mononuclear cells, induction of GM-CSF secretion, inhibition of early hematopoiesis, and down-regulating activated T cells by inducing apoptosis. TNFR1 and TNFR2 also are produced as soluble forms by proteolytic cleavage (sTNFR). Increased levels of sTNFRs have been found in inflammatory and infectious diseases.

TNF/TNFRs are targets for many viruses. Viruses can bind to and sequester host cytokines, such as TNF, thus allowing the virus to escape the immune system. Many viruses encode proteins that mimic TNFR by binding TNF or that are viral homologs of TNFR. Viruses can upregulate TNF gene activity and/or expression, modulate TNF/TNFR effects, and bind to TNFR. TNFR isoforms, such as described herein, can be used to modulate TNFRs, including viral TNFR homologs and mimics. Examples of viruses that interact with TNF/TNFRs and are targets for TNFR isoforms include, but are not limited to, DNA viruses including Myxoma virus, Vaccinia virus, Tanapox virus, Epstein-Barr virus, Herpes simplex virus, Cytomegalovirus, Herpesvirus saimiri, Hepatitis B virus, African swine fever virus and Parovirus, and RNA viruses including Human Immune deficiency virus (HIV), Hepatitis C virus, Influenza virus, Respiratory syncytial virus, Measles virus, Vesicular stomatitis virus, Dengue virus and Ebola virus (see for example, Herbein et al. (2000) Proc Soc Exp Biol Med. 223(3):241-57). Exemplary TNFR isoforms include isoforms of TNFR1 such as set forth in SEQ ID NO: 95.

a. TNFR1

The TNFR1 polypeptide set forth as SEQ ID NO:280 (GenBank No. NP_{—}001056) is characterized by three TNFR c6 domains (between amino acids 44-81, amino acids 84-125, and amino acids 127-166), a transmembrane domain between amino acids 212-234, and a death domain between amino acids 357-441 within the cytoplasmic tail. The TNFR c6 domains are cysteine-rich domains at the N-terminal region that can be subdivided into repeats containing six conserved cysteines, all of which are involved in intrachain disulfide bonds. Death domains are characteristic of the TNFR1 receptor family and are involved in initiating apoptosis and NF-κB and other signaling pathways upon ligand binding.

TNFR1 polypeptides include allelic variants of TNFR1. In one example, an allelic variant contains one or more amino acids changes compared to SEQ ID NO: 280. For example, one or more amino acid variations can occur in the c6 domains of TNFR1. An allelic variant can include single nucleotide polymorphisms (SNP) at position 75 (SNP No: 4149637) where, for example, P can be replaced by I, or at position 121 (SNP No. 4149584) where, for example, R can be replaced by Q. An amino acid variation also can occur at position 305 where, for example, P can be replaced by T. An exemplary TNFR1 allelic variant containing one or more amino acid changes described above is set forth as SEQ ID NO: 311.

b. TNFR2

TNFR2 (GenBank No. NP_{—}001057 set forth as SEQ ID NO:281) is characterized by three TNFR c6 domains between amino acids 40-75, amino acids 78-118 and amino acids 120-161 and a transmembrane domain between amino acids 258-280. TNFR2 proteins include allelic variants of TNFR2. In one example, an allelic variant contains one or more amino acids changes compared to SEQ ID NO: 281. For example, one or more amino acid variations can occur in the transmembrane domain. An allelic variant can include single nucleotide polymorphisms at position 295 (SNP No: 5746032) where, for example, Q can be replaced by R. An amino acid variation also can occur at position 187 (SNP No: 5746025) where, for example, V can be replaced by M, or at position 196 (SNP No: 1061622) where, for example, M can be replaced by R, or at position 232 (SNP No: 5746026) where, for example, E can be replaced by K, or at position 236 (SNP No: 5746027) where, for example, A can be replaced by T, or at position 264 (SNP No: 5746031) where, for example, L can be replaced by P. In one example, an allelic variant includes one or more amino acid changes compared to SEQ ID NO:281 and the variant exhibits a change in a biological activity. Allelic variants, for example in the context of a wildtype or predominant form of the receptor, can be associated with a disease or condition. For example, amino acid changes occurring at position 196, for example, can be associated with autoimmune disease such as rheumatoid arthritis and acute graft-versus-host disease and diseases associated with polycystic ovary syndrome and hyperandrogenism. An exemplary TNFR2 allelic variant containing one or more amino acid changes described above is set forth as SEQ ID NO: 312.

Exemplary TNFR2 isoforms lack one or more domains or a part thereof compared to a cognate TNFR2 such as set forth in SEQ ID NO:281. The exemplary TNFR2 isoform set forth as SEQ ID NO:95 lacks a transmembrane domain. Additionally, this isoform is characterized by TNFR c6 domains between amino acids 40-75 and amino acids 78-118 as well as a portion of a third c6 domain between amino acids 120-152.

### G. Methods of Producing Nucleic Acid Encoding CSR Isoforms and Methods of Producing CSR Isoform Polypeptides

Exemplary methods for generating CSR isoform nucleic acid molecules and polypeptides are provided herein. Such methods include in vitro synthesis methods for nucleic acid molecules such as PCR, synthetic gene construction and in vitro ligation of isolated and/or synthesized nucleic acid fragments. CSR isoform nucleic acid molecules also can be isolated by cloning methods, including PCR of RNA and DNA isolated from cells and screening of nucleic acid molecule libraries by hybridization and/or expression screening methods.

CSR isoform polypeptides can be generated from CSR isoform nucleic acid molecules using in vitro and in vivo synthesis methods. CSR isoforms can be expressed in any organism suitable to produce the required amounts and forms of isoform needed for administration and treatment. Expression hosts include prokaryotic and eukaryotic organisms such as E. coli, yeast, plants, insect cells, mammalian cells, including human cell lines and transgenic animals. CSR isoforms also can be isolated from cells and organisms in which they are expressed, including cells and organisms in which isoforms are produced recombinantly and those in which isoforms are synthesized without recombinant means such as genomically-encoded isoforms produced by alternative splicing events.

1. Synthetic Genes and Polypeptides

CSR isoform nucleic acid molecules and polypeptides can be synthesized by methods known to one of skill in the art using synthetic gene synthesis. In such methods, a polypeptide of a CSR isoform is “back-translated” to generate one or more nucleic acid molecules encoding an isoform. The back-translated nucleic acid molecule is then synthesized as one or more DNA fragments such as by using automated DNA synthesis technology. The fragments are then operatively linked to form a nucleic acid molecule encoding an isoform. Nucleic acid molecules also can be joined with additional nucleic acid molecules such as vectors, regulatory sequences for regulating transcription and translation and other polypeptide-encoding nucleic acid molecules. Isoform-encoding nucleic acid molecules also can be joined with labels such as for tracking, including radiolabels, and fluorescent moieties.

The process of backtranslation uses the genetic code to obtain a nucleotide gene sequence for any polypeptide of interest, such as a CSR isoform. The genetic code is degenerate, 64 codons specify 20 amino acids and 3 stop codons. Such degeneracy permits flexibility in nucleic acid design and generation, allowing for example restriction sites to be added to facilitate the linking of nucleic acid fragments and the placement of unique identifier sequences within each synthesized fragment. Degeneracy of the genetic code also allows the design of nucleic acid molecules to avoid unwanted nucleotide sequences, including unwanted restriction sites, splicing donor or acceptor sites, or other nucleotide sequences potentially detrimental to efficient translation. Additionally, organisms sometimes favor particular codon usage and/or a defined ratio of GC to AT nucleotides. Thus, degeneracy of the genetic code permits design of nucleic acid molecules tailored for expression in particular organisms or groups of organisms. Additionally, nucleic acid molecules can be designed for different levels of expression based on optimizing (or non-optimizing) of the sequences. Back-translation is performed by selecting codons that encode a polypeptide. Such processes can be performed manually using a table of the genetic code and a polypeptide. Alternatively, computer programs, including publicly available software can be used to generate back-translated nucleic acid sequences.

To synthesize a back-translated nucleic acid molecule, any method available in the art for nucleic acid synthesis can be used. For example, individual oligonucleotides corresponding to fragments of a CSR isoform-encoding sequence of nucleotides are synthesized by standard automated methods and mixed together in an annealing or hybridization reaction. Such oligonucleotides synthesized by such annealing result in the self-assembly of the gene from the oligonucleotides using overlapping single-stranded overhangs formed upon duplexing complementary sequences, generally about 100 nucleotides in length. Single nucleotide “nicks” in the duplex DNA are sealed using ligation, for example with bacteriophage T4 DNA ligase. Restriction endonuclease linker sequences can for example, then be used to insert the synthetic gene into any one of a variety of recombinant DNA vectors suitable for protein expression. In another, similar method, a series of overlapping oligonucleotides are prepared by chemical oligonucleotide synthesis methods. Annealing of these oligonucleotides results in a gapped DNA structure. DNA synthesis catalyzed by enzymes such as DNA polymerase I can be used to fill in these gaps, and ligation is used to seal any nicks in the duplex structure. PCR and/or other DNA amplification techniques can be applied to amplify the formed linear DNA duplex.

Additional nucleotide sequences can be joined to a CSR isoform-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to an isoform-encoding nucleic acid molecule. Examples of such sequences include, but are not limited to, promoter sequences designed to facilitate intracellular protein expression, and secretion sequences designed to facilitate protein secretion. Additional nucleotide sequences such as sequences specifying protein binding regions also can be linked to isoform-encoding nucleic acid molecules. Such regions include, but are not limited to, sequences to facilitate uptake of an isoform into specific target cells, or otherwise enhance the pharmacokinetics of the synthetic gene.

CSR isoforms also can be synthesized using automated synthetic polypeptide synthesis. Cloned and/or in silico-generated polypeptides can be synthesized in fragments and then chemically linked. Alternatively, isoforms can be synthesized as a single polypeptide. Such polypeptides then can be used in the assays and treatment administrations described herein.

2. Methods of Cloning and Isolating CSR Isoforms

CSR isoforms can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding an isoform, including for example, polymerase chain reaction (PCR) methods. A nucleic acid containing material can be used as a starting material from which an isoform-encoding nucleic acid molecule can be isolated. For example, DNA and mRNA preparations, cell extracts, tissue extracts, fluid samples (e.g. blood, serum, saliva), samples from healthy and/or diseased subjects can be used in amplification methods. Nucleic acid libraries also can be used as a source of starting material. Primers can be designed to amplify an isoform. For example, primers can be designed based on expressed sequences from which an isoform is generated. Primers can be designed based on back-translation of an isoform amino acid sequence. Nucleic acid molecules generated by amplification can be sequenced and confirmed to encode an isoform.

Nucleic acid molecules encoding isoforms also can be isolated using library screening. For example, a nucleic acid library representing expressed RNA transcripts as cDNA molecules can be screened by hybridization with nucleic acid molecules encoding CSR isoforms or portions thereof. For example, an intron sequence or portion thereof from a CSR gene can be used to screen for intron retention containing molecules based on hybridization to homologous sequences. Expression library screening can be used to isolate nucleic acid molecules encoding a CSR isoform. For example, an expression library can be screened with antibodies that recognize a specific isoform or a portion of an isoform. Antibodies can be obtained and/or prepared which specifically bind to a CSR isoform or a region or peptide contained in an isoform. Antibodies which specifically bind to an isoform can be used to screen an expression library containing nucleic acid molecules encoding an isoform, such as an intron fusion protein. Methods of preparing and isolating antibodies, including polyclonal and monoclonal antibodies and fragments therefrom are well known in the art. Methods of preparing and isolating recombinant and synthetic antibodies also are well known in the art. For example, such antibodies can be constructed using solid phase peptide synthesis or can be produced recombinantly, using nucleotide and amino acid sequence information of the antigen binding sites of antibodies that specifically bind to a candidate polypeptide. Antibodies also can be obtained by screening combinatorial libraries containing variable heavy chains and variable light chains, or antigen-binding portions thereof. Methods of preparing, isolating and using polyclonal, monoclonal and non-natural antibodies are reviewed, for example, in Kontermann and Dubel, eds. (2001) “Antibody Engineering” Springer Verlag; Howard and Bethell, eds. (2001) “Basic Methods in Antibody Production and Characterization” CRC Press; and O'Brien and Aitkin, eds. (2001) “Antibody Phage Display” Humana Press. Such antibodies also can be used to screen for the presence of an isoform polypeptide, for example, to detect the expression of a CSR isoform in a cell, tissue or extract.

3. Synthetic Isoforms

A variety of synthetic forms of the isoforms are provided. Included among them are conjugates in which the isoform or intron-encoded portion thereof is linked directly or via linker to another agent, such as a targeting agent or to a molecule the present or provides the intron-encoded portion or isoform portion to the CSR so that an activity of the CSR is modulated. Other synthetic forms include chimeras in which the extracellular domain portion and C-terminal portion, such as an intron-encoded portion, are from different isoforms. Also provided are “peptidomimetic” isoforms in which one or more bonds in the peptide backbone is (are) replaced by a bioisotere or other bond such that the resulting polypeptide peptidomimetic has improved properties, such as resistance to proteases, compared to the unmodified form.

a. Isoform Conjugates

CSR isoforms also can be provided as conjugates between the isoform and another agent. The conjugate can be used to target to a receptor with which the isoform interacts and/or to another targeted receptor for delivery of isoform. Such conjugates include linkage of a CSR isoform to a targeted agent and/or targeting agent. Conjugates can be produced by any suitable method including chemical conjugation or by expression of fusion proteins in which, for example, DNA encoding a targeted agent or targeting agent, with or without a linker region, is operatively linked to DNA encoding an RTK isoform. Conjugates also can be produced by chemical coupling, typically through disulfide bonds between cysteine residues present in or added to the components, or through amide bonds or other suitable bonds. Ionic or other linkages also are contemplated.

Pharmaceutical compositions can be prepared that contain CSR isoform conjugates and treatment effected by administering a therapeutically effective amount of a conjugate, for example, in a physiologically acceptable excipient. CSR isoform conjugates also can be used in in vivo therapy methods such as by delivering a vector containing a nucleic acid encoding a CSR isoform conjugate as a fusion protein.

Conjugates can contain one or more CSR isoforms linked, either directly or via a linker, to one or more targeted agents: (CSR isoform)n, (L)q, and (targeted agent)m in which at least one CSR isoform is linked directly or via one or more linkers (L) to at least one targeted agent. Such conjugates also can be produced with any portion of a CSR isoform sufficient to bind to a target, such as a target cell type for treatment. Any suitable association among the elements of the conjugate and any number of elements where n, and m are integer greater than 1 and q is zero or any integer greater then 1, is contemplated as long as the resulting conjugates interacts with a targeted CSR or targeted cell type.

Examples of a targeted agent include drugs and other cytotoxic molecules such as toxins that act at or via the cell surface and those that act intracellularly. Examples of such moieties, include radionuclides, radioactive atoms that decay to deliver, e.g., ionizing alpha particles or beta particles, or X-rays or gamma rays, that can be targeted when coupled to a CSR isoform. Other examples include chemotherapeutics that can be targeted by coupling with an isoform. For example, geldanamycin targets proteosomes. An isoform-geldanamycin molecule can be directed to intracellular proteosomes, degrading the targeted isoform and liberating geldanamycin at the proteosome. Other toxic molecules include toxins, such as ricin, saporin and natural products from conches or other members of phylum mollusca. Another example of a conjugate with a targeted agent is a CSR isoform coupled, for example as a protein fusion, with an antibody or antibody fragment. For example, an isoform can be coupled to an Fc fragment of an antibody that binds to a specific cell surface marker to induce killer T cell activity in neutrophils, natural killer cells, and macrophages. A variety of toxins are well known to those of skill in the art.

Conjugates can contain one or more CSR isoforms linked, either directly or via a linker, to one or more targeting agents: (CSR isoform)n, (L)q, and (targeting agent)m in which at least one CSR isoform is linked directly or via one or more linkers (L) to at least one targeting agent. Any suitable association among the elements of the conjugate and any number of elements where n, and m are integer greater than 1 and q is zero or any integer greater then 1, is contemplated as long as the resulting conjugates interacts with a target, such as a targeted cell type.

Targeting agents include any molecule that targets a CSR isoform to a target such as a particular tissue or cell type or organ. Examples of targeting agents include cell surface antigens, cell surface receptors, proteins, lipids and carbohydrate moieties on the cell surface or within the cell membrane, molecules processed on the cell surface, secreted and other extracellular molecules. Molecules useful as targeting agents include, but are not limited to, an organic compound; inorganic compound; metal complex; receptor; enzyme; antibody; protein; nucleic acid; peptide nucleic acid; DNA; RNA; polynucleotide; oligonucleotide; oligosaccharide; lipid; lipoprotein; amino acid; peptide; polypeptide; peptidomimetic; carbohydrate; cofactor; drug; prodrug; lectin; sugar; glycoprotein; biomolecule; macromolecule; biopolymer; polymer; and other such biological materials. Exemplary molecules useful as targeting agents include ligands for receptors, such as proteinaceous and small molecule ligands, and antibodies and binding proteins, such as antigen-binding proteins.

Alternatively, the CSR isoform, which specifically interacts with a particular receptor (or receptors) is the targeting agent and it is linked to targeted agent, such as a toxin, drug or nucleic acid molecule. The nucleic acid molecule can be transcribed and/or translated in the targeted cell or it can be regulatory nucleic acid molecule.

The CSR and be linked directly to the targeted (or targeting agent) or via a linker. Linkers include peptide and non-peptide linkers and can be selected for functionality, such as to relieve or decrease stearic hindrance caused by proximity of a targeted agent or targeting agent to a CSR isoform and/or increase or alter other properties of the conjugate, such as the specificity, toxicity, solubility, serum stability and/or intracellular availability and/or to increase the flexibility of the linkage between a CSR isoform and a targeted agent or targeting agent. Examples of linkers and conjugation methods are known in the art (see, for example, WO 00/04926). CSRs also can be targeted using liposomes and other such moieties that direct delivery of encapsulated or entrapped molecules.

b. Chimeric and Synthetic Intron Fusion Polypeptides

Also provided are chimeric and synthetic intron fusion polypeptides. These contain an intron from an intron fusion polypeptide operatively linked at the N-terminus to another polypeptide or other molecule such that the resulting molecule modulates the activity of a CSR, particularly an RTK, including any involved in pathways that participate in the inflammatory response, angiogenesis, neovascularization and/or cell proliferation. Included among these synthetic “polypeptides” are chimeric intron fusion polypeptides in which the N-terminus from the extracellular domain of a CSR is linked to the intron of an intron fusion protein, such as intron 8 of a herstatin (see, e.g., SEQ ID Nos. 320-345). Exemplary herstatins are set forth in SEQ ID Nos. 320-359. Table 3A below identifies the sequences. Other herstatin variants include allelic variants, particularly those with variation in the extracellular domain portion.

TABLE 3A

| | | SEQ ID NO | SEQ ID NO |
|---|---|---|---|
| Variant | Encoded Intron 8 | (nucleotide) | (amino acid) |
| Herstatin prominent | AA: 341-419 | | 320 |
| Intron 8 prominent- molecule in a bottle | | | 321 |
| Herstatin variant (AA 342: Thr or Ser) | AA: 341-419 | | 322 |
| Herstatin variant (AA 345: Leu or Pro | AA: 341-419 | | 323 |
| Herstatin variant (AA 346: Pro or Leu) | AA: 341-419 | | 324 |
| Herstatin variant (AA 356: Leu or Gln) | AA 341-419 | | 325 |
| Herstatin variant (AA 358: Met or Leu) | AA 341-419 | | 326 |
| Herstatin variant (AA 361: Gly, Asp, Ala, or Val) | AA 341-419 | | 327 |
| Herstatin variant (AA 376: Leu or Ile) | AA 341-419 | | 328 |
| Herstatin variant (AA 394: Pro or Arg) | AA 341-419 | | 329 |
| Herstatin variant (AA 404: Pro or Leu) | AA 341-419 | | 330 |
| Herstatin variant (AA 413: Asp or Asn) | AA 341-419 | | 331 |
| Herstatin variant (AA 357: Arg or Cys) | AA 341-419 | | 332 |
| Herstatin variant (AA 371: Arg or Ile) | AA 341-419 | | 333 |
| Intron 8 variant (AA 2: Thr or Ser) | | | 334 |
| Intron 8 variant (AA 5: Leu or Pro) | | | 335 |
| Intron 8 variant (AA 6: Pro or Leu) | | | 336 |
| Intron 8 variant (AA 16: Leu or Gln) | | | 337 |
| Intron 8 variant (AA 18: Met or Leu) | | | 338 |
| Intron 8 variant (AA 21: Gly, Asp, Ala, or Val) | | | 339 |
| Intron 8 variant (AA 36: Leu or Ile) | | | 340 |
| Intron 8 variant (AA 54: Pro or Arg) | | | 341 |
| Intron 8 variant (AA 64: Pro or Leu) | | | 342 |
| Intron 8 variant (AA 73: Asp or Asn) | | | 343 |
| Intron 8 variant (AA 17: Arg or Cys) | | | 344 |
| Intron 8 variant (AA 31: Arg or Ile) | | | 345 |
| Intron 8 prominent- molecule in a bottle | | 346 | |
| Intron 8 variant (nt 4: n = T) | | 347 | |
| Intron 8 variant (nt 14: n = C ) | | 348 | |
| Intron 8 variant (nt: 17: n = T) | | 349 | |
| Intron 8 variant (nt 47 = A) | | 350 | |
| Intron 8 variant (nt 54 = A) | | 351 | |
| Intron 8 variant (nt 62: n = C, T, A) | | 352 | |
| Intron 8 variant (nt 106 = A) | | 353 | |
| Intron 8 variant (nt 161 = G) | | 354 | |
| Intron 8 variant (nt 191: n = T) | | 355 | |
| Intron 8 variant (nt 217: C) | | 356 | |
| Intron 8 variant (nt 17: n = T and nt 217: n = C) | | 357 | |
| Intron 8 variant (nt 49: n = T) | | 358 | |
| Intron 8 variant (nt 92: n = T) | | 359 | |

The N-terminus portion can be linked to a C-terminus (intron-encoded portion) of the synthetic intron fusion protein directly or via a linker, such as a polypeptide linker or a chemical linker. Linkage can be effected by recombinant expression of a fusion protein where there is no linker or where the linker is a polypeptide. Chemical synthesis also can be employed. When the linker is not a polypeptide, linkage can be effected chemically.

Any suitable linker can be selected so long as the resulting molecule interacts with a CSR and modulates, typically inhibits, its activity. Linkers can be selected to add a desirable property, such as to increase serum stability, solubility and/or intracellular concentration and to reduce steric hindrance caused by close proximity when one or more linkers is (are) inserted between the N-terminal portion and intron-encoded portion. The resulting molecule is designed or selected to retain the ability to modulate the activity of a CSR, particularly RTKs, including any involved in pathways that are involved in inflammatory responses, neovascularization, angiogenesis and cell proliferation.

Linkers include chemical linkers and peptide linkers, such as peptides that increase flexibility or solubility of the linked moieties, and chemical linkers. For example linkers can be inserted using heterobifunctional reagents, such as those described below, or, can be linked by linking DNA encoding polypeptide linker to the DNA encoding the N-terminal (and/or C-terminal portion) and expressing the resulting chimera. In addition, where no linker is present the N-terminus can be linked directly to the intron encoded portion. In some embodiments, the N-terminus portion can be replaced by non-peptidic moiety that provides sufficient steric hindrance and bulk to permit the intron-encoded portion to interact with and modulate the activity of a receptor. As noted above, the N-terminus also can be selected to target the intron-encoded portion to selected CSRs or a selected CSR.

Exemplary linkers include, but are not limited to, (Gly4Ser)n, (Ser4Gly)n and (AlaAlaProAla)n (see, SEQ ID NO: 319) in which n is 1 to 4, such as 1, 2, 3 or 4, such as:

(1) Gly4Ser with NocI ends SEQ ID NO. 315
CCATGGGCGG CGGCGGCTCT GCCATGG
(2) (Gly4Ser)2 with NcoI ends SEQ ID NO. 316
CCATGGGCGG CGGCGGCTCT GGCGGCGGCG GCTCTGCCAT GG
(3) (Ser4Gly)4 with NcoI ends SEQ ID NO. 317
CCATGGCCTC GTCGTCGTCG GGCTCGTCGT CGTCGGGCTC
GTCGTCGTCG GGCTCGTCGT CGTCGGGCGC CATGG
(4) (Ser4Gly)2 with NcoI ends SEQ ID NO. 318
CCATGGCCTC GTCGTCGTCG GGCTCGTCGT CGTCGGGCGC CATGG
(5) (AlaAlaProAla)n, where n is 1 to 4, such as 2
or 3 (see, SEQ ID NO.:319)

c. Heterobifunctional Cross-Linking Reagents

Numerous heterobifunctional cross-linking reagents that are used to form covalent bonds between amino groups and thiol groups and to introduce thiol groups into proteins, are known to those of skill in this art (see, e.g., the PIERCE CATALOG, ImmunoTechnology Catalog & Handbook, 1992-1993, which describes the preparation of and use of such reagents and provides a commercial source for such reagents; see, also, e.g., Cumber et al. (1992) Bioconjugate Chem. 3:397-401; Thorpe et al. (1987) Cancer Res. 47:5924-5931; Gordon et al. (1987) Proc. Natl. Acad Sci. 84:308-312; Walden et al. (1986) J. Mol. Cell Immunol. 2:191-197; Carlsson et al. (1978) Biochem. J. 173: 723-737; Mahan et al. (1987) Anal. Biochem. 162:163-170; Wawryznaczak et al. (1992) Br. J. Cancer 66:361-366; Fattom et al. (1992) Infection & Immun. 60:584-589). These reagents may be used to form covalent bonds between the N-terminal portion and C-terminus intron-encoded portion or between each of those portions and a linker. These reagents include, but are not limited to: N-succinimidyl-3-(2-pyridyldithio)propionate (SPDP; disulfide linker); sulfosuccinimidyl 6-[3-(2-pyridyldithio)propion [symbol] amido]hexanoate (sulfo-LC-SPDP); succinimidyloxycarbonyl-α-methyl benzyl thiosulfate (SMBT, hindered disulfate linker); succinimidyl 6-[3-(2-pyridyldithio)propionami [symbol] do] [symbol] hexanoate (LC-SPDP); sulfosuccinimidyl 4-(N-maleimidomethyl)cyclohexane-1-carboxylate (sulfo-SMCC); succinimi [symbol] dyl 3-(2-pyridyldithio)butyrate (SPDB; hindered disulfide bond linker); sulfosuccinimidyl 2-(7-azido-4-methylcoumarin-3-acetamide)ethyl-1,3′-dithiopropionate (SAED); sulfo-succinimidyl 7-azido-4-methylcoumarin-3-acetate (SAMCA); sulfosuccinimidyl-6-[alpha-methyl-alpha-(2-pyridyldithio)toluamido]-hexanoate (sulfo-LC-SMPT); 1,4-di-[3′-(2′-pyridyldithio)propion-amido]butane (DPDPB); 4-succinimidyloxycarbonyl-α-methyl-α-(2-pyridylthio)toluene (SMPT, hindered disulfate linker); sulfosuccinimidyl-6-[α-methyl-α-(2-pyrimiyldithio)toluamido]hexanoate (sulfo-LC-SMPT); m-maleimidobenzoyl-N-hydroxy-succinimide ester (MBS); m-maleimidobenzoyl-N-hydroxysulfo-succinimide ester (sulfo-MBS); N-succinimidyl(4-iodoacetyl)aminobenzoate (SIAB; thioether linker); sulfosuccinimidyl-(4-iodoacetyl)amino benzoate (sulfo-SIAB); succinimidyl-4-(p-maleimi-dophenyl)butyrate (SMPB); sulfosuccinimidyl4-(p-maleimido-phenyl)butyrate (sulfo-SMPB); azidobenzoyl hydrazide (ABH). These linkers, for example, can be used in combination with peptide linkers, such as those that increase flexibility or solubility or that provide for or eliminate steric hindrance. Any other linkers known to those of skill in the art for linking a polypeptide molecule to another molecule can be employed. General properties are such that the resulting molecule is biocompatible (for administration to animals, including humans) and such that the resulting molecule modulates the activity of a CSR.

4. Expression Systems

CSR isoforms, including natural and combinatorial intron fusion proteins, can be produced by any method known to those of skill in the art including in vivo and in vitro methods. CSR isoforms can be expressed in any organism suitable to produce the required amounts and forms of CSR isoforms needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Many expression vectors are available and known to those of skill in the art and can be used for expression of CSR isoforms. The choice of expression vector will be influenced by the choice of host expression system. In general, expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vector.

CSR isoforms also can be utilized or expressed as protein fusions. For example, an isoform fusion can be generated to add additional functionality to an isoform. Examples of isoform fusion proteins include, but are not limited to, fusions of a signal sequence, a tag such as for localization, e.g. a his₆ tag or a myc tag, or a tag for purification, for example, a GST fusion, and a sequence for directing protein secretion and/or membrane association.

a. Prokaryotic Expression

Prokaryotes, especially E. coli, provide a system for producing large amounts of proteins such as CSR isoforms. Transformation of E. coli is a simple and rapid technique well known to those of skill in the art. Expression vectors for E. coli can contain inducible promoters, such promoters are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λPL promoter.

Isoforms can be expressed in the cytoplasmic environment of E. coli. The cytoplasm is a reducing environment and for some molecules, this can result in the formation of insoluble inclusion bodies. Reducing agents such as dithiothreotol and β-mercaptoethanol and denaturants, such as guanidine-HCl and urea can be used to resolubilize the proteins. An alternative approach is the expression of CSR isoforms in the periplasmic space of bacteria which provides an oxidizing environment and chaperonin-like and disulfide isomerases and can lead to the production of soluble protein. Typically, a leader sequence is fused to the protein to be expressed which directs the protein to the periplasm. The leader is then removed by signal peptidases inside the periplasm. Examples of periplasmic-targeting leader sequences include the pe1B leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility, typically temperatures between 25° C. and 37° C. are used. Typically, bacteria produce aglycosylated proteins. Thus, if proteins require glycosylation for function, glycosylation can be added in vitro after purification from host cells.

b. Yeast

Yeasts such as Saccharomyces cerevisae, Schizosaccharomyces pombe, Yarrowia lipolytica, Kluyveromyces lactis and Pichia pastoris are well known yeast expression hosts that can be used for production of CSR isoforms. Yeast can be transformed with episomal replicating vectors or by stable chromosomal integration by homologous recombination. Typically, inducible promoters are used to regulate gene expression. Examples of such promoters include GAL1, GAL7 and GAL5 and metallothionein promoters, such as CUP1, AOX1 or other Pichia or other yeast promoter. Expression vectors often include a selectable marker such as LEU2, TRP1, HIS3 and URA3 for selection and maintenance of the transformed DNA. Proteins expressed in yeast are often soluble. Co-expression with chaperonins such as Bip and protein disulfide isomerase can improve expression levels and solubility. Additionally, proteins expressed in yeast can be directed for secretion using secretion signal peptide fusions such as the yeast mating type alpha-factor secretion signal from Saccharomyces cerevisae and fusions with yeast cell surface proteins such as the Aga2p mating adhesion receptor or the Arxula adeninivorans glucoamylase. A protease cleavage site such as for the Kex-2 protease, can be engineered to remove the fused sequences from the expressed polypeptides as they exit the secretion pathway. Yeast also is capable of glycosylation at Asn-X-Ser/Thr motifs.

c. Insect Cells

Insect cells, particularly using baculovirus expression, are useful for expressing polypeptides such as CSR isoforms. Insect cells express high levels of protein and are capable of most of the post-translational modifications used by higher eukaryotes. Baculovirus have a restrictive host range which improves the safety and reduces regulatory concerns of eukaryotic expression. Typical expression vectors use a promoter for high level expression such as the polyhedrin promoter of baculovirus. Commonly used baculovirus systems include the baculoviruses such as Autographa californica nuclear polyhedrosis virus (AcNPV), and the bombyx mori nuclear polyhedrosis virus (BmNPV) and an insect cell line such as Sf9 derived from Spodoptera frugiperda, Pseudaletia unipuncta (A7S) and Danaus plexippus (DpN1). For high-level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schnieder 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express CSR isoforms. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. Such vectors often include transcriptional promoter-enhancers for high-level expression, for example the SV40 promoter-enhancer, the human cytomegalovirus (CMV) promoter and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha fetoprotein, alpha 1 antitrypsin, beta globin, myelin basic protein, myosin light chain 2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase and thymidine kinase. Fusion with cell surface signaling molecules such as TCR-ξ and Fc_{ε}RI-γ can direct expression of the proteins in an active state on the cell surface.

Many cell lines are available for mammalian expression including mouse, rat human, monkey, chicken and hamster cells. Exemplary cell lines include but are not limited to CHO, Balb/3T3, HeLa, MT2, mouse NS0 (nonsecreting) and other myeloma cell lines, hybridoma and heterohybridoma cell lines, lymphocytes, fibroblasts, Sp2/0, COS, NIH3T3, HEK293, 293S, 2B8, and HKB cells. Cell lines also are available that are adapted to serum-free media which facilitates purification of secreted proteins from the cell culture media. One such example is the serum free EBNA-1 cell line (Pham et al., (2003) Biotechnol. Bioeng. 84:332-42.)

e. Plants

Transgenic plant cells and plants can be used to express CSR isoforms. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline syntase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Transgenic plant cells also can include algae engineered to produce CSR isoforms (see for example, Mayfield et al. (2003) PNAS 100:438-442). Because plants have different glycosylation patterns than mammalian cells, this can influence the choice of CSR isoforms produced in these hosts.

5. Engineered CSR Isoforms

CSR isoforms can be designed and produced with one or more modified properties. These properties include but are not limited to increased protein stability, such as an increased protein half-life, increased thermal tolerance and/or resistance to one or more proteases. For example, a CSR isoform can be modified to increase protein stability in vitro and/or in vivo. In vivo stability can include protein stability under particular administration conditions such as stability in blood, saliva, and/or digestive fluids.

a. Modified Proteins

CSR isoforms can be modified using any methods known in the art for modification of proteins. Such methods include site-directed and random mutagenesis. Non-natural amino acids and/or non-natural covalent bonds between amino acids of the polypeptide can be introduced into a CSR isoform to increase protein stability. In such modified CSR isoforms, the biological function of the isoform can remain unchanged compared to the unmodified isoform. Assays such as the assays for biological function provided herein and known in the art can be used to assess the biological function of a modified CSR isoform

b. Peptidomimetic Isoforms.

Also provided are “peptidomimetic” isoforms in which one or more bonds in the peptide backbone (or other bond(s)) is (are) replaced by a bioisotere or other bond such that the resulting polypeptide peptidomimetic has improved properties, such as resistance to proteases, compared to the unmodified form.

### H. Assays to Assess or Monitor Isoform Activities or Affects on CSR Activities

CSR isoforms can exhibit alterations in structure or in one more activities compared to a full-length, wildtype or predominant form of a receptor. In addition, the CSR isoforms can alter (modulate) the activity of a CSR. All such isoforms are candidate therapeutics.

Where the isoforms exhibits a difference in an activity, in vitro and in vivo assays can be used to monitor or screen CSR isoforms. In vitro and in vivo assays also can be used to screen CSR isoforms to identify or select those that modulate the activity of a particular receptor or pathway. Such assays are well known to those of skill in the art. One of skill in the art can test a particular isoform for interaction with a CSR or a CSR ligand and/or test to assess any change in activity compared to a CSR. Some are exemplified herein.

Exemplary in vitro and in vivo assays are provided herein for comparison of an activity of an RTK isoform to an activity of a wildtype or predominant form of an RTK. Many of the assays are applicable to other CSRs and CSR isoforms. In addition, numerous assays, such as assays for kinase activities and cell proliferation activities of CSRs are known to one of skill in the art. Assays for activities of RTK isoforms and RTKs include, but are not limited to, kinase assays, homodimerization and heterodimerization assays, protein:protein interaction assays, structural assays, cell signaling assays and in vivo phenotyping assays. Assays also include employing animal models, including disease models in which an activity can be observed and/or measured or otherwise assessed. Dose response curves of a CSR isoform in such assays can be used to assess modulation of biological activities and as well as to determine therapeutically effective amounts of a CSR isoform for administration. Assays for RTK isoforms and RTKs include, but are not limited to, kinase assays, homodimerization and heterodimerization assays, protein:protein interaction assays, structural assays, cell signaling assays and in vivo phenotyping assays. Assays for TNFRs include, but are not limited, trimerization assays, localization assays such as membrane localization assays, protein:protein interaction assays, structural assays, cell signaling assays and in vivo phenotyping assays. Exemplary assays are described below.

1. Kinase Assays

Kinase activity can be detected and/or measured directly and indirectly. For example, antibodies against phosphotyrosine can be used to detect phosphorylation of an RTK, RTK isoform, an RTK:RTK isoform complex and phosphorylation of other proteins and signaling molecules. For example, activation of tyrosine kinase activity of an RTK can be measured in the presence of a ligand for an RTK. Transphosphorylation can be detected by anti-phosphotyrosine antibodies. Transphosphorylation can be measured and/or detected in the presence and absence of an RTK isoform, thus measuring the ability of an RTK isoform to modulate the transphosphorylation of an RTK. Briefly, cells expressing an RTK isoform or that have been exposed to an RTK isoform, are treated with ligand. Cells are lysed and protein extracts (whole cell extracts or fractionated extracts) are loaded onto a polyacrylamide gel, separated by electrophoresis and transferred to membrane, such as used for western blotting. Immunoprecipitation with anti-RTK antibodies also can be used to fractionate and isolate RTK proteins before performing gel electrophoresis and western blotting. The membranes can be probed with anti-phosphotyrosine antibodies to detect phosphorylation as well as probed with anti-RTK antibodies to detect total RTK protein. Control cells, such as cells not expressing RTK isoform and cells not exposed to ligand can be subjected to the same procedures for comparison.

Tyrosine phosphorylation also can be measured directly, such as by mass spectroscopy. For example, the effect of an RTK isoform on the phosphorylation state of an RTK can be measured, such as by treating intact cells with various concentrations of an RTK isoform and measuring the effect on activation of an RTK. The RTK can be isolated by immunoprecipitation and trypsinized to produce peptide fragments for analysis by mass spectroscopy. Peptide mass spectroscopy is a well-established method for quantitatively determining the extent of tyrosine phosphorylation for proteins; phosphorylation of tyrosine increases the mass of the peptide ion containing the phosphotyrosine, and this peptide is readily separated from the non-phosphorylated peptide by mass spectroscopy.

For example, tyrosine-1139 and tyrosine-1248 are known to be autophosphorylated in the ErbB2 RTK. Trypsinized peptides can be empirically determined or predicted based on polypeptide, for example by using ExPASy-PeptideMass program. The extent of phosphorylation of tyrosine-1139 and tyrosine-1248 can be determined from the mass spectroscopy data of peptides containing these tyrosines. Such assays can be used to assess the extent of auto-phosphorylation of an RTK isoform and the ability of an RTK isoform to transphosphorylate an RTK.

2. Complexation

Complexation, such as dimerization of RTKs and RTK isoforms and trimerization of TNFRs and TNFR isoforms, can be detected and/or measured. For example, isolated polypeptides can be mixed together, subjected to gel electrophoresis and western blotting. CSRs and/or CSR isoforms also can be added to cells and cell extracts, such as whole cell or fractionated extracts, and can be subjected to gel electrophoresis and western blotting. Antibodies recognizing the polypeptides can be used to detect the presence of monomers, dimers and other complexed forms. Alternatively, labeled CSRs and/or labeled CSR isoforms can be detected in the assays.

For example, such assays can be used to compare homodimerization of an RTK or heterodimerization of two or more RTKs in the presence and absence of an RTK isoform. Assays also can be performed to assess homodimerization of an RTK isoform and/or its ability to heterodimerize with an RTK. For example an ErbB2 RTK isoform can be assessed for its ability to heterodimerize with ErbB2, ErbB3 and ErbB4. Additionally, an ErbB2 RTK isoform can be assessed for its ability to modulate the ability of ErbB2 to homodimerize with itself.

3. Ligand Binding

Generally, CSRs bind to one or more ligands. Ligand binding modulates the activity of the receptor and thus modulates, for example, signaling within a signal transduction pathway. Ligand binding of a CSR isoform and ligand binding of a CSR in the presence of a CSR isoform can be measured. For example, labeled ligand such as radiolabeled ligand can be added to purified or partially purified CSR in the presence and absence (control) of a CSR isoform. Immunoprecipitation and measurement of radioactivity can be used to quantify the amount of ligand bound to a CSR in the presence and absence of a CSR isoform. A CSR isoform also can be assessed for ligand binding such as by incubating a CSR isoform with labeled ligand and determining the amount of labeled ligand bound by a CSR isoform, for example, compared to an amount bound by a wildtype or predominant form of a corresponding CSR.

4. Cell Proliferation Assays

A number of RTKs, for example VEGFR, are involved in cell proliferation. Effects of an RTK isoform on cell proliferation can be measured. For example, ligand can be added to cells expressing an RTK. An RTK isoform can be added to such cells before, concurrently or after ligand addition and effects on cell proliferation measured. Alternatively an RTK isoform can be expressed in such cell models, for example using an adenovirus vector. For example, a VEGFR isoform is added to endothelial cells expressing VEGFR. Following isoform addition, VEGF ligand is added and the cells are incubated at standard growth temperature (e.g. 37° C.) for several days. Cells are trypsinized, stained with trypan blue and viable cells are counted. Cells not exposed to VEGFR isoform and/or ligand are used as controls for comparison. Other suitable controls can be employed.

5. Cell Disease Model Assays

Cells from a disease or condition or that can be modulated to mimic a disease or condition can be used to measure/and or detect the effect of an CSR isoform. Numerous animal and in vitro disease models are known to those of skill in the art. For example, a CSR isoform is added or expressed in cells and a phenotype is measured or detected in comparison to cells not exposed to or not expressing a CSR isoform. Such assays can be used to measure effects including effects on cell proliferation, metastasis, inflammation, angiogenesis, pathogen infection and bone resorption.

For example, effects of a MET isoform can be measured using such assays. A liver cell model such as HepG2 liver cells can be used to monitor the infectivity of malaria in culture by sporozoites. An RTK isoform such as a MET isoform can be added to the cells and/or expressed in the cells. Infection of such cells with malaria sporozoites is then measured, such as by staining and counting the EEFs (exoerythrocytic forms) of the sporozoite that are produced as a result of infection Carrolo et al. (2003) Nat Med 9(11):1363-1369. Effects of an RTK isoform can be assessed by comparing results to cells not exposed or expressing an RTK isoform and/or uninfected cells.

Effects of a CSR isoform also can be measured in angiogenesis. For example, tubule formation by endothelial cells such as human umbilical vein endothelial cells (HUVEC) in vitro can be used as an assay to measure angiogenesis and effects on angiogenesis. Addition of varying amounts of a CSR isoform to an in vitro angiogenesis assay is a method suitable for screening the effectiveness of a CSR isoform as a modulator of angiogenesis.

Bone resorption can be measured in cell culture to measure effectiveness of an RTK-isoform, such as by using osteoclast cultures. Osteoclasts are highly differentiated cells of hematopoietic origin that resorb bone in the organism, and are able to resorb bone from bone slices in vitro. Methods for cell culture of osteoclasts and quantitative techniques for measuring bone resorption in osteoclast cell culture have been described in the art. For example, mononuclear cells can be isolated from human peripheral blood and cultured. Addition and/or expression of a CSR isoform can be used to assess effects on osteoclast formation such as by measuring multinucleated cells positive for tartrate-resistant acid phosphatase and resorbed area and collagen fragments released from bone slices. Dose response curves can be used to determine therapeutically effective amounts of a CSR isoform necessary to modulate bone resorption.

6. Animal Models

Animal models can be used to assess the effect of a CSR isoform. In one example, animal models of disease can be studied to determine if introduction of a CSR isoform affects the disease. For example, CSR isoform effects on tumor formation including cancer cell proliferation, migration and invasiveness can be measured. In one such assay, cancer cells such as ovarian cancer cells are infected with an adenovirus expressing a CSR isoform. After a culturing period in vitro, cells are trypsinized, suspended in a suitable buffer and injected into mice (e.g., subcutaneously into flanks and shoulders of model mice such as Balb/c nude mice). Tumor growth is monitored over time. Control cells, not expressing a CSR isoform, can be injected into mice for comparison. Similar assays can be performed with other cell types and animal models, for example, NIH3T3 cells, murine lung carcinoma (LLC) cells, primary Pancreatic Adenocarcinoma (PANC-1) cells, TAKA-1 pancreatic ductal cells, and C57BL/6 mice and SCID mice. In a further example, effects of CSR isoforms on ocular disorders can be assessed using assays such as a corneal micropocket assay. Briefly, mice receive cells expressing a CSR isoform (or control) by injection 2-3 days before the assay. Subsequently, the mice are anesthetized, and pellets of a ligand are implanted into the corneal micropocket of the eyes. Neovascularization is then measured, for example, 5 days following implantation. The effect of a CSR isoform on angiogenesis and eye phenotype compared to a control is then assessed. In an additional example, effects of a CSR isoform in a model of collagen type II-induced arthritis (CIA) can be assessed by intraperitoneal injection of SCID mice with splenocytes from DBA/1 mice that have been transduced with a retroviral vector containing the cDNA of a CSR isoform or unmodified splenocytes. Mice that receive unmodified splenocytes develop arthritis within 11-13 days and can be used as a reference control to determine effects of CSR isoform-expressing splenocytes on the development of arthritis as assessed, for example, by clinical, histological, or immunological (i.e. antibody levels) parameters of arthritis.

Effects of CSR isoforms on animal models of disease additionally can be assessed by the administration of purified or recombinant forms of a CSR isoform. For example, wound healing can be assessed in a model of impaired wound healing utilizing genetically diabetic db+/db+ mice whereby full-thickness excisional wounds are created on the backs of diabetic mice. Following treatment with a CSR isoform, either topically or systemically, wound healing can be assessed by analyzing for wound closure, inflammatory cell infiltration at the site of the wound, and expression of inflammatory cytokines. The effects of CSR isoforms on wound healing can be assessed over time and effects can be compared to mice that receive a control treatment, for example a vehicle only control. In a further example, a recombinant CSR isoform can be administered in a model of pulmonary fibrosis induced by bleomycin or silica to determine if lung fibrosis is reduced as assessed, for example, by analysis of histological sections for lung damage and by assaying for effects on bleomycin/silica induced increases of lung hydroxyproline content.

Animals deficient in a CSR isoform also can be used to monitor the biological activity of a CSR isoform. For example an isoform-specific disruption can made by creating a targeted construct whereby upstream from an IRES-LacZ cassette, translational stop codons are introduced within the appropriate reading frame to ensure that the receptor protein terminates early. Alternatively, a LoxP/Cre recombination strategy can be used. Following confirmation of the targeted disruption, the consequences of a deficiency in a CSR isoform can be established by analyzing the phenotype of the deficient mice compared to wildtype mice including the development of various organs such as, for example, lung, limbs, eyelids, anterior pituitary gland, and pancreas. In addition, by histology or isolation of specific cell populations, other parameters, such as apoptosis or cell proliferation, can be assessed to determine if there is a difference between animals or isolated cells lacking the CSR isoform compared to wildtype CSR. Components of signaling cascades and expression of downstream genes also can be assessed to determine if the absence of a CSR isoform affects receptor signaling and gene expression.

### I. Preparation, Formulation and Administration of CSR Isoforms and CSR Isoform Compositions

CSR isoforms and CSR isoform compositions, including RTK and TNFR isoforms and RTK and TNFR isoform compositions, can be formulated for administration by any route known to those of skill in the art including intramuscular, intravenous, intradermal, intraperitoneal injection, subcutaneous, epidural, nasal oral, rectal, topical, inhalational, buccal (e.g., sublingual), and transdermal administration or any route. CSR isoforms can be administered by any convenient route, for example by infusion or bolus injection, by absorption through epithelial or mucocutaneous linings (e.g., oral mucosa, rectal and intestinal mucosa, etc.) and can be administered with other biologically active agents, either sequentially, intermittently or in the same composition. Administration can be local, topical or systemic depending upon the locus of treatment. Local administration to an area in need of treatment can be achieved by, for example, but not limited to, local infusion during surgery, topical application, e.g., in conjunction with a wound dressing after surgery, by injection, by means of a catheter, by means of a suppository, or by means of an implant. Administration also can include controlled release systems including controlled release formulations and device controlled release, such as by means of a pump. The most suitable route in any given case will depend on the nature and severity of the disease or condition being treated and on the nature of the particular composition which is used.

Various delivery systems are known and can be used to administer CSR isoforms, such as but not limited to, encapsulation in liposomes, microparticles, microcapsules, recombinant cells capable of expressing the compound, receptor mediated endocytosis, and delivery of nucleic acid molecules encoding CSR isoforms such as retrovirus delivery systems.

Pharmaceutical compositions containing CSR isoforms can be prepared. Generally, pharmaceutically acceptable compositions are prepared in view of approvals for a regulatory agency or other prepared in accordance with generally recognized pharmacopeia for use in animals and in humans. Pharmaceutical compositions can include carriers such as a diluent, adjuvant, excipient, or vehicle with which an isoform is administered. Such pharmaceutical carriers can be sterile liquids, such as water and oils, including those of petroleum, animal, vegetable or synthetic origin, such as peanut oil, soybean oil, mineral oil, and sesame oil. Water is a typical carrier when the pharmaceutical composition is administered intravenously. Saline solutions and aqueous dextrose and glycerol solutions also can be employed as liquid carriers, particularly for injectable solutions. Compositions can contain along with an active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polyinylpyrrolidine, celluloses and derivatives thereof, povidone, crospovidones and other such binders known to those of skill in the art. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, and ethanol. A composition, if desired, also can contain minor amounts of wetting or emulsifying agents, or pH buffering agents, for example, acetate, sodium citrate, cyclodextrine derivatives, sorbitan monolaurate, triethanolamine sodium acetate, triethanolamine oleate, and other such agents. These compositions can take the form of solutions, suspensions, emulsion, tablets, pills, capsules, powders, and sustained release formulations. A composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, and other such agents. Examples of suitable pharmaceutical carriers are described in “Remington's Pharmaceutical Sciences” by E. W. Martin. Such compositions will contain a therapeutically effective amount of the compound, generally in purified form, together with a suitable amount of carrier so as to provide the form for proper administration to the patient. The formulation should suit the mode of administration.

Formulations are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable derivatives thereof. Pharmaceutically therapeutically active compounds and derivatives thereof are typically formulated and administered in unit dosage forms or multiple dosage forms. Each unit dose contains a predetermined quantity of therapeutically active compound sufficient to produce the desired therapeutic effect, in association with the required pharmaceutical carrier, vehicle or diluent. Examples of unit dose forms include ampoules and syringes and individually packaged tablets or capsules. Unit dose forms can be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses that are not segregated in packaging.

Dosage forms or compositions containing active ingredient in the range of 0.005% to 100% with the balance made up from non-toxic carrier can be prepared. For oral administration, pharmaceutical compositions can take the form of, for example, tablets or capsules prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well-known in the art.

Pharmaceutical preparation also can be in liquid form, for example, solutions, syrups or suspensions, or can be presented as a drug product for reconstitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid).

Formulations suitable for rectal administration can be provided as unit dose suppositories. These can be prepared by admixing the active compound with one or more conventional solid carriers, for example, cocoa butter, and then shaping the resulting mixture.

Formulations suitable for topical application to the skin or to the eye include ointments, creams, lotions, pastes, gels, sprays, aerosols and oils. Exemplary carriers include Vaseline, lanoline, polyethylene glycols, alcohols, and combinations of two or more thereof. The topical formulations also can contain 0.05 to 15, 20, 25 percent by weight of thickeners selected from among hydroxypropyl methyl cellulose, methyl cellulose, polyvinylpyrrolidone, polyvinyl alcohol, poly(alkylene glycols), poly/hydroxyalkyl, (meth)acrylates or poly(meth)acrylamides. A topical formulation is often applied by instillation or as an ointment into the conjunctival sac. It also can be used for irrigation or lubrication of the eye, facial sinuses, and external auditory meatus. It also can be injected into the anterior eye chamber and other places. A topical formulation in the liquid state can be also present in a hydrophilic three-dimensional polymer matrix in the form of a strip or contact lens, from which the active components are released.

For administration by inhalation, the compounds for use herein can be delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of, e.g., gelatin, for use in an inhaler or insufflator can be formulated containing a powder mix of the compound and a suitable powder base such as lactose or starch.

Formulations suitable for buccal (sublingual) administration include, for example, lozenges containing the active compound in a flavored base, usually sucrose and acacia or tragacanth; and pastilles containing the compound in an inert base such as gelatin and glycerin or sucrose and acacia.

Pharmaceutical compositions of CSR isoforms can be formulated for parenteral administration by injection, e.g., by bolus injection or continuous infusion. Formulations for injection can be presented in unit dosage form, e.g., in ampoules or in multi-dose containers, with an added preservative. The compositions can be suspensions, solutions or emulsions in oily or aqueous vehicles, and can contain formulatory agents such as suspending, stabilizing and/or dispersing agents. Alternatively, the active ingredient can be in powder form for reconstitution with a suitable vehicle, e.g., sterile pyrogen-free water or other solvents, before use.

Formulations suitable for transdermal administration can be presented as discrete patches adapted to remain in intimate contact with the epidermis of the recipient for a prolonged period of time. Such patches suitably contain the active compound as an optionally buffered aqueous solution of, for example, 0.1 to 0.2M concentration with respect to the active compound. Formulations suitable for transdermal administration also can be delivered by iontophoresis (see, e.g., Pharmaceutical Research 3(6), 318 (1986)) and typically take the form of an optionally buffered aqueous solution of the active compound.

Pharmaceutical compositions also can be administered by controlled release means and/or delivery devices (see, e.g., in U.S. Pat. Nos. 3,536,809; 3,598,123; 3,630,200; 3,845,770; 3,847,770; 3,916,899; 4,008,719; 4,687,610; 4,769,027; 5,059,595; 5,073,543; 5,120,548; 5,354,566; 5,591,767; 5,639,476; 5,674,533 and 5,733,566).

In certain embodiments, liposomes and/or nanoparticles may also be employed with CSR isoform administration. Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 .ANG., containing an aqueous solution in the core.

Phospholipids can form a variety of structures other than liposomes when dispersed in water, depending on the molar ratio of lipid to water. At low ratios, the liposomes form. Physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

Liposomes interact with cells via different mechanisms: Endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. Varying the liposome formulation can alter which mechanism is operative, although more than one may operate at the same time. Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use herein, and such particles can be easily made.

Administration methods can be employed to decrease the exposure of CSR isoforms to degradative processes, such as proteolytic degradation and immunological intervention via antigenic and immunogenic responses. Examples of such methods include local administration at the site of treatment. Pegylation of therapeutics has been reported to increase resistance to proteolysis; increase plasma half-life, and decrease antigenicity and immunogenicity. Examples of pegylation methodologies are known in the art (see for example, Lu and Felix, Int. J. Peptide Protein Res., 43: 127-138, 1994; Lu and Felix, Peptide Res., 6: 142-6, 1993; Felix et al., Int. J. Peptide Res., 46: 253-64, 1995; Benhar et al., J. Biol. Chem., 269: 13398-404, 1994; Brumeanu et al., J Immunol., 154: 3088-95, 1995; see also, Caliceti et al. (2003) Adv. Drug Deliv. Rev. 55(10):1261-77 and Molineux (2003) Pharmacotherapy 23 (8 Pt 2):3S-8S). Pegylation also can be used in the delivery of nucleic acid molecules in vivo. For example, pegylation of adenovirus can increase stability and gene transfer (see, e.g., Cheng et al. (2003) Pharm. Res. 20(9): 1444-51).

Desirable blood levels can be maintained by a continuous infusion of the active agent as ascertained by plasma levels. It should be noted that the attending physician would know how to and when to terminate, interrupt or adjust therapy to lower dosage due to toxicity, or bone marrow, liver or kidney dysfunctions. Conversely, the attending physician would also know how to and when to adjust treatment to higher levels if the clinical response is not adequate (precluding toxic side effects). administered, for example, by oral, pulmonary, parental (intramuscular, intraperitoneal, intravenous (IV) or subcutaneous injection), inhalation (via a fine powder formulation), transdermal, nasal, vaginal, rectal, or sublingual routes of administration and can be formulated in dosage forms appropriate for each route of administration (see, e.g., International PCT application Nos. WO 93/25221 and WO 94/17784; and European Patent Application 613,683).

A CSR isoform is included in the pharmaceutically acceptable carrier in an amount sufficient to exert a therapeutically useful effect in the absence of undesirable side effects on the patient treated. Therapeutically effective concentration can be determined empirically by testing the compounds in known in vitro and in vivo systems, such as the assays provided herein.

The concentration a CSR isoform in the composition will depend on absorption, inactivation and excretion rates of the complex, the physicochemical characteristics of the complex, the dosage schedule, and amount administered as well as other factors known to those of skill in the art. The amount of a CSR isoform to be administered for the treatment of a disease or condition, for example cancer, autoimmune disease and infection can be determined by standard clinical techniques. In addition, in vitro assays and animal models can be employed to help identify optimal dosage ranges. The precise dosage, which can be determined empirically, can depend on the route of administration and the seriousness of the disease. Suitable dosage ranges for administration can range from about 0.01 pg/kg body weight to 1 mg/kg body weight and more typically 0.05 mg/kg to 200 mg/kg CSR isoform: patient weight.

A CSR isoform can be administered at once, or can be divided into a number of smaller doses to be administered at intervals of time. CSR isoforms can be administered in one or more doses over the course of a treatment time for example over several hours, days, weeks, or months. In some cases, continuous administration is useful. It is understood that the precise dosage and duration of treatment is a function of the disease being treated and can be determined empirically using known testing protocols or by extrapolation from in vivo or in vitro test data. It is to be noted that concentrations and dosage values also can vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or use of compositions and combinations containing them.

### J. In Vivo Expression of CSR Isoforms and Gene Therapy

CSR isoforms can be delivered to cells and tissues by expression of nucleic acid molecules. CSR isoforms can be administered as nucleic acid molecules encoding a CSR isoform, including ex vivo techniques and direct in vivo expression.

1. Delivery of Nucleic Acids

Nucleic acids can be delivered to cells and tissues by any method known to those of skill in the art.

a. Vectors—Episomal and Integrating

Methods for administering CSR isoforms by expression of encoding nucleic acid molecules include administration of recombinant vectors. The vector can be designed to remain episomal, such as by inclusion of an origin of replication or can be designed to integrate into a chromosome in the cell.

CSR isoforms also can be used in ex vivo gene expression therapy using non-viral vectors. For example, cells can be engineered to express a CSR isoform, such as by integrating a CSR isoform encoding-nucleic acid into a genomic location, either operatively linked to regulatory sequences or such that it is placed operatively linked to regulatory sequences in a genomic location. Such cells then can be administered locally or systemically to a subject, such as a patient in need of treatment.

Viral vectors, include, for example adenoviruses, herpes viruses, retroviruses and others designed for gene therapy can be employed. The vectors can remain episomal or can integrate into chromosomes of the treated subject. A CSR isoform can be expressed by a virus, which is administered to a subject in need of treatment. Virus vectors suitable for gene therapy include adenovirus, adeno-associated virus, retroviruses, lentiviruses and others noted above. For example, adenovirus expression technology is well-known in the art and adenovirus production and administration methods also are well known. Adenovirus serotypes are available, for example, from the American Type Culture Collection (ATCC, Rockville, Md.). Adenovirus can be used ex vivo, for example, cells are isolated from a patient in need of treatment, and transduced with a CSR isoform-expressing adenovirus vector. After a suitable culturing period, the transduced cells are administered to a subject, locally and/or systemically. Alternatively, CSR isoform-expressing adenovirus particles are isolated and formulated in a pharmaceutically-acceptable carrier for delivery of a therapeutically effective amount to prevent, treat or ameliorate a disease or condition of a subject. Typically, adenovirus particles are delivered at a dose ranging from 1 particle to 1014 particles per kilogram subject weight, generally between 106 or 108 particles to 1012 particles per kilogram subject weight. In some situations it is desirable to provide a nucleic acid source with an agent that targets cells, such as an antibody specific for a cell surface membrane protein or a target cell, or a ligand for a receptor on a target cell.

A CSR isoform can be expressed by a virus and the virus administered to a subject in need of treatment. Virus vectors suitable for gene therapy include, for example, adenovirus, adeno-associated virus, retroviruses, lentiviruses Adenovirus expression technology is well-known in the art and adenovirus production and administration methods also are well known. Adenovirus serotypes are available, for example, from the American Type Culture Collection (ATCC, Rockville, Md.). Adenovirus can be used ex vivo, for example, cells are isolated from a patient in need of treatment, and transduced with a CSR isoform-expressing adenovirus vector. After a suitable culturing period, the transduced cells are administered to a subject, locally and/or systemically. As another example, CSR isoform-expressing adenovirus particles are isolated and formulated in a pharmaceutically-acceptable carrier for delivery of a therapeutically effective amount to prevent, treat or ameliorate a disease or condition of a subject. Typically, adenovirus particles are delivered at a dose ranging from 1 particle to 1014 particles per kilogram subject weight, generally between 106 or 108 particles to 1012 particles per kilogram subject weight. In some situations it is desirable to provide a nucleic acid source with an agent that targets cells, such as an antibody specific for a cell surface membrane protein or a target cell, or a ligand for a receptor on a target cell. Where liposomes are employed, proteins which bind to a cell surface membrane protein associated with endocytosis may be used for targeting and/or to facilitate uptake, e.g. capsid proteins or fragments thereof tropic for a particular cell type, antibodies for proteins which undergo internalization in cycling, and proteins that target intracellular localization and enhance intracellular half-life.

b. Artificial Chromosomes and Other Non-Viral Vector Delivery Methods

CSR isoforms also can be used in ex vivo gene expression therapy using non-viral vectors. For example, cells can be engineered which express a CSR isoform, such as by integrating a CSR isoform sequence into a genomic location, either operatively linked to regulatory sequences or such that it is placed operatively linked to regulatory sequences in a genomic location. Such cells then can be administered locally or systemically to a subject, such as a patient in need of treatment.

The nucleic acid molecules can be introduced into artificial chromosomes and other non-viral vectors. Artificial chromosomes (see, e.g., U.S. Pat. No. 6,077,697 and PCT International PCT application No. WO 02/097059) can be engineered to encode and express the isoform.

c. Liposomes and Other Encapsulated Forms and Administration of Cells Containing the Nucleic Acids

The nucleic acids can be encapsulated in a vehicle, such as a liposome, or introduced into a cells, such as a bacterial cell, particularly an attenuated bacterium or introduced into a viral vector. For example, when liposomes are employed, proteins that bind to a cell surface membrane protein associated with endocytosis can be used for targeting and/or to facilitate uptake, e.g. capsid proteins or fragments thereof tropic for a particular cell type, antibodies for proteins which undergo internalization in cycling, and proteins that target intracellular localization and enhance intracellular half-life.

2. In Vitro and Ex Vivo Delivery

For ex vivo and in vivo methods, nucleic acid molecules encoding the CSR isoform is introduced into cells that are from a suitable donor or the subject to be treated. In vivo expression of a CSR isoform can be linked to expression of additional molecules. For example, expression of a CSR isoform can be linked with expression of a cytotoxic product such as in an engineered virus or expressed in a cytotoxic virus. Such viruses can be targeted to a particular cell type that is a target for a therapeutic effect. The expressed CSR isoform can be used to enhance the cytotoxicity of the virus.

In vivo expression of a CSR isoform can include operatively linking a CSR isoform encoding nucleic acid molecule to specific regulatory sequences such as a cell-specific or tissue-specific promoter. CSR isoforms also can be expressed from vectors that specifically infect and/or replicate in target cell types and/or tissues. Inducible promoters can be use to selectively regulate CSR isoform expression.

Cells into which a nucleic acid can be introduced for purposes of therapy encompass any desired, available cell type appropriate for the disease or condition to be treated, including but not limited to epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells, e.g., such as stem cells obtained from bone marrow, umbilical cord blood, peripheral blood, fetal liver, and other sources thereof. Tumor cells also can be target cells for in vivo expression of CSR isoforms. Cells used for in vivo expression of an isoform also include cells autologous to the patient. Such cells can be removed from a patient, nucleic acids for expression of a CSR isoform introduced, and then administered to a patient such as by injection or engraftment.

Techniques suitable for the transfer of nucleic acid into mammalian cells in vitro include the use of liposomes and cationic lipids (e.g., DOTMA, DOPE and DC-Chol), electroporation, microinjection, cell fusion, DEAE-dextran, and calcium phosphate precipitation methods. Methods of DNA delivery can be used to express CSR isoforms in vivo. Such methods include liposome delivery of nucleic acids and naked DNA delivery, including local and systemic delivery such as using electroporation, ultrasound and calcium-phosphate delivery. Other techniques include microinjection, cell fusion, chromosome-mediated gene transfer, microcell-mediated gene transfer and spheroplast fusion.

For ex vivo treatment, cells from a donor compatible with the subject to be treated or cells from the subject to be treated are removed, the nucleic acid is introduced into these isolated cells and the modified cells are administered to the subject.

Treatment includes direct administration, such as, for example, encapsulated within porous membranes, which are implanted into the patient (see, e.g. U.S. Pat. Nos. 4,892,538 and 5,283,187). Techniques suitable for the transfer of nucleic acid into mammalian cells in vitro include the use of liposomes and cationic lipids (e.g., DOTMA, DOPE and DC-Chol), electroporation, microinjection, cell fusion, DEAE-dextran, and calcium phosphate precipitation methods. Methods of DNA delivery can be used to express CSR isoforms in vivo. Such methods include liposome delivery of nucleic acids and naked DNA delivery, including local and systemic delivery such as using electroporation, ultrasound and calcium-phosphate delivery. Other techniques include microinjection, cell fusion, chromosome-mediated gene transfer, microcell-mediated gene transfer and spheroplast fusion.

In vivo expression of a CSR isoform can be linked to expression of additional molecules. For example, expression of a CSR isoform can be linked with expression of a cytotoxic product such as in an engineered virus or expressed in a cytotoxic virus. Such viruses can be targeted to a particular cell type that is a target for a therapeutic effect. The expressed CSR isoform can be used to enhance the cytotoxicity of the virus.

In vivo expression of a CSR isoform can include operatively linking a CSR isoform encoding nucleic acid molecule to specific regulatory sequences such as a cell-specific or tissue-specific promoter. CSR isoforms also can be expressed from vectors that specifically infect and/or replicate in target cell types and/or tissues. Inducible promoters can selectively regulate CSR isoform expression.

3. Systemic, Local and Topical Delivery

Nucleic acid molecules, as naked nucleic acids or in vectors, artificial chromosomes, liposomes and other vehicles can be administered to the subject by systemic administration, topical, local and other routes of administration. When systemic and in vivo, the nucleic acid molecule or vehicle containing the nucleic acid molecule can be targeted to a cell.

Administration also can be direct, such as by administration of a vector or cells that typically targets a cell or tissue. For example, tumor cells and proliferating cells can be targeted cells for in vivo expression of CSR isoforms. Cells used for in vivo expression of an isoform also include cells autologous to the patient. Such cells can be removed from a patient, nucleic acids for expression of a CSR isoform introduced, and then administered to a patient such as by injection or engraftment.

### K. CSRs and Angiogenesis

CSRs participate in pathways involved in a variety of pathways, including those that participate in angiogenesis, cell proliferation, inflammatory responses, and neovascularization among others. Angiogenesis is a process by which new blood vessels are formed. It occurs in healthy individuals, such as during wound healing and in aberrant conditions, such as in tumors. It occurs for example, in a healthy body in would healing and for restoring blood flow to tissues after injury or insult. Angiogenesis is a component of tumorigenesis, which requires the growth of blood cells to feed the growing tumorous mass. In females, angiogenesis also occurs during the monthly reproductive cycle to rebuild the uterus lining, to mature the egg during ovulation and during pregnancy to build the placenta.

Angiogenesis is controlled through a series of “on” and “off” switches. The primary “on” switches are angiogenesis-stimulating growth factors. The primary “off switches” are angiogenesis inhibitors. When angiogenic growth factors are produced in excess of angiogenesis inhibitors, the balance can be in favor of blood vessel growth. When inhibitors are present in excess of stimulators, angiogenesis is stopped. A healthy body maintains a balance of angiogenesis modulators. A number of angiogenic growth factors are known. These include, for example, angiogenin, angiopoietin-1, Del-1, fibroblast growth factors: acidic (aFGF) and basic (bFGF), follistatin, granulocyte colony-stimulating factor (G-CSF), hepatocyte growth factor (HGF), scatter factor (SF), interleukin-8 (IL-8), leptin, midkine, placental growth factor, platelet-derived endothelial cell growth factor (PD-ECGF), platelet-derived growth factor-BB (PDGF-BB), pleiotrophin (PTN), progranulin, proliferin, transforming growth factor-alpha (TGF-alpha), transforming growth factor-beta (TGF-beta), tumor necrosis factor-alpha (TNF-alpha), and vascular endothelial growth factor (VEGF)/vascular permeability factor (VPF).

1. Angiogenesis and Disease

Cellular receptors for angiogenic factors (positive and negative) can act as points of intervention in multiple disease processes, for example, in diseases and conditions where the balance of angiogenic growth factors has been altered and/or the amount or timing of angiogenesis is altered. For example, in some situations ‘too much’ angiogenesis can be detrimental, such as angiogenesis that supplies blood to tumor foci, in inflammatory responses and other aberrant angiogenic-related conditions. The growth of tumors, or sites of proliferation in chronic inflammation, generally requires the recruitment of neighboring blood vessels and vascular endothelial cells to support their metabolic requirements. This is because the diffusion is limited for oxygen in tissues. Exemplary conditions that require angiogenesis include, but are not limited to solid tumors and hematologic malignancies such as lymphomas, acute leukemia, and multiple myeloma, where increased numbers of blood vessels are observed in the pathologic bone marrow.

A critical element in the growth of primary tumors and formation of metastatic sites is the angiogenic switch: the ability of the tumor or inflammatory site to promote the formation of new capillaries from preexisting host vessels. The angiogenic switch, as used in this context, refers to disease-associated angiogenesis required for the progression of cancer and inflammatory diseases, such as rheumatoid arthritis. It is a switch that activates a cascade of physiological activities that finally result in the extension of new blood vessels to support the growth of diseased tissue. Stimuli for neo-angiogenesis include hypoxia, inflammation, and genetic lesions in oncogenes or tumor suppressors that alter disease cell gene expression.

Angiogenesis also plays a role in inflammatory diseases. These diseases have a proliferative component, similar to a tumor focus. In rheumatoid arthritis, one component of this is characterized by aberrant proliferation of synovial fibroblasts, resulting in pannus formation. The pannus is composed of synovial fibroblasts which have some phenotypic characteristics with transformed cells. As a pannus grows within the joint it expresses many proangiogenic signals, and experiences many of the same neo-angiogenic requirements as a tumor. The need for additional blood supply, neoangiogenesis, is critical. Similarly, many chronic inflammatory conditions also have a proliferative component in which some of the cells composing it may have characteristics usually attributed to transformed cells.

Another example of a condition involving excess angiogenesis is diabetic retinopathy (Lip et al. Br J Ophthalmology 88: 1543, 2004)). Diabetic retinopathy has angiogenic, inflammatory and proliferative components; overexpression of VEGF, and angiopoietin-2 are common. This overexpression is likely required for disease-associated remodeling and branching of blood vessels, which then supports the proliferative component of the disease.

2. Angiogenesis

Angiogenesis includes several steps, including the recruitment of circulating endothelial cell precursors (CEPs), stimulation of new endothelial cell (EC) growth by growth factors, the degradation of the ECM by proteases, proliferation of ECs and migration into the target, which could be a tumor site or another proliferative site caused by inflammation. This results in the eventual formation of new capillary tubes. Such blood vessels are not necessarily normal in structure. They may have chaotic architecture and blood flow. Due to an imbalance of angiogenic regulators such as vascular endothelial growth factor, (VEGF) and angiopoietins, the new vessels supplying tumorous or inflammatory sites are tortuous and dilated with an uneven diameter, excessive branching, and shunting. Blood flow is variable, with areas of hypoxia and acidosis leading to the selection of variants that are resistant to hypoxia-induced apoptosis (often due to the loss of p53 expression); and enhanced production of proangiogenic signals. Disease-associated vessel walls have numerous openings, widened interendothelial junctions, and discontinuous or absent basement membrane; this contributes to the high vascular permeability of these vessels and, together with lack of functional lymphatics/drainage, causes interstitial hypertension. Disease-associated blood vessels may lack perivascular cells such as pericytes and smooth muscle cells that normally regulate vasoactive control in response to tissue metabolic needs. Unlike normal blood vessels, the vascular lining of tumor vessels is not a homogenous layer of ECs but often consists of a mosaic of ECs and tumor cells; the concept of cancer cell-derived vascular channels, which may be lined by ECM secreted by the tumor cells, is referred to as vascular mimicry.

A similar situation occurs where blood vessels rapidly invade sites of acute inflammation. The ECs of angiogenic blood vessels are unlike quiescent ECs found in adult vessels, where only 0.01% of ECs are dividing. During tumor angiogenesis, ECs are highly proliferative and express a number of plasma membrane proteins that are characteristic of activated endothelium, including growth factor receptors and adhesion molecules such as integrins. Tumors utilize a number of mechanisms to promote their vascularization, and in each case they subvert normal angiogenic processes to suit this purpose. For this reason, increased production of angiogenic factors, both proliferative with respect to endothelium; and structural (allowing for increased branching of the neovasculature) are likely to occur in disease foci, as in cancer or chronic inflammatory disease.

3. Cell Surface Receptors in Angiogenesis

Cell surface receptors including RTKs, and their ligands play a role in the regulation of angiogenesis (see for example, FIG. 1). Angiogenic endothelium expresses a number of receptors not found on resting endothelium. These include receptor tyrosine kinases (RTK) and integrins that bind to the extracellular matrix and mediate endothelial cells adhesion, migration, and invasion.

Endothelial cells (ECs) also express RTK (i.e., the FGF and PDGF receptors) that are found on many other cell types. Functions mediated by activated RTK include proliferation, migration, and enhanced survival of endothelial cells, as well as regulation of the recruitment of perivascular cells and bloodborne circulating endothelial precursors and hematopoietic stem cells to the tumor. One example of a CSR involved in angiogenesis is VEGFR. VEGFR-1 receptors and VEGF-A ligand are involved in cell proliferation, migration and differentiation in angiogenesis. VEGF-A is a heparin-binding glycoprotein with at least four isoforms that regulate blood vessel formation by binding to RTKs, VEGFR-1 and VEGFR-2. These VEGF receptors are expressed on all ECs in addition to a subset of hematopoietic cells. VEGFR-2 regulates EC proliferation, migration, and survival, while VEGFR-1 may act as an antagonist of R1 in ECs but also can plays a role in angioblast differentiation during embryogenesis.

Additional signaling pathways also are involved in angiogenesis. The angiopoietin, Ang1, produced by stromal cells, binds to the EC RTK TEK and promotes the interaction of ECs with the ECM and perivascular cells, such as pericytes and smooth muscle cells, to form tight, non-leaky vessels. PDGF and basic fibroblast growth factor (bFGF) help to recruit these perivascular cells. Ang1 is required for maintaining the quiescence and stability of mature blood vessels and prevents the vascular permeability normally induced by VEGF and inflammatory cytokines.

Proangiogenic cytokines, chemokines, and growth factors secreted by stromal cells or inflammatory cells make important contributions to neovascularization, including bFGF, transforming growth factor-alpha, TNF-alpha, and IL-8. In contrast to normal endothelium, angiogenic endothelium overexpresses specific members of the integrin family of ECM-binding proteins that mediate EC adhesion, migration, and survival. Integrins mediate spreading and migration of ECs and are required for angiogenesis induced by VEGF and bFGF, which in turn can upregulate EC integrin expression. EC adhesion molecules can be upregulated (i.e., by VEGF, TNF-alpha). VEGF promotes the mobilization and recruitment of circulating endothelial cell precursors (CEPs) and hematopoietic stem cells (HSCs) to tumors where they colocalize and appear to cooperate in neovessel formation. CEPs express VEGFR-2, while HSCs express VEGFR-1, a receptor, or VEGF and P1GF. Both CEPs and HSCs are derived from a common precursor, the hemangioblast. CEPs are thought to differentiate into ECs, whereas the role of HSC-derived cells (such as tumor-associated macrophages) may be to secrete angiogenic factors required for sprouting and stabilization of ECs (VEGF, bFGF, angiopoietins) and to activate MMPs, resulting in ECM remodeling and growth factor release. In mouse tumor models and in human cancers, increased numbers of CEPs and subsets of VEGFR-1 or VEGFR-expressing HSCs can be detected in the circulation, which may correlate with increased levels of serum VEGF.

4. Tumor and Inflammatory Diseases

Tumors secrete trophic angiogenic molecules, such as VEGF family of endothelial growth factors, that induce the proliferation and migration of host ECs into the tumor. Tumor vessels appear to be more dependent on VEGFR signaling for growth and survival than normal ECs. Sprouting in normal and pathogenic angiogenesis is regulated by three families of transmembrane RTKs expressed on ECs and their ligand: VEGFs, angiopoietins, and ephrins, which are produced by tumor cells, inflammatory cells, or stromal cells in the microenvironment of the disease site. Tumor or inflammatory disease-associated angiogenesis is a complex process involving many different cell types that proliferate, migrate, invade, and differentiate in response to signals from microenvironment. Endothelial cells (ECs) sprout from host vessels in response to VEGF, bFGF, Ang2, and other proangiogenic stimuli. Sprouting is stimulated by VEGF/VEGFR-2, Ang2/TEK, and integrin/extracellular matrix (ECM) interactions. Bone marrow-derived circulating endothelial precursors (CEPs) migrate to the tumor in response to VEGF and differentiate into ECs, while hematopoietic stem cells differentiate into leukocytes, including tumor/disease site-associated macrophages that secrete angiogenic growth factors and produce MMPs that remodel the ECM and release bound growth factors.

When tumor cells arise in or metastasize to an avascular area, they grow to a size limited by hypoxia and nutrient deprivation. This condition, also likely to occur in other localized proliferative diseases, leads to the selection of cells that produce angiogenic factors. Hypoxia, a key regulator of tumor angiogenesis, causes the transcriptional induction of the gene(s) encoding VEGF by a process that involves stabilization of the transcription factor hypoxia-inducible factor (HIF) 1. Under normoxic conditions, EC HIF-1 levels are maintained at a low level by proteasome-mediated destruction regulated by a ubiquitin E3-ligase encoded by the VHL (Von Hippel-Lindau) tumor-suppressor locus. However, under hypoxic conditions, the HIF-1 protein is not hydroxylated and association with VHL does not occur; therefore HIF-1 levels increase, and target genes including VEGF, nitric oxide synthetase (NOS), and Ang2 are induced. Loss of the VHL genes, as occurs in familial and sporadic renal cell carcinomas, also results in HIF-1 stabilization and induction of VEGF. Most tumors have hypoxic regions due to poor blood flow, and tumor cells in these areas stain positive for HIF-1 expression. These are conditions that lead to the de novo formation of blood vessels from differentiating endothelial cells, as occurs during embryonic development, and angiogenesis under normal (wound healing, corpus luteum formation) and pathologic processes (tumor angiogenesis, inflammatory conditions such as rheumatoid arthritis).

For diseased cell-derived VEGF, such as may be produced by a growing tumor focus or by pannus formation in rheumatoid arthritis, to initiate sprouting from host vessels, the stability conferred by the Ang1/TEK pathway must be perturbed; this occurs by the secretion of Ang2 by ECs that are undergoing active remodeling. Ang2 binds to TEK and is a competitive inhibitor of Ang1 action: under the influence of Ang2, preexisting blood vessels become more responsive to remodeling signals, with less adherence of ECs to stroma and associated perivascular cells and more responsiveness to VEGF. Therefore, Ang2 is required at early stages of neoangiogenesis for destabilizing the vasculature by making host ECs more sensitive to angiogenic signals. Since tumor ECs are blocked by Ang2, there is no stabilization by the Ang1/TEK interaction, and tumor blood vessels are leaky, hemorrhagic, and have poor association of ECs with underlying stroma. Sprouting tumor ECs express high levels of the transmembrane protein Ephrin-B2 and its receptor, the RTK EPH whose signaling appears to work with the angiopoietins during vessel remodeling. During embryogenesis, EPH receptors are expressed on the endothelium of primordial venous vessels while the transmembrane ligand ephrin-B2 is expressed by cells of primordial arteries; the reciprocal expression may regulate differentiation and patterning of the vasculature.

Development of tumor lymphatics also is associated with expression of cell surface receptors, including VEGFR-3 and its ligands VEGF-C and VEGF-D. The role of these vessels in tumor cell metastasis to regional lymph nodes remains to be determined, since, as discussed above, interstitial pressures within tumors are high and most lymphatic vessels may exist in a collapsed and nonfunctional state. However, VEGF-C levels in primary human tumors, including lung, prostate, and colorectal cancers, correlate significantly with metastasis to regional lymph nodes, and therefore it is possible that expression of VEGF-C,D/R3 may contribute to disease spreading by maintaining an exit for tumor cells from the primary site to lymph nodes and beyond.

5. Cell Surface Receptors and Treatment of Angiogenic Diseases and Conditions

Modulation of angiogenesis, neovascularization and/or cell proliferation can be used to treat diseases and conditions in which angiogenesis plays a role. For example, angiogenesis inhibitors can function by targeting the critical molecular pathways involved in EC proliferation, migration, and/or survival, many of which are unique to the activated endothelium in tumors. Inhibition of growth factor and adhesion-dependent signaling pathways can induce EC apoptosis with concomitant inhibition of tumor growth. ECs comprising the tumor vasculature are genetically stable and do not share genetic changes with tumor cells; the EC apoptosis pathways are therefore intact. Each EC of a tumor vessel helps provide nourishment to many tumor cells, and although tumor angiogenesis can be driven by a number of exogenous proangiogenic stimuli, experimental data indicate that blockade of a single growth factor (e.g., VEGF) can inhibit tumor-induced vascular growth. Because tumor blood vessels are distinct from normal ones, they may be selectively destroyed without affecting normal vessels.

Because cell surface receptors are involved in the regulation of angiogenesis, they can be therapeutic targets for treatment of diseases and conditions involving angiogenesis. Provided herein are CSR isoforms that can modulate one or more steps in the angiogenic process. CSR isoforms can be administered singly, in parallel or in other combinations. For instance, angiogenesis induced by bFGF can be blocked by inhibitors of the bFGFR such as a CSR isoform, and this can in turn inhibit activation of the VEGF pathway. The VEGFR pathway also can be blocked by a VEGFR isoform. CSR isoforms that modulate Ang/TEK and Ephrin/EPH pathways also can be administered to modulate angiogenesis. CSR isoforms that act as antagonists of the activity of VEGFR, bFGF, Ang2, TNF-alpha, TGF-alpha, and other factors such as ephrin antagonists, can be administered. These ligands and their receptors are required for the attraction of new endothelial cells, and/or their structural transformation into blood vessels by differentiation from circulating endothelial precursors (CEPs) or by inhibiting either tube formation or the needed branching. Hence, antagonizing one or more of these factors can inhibit the development and progression of cancer and inflammatory disease. As described herein, CSR isoforms can be administered as therapeutics for such diseases and conditions.

### L. Exemplary Treatments and Studies with CSR Isoforms

Provided herein are methods of treatment with CSR isoforms for diseases and conditions. CSR isoforms such as RTK isoforms and TNFR isoforms can be used in the treatment of a variety of diseases and conditions, including those described herein. Treatment can be effected by administering by suitable route formulations of the polypeptides, which can be provided in compositions as polypeptides and can be linked to targeting agents, for targeted delivery or encapsulated in delivery vehicles, such as liposomes. Alternatively, nucleic acids encoding the polypeptides can be administered as naked nucleic acids or in vectors, particularly gene therapy vectors. Gene therapy can be effected by any method known to those of skill in the art. Gene therapy can be effected in vivo by directly administering the nucleic acid or vector. For example, the nucleic acids can be delivered systemically, locally, topically or by any suitable route. The vectors or nucleic acids can be targeted by including targeting agents in delivery vehicle, such as a virus or liposome, or they can be conjugated to a targeting agent, such as an antibody. The vectors or nucleic acids can be introduced into cells ex vivo by removing cells from a subject or suitable donor, introducing the vector or nucleic acid into the cells and then introducing the modified cells into the subject.

The CSR isoforms provided herein can be used for treating a variety of disorders, particularly proliferative, immune and inflammatory disorders. Treatments, include, but are not limited to treatment of angiogenesis-related diseases and conditions including ocular diseases, atherosclerosis, cancer and vascular injuries, neurodegenerative diseases, including Alzheimer's disease, inflammatory diseases and conditions, including atherosclerosis, diseases and conditions associated with cell proliferation including cancers, and smooth muscle cell-associated conditions, and various autoimmune diseases. Exemplary treatments and preclinical studies are described for treatments and therapies with RTK and TNFR isoforms. Such descriptions are meant to be exemplary only and are not limited to a particular RTK or TNFR isoform. The particular treatment and dosage can be determined by one of skill in the art. Considerations in assessing treatment include, the disease to be treated, the severity and course of the disease, whether the molecule is administered for preventive or therapeutic purposes, previous therapy, the patient's clinical history and response to therapy, and the discretion of the attending physician.

1. Angiogenesis-Related Conditions

RTK isoforms including, but not limited to, VEGFR, PDGFR, TIE/TEK, EGFR, and EphA and TNFR isoforms including TNFR1 and TNFR2 can be used in treatment of angiogenesis-related diseases and conditions, such as ocular diseases and conditions, including ocular diseases involving neovascularization. Ocular neovascular disease is characterized by invasion of new blood vessels into the structures of the eye, such as the retina or cornea. It is the most common cause of blindness and is involved in approximately twenty eye diseases. In age-related macular degeneration, the associated visual problems are caused by an ingrowth of choroidal capillaries through defects in Bruch's membrane with proliferation of fibrovascular tissue beneath the retinal pigment epithelium. Angiogenic damage also is associated with diabetic retinopathy, retinopathy of prematurity, corneal graft rejection, neovascular glaucoma and retrolental fibroplasia. Other diseases associated with corneal neovascularization include, but are not limited to, epidemic keratoconjunctivitis, Vitamin A deficiency, contact lens overwear, atopic keratitis, superior limbic keratitis, pterygium keratitis sicca, sjogrens, acne rosacea, phylectenulosis, syphilis, Mycobacteria infections, lipid degeneration, chemical burns, bacterial ulcers, fungal ulcers, Herpes simplex infections, Herpes zoster infections, protozoan infections, Karposi sarcoma, Mooren ulcer, Terrien's marginal degeneration, marginal keratolysis, rheumatoid arthritis, systemic lupus, polyarteritis, trauma, Wegeners sarcoidosis, Scleritis, Steven's Johnson disease, periphigoid radial keratotomy, and corneal graph rejection. Diseases associated with retinal/choroidal neovascularization include, but are not limited to, diabetic retinopathy, macular degeneration, sickle cell anemia, sarcoid, syphilis, pseudoxanthoma elasticum, Pagets disease, vein occlusion, artery occlusion, carotid obstructive disease, chronic uveitis/vitritis, mycobacterial infections, Lyme's disease, systemic lupus erythematosus, retinopathy of prematurity, Eales disease, Bechets disease, infections causing a retinitis or choroiditis, presumed ocular histoplasmosis, Bests disease, myopia, optic pits, Stargardt's disease, pars planitis, chronic retinal detachment, hyperviscosity syndromes, toxoplasmosis, trauma and post-laser complications. Other diseases include, but are not limited to, diseases associated with rubeosis (neovascularization of the angle) and diseases caused by the abnormal proliferation of fibrovascular or fibrous tissue including all forms of proliferative vitreoretinopathy.

RTK and TNFR isoform therapeutic effects on angiogenesis such as in treatment of ocular diseases can be assessed in animal models, for example in cornea implants, such as described herein. For example, modulation of angiogenesis such as for an RTK can be assessed in a nude mouse model such as epidermoid A431 tumors in nude mice and VEGF- or PIGF-transduced rat C6 gliomas implanted in nude mice. CSR isoforms can be injected as protein locally or systemically. Alternatively cells expressing CSR isoforms can be inoculated locally or at a site remote to the tumor. Tumors can be compared between control treated and CSR isoform treated models to observe phenotypes of tumor inhibition including poorly vascularized and pale tumors, necrosis, reduced proliferation and increased tumor-cell apoptosis. In one such treatment, Flt-1 isoforms are used to treat ocular disease and assessed in such models.

Examples of ocular disorders that can be treated with TIE/TEK isoforms are eye diseases characterized by ocular neovascularization including, but not limited to, diabetic retinopathy (a major complication of diabetes), retinopathy of prematurity (this devastating eye condition, that frequently leads to chronic vision problems and carries a high risk of blindness, is a severe complication during the care of premature infants), neovascular glaucoma, retinoblastoma, retrolental fibroplasia, rubeosis, uveitis, macular degeneration, and corneal graft neovascularization. Other eye inflammatory diseases, ocular tumors, and diseases associated with choroidal or iris neovascularization also can be treated with TIE/TEK isoforms.

PDGFR isoforms also can be used in the treatment of proliferative vitreoretinopathy. For example, an expression vector such as a retroviral vector is constructed containing a nucleic acid molecule encoding a PDGFR isoform. Rabbit conjunctival fibroblasts (RCFs) are produced which contain the expression vector by transfection, such as for a retrovirus vector, or by transformation, such as for a plasmid or chromosomal based vector. Expression of PDGFR isoform can be monitored in cells by means known in the art including use of an antibody which recognizes PDGFR isoform and by use of a peptide tag (e.g. a myc tag) and corresponding antibody. RCFs are injected into the vitreous part of an eye. For example, in a rabbit animal model, approximately 1×10⁵ RCFs are injected by gas vitreomy. Retrovirus expressing PDGFR isoform, ˜2×10⁷ CFU is injected on the same day. Effects on proliferative vitreoretinopathy can be observed, for example, 2-4 weeks following surgery, such as attenuation of the disease symptoms.

EphA isoforms can be used to treat diseases or conditions with misregulated and/or inappropriate angiogenesis, such as in eye diseases. For example, an EphA isoform can be assessed in an animal model such as a mouse corneal model for effects on ephrinA-1 induced angiogenesis. Hydron pellets containing ephrinA-1 alone or with EphA isoform protein are implanted in mouse cornea. Visual observations are taken on days following implantation to observe EphA isoform inhibition or reduction of angiogenesis. Anti-angiogenic treatments and methods such as described for VEGFR isoforms are applicable to EphA isoforms.

2. Angiogenesis Related Atherosclerosis

RTK isoforms, for example VEGFR Flt-1 and TIE/TEK isoforms, can be used to treat angiogenesis conditions related to atherosclerosis such as neovascularization of atherosclerosis plaques. Plaques formed within the lumen of blood vessels have been shown to have angiogenic stimulatory activity. VEGF expression in human coronary atherosclerotic lesions is associated with the progression of human coronary atherosclerosis.

Animal models can be used to assess RTK isoforms in treatment of atherosclerosis. Apolipoprotein-E deficient mice (ApoE^{-/-}) are prone to atherosclerosis. Such mice are treated by injecting an RTK isoform, for example a VEGFR isoform, such as a Flt-1 intron fusion protein over a time course such as for 5 weeks starting at 5, 10 and 20 weeks of age. Lesions at the aortic root are assessed between control ApoE^{-/-} mice and isoform-treated ApoE^{-/-} mice to observe reduction of atherosclerotic lesions in isoform-treated mice.

3. Additional Angiogenesis-Related Treatments

RTK isoforms such as VEGFR isoforms, for example, Flt1 isoforms, and EphA isoforms also can be used to treat angiogenic and inflammatory-related conditions such as proliferation of synoviocytes, infiltration of inflammatory cells, cartilage destruction and pannus formation, such as are present in rheumatoid arthritis (RA). An autoimmune model of collagen type-II induced arthritis, such as polyarticular arthritis induced in mice, can be used as a model for human RA. Mice treated with a VEGFR isoform, such as by local injection of protein, can be observed for reduction of arthritic symptoms including paw swelling, erythema and ankylosis. Reduction of synovial angiogenesis and synovial inflammation also can be observed.

Other angiogenesis-related conditions amenable to treatment with VEGFR isoforms include hemangioma. One of the most frequent angiogenic diseases of childhood is the hemangioma. In most cases, the tumors are benign and regress without intervention. In more severe cases, the tumors progress to large cavernous and infiltrative forms and create clinical complications. Systemic forms of hemangiomas, the hemangiomatoses, have a high mortality rate. Many cases of hemangiomas exist that cannot be treated or are difficult to treat with therapeutics currently in use.

VEGFR isoforms can be employed in the treatment of such diseases and conditions where angiogenesis is responsible for damage such as in Osler-Weber-Rendu disease, or hereditary hemorrhagic telangiectasia. This is an inherited disease characterized by multiple small angiomas, tumors of blood or lymph vessels. The angiomas are found in the skin and mucous membranes, often accompanied by epistaxis (nosebleeds) or gastrointestinal bleeding and sometimes with pulmonary or hepatic arteriovenous fistula. Diseases and disorders characterized by undesirable vascular permeability also can be treated by VEGFR isoforms. These include edema associated with brain tumors, ascites associated with malignancies, Meigs' syndrome, lung inflammation, nephrotic syndrome, pericardial effusion and pleural effusion.

Angiogenesis also is involved in normal physiological processes such as reproduction and wound healing. Angiogenesis is an important step in ovulation and also in implantation of the blastula after fertilization. Modulation of angiogenesis by VEGFR isoforms can be used to induce amenorrhea, to block ovulation or to prevent implantation by the blastula. VEGFR isoforms also can be used in surgical procedures. For example, in wound healing, excessive repair or fibroplasia can be a detrimental side effect of surgical procedures and may be caused or exacerbated by angiogenesis. Adhesions are a frequent complication of surgery and lead to problems such as small bowel obstruction.

PDGFR isoforms can be used in the regulation of neointima formation after arterial injury such as in arterial surgery. For example PDGFR-B isoforms can be used to regulate PDGF-BB induced cell proliferation such as involved in neointima formation. PDGFR isoforms can be assessed for example, in a balloon-injured rooster femoral artery model. An adenovirus vector expressing a PDGFR isoform is constructed and transduced in vivo in the arterial model. Neointima-associated thrombosis is assessed in the transduced arteries to observe reduction compared with controls.

RTK isoforms useful in treatment of angiogenesis-related diseases and conditions also can be used in combination therapies such as with anti-angiogenesis drugs, molecules which interact with other signaling molecules in RTK-related pathways, including modulation of VEGFR ligands. For example, the known anti-rheumatic drug, bucillamine (BUC), was shown to include within its mechanism of action the inhibition of VEGF production by synovial cells. Anti-rheumatic effects of BUC are mediated by suppression of angiogenesis and synovial proliferation in the arthritic synovium through the inhibition of VEGF production by synovial cells. Combination therapy of such drugs with VEGFR isoforms can allow multiple mechanisms and sites of action for treatment.

4. Cancers

RTK isoforms such as isoforms of EGFR, TIE/TEK, VEGFR and FGFR can be used in treatment of cancers. RTK isoforms including, but not limited to, EGFR RTK isoforms, such as ErbB2 and ErbB3 isoforms, VEGFR isoforms such as Flt1 isoforms, FGFR isoforms such as FGFR-4 isoforms, and EphA1 isoforms can be used to treat cancer. Examples of cancer to be treated herein include, but are not limited to, carcinoma, lymphoma, blastoma, sarcoma, and leukemia or lymphoid malignancies. Additional examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Combination therapies can be used with EGFR isoforms including anti-hormonal compounds, cardioprotectants, and anti-cancer agents such as chemotherapeutics and growth inhibitory agents.

Cancers treatable with EGFR isoforms generally are those that express an EGFR receptor or a receptor with which an EGF ligand interacts. Such cancers are known to those of skill in the art and/or can be identified by any means known in the art for detecting EGFR expression. An example of an ErbB2 expression diagnostic/prognostic assay available includes HERCEPTEST.RTM. (Dako). Paraffin embedded tissue sections from a tumor biopsy are subjected to the IHC assay and accorded a ErbB2 protein staining intensity criteria. Tumors accorded with less than a threshold score can be characterized as not overexpressing ErbB2, whereas those tumors with greater than or equal to a threshold score can be characterized as overexpressing ErbB2. In one example of treatment, ErbB2-overexpressing tumors are assessed as candidates for treatment with an EGFR isoform such as an ErbB2 isoform.

Isoforms provided herein can be used for treatment of cancers. For example, TIE/TEK isoforms can be used in the treatment of cancers such as by modulating tumor-related angiogenesis. Vascularization is involved in regulating cancer growth and spread. For example, inhibition of angiogenesis and neovascularization inhibits solid tumor growth and expansion. Tie/Tek receptors such as TEK have been shown to influence vascular development in normal and cancerous tissues. TIE/TEK isoforms can be used as an inhibitor of tumor angiogenesis. A TIE/TEK isoform is produced such as by expression of the protein in cells. For example, secreted forms of TIE/TEK isoform can be expressed in cells and harvested from the media. Protein can be purified or partially-purified by biochemical means known in the art and by uses of antibody purification, such as antibodies raised against TIE/TEK isoform or a portion thereof or by use of a tagged TIE/TEK isoform and a corresponding antibody. Effects on angiogenesis can be monitored in an animal model such as by treating rat cornea with TIE/TEK isoform formulated as conditioned media in hydron pellets surgically implanted into a micropocket of a rat cornea or as purified protein (e.g. 100 μg/dose) administered to the window chamber. For example, rat models such as F344 rats with avascular corneas can be used in combination with tumor-cell conditioned media or by implanting a fragment of a tumor into the window chamber of an eye to induce angiogenesis. Corneas can be examined histologically to detect inhibition of angiogenesis induced by tumor-cell conditioned media. TIE/TEK isoforms also can be used to treat malignant and metastatic conditions such as solid tumors, including primary and metastatic sarcomas and carcinomas.

FGFR-4 isoforms can be used to treat cancers, for example pituitary tumors. Animal models can be used to mimic progression of human pituitary tumor progress. For example, an N-terminally shortened form of FGFR, ptd-FGFR-4, expressed in transgenic mice recapitulates pituitary tumorigenesis (Ezzat et al. (2002) J. Clin. Invest. 109:69-78), including pituitary adenoma formation in the absence of prolonged and massive hyperplasia. FGFR-4 isoforms can be administered to ptd-FGFR-4 mice and the pituitary architecture and course of tumor progression compared with control mice.

5. Alzheimer's Disease

Receptor isoforms, such EGFR isoforms, also can be used to treat inflammatory conditions and other conditions involving such responses, such as Alzheimer's disease and related conditions. A variety of mouse models are available for human Alzheimer's disease including transgenic mice overexpressing mutant amyloid precursor protein and mice expressing familial autosomal dominant-linked PS1 and mice expressing both proteins (PS1 M146L/APPK670N:M671L). Alzheimer's models are treated such as by injection of ErbB isoforms. Plaque development can be assessed such as by observation of neuritic plaques in the hippocampus, entorhinal cortex, and cerebral cortex, using staining and antibody immunoreactivity assays.

6. Smooth Muscle Proliferative-Related Diseases and Conditions

CSR isoforms, including EGFR isoforms, such as ErbB isoforms, can be employed for the treatment of a variety of diseases and conditions involving smooth muscle cell proliferation in a mammal, such as a human. An example is treatment of cardiac diseases involving proliferation of vascular smooth muscle cells (VSMC) and leading to intimal hyperplasia such as vascular stenosis, restenosis resulting from angioplasty or surgery or stent implants, atherosclerosis and hypertension. In such conditions, an interplay of various cells and cytokines released act in autocrine, paracrine or juxtacrine manner, which result in migration of VSMCs from their normal location in media to the damaged intima. The migrated VSMCs proliferate excessively and lead to thickening of intima, which results in stenosis or occlusion of blood vessels. The problem is compounded by platelet aggregation and deposition at the site of lesion. Alpha-thrombin, a multifunctional serine protease, is concentrated at sites of vascular injury and stimulates VSMC proliferation. Following activation of this receptor, VSMCs produce and secrete various autocrine growth factors, including PDGF-AA, HB-EGF and TGF. EGFRs are involved in signal transduction cascades that ultimately result in migration and proliferation of fibroblasts and VSMCs, as well as stimulation of VSMCs to secrete various factors that are mitogenic for endothelial cells and induction of chemotactic responses in endothelial cells. Treatment with EGFR isoforms can be used to modulate such signaling and responses.

EGFR isoforms such as ErbB2 and ErbB3 isoforms can be used to treat conditions where EGFRs such as ErbB2 and ErbB3 modulate bladder SMCs, such as bladder wall thickening that occurs in response to obstructive syndromes affecting the lower urinary tract. EGFR isoforms can be used in controlling proliferation of bladder smooth muscle cells, and consequently in the prevention or treatment of urinary obstructive syndromes.

EGFR isoforms can be used to treat obstructive airway diseases with underlying pathology involving smooth muscle cell proliferation. One example is asthma which manifests in airway inflammation and bronchoconstriction. EGF has been shown to stimulate proliferation of human airway SMCs and is likely to be one of the factors involved in the pathological proliferation of airway SMCs in obstructive airway diseases. EGFR isoforms can be used to modulate effects and responses to EGF by EGFRs.

7. Inflammatory Diseases

CSR isoforms such as TNFR isoforms can be used in the treatment of inflammatory diseases including central nervous system diseases (CNS), autoimmune diseases, airway hyper-responsiveness conditions such as in asthma, rheumatoid arthritis and inflammatory bowel disease.

TNF-α and LT are proinflammatory cytokines and critical mediators in inflammatory responses in diseases and conditions such as multiple sclerosis. TNF-α and LT-α are produced by infiltrating lymphocytes and macrophages and additionally by activated CNS parenchymal cells, microglial cells and astrocytes. In MS patients, TNF-α is overproduced in serum and cerebrospinal fluid. In lesions, TNF-α and TNFR are extensively expressed. TNF-α and LT-α can induce selective toxicity of primary oligodendrocytes and induce myelin damage in CNS tissues. Thus, these two cytokines have been implicated in demyelination.

Experimental autoimmune encephalomyelitis (EAE) can serve as a model for multiple sclerosis (MS)(see for example, Probert et al. (2000) Brain 123: 2005-2019). EAE can be induced in a number of genetically susceptible species by immunization with myelin and myelin components such as myelin basic protein, proteolipid protein and myelin oligodendrocyte glycoprotein (MOG). For example, MOG-induced EAE recapitulates essential features of human MS including the chronic, relapsing clinical disease course of the pathohistological triad of inflammation, reactive gliosis, and the formation of large confluent demyelinated plaques. Additional MS models include transgenic mice overexpressing TNF α, which model non-autoimmune mediated MS. Transgenic mice are engineered to express TNF-α locally in glial cells; human and murine TNF-α trigger MS-like symptoms. TNFR isoforms can be assessed in EAE animal models. Isoforms are administered, such as by injection, and the course and progression of symptoms is monitored compared to control animals.

Cytokines such as TNF-α also are involved in airway smooth muscle contractile properties. TNFR1 and TNFR2 play a role in modulating biological affects in airway smooth muscle. TNFR2 modulates calcium homeostasis and thereby modulates airway smooth muscle hyper-responsiveness. TNFR1 modulates effects of TNF-α in airway smooth muscle. Airway smooth muscle responses can be assessed in murine tracheal rings induced with carbachol. Effects, such as carbachol-induced contraction, in the presence and absence of TNF-α can be monitored. TNFR isoforms can be added to tracheal rings to assess the effects of isoforms on airway smooth muscle.

TNF-α/TNFRs modulate inflammation in diseases such as rheumatoid arthritis (RA) (Edwards et al. (2003) Adv Drug Deliv. Rev. 55(10):1315-36). TNFR isoforms, including TNFR1 isoforms, can be used to treat RA. For example, TNFR isoforms can be injected locally or systemically. Isoforms can be dosed daily or weekly. PEGylated TNFR isoforms can be used to reduce immunogenicity. Primate models are available for RA treatments. Response of tender and swollen joints can be monitored in subjects treated with TNFR isoforms and controls to assess TNFR isoform treatment.

8. Combination Therapies

CSR isoforms such as RTK isoforms can be used in combination with each other and with other existing drugs and therapeutics to treat diseases and conditions. For example, as described herein a number of RTK-isoforms can be used to treat angiogenesis-related conditions and diseases and/or control tumor proliferation. Such treatments can be performed in conjunction with anti-angiogenic and/or anti-tumorigenic drugs and/or therapeutics. Examples of anti-angiogenic and anti-tumorigenic drugs and therapies useful for combination therapies include tyrosine kinase inhibitors and molecules capable of modulating tyrosine kinase signal transduction including, but not limited to, 4-aminopyrrolo[2,3-d]pyrimidines (see for example, U.S. Pat. No. 5,639,757), and quinazoline compounds and compositions (e.g., U.S. Pat. No. 5,792,771). Other compounds useful in combination therapies include steroids such as the angiostatic 4,9(11)-steroids and C21-oxygenated steroids, angiostatin, endostatin, vasculostatin, canstatin and maspin, angiopoietins, bacterial polysaccharide CM101 and the antibody LM609 (U.S. Pat. No. 5,753,230), thrombospondin (TSP-1), platelet factor 4 (PF4), interferons, metalloproteinase inhibitors, pharmacological agents including AGM-1470/TNP-470, thalidomide, and carboxyamidotriazole (CAI), cortisone such as in the presence of heparin or heparin fragments, anti-Invasive Factor, retinoic acids and paclitaxel (U.S. Pat. No. 5,716,981; incorporated herein by reference), shark cartilage extract, anionic polyamide or polyurea oligomers, oxindole derivatives, estradiol derivatives and thiazolopyrimidine derivatives.

Treatment of cancers including treatment of cancers overexpressing an EGFR can include combination therapy with an anticancer agent, a chemotherapeutic agent and growth inhibitory agent, including coadministration of cocktails of different chemotherapeutic agents. Examples of chemotherapeutic agents include taxanes (such as paclitaxel and doxetaxel) and anthracycline antibiotics. Preparation and dosing schedules for such chemotherapeutic agents may be used according to manufacturers' instructions or as determined empirically by the skilled practitioner. Preparation and dosing schedules for such chemotherapy also are described in Chemotherapy Service Ed., M. C. Perry, Williams & Wilkins, Baltimore, Md. (1992).

Additional compounds can be used in combination therapy with RTK isoforms. Anti-hormonal compounds can be used in combination therapies, such as with EGFR isoforms. Examples of such compounds include an anti-estrogen compound such as tamoxifen; an anti-progesterone such as onapristone and an anti-androgen such as flutamide, in dosages known for such molecules. It also can be beneficial to also coadminister a cardioprotectant (to prevent or reduce myocardial dysfunction that can be associated with therapy) or one or more cytokines. In addition to the above therapeutic regimes, the patient may be subjected to surgical removal of cancer cells and/or radiation therapy.

Adjuvants and other immune modulators can be used in combination with CSR isoforms in treating cancers, for example to increase immune response to tumor cells. Combination therapy can increase the effectiveness of treatments and in some cases, create synergistic effects such the combination is more effective than the additive effect of the treatments separately. Examples of adjuvants include, but are not limited to, bacterial DNA, nucleic acid fraction of attenuated mycobacterial cells (BCG; Bacillus-Calmette-Guerin), synthetic oligonucleotides from the BCG genome, and synthetic oligonucleotides containing CpG motifs (CpG ODN; Wooldridge et al. (1997) Blood 89:2994-2998), levamisole, aluminum hydroxide (alum), BCG, Incomplete Freud's Adjuvant (IFA), QS-21 (a plant derived immunostimulant), keyhole limpet hemocyanin (KLH), and dinitrophenyl (DNP). Examples of immune modulators include but are not limited to, cytokines such as interleukins (e.g., IL-2, IL-3, IL-4, IL-5, IL-6, IL-7, IL-9, IL-10, IL-11, IL-12, IL-13, IL-15, IL-16, IL-17, IL-18, IL-1α, IL-1β, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligand, CD27 ligand, CD30 ligand, 4-1BBL, Trail), and MIF, interferon, cytokines such as IL-2 and IL-12; and chemotherapy agents such as methotrexate and chlorambucil.

9. Preclinical Studies

Model animal studies can be used in preclinical evaluation of RTK isoforms that are candidate therapeutics. Parameters that can be assessed include, but are not limited to efficacy and concentration-response, safety, pharmacokinetics, interspecies scaling and tissue distribution. Model animal studies include assays such as described herein as well as those known to one of skill in the art. Animal models can be used to obtain data that then can be extrapolated to human dosages for design of clinical trials and treatments with RTK isoforms. For example, efficacy and concentration-response VEGFR inhibitors in tumor-bearing mice can be extrapolated to human treatment (Mordenti et al., (1999) Toxicol Pathol. January-February; 27(1):14-21) in order to define clinical dosing regimens effective to maintain a therapeutic inhibitor, such as an antibody against VEGFR for human use in the required efficacious range. Similar models and dose studies can be applied to VEGFR isoform dosage determination and translation into appropriate human doses, as well as other techniques known to the skilled artisan. Preclinical safety studies and preclinical pharmacokinetics can be performed, for example in monkeys, mice, rats and rabbits. Pharmacokinetic data from mice, rats and monkeys has been used to predict the pharmacokinetics of the counterpart therapeutic in human using allometric scaling. Accordingly, appropriate dosage information can be determined for the treatment of human pathological conditions, including rheumatoid arthritis, ocular neovascularization and cancer. A humanized version of the anti-VEGF antibody has been employed in clinical trials as an anti-cancer agent (Brem, (1998) Cancer Res. 58(13):2784-92; Presta et al., (1997) Cancer Res. 57(20):4593-9) and such clinical data also can be considered as a reference source when designing therapeutic doses for VEGFR isoforms.

### M. Combination Therapies

CSR isoforms, including those provided herein, can be used in combination with each other, with other cell surface receptor isoforms, such as a herstatin or any described, for example, in U.S. application Ser. Nos. 09/942,959, 09/234,208, 09/506,079; U.S. Provisional Application Ser. Nos. 60/571,289, 60/580,990 and 60/666,825; and U.S. Pat. No. 6,414,130, published International PCT application Nos. WO 00/44403, WO 01/61356, WO 2005/016966, including but not limited, to those set forth in SEQ ID Nos. 320-359; and/or with other existing drugs and therapeutics to treat diseases and conditions, particularly those involving aberrant angiogenesis and/or neovascularization, including, but not limited to, cancers and other proliferative disorders, inflammatory diseases, autoimmune disorders, as set forth herein and known to those of skill in the art.

For example, a CSR isoform, such as a VEGF isoform, can be administered with an agent for treatment of diabetes. Such agents include agents for the treatment of any or all conditions such as diabetic periodontal disease, diabetic vascular disease, tubulointerstitial disease and diabetic neuropathy. In another example, a CSR isoform is administered with an agent that treats cancers including squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. Any of the CSR isoforms can be administered in combination with two or more agents for treatment of a disease or a condition.

Adjuvants and other immune modulators can be used in combination with isoforms in treating cancers, for example to increase immune response to tumor cells. Combination therapy can increase the effectiveness of treatments and in some cases, create synergistic effects such the combination is more effective than the additive effect of the treatments separately. Examples of adjuvants include, but are not limited to, bacterial DNA, nucleic acid fraction of attenuated mycobacterial cells (BCG; Bacillus-Calmette-Guerin), synthetic oligonucleotides from the BCG genome, and synthetic oligonucleotides containing CpG motifs (CpG ODN; Wooldridge et al. (1997) Blood 89:2994-2998), levamisole, aluminum hydroxide (alum), BCG, Incomplete Freud's Adjuvant (IFA), QS-21 (a plant derived immunostimulant), keyhole limpet hemocyanin (KLH), and dinitrophenyl (DNP). Examples of immune modulators include but are not limited to, cytokines such as interleukins (e.g., IL-2, IL-3, IL-4, IL-5, IL-6, IL-7, IL-9, IL-10, IL-11, IL-12, IL-13, IL-15, IL-16, IL-17, IL-18, IL-1α, IL-1β, and IL-1 RA), granulocyte colony stimulating factor (G-CSF), granulocyte-macrophage colony stimulating factor (GM-CSF), oncostatin M, erythropoietin, leukemia inhibitory factor (LIF), interferons, B7.1 (also known as CD80), B7.2 (also known as B70, CD86), TNF family members (TNF-α, TNF-β, LT-β, CD40 ligand, Fas ligand, CD27 ligand, CD30 ligand, 4-1BBL, Trail), and MIF, interferon, cytokines such as IL-2 and IL-12; and chemotherapy agents such as methotrexate and chlorambucil.

Combinations of different CSR isoforms including with herstatins and other agents, can be used for treating cancers and other disorders involving aberrant angiogenesis (see, e.g. FIG. 1 outlining targets in the angiogenesis and neovascularization pathway for such polypeptides and those described herein and in the above-noted copending and published applications U.S. application Ser. Nos. 09/942,959, 09/234,208, 09/506,079; U.S. Provisional Application Ser. Nos. 60/571,289, 60/580,990 and 60/666,825; and U.S. Pat. No. 6,414,130, published International PCT application Nos. WO 00/44403, WO 01/61356, WO 2005/016966 are provided. The cell surface receptors include receptor tyrosine kinases, such as members of the VEGFR, FGFR, PDGFR (including Rα, Rβ, CSF1R, Kit), MET (including c-Met, c-RON), TEK and EphA2 families. These also include ErbB2, ErbB3, ErbB4, DDR1, DDR2, EPHA, EPHB, FGFR-2, FGFR-3, FGFR-4, MET, PDGFR, TEK, Tie-1, KIT, ErbB2, VEGFR-1, VEGFR-2, VEGFR-3, Flt1, Flt3, TNFR1, TNFR2, RON, CSFR. Exemplary of such isoforms are the herstatins (see, SEQ ID Nos. 320-345), polypeptides that include the intron portion of a herstatin as well as any isoforms provided herein. The combinations of isoforms and/or drug agent selected is a function of the disease to be treated and is based upon consideration of the target tissues and cells and receptors expressed thereon.

The combinations, for example, can target two or more cell surface receptors or steps in the angiogenic and/or endothelial cell maintenance pathways or can target two or more cell surface receptors or steps in a disease process, such as any which one or both of these pathways are implicated, such as inflammatory diseases, tumors and all other noted herein and known to those of skill in the art. The two or more agents can be administered as a single composition or can be administered as two or more compositions (where there are more than two agents) simultaneously, intermittently or sequentially. They can be packaged as a kit that contains two or more compositions separately or as a combined composition and optionally with instructions for administration and/or devices for administration, such as syringes.

The following examples are included for illustrative purposes only and are not intended to limit the scope of the invention.

### N. EXAMPLES

### Example 1

### Method for Cloning CSR Isoforms

A. Preparation of Messenger RNA

mRNA isolated from major human tissue types from healthy or diseased tissues or cell lines were purchased from Clontech (BD Biosciences, Clontech, Palo Alto, Calif.) and Stratagene (La Jolla, Calif.). Equal amounts of mRNA were pooled and used as templates for reverse transcription-based PCR amplification (RT-PCR).

B. cDNA Synthesis

mRNA was denatured at 70° C. in the presence of 40% DMSO for 10 min and quenched on ice. First-strand cDNA was synthesized with either 200 ng oligo(dT) or 20 ng random hexamers in a 20-μl reaction containing 10% DMSO, 50 mM Tris-HCl (pH 8.3), 75 mM KCl, 3 mM MgCl₂, 10 mM DTT, 2 mM each dNTP, 5 μg mRNA, and 200 units of Stratascript reverse transcriptase (Stratagene, La Jolla, Calif.). After incubation at 37° C. for 1 h, the cDNA from both reactions were pooled and treated with 10 units of RNase H (Promega, Madison, Wis.).

C. PCR Amplification

Gene-specific PCR primers were selected using the Oligo 6.6 software (Molecular Biology Insights, Inc., Cascade, Colo.) and synthesized by Qiagen-Operon (Richmond, Calif.). The forward primers flank the start codon. The reverse primers flank the stop codon or were chosen from regions at least 1.5 kb downstream from the start codon (see Table 4). Each PCR reaction contained 10 ng of reverse-transcribed cDNA, 0.025 U/μl TaqPlus (Stratagene), 0.0035 U/μl PfuTurbo (Stratagene), 0.2 mM dNTP (Amersham, Piscataway, N.J.), and 0.2 μM forward and reverse primers in a total volume of 50 μl. PCR conditions were 35 cycles and 94.5° C. for 45 s, 58° C. for 50 s, and 72° C. for 5 min. The reaction was terminated with an elongation step of 72° C. for 10 min.

TABLE 3B
LIST OF GENES FOR CLONING CSR Isoforms

| Family | Member | nt ACC. # | Catalytic Domain | SEQ ID NO: | ORF | prt ACC. # | SEQ ID NO: |
|---|---|---|---|---|---|---|---|
| PDGFR | CSF1R | NM_005211 | 2012-3208 | 162 | 293-3211 | NP_005202 | 249 |
| | Flt3 | NM_004119 | 1861-2886 | 244 | 58-3039 | NP_004110 | 272 |
| | KIT | NM_000222 | 1762-2799 | 1 | 22-2952 | NP_000213 | 273 |
| | PDGFR-A | NM_006206 | 2147-3253 | 246 | 395-3664 | NP_006197 | 275 |
| | PDGFR-B | NM_002609 | 2133-3215 | 163 | 357-3677 | NP_002600 | 276 |
| DDR | DDR1 | NM_013993 | 2149-3057 | 156 | 337-3078 | NP_054699 | 250 |
| | DDR2 | NM_006182 | 2022-2900 | 227 | 354-2921 | NP_006173 | 251 |
| EPH | EphA1 | NM-005232 | 1939-2736 | 165 | 88-3018 | NP_005223 | 253 |
| | EphA2 | NM-004431 | 1956-2759 | 229 | 138-3068 | NP_004422 | 254 |
| | EphA3 | NM-005233 | 2086-2859 | 230 | 226-3177 | NP_005224 | 255 |
| | EphA4 | NM_004438 | 1885-2685 | 231 | 43-3003 | NP_004429 | 256 |
| | EphA5 | L36644 | 1259-1460 | 232 | 1-2976 | AAA74245 | 257 |
| | EphA6 | AL133666 | 691-1332 | 233 | 343-1347 | CAB63775 | 258 |
| | EphA7 | NM_004440 | 2092-2892 | 234 | 214-3210 | NP_004431 | 259 |
| | EphA8 | NM_020526 | 2028-2801 | 235 | 126-3143 | NP_065387 | 260 |
| | EphB1 | NM_004441 | 2051-2857 | 166 | 215-3169 | NP_004432 | 261 |
| | EphB2 | AF025304 | 1886-2681 | 236 | 26-3193 | AAB94602 | 262 |
| | EphB3 | NM_004443 | 2316-3122 | 237 | 438-3434 | NP_004434 | 263 |
| | EphB4 | NM_004444 | 2200-3006 | 238 | 376-3339 | NP_004435 | 264 |
| | EphB6 | NM_004445 | 2761-3498 | 239 | 799-3819 | NP_004436 | 265 |
| ERB | ErbB2 | NM_004448 | 2396-3164 | 240 | 239-4006 | NP_004439 | 266 |
| | ErbB3 | NM_001982 | 2318-3086 | 241 | 194-4222 | NP_001973 | 267 |
| | EGFR | NM_005228 | 2380-3148 | 228 | 247-3879 | NP_005219 | 252 |
| FGFR | FGFR-1 | M34641 | 1435-2263 | 164 | 10-2472 | AAA35835 | 268 |
| | FGFR-2 | NM_000141 | 2009-2872 | 242 | 593-3058 | NP_000132 | 269 |
| | FGFR-3 | NM_000142 | 1429-2292 | 243 | 40-2460 | NP_000133 | 270 |
| | FGFR-4 | NM_002011 | 1534-2394 | 2 | 157-2565 | NP_002002 | 271 |
| MET | MET | NM_000245 | 3419-4198 | 245 | 188-4360 | NP_000236 | 274 |
| | RON | NM_002447 | 3242-4260 | 159 | 29-4231 | NP_002438 | 277 |
| TEK | TEK | NM_000459 | 2603-3433 | 160 | 149-3523 | NP_000450 | 278 |
| | Tie-1 | NM_005424 | 2579-3409 | 161 | 80-3496 | NP_005415 | 279 |
| TNFR | TNFR1 | NM_001065 | 1323-1598(DD) | 247 | 282-1649 | NP_001056 | 280 |
| | TNFR2 | NM_001066 | n/a | 3 | 90-1475 | NP_001057 | 281 |
| VEGFR | VEGFR-1 | NM_002019 | 2704-3702 | 157 | 250-4266 | NP_002010 | 282 |
| | VEGFR-2 | NM_002253 | 2779-3792 | 248 | 304-4374 | NP_002244 | 283 |
| | VEGFR-3 | NM_002020 | 2530-3525 | 158 | 22-3918 | NP_002011 | 284 |

TABLE 4
PRIMERS FOR PCR CLONING.

| | | |
|---|---|---|
| SEQ | | |
| ID | | |
| NO | Primer | Sequence |

| | | |
|---|---|---|
| 4 | CSFIR_F1 | CTG CCA CTT CCC CAC CGA GG |
| 5 | DDR1_F1 | GGG ATC AGG AGC TAT GGG ACC A |
| 6 | DDR2_F1 | CTG AGA TGA TCC TGA TTC CCA GAA |
| 7 | EphA1_F1 | GGA GCT ATG GAG CGG CGC TG |
| 8 | EphA2_F1 | AGC GAG AAG CGC GGC ATG GA |
| 9 | EphA3_F1 | CAC CAG CAA CAT GGA TTG TCA GC |
| 10 | EphA4_F1 | CGA ACC ATG GCT GGG ATT TTC TA |
| 11 | EphA7_F1 | ATA AAA CCT GCT CAT GCA CCA TG |
| 12 | EphB1_F1 | GCG ATG GCC CTG GAT TAT CTA |
| 13 | EphB2_F1 | CCC CGG GAA GCG CAG CCA |
| 14 | EphB3_F1 | GCT CCT AGA GCT GCC ACG GC |
| 15 | EphB4_F1 | GAT CCT ACC CGA GTG AGG CGG |
| 16 | CSFIR_R1 | GGG CTC CTG CAG AGA TGG GTA |
| 17 | DDR1_R1 | AGA GCC ATT GGG GAC ACA GGG A |
| 18 | DDR2_R1 | AGC CTG ACT CCT CCT CCC CTG |
| 19 | EphA1_R1 | AGC TCT GTC AGC AAG ACC CTG G |
| 20 | EphA2_R1 | AGG TGG TGT CTG GGG CCA GGT C |
| 21 | EphA3_R1 | GTC AGG CTT GAG GCT ACT GAT GG |
| 22 | EphA4_R1 | AAC ATA GGA AGT GAG AGG GTT CAG G |
| 23 | EphA7_R1 | ACT CCA TTG GGA TGC TCT GGT TC |
| 24 | EphB1_R1 | AGC CCA TCA ATC CTT GCT GTG |
| 25 | EphB2_R1 | GCG TGC CCG CAC CTG GAA GA |
| 26 | EphB3_R1 | GCT GGT CAC TGT GGA GGC GA |
| 27 | EphB4_R1 | GGT AGC TGG CTC CCC GCT TCA |
| 28 | CSFIR_R2 | CCG AGG GTC TTA CCA AAC TGC |
| 29 | DDR1_R2 | AAG CGG AGT CGA GAT CGA GGG A |
| 30 | DDR2_R2 | GGG GAA CTC CTC CAC AGC CA |
| 31 | EphA1_R2 | CGG GTA AAG TCC AAG GCT CCC |
| 32 | EphA2_R2 | GAC ACA GGA TGG ATG GAT CTC GG |
| 33 | EphA3_R2 | ATC AAT GGA TAT GTT GGT GGC ATC |
| 34 | EphA4_R2 | AGG ATG CGT CAA TTT CTT TGG CA |
| 35 | EphA7_R2 | CTG CAC CAA TCA CAC GCT CAA |
| 36 | EphB1_R2 | ATC AAT CTC CTT GGC AAA CTC C |
| 37 | EphB2_R2 | GCC CAT GAT GGA GGC TTC GC |
| 38 | EphB3_R2 | ACG CAG GAC ACG TCG ATC TCC |
| 39 | EphB4_R2 | ACC TGC ACC AAT CAC CTC TTC AA |
| 40 | EphB6_F1 | AGA GTG GCG GGC ATG GTG TG |
| 41 | EphB6_R1 | GCG GAG CTG ATA GTC CAG GAT G |
| 42 | EphB6_R2 | CCT GTC CCA ATG ACC TCC TCA A |
| 43 | EphA6_F1 | GGA GAT GAA AGA CTC TCC ATT TCA AG |
| 44 | FGFR-1_F1 | ATT CGG GAT GTG GAG CTG GA |
| 45 | FGFR-2_F1 | AGG ACC GGG GAT TGG TAC CG |
| 46 | FGFR-3_F1 | CAT GGG CGC CCC TGC CTG |
| 47 | FGFR-4_F1 | AGA AGG AGA TGC GGC TGC TG |
| 48 | TNFR1(p55)_F1 | AGC TGT CTG GCA TGG GCC TCT C |
| 49 | TNFR2(p75)_F1 | ACC GGA CCC CGC CCG CAC |
| 50 | EphA6_R1 | ATCT TAG ACC GAC AGA AAA TTT GGC |
| 51 | FGFR-1_R1 | CAA GGG ACC ATC CTG CGT GC |
| 52 | FGFR-2_R1 | AGG GGC TTG CCC AGT GTC AG |
| 53 | FGFR-3_R1 | GCT CCC ATT TGG GGT CGG CA |
| 54 | FGFR-4_R1 | CGG GGG AAC TCC CAT AGT GG |
| 55 | TNFR1(p55)_R1 | GGC GCA GCC TCA TCT GAG AAG A |
| 56 | TNFR2(p75)_R1 | CAC AGC CCA CAC CGG CCT GG |
| 57 | Flt3_F1 | GGA GGC CAT GCC GGC GTT G |
| 58 | KIT-F1 | CGC AGC TAC CGC GAT GAG AGG |
| 59 | MET_F1 | CTC ATA ATG AAG GCC CCC GC |
| 60 | PDGFR-A_F1 | AAG TTT CCC AGA GCT ATG GGG A |
| 61 | PDGFR-B_F1 | AGC AGC AAG GAC ACC ATG CG |
| 62 | RON_F1 | GGT CCC AGC TCG CCT CGA TG |
| 63 | TEK_F1 | AGA TTT GGG GAA GCA TGG ACT C |
| 64 | Tie-1_F1 | CGG CCT CTG GAG TAT GGT CTG |
| 65 | VEGFR-1_F1 | CAT GGT CAG CTA CTG GGA CAC C |
| 66 | VEGFR-2_F1 | AGG TGC AGG ATG CAG AGC AAG |
| 67 | VEGFR-3_F1 | AGC GGC CGG AGA TGC AGC G |
| 68 | Flt3_R1 | CTG CTC GAC ACC CAC TGT CCA |
| 69 | KIT-R1 | GCA GAA GTC TTG CCC ACA TCG |
| 70 | MET_R1 | CTT CGT GAT CTT CTT CCC AGT GA |
| 71 | PDGFR-A_R1 | AGA TTC TTA GCC AGG CAT CGC A |
| 72 | PDGFR-B_R1 | AGC GCA CCG ACA GTG GCC GA |
| 73 | RON_R1 | GCA CGG GCT GCC CAC TGT CA |
| 74 | TEK_R1 | CTG TCC GAG GTT CCA AAT AGT TGA |
| 75 | Tie-1_R1 | CGT TCT CAC TGG GGT CCA CCA |
| 76 | VEGFR-1_R1 | ATT ATT GCC ATG CGC TGA GTG A |
| 77 | VEGFR-2_R1 | GCC GCT TGG ATA ACA AGG GTA |
| 78 | VEGFR-3_R1 | AAC TCG GTC CAG GTG TCC AGG C |
| 79 | Flt3_R2 | CTT GGA AAC TCC CAT TTG AGA TCA |
| 80 | KIT-R2 | ACA ACC TTC CCG AAA GCT CCA |
| 81 | MET_R2 | ACT ACA TGC TGC ACT GCC TGG A |
| 82 | PDGFR-A_R2 | CCC GAC CAA GCA CTA GTC CAT C |
| 83 | PDGFR-B_R2 | CCA GAG CCG AGG GTG CGT CC |
| 84 | RON_R2 | CAG GTC ATT CAG GTT GGG AGG A |
| 85 | TEK_R2 | ATT TGA TGT CAT TCC AGT CAA GCA |
| 86 | Tie-1_R2 | AGC ACT GGG TAG CTC AGG GGC |
| 87 | VEGFR-1_R2 | AAC TCC CAC TTG CTG GCA TCA |
| 88 | VEGFR-2_R2 | AAT TCC CAT TTG CTG GCA TCA |
| 89 | VEGFR-3_R2 | ATT CCC ACT GGC TGG CAT CGT A |

D. Cloning and Sequencing of PCR Products

PCR products were electrophoresed on a 1% agarose gel, and DNA from detectable bands was stained with Gelstar (BioWhitaker Molecular Application, Walkersville, Md.). The DNA bands were extracted with the QiaQuick gel extraction kit (Qiagen, Valencia, Calif.), ligated into the pDrive UA-cloning vector (Qiagen), and transformed into Escherichia coli. Recombinant plasmids were selected on LB agar plates containing 100 μg/ml carbenicillin. For each transfection, 192 colonies were randomly picked and their cDNA insert sizes were determined by PCR with M13 forward and reverse vector primers. Representative clones from PCR products with distinguishable molecular masses as visualized by fluorescence imaging (Alpha Innotech, San Leandro, Calif.) were then sequenced from both directions with vector primers (M13 forward and reverse). All clones were sequenced entirely using custom primers for directed sequencing completion across gapped regions.

E. Sequence Analysis

Computational analysis of alternative splicing was performed by alignment of each cDNA sequence to its respective genomic sequence using SIM4 (a computer program for analysis of splice variants). Only transcripts with canonical (e.g. GT-AG) donor-acceptor splicing sites were considered for analysis. Clones encoding CSR isoforms were studied further (see below, Table 5).

F. Targeted Cloning and Expression

Computational analysis of public EST databases identified potential splice variants with intron retention or insertion. Cloning of potential splice variants identified by EST database analysis were performed by RT-PCR using primers flanking the open reading frame as described above.

Sequence-verified CSR isoform encoding cDNA molecules were and can be subcloned into a replication-deficient recombinant adenoviral vector under control of the CMV promoter, following the manufacturer's instruction (Invitrogen, Cat# K4930-00). The recombinant adenoviruses were produced using 293A cells (Invitrogen). Supernatants from the infected 293 cells were analyzed by immunoblotting using an appropriate antibody.

G. Exemplary CSR Isoforms

Exemplary CSR isoforms, prepared using the methods described herein, are set forth below in Table 5. Nucleic acid molecules encoding CSR isoforms are provided and include those that contain sequences of nucleotides or ribonucleotides or nucleotide or ribonucleotide analogs as set forth in any of SEQ ID NOS: 92, 94, 96, 114, 116, 118, 120, 122, 124, 126, 128, 130, 132, 134, 136, 138, 140, 142, 144, 146, 148, 150, 152, 154, 167, 169, 171, 173, 175, 177, 179, 181, 183, 185, 187, 189, 191, 193, 195, 197, 199, 201, 203, 205, 207, 209, 211, 213, 215, 217, 219, 221, 223, and 225. The amino acid sequences of exemplary CSR isoform polypeptides are set forth in any of SEQ ID NOS: 91, 93, 95, 115, 117, 119, 121, 123, 125, 127, 129, 131, 133, 135, 137, 139, 141, 143, 145, 147, 149, 151, 153, 155, 168, 170, 172, 174, 176, 178, 180, 182, 184, 186, 188, 190, 182, 184, 196, 198, 200, 202, 204, 206, 208, 210, 212, 214, 216, 218, 220, 222, 224, and 226.

TABLE 5
CSR Isoforms

| | | | | SEQ ID |
|---|---|---|---|---|
| Gene | ID | Type | Length | NOS |
| FGFR-4 | SR002_A11 | Intron | 72 aa | 90-91 |
| | | fusion | | |
| KIT | SR002_H01 | Intron | 413 aa | 92-93 |
| | | fusion | | |
| TNFR2 | SR003_H02 | Intron | 155 aa | 94-95 |
| | | fusion | | |
| DDR1 | SR005_A11 | Exon | 286 aa | 114-115 |
| | | deletion | | |
| DDR1 | SR005_A10 | Exon | 243 aa | 116-117 |
| | | deletion | | |
| FGFR-1 | SR001_E12 | Exon | 228 aa | 118-119 |
| | | deletions | | |
| FGFR-4 | SR002_A10 | Intron | 446 aa | 120-121 |
| | | fusion | | |
| VEGFR-1 | SR004_C05 | Intron | 174 aa | 122-123 |
| | | fusion | | |
| VEGFR-3 | SR007_E10 | Exon | 227 aa | 124-125 |
| | | short | | |
| VEGFR-3 | SR007_F05 | Exon | 295 aa | 126-127 |
| | | deletion | | |
| RON | SR004_C11 | Intron | 495 aa | 128-129 |
| | | fusion | | |
| TEK | SR007_G02 | Intron | 367 aa | 130-131 |
| | | fusion, | | |
| | | exon | | |
| | | shorten | | |
| TEK | SR007_H03 | Exon | 468 aa | 132-133 |
| | | deletion, | | |
| | | Intron | | |
| | | fusion | | |
| Tie-1 | SR006_A04 | Intron | 251 aa | 134-135 |
| | | fusion | | |
| Tie-1 | SR006_B07 | Intron | 379 aa | 136-137 |
| | | fusion | | |
| Tie-1 | SR006_B06 | Intron | 161 aa | 138-139 |
| | | fusion | | |
| Tie-1 | SR006_B12 | Intron | 414 aa | 140-141 |
| | | fusion | | |
| Tie-1 | SR006_B10 | Exon | 317 aa | 142-143 |
| | | deletion | | |
| CSF1R | SR005_A06 | Exon | 306 aa | 144-145 |
| | | deletion | | |
| PDGFR-B | SR007_C09 | Exon | 336 aa | 146-147 |
| | | shorten | | |
| | | (4 bp) | | |
| EphA1 | SR004_G03 | Intron | 474 aa | 148-149 |
| | | fusion | | |
| EphA1 | SR004_G07 | Intron | 311 aa | 150-151 |
| | | fusion, | | |
| | | exon | | |
| | | deletion | | |
| EphA1 | SR004_H03 | Intron | 490 aa | 152-153 |
| | | fusion | | |
| EphB1 | SR005_D06 | Exon | 242 aa | 154-155 |
| | | shorten | | |
| EphA2 | SR016_E12 | Intron | 497 aa | 167-168 |
| | | fusion | | |
| EphB4 | SR012_C08 | Exon | 306 aa | 169-170 |
| | | deletion | | |
| EphB4 | SR012_D11 | Exon | 516 aa | 171-172 |
| | | shorten | | |
| EphB4 | SR012_E11 | Exon | 414 aa | 173-174 |
| | | shorted | | |
| FGFR-1 | SR022_C02 | Exon | 320 aa | 175-176 |
| | | deletion, | | |
| | | intron | | |
| | | fusion | | |
| FGFR-2 | SR022_C10 | Intron | 266 aa | 177-178 |
| | | fusion | | |
| FGFR-2 | SR022_C11 | Intron | 317 aa | 179-180 |
| | | fusion | | |
| FGFR-2 | SR022_D04 | Exon | 281 aa | 181-182 |
| | | deletion, | | |
| | | intron | | |
| | | fusion | | |
| FGFR-2 | SR022_D06 | Intron | 396 aa | 183-184 |
| | | fusion | | |
| MET | SR020_C10 | Intron | 413 aa | 185-186 |
| | | fusion | | |
| MET | SR020_C12 | Intron | 468 aa | 187-188 |
| | | fusion | | |
| MET | SR020_D04 | Intron | 518 aa | 189-190 |
| | | fusion | | |
| MET | SR020_D07 | Intron | 596 aa | 191-192 |
| | | fusion | | |
| MET | SR020_D11 | Intron | 408 aa | 193-194 |
| | | fusion | | |
| MET | SR020_E11 | Intron | 621 aa | 195-196 |
| | | fusion | | |
| MET | SR020_F08 | Intron | 664 aa | 197-198 |
| | | fusion | | |
| MET | SR020_F11 | Intron | 719 aa | 199-200 |
| | | fusion | | |
| MET | SR020_F12 | Intron | 697 aa | 201-202 |
| | | fusion | | |
| MET | SR020_G03 | Exon | 691 aa | 203-204 |
| | | shorten, | | |
| | | intron | | |
| | | fusion | | |
| MET | SR020_G07 | Intron | 661 aa | 205-206 |
| | | fusion | | |
| MET | SR020_H03 | Intron | 755 aa | 207-208 |
| | | fusion | | |
| MET | SR020_H06 | Intron | 823 aa | 209-210 |
| | | fusion | | |
| MET | SR020_H07 | Intron | 877 aa | 211-212 |
| | | fusion | | |
| MET | SR020_H08 | Exon | 764 aa | 213-214 |
| | | deletion, | | |
| | | intron | | |
| | | fusion | | |
| RON | SR014_C01 | Intron | 541 aa | 215-216 |
| | | fusion | | |
| RON | SR014_C09 | Intron | 908 aa | 217-218 |
| | | fusion | | |
| RON | SR014_E12 | Intron | 647 aa | 219-220 |
| | | fusion | | |
| Tie-1 | SR016_G03 | Intron | 751 aa | 221-222 |
| | | fusion | | |
| VEGFR-1 | SR01_C02 | Intron | 541 aa | 100 |
| | | fusion | | |
| VEGFR-2 | SR015_F01 | Exon | 712 aa | 223-224 |
| | | shorten | | |
| VEGFR-3 | SR015_G09 | Intron | 765 aa | 225-226 |
| | | fusion | | |

### Example 2

### CSR Isoform Expression Assays

A. Analysis of mRNA Expression

Expression of the cloned CSR isoforms were determined by RT-PCR (or quantitative PCR) in various tissues including: brain, heart, kidney, placenta, prostate, spleen, spinal cord, trachea, testis, uterus, fetal brain, fetal liver, adrenal gland, liver, lung, small intestine, salivary gland, skeletal muscle, thymus, thyroid and a variety of tumor tissues including: breast, colon, kidney, lung, ovary, stomach, uterus, MDA435 and HEPG2. PCR primers (such as set forth in Example 1, Table 4) were selected within the exclusive regions of retained introns or alternative exons, such that only the soluble receptor-specific signals were amplified. Each PCR reaction was performed with 2 cycle numbers (e.g. 32 versus 38 cycles) for the purpose of getting semi-quantitative results. Expression of each cloned CSR isoform was compared to the expression of the corresponding wildtype membrane receptor.

EphA2 (GenBank No. NM_{—}004431 or SEQ ID NO: 229) mRNA is highly expressed in brain, heart, kidney, placenta, prostate, spleen, spinal cord, trachea, testis, uterus, fetal brain, fetal liver, adrenal gland, liver, lung, small intestine, salivary gland, skeletal muscle, thymus, and thyroid as well as expressed in the following tumor tissues: breast, colon, kidney, lung, ovary, stomach, uterus, MDA435 and HEPG2. Soluble EphA2 (SEQ ID NO: 167) mRNA is highly expressed in the trachea, lung, small intestine, and salivary gland and to a lesser extent expressed in kidney, placenta, fetal brain, fetal liver, adrenal gland, skeletal muscle, thymus, brain, heart, spleen, spinal cord, uterus, and liver as well as highly expressed in stomach tumor and to a lesser extent in colon, kidney, lung, ovary, uterus, MDA435 and HEPG2 tumor tissues.

FGFR-4 (GenBank No. NM_{—}002011 set forth as SEQ ID NO: 2) mRNA is expressed in a variety of human tissues, including brain, heart, kidney, placenta, prostate, spleen, spinal cord, trachea, testis, uterus, fetal brain, fetal liver, adrenal gland, liver, lung, small intestine, salivary gland, skeletal muscle, thymus, and thyroid. FGFR-4 mRNA also is expressed in the following tumor tissues: breast, colon, kidney, lung, ovary, stomach, uterus, and HEPG2. Soluble FGFR-4 (SEQ ID NO: 120) mRNA is highly expressed in the kidney, spleen, testis, fetal brain, fetal liver, adrenal gland, liver, lung, small intestine and to a lesser extent expressed in brain, heart, placenta, prostate, spinal cord, trachea, uterus, skeletal muscle, thymus and thyroid. Soluble FGFR-4 (SEQ ID NO: 120) mRNA also is highly expressed in kidney and stomach tumor tissue and to a lesser extent in breast, colon, lung, ovary, and HEPG2 tumor tissues.

RON (GenBank No. NM_{—}002447 set forth as SEQ ID NO: 159) mRNA is highly expressed in trachea, testis, fetal brain, lung, small intestine, and thymus as well as being expressed in salivary gland, kidney, placenta, heart, prostate, thyroid and to a lesser extent brain, spleen, spinal cord, uterus, fetal liver, adrenal gland, liver, and skeletal muscle. RON mRNA also is expressed in the following tumor tissues: breast, colon, lung, ovary, stomach, HEPG2 and to a lesser extent in kidney and uterus tumor tissue. Soluble RON (SEQ ID NO:128) mRNA is highly expressed in colon and stomach tumor tissues. Soluble RON (SEQ ID NO:128) mRNA is expressed to a lesser extent in trachea, small intestine and thymus as well as in breast, lung, and ovary tumor tissues. Soluble RON (SEQ ID NO:219) mRNA is highly expressed in prostate, trachea, fetal brain, lung, small intestine, thymus as well as breast, colon, lung, ovary, and stomach tumor tissues. Soluble RON (SEQ ID NO:219) mRNA also is expressed to a lesser extent in brain, heart, kidney, placenta, spleen, spinal cord, testis, uterus, fetal liver, adrenal gland, liver, salivary gland, skeletal muscle, thyroid as well as kidney, uterus, MDA435 and HEPG2 tumor tissues. Soluble RON (SEQ ID NO:217) mRNA is highly expressed in trachea, lung, small intestine, thymus as well as breast and colon tumor tissues. Soluble RON (SEQ ID NO:217) mRNA is expressed to a lesser extent in brain, heart, kidney, placenta, prostate, spleen, testis, uterus, fetal brain, salivary gland, thyroid as well as lung, ovary, and stomach tumor tissues.

TEK (GenBank No. NM_{—}000459 set forth as SEQ ID NO:160) mRNA is highly expressed in heart, kidney, placenta, spleen, lung as well as colon, kidney, lung, and ovary tumor tissues. TEK mRNA also is expressed to a lesser extent in brain, prostate, spinal cord, trachea, testis, uterus, fetal brain, fetal liver, adrenal gland, liver, small intestine, skeletal muscle, thymus, thyroid as well as breast and stomach tumor tissues. Soluble TEK (SEQ ID NO:132) mRNA has low level expression in heart and kidney, as well as colon tumor tissues.

VEGFR-1 (GenBank No. NM_{—}002019 set forth as SEQ ID NO:157) mRNA is highly expressed in brain, heart, kidney, placenta, prostate, spleen, spinal cord, testis, uterus, fetal brain, fetal liver, adrenal gland, lung, small intestine, skeletal muscle and to a lesser extent in trachea, liver, salivary gland, thymus and thyroid. VEGFR-1 mRNA also is highly expressed in colon, kidney, lung and ovary tumor tissues and to a lesser extent expressed in breast and stomach tumor tissues. Soluble VEGFR-1 (SEQ ID NO:100) mRNA has low level expression in stomach tumor tissues.

VEGFR-3 (GenBank No. NM_{—}002020 set forth as SEQ ID NO:158) mRNA is highly expressed in heart, kidney, placenta, spleen, fetal brain, fetal liver, lung, small intestine as well as breast, colon, kidney, lung, ovary, stomach and uterus tumor tissues. VEGFR-3 (SEQ ID NO:158) mRNA is to a lesser extent expressed in brain, prostate, spinal cord, trachea, testis, uterus, adrenal gland, liver, salivary gland, skeletal muscle, thymus, thyroid. Soluble VEGFR-3 (SEQ ID NO:225) mRNA is highly expressed in placenta, adrenal gland, lung, small intestine as well as breast, kidney, lung tumor tissues. Soluble VEGFR-3 (SEQ ID NO:225) mRNA also is expressed to a lesser extent in brain, heart, kidney, prostate, spleen, spinal cord, trachea, testis, uterus, fetal brain, fetal liver, liver, salivary gland, skeletal muscle, thymus, and thyroid as well as colon, ovary, stomach, and uterus tumor tissues.

In summary, expression of mRNA was detectable for all CSR isoforms, but in general was lower than that of the membrane receptor isoforms.

B. Cell Secretion of Soluble Receptors

Putative CSR isoforms were analyzed in cultured human cells to assess secreted isoforms. Splice variant cDNA molecules encoding candidate CSR isoforms were subcloned into a mammalian expression vector (pcDNA3.1MycHis vector (Invitrogen, Carlsbad, Calif.) fused in frame with the Myc-His tag at the C-terminus of the protein to facilitate their detection.

Human embryonic kidney 293T cells were seeded at 2×10⁶ cells/well in a 6-well plate and maintained in Dulbecco's modified Eagle's medium and 10% fetal bovine serum (Invitrogen). Cells were transfected using LipofectAMINE 2000 (Invitrogen) following the manufacturer's instructions. On the day of transfection, 5 μg plasmid DNA was mixed with 15 μl of LipofectAMINE 2000 in 0.5 ml of the serum-free DMEM. The mixture was incubated for 20 minutes at room temperature before it was added to the cells. Cells were incubated at 37° C. in a CO₂ incubator for 48 hours. To study the transgene expression, the supernatants were collected and the cells were lysed in PBS buffer containing 0.2% of Triton X-100. Both the cell lysates and the supernatants were assayed for the transgene expression.

Ni-agarose NTA (Qiagen) was used for purifying His6-tagged proteins under native conditions following the manufacturer's instructions. Purified His6-tagged proteins were eluted and separated on SDS-polyacrylamide gels for immunoblotting using anti-Myc antibodies (both from Invitrogen). Antibodies were diluted 1:5000.

Expression of the secreted CSR isoforms was detected in cell lysates and conditioned media by Western blot using an anti-Myc antibody (Invitrogen) FGFR-4 (SEQ ID NO: 121), RON (SEQ ID NOS: 129, 216, 218, 220), VEGFR-2 (SEQ ID NO: 224), VEGFR-3 (SEQ ID NO: 127), EphA2 (SEQ ID NO:168), EphA1 (SEQ ID NOS: 153, 149), TEK (SEQ ID NOS: 131, 133), and Tie-1 (SEQ ID NO: 222) protein was detected in cell lysates and Tie-1 (SEQ ID NO: 222), VEGFR-2 (SEQ ID NO: 224), VEGFR-3 (SEQ ID NO: 127) and EphA2 (SEQ ID NO:168) protein was detected in conditioned medium.

C. Receptor Binding

Co-immunoprecipitation assays were performed to show binding of CSR isoforms and secreted isoforms to their respective membrane anchored fill-length receptors (see, for example, Jin et al. J Biol Chem 2004, 279:1408 and Jin et al. J Biol Chem 2004, 279:14179). Human embryo kidney 293T cells were transiently transfected with the recombinant pcDNA 3.1(MycHis) plasmid expressing soluble VEGFR-3 (as described above). Forty-eight hours after transfection, conditioned medium was collected and binding of VEGF-D was assessed. Conditioned medium (100 μl) from transfected 293T cells was incubated with VEGF-D (100 ng) in the presence or absence of 2 μg of soluble VEGFR-1-Fc or VEGFR-3-Fc (R&D Systems) for one hour. Protein complexes were immunoprecipitated with 0.2 μg/reaction of anti-VEGF-D antibodies (R&D Systems) and separated on a denaturing protein gel probed with anti-Myc antibody. The Western blot showed protein binding between sVEGF3-Myc and VEGF-D. Furthermore, 5× molar excess of a sVEGFR-3-Fc reduced binding whereas the presence of sVEGFR-1-Fc had little to no effect on binding.

D. Proliferation Assays

A biological activity of CSR isoforms was assessed by measuring their effect on cell proliferation. HUVEC cells (Clonitix) at passage 4 were seeded into DMEM/10% FBS at a density of 4,000 cells/well in a 96-well plate. Cells were treated with or without 0.5 nM of VEGF-A (R&D Systems) in the presence or absence of 2.5 nM of sVEGFR-1-Fc, 2.5 nM of sVEGFR-2-Fc, or 1.6-12.5 nM of the purified sVEGFR2. The treated cells were cultured for 7 days in standard cell culture conditions. Cell proliferation was determined in triplicate samples using CyQuant Fluorescence Assay Kit (Invitrogen Catalog #C7026) according to manufacturer's instructions. 0.5 nM of VEGF-A induced HUVEC proliferation. sVEGFR-1-Fc (2.5 nM) and sVEGFR-2-Fc (2.5 nM) each inhibited VEGFA-induced HUVEC proliferation. Soluble VEGFR-2 (SEQ ID NO: 224) inhibited VEGF-A-induced HUVEC proliferation in a dose-dependent manner.

Since modifications will be apparent to those of skill in this art, it is intended that this invention be limited only by the scope of the appended claims.

1. An isolated polypeptide, comprising at least one domain of an EphA receptor, wherein the polypeptide comprises an ephrin ligand binding domain and the polypeptide lacks one or more amino acids corresponding to a transmembrane domain of the EphA receptor whereby the membrane localization of the polypeptide is reduced or abolished compared to the EphA receptor.

2. A polypeptide of claim 1, wherein the EphA receptor is selected from the group consisting of EphA1, EphA2, EphA3, EphA4, EphA5, EphA6, EphA7, and EphA8.

3. A polypeptide of claim 2, wherein the EphA receptor comprises a sequence of amino acids set forth in any of SEQ ID NO: 253-260 or is an allelic variant thereof.

4. A polypeptide of claim 3, wherein the allelic variant comprises one or more of the allelic variations set forth in any one of SEQ ID NOS: 289-293.

5. A polypeptide of claim 1, wherein the polypeptide lacks all or part of a protein kinase domain compared to the EphA receptor.

6. A polypeptide of claim 1, wherein the polypeptide lacks all or part of a Sterile Alpha Motif domain (SAM) compared to the EphA receptor.

7. A polypeptide of claim 1, comprising at least one domain of an EphA1 receptor as set forth in SEQ ID NO:253.

8. A polypeptide of claim 7 that comprises an intron-encoded sequence of amino acids, wherein the intron is from a gene encoding the EphA1 receptor.

9. A polypeptide of claim 7, wherein the polypeptide comprises at least one domain of the EphA1 receptor operatively linked to at least one amino acid encoded by an intron of a gene encoding the EphA1 receptor.

10. A polypeptide of claim 7, wherein the polypeptide lacks one or more amino acids of a protein kinase domain of the EphA1 receptor, whereby the kinase activity of the polypeptide is reduced or abolished compared to the EphA1 receptor.

11. A polypeptide of claim 10, wherein the polypeptide has at least 80% sequence identity with a sequence of amino acids set forth in any of SEQ ID NOS: 149, 151 and 153.

12. A polypeptide of claim 11 that comprises the sequence of amino acid set forth in any of SEQ ID NOS: 149, 151 and 153 or is an allelic variant thereof.

13. A polypeptide of claim 12, wherein the allelic variant comprises one or more amino acids of the allelic variations as set forth in SEQ ID NO: 289.

14. A polypeptide of claim 7, wherein the polypeptide contains the same number of amino acids as set forth in any of SEQ ID NOS: 149, 151 and 153.

15. A polypeptide of claim 1, comprising at least one domain of an EphA2 receptor as set forth in SEQ ID NO: 254, wherein the polypeptide lacks one or more amino acids of a transmembrane domain and protein kinase domain compared to the EphA2 receptor, whereby the membrane localization and the protein kinase activity of the polypeptide are reduced or abolished compared to the EphA2 receptor.

16. A polypeptide of claim 15 that comprises an intron-encoded sequence of amino acids, wherein the intron is from a gene encoding an EphA2 receptor.

17. A polypeptide of claim 15, wherein the polypeptide comprises at least one domain of the EphA2 receptor operatively linked to at least one amino acid encoded by an intron of a gene encoding an EphA2 receptor.

18. A polypeptide of claim 15, wherein the polypeptide lacks one or more amino acids of a fibronectin domain compared to the EphA2 receptor.

19. A polypeptide of claim 18, wherein the polypeptide has at least 80% sequence identity with a sequence of amino acids as set forth in SEQ ID NO: 168.

20. A polypeptide of claim 19 that comprises the sequence of amino acids set forth in SEQ ID NO: 168 or an allelic variant thereof.

21. A polypeptide of claim 20, wherein the allelic variant comprises one or more amino acids of the allelic variations as set forth in SEQ ID NO: 290.

22. A polypeptide of claim 15, wherein the polypeptide contains the same number of amino acids as set forth in SEQ ID NO: 168.

23. An isolated polypeptide, comprising at least one domain of an EphB receptor, wherein the polypeptide lacks one or more amino acids of a transmembrane domain compared to the EphB receptor, whereby the membrane localization of the polypeptide is reduced or abolished compared to the EphB receptor.

24. A polypeptide of claim 23, wherein the EphB receptor is selected from the group consisting of EphB1, EphB2, EphB3, EphB4, EphB5, and EphB6.

25. A polypeptide of claim 24, wherein the EphB receptor comprises a sequence of amino acids as set forth in any one of SEQ ID NOS: 261-265 or an allelic variant thereof.

26. A polypeptide of claim 25, wherein the allelic variant comprises one or more of the allelic variations as set forth in any one of SEQ ID NOS: 294-298.

27. A polypeptide of claim 23, wherein the polypeptide lacks one or more amino acids of a protein kinase domain of the EphB receptor, whereby the protein kinase activity of the polypeptide is reduced or abolished compared to the EphB receptor.

28. A polypeptide of claim 23, wherein the polypeptide lacks one or more amino acids of a Sterile Alpha Motif domain (SAM) of the EphB receptor.

29. A polypeptide of claim 23, wherein the polypeptide comprises an ephrin ligand and binding domain.

30. A polypeptide of claim 23, wherein the polypeptide lacks one or more amino acids of a fibronectin domain of the EphB receptor.

31. A polypeptide of claim 23, wherein the polypeptide comprises an intron-encoded sequence of amino acids, wherein the intron is from a gene encoding the EphB receptor.

32. A polypeptide of claim 31, wherein the polypeptide comprises at least one domain of the EphB receptor operatively linked to at least one amino acid encoded by an intron of a gene encoding the EphB receptor.

33. A polypeptide of claim 23, wherein the polypeptide has at least 80% sequence identity with a sequence of amino acids as set forth in any of SEQ ID NOS: 155, 170, 172 and 174.

34. A polypeptide of claim 33 that comprises the sequence of amino acids as set forth in any of SEQ ID NOS: 155, 170, 172 and 174 or an allelic variant thereof.

35. A polypeptide of claim 34, wherein the allelic variant comprises one or more amino acids of the allelic variations as set forth in SEQ ID NOS: 294 or 297.

36. A polypeptide of claim 23, wherein the polypeptide contains the same number of amino acids as set forth in any of SEQ ID NOS: 155, 170, 172 and 174.

37. An isolated polypeptide, comprising at least one domain of an FGFR-1, wherein the polypeptide comprises an immunoglobulin domain corresponding to amino acids 253-357 of the FGFR-1 set forth in SEQ ID NO: 268 and lacks all of a transmembrane domain corresponding to amino acids 375-397 of the FGFR-1.

38. A polypeptide of claim 37 that comprises an intron-encoded sequence of amino acids, wherein the intron is from a gene encoding the FGFR-1.

39. A polypeptide of claim 37, wherein the polypeptide comprises at least one domain of the FGFR-1 operatively linked to at least one amino acid encoded by an intron of a gene encoding an FGFR-1.

40. A polypeptide of claim 37, wherein the polypeptide lacks one or more amino acids of a protein kinase domain of the FGFR-1, whereby the protein kinase activity of the polypeptide is reduced or abolished compared to the FGFR-1.

41. A polypeptide of claim 37, wherein the polypeptide comprises one or more amino acids of an immunoglobulin domain corresponding to amino acids 156-246 of the FGFR-1.

42. A polypeptide of claim 37, wherein the polypeptide has at least 80% sequence identity with a sequence of amino acids set forth in SEQ ID NOS: 119 or 176.

43. A polypeptide of claim 42 that comprises the sequence of amino acids as set forth in any of SEQ ID NOS: 119 and 176 or an allelic variant thereof.

44. A polypeptide of claim 43, wherein the allelic variant comprises one or more amino acids of the allelic variations as set forth in SEQ ID NO: 300.

45. A polypeptide of claim 37, wherein the polypeptide contains the same number of amino acids as set forth in SEQ ID NOS: 119 or 176.

46. An isolated polypeptide, comprising at least one domain of a fibroblast growth factor receptor-2 (FGFR-2), wherein:
the FGFR-2 comprises a sequence of amino acids set forth in SEQ ID NO: 269;
the polypeptide lacks a transmembrane domain and a protein kinase domain compared to the FGFR-2, whereby membrane localization and protein kinase activity of the polypeptide is reduced or abolished compared to the FGFR-2; and
the polypeptide has at least 80% sequence identity with a sequence of amino acids set forth in SEQ ID NOS: 178, 180, 182 and 184.

47. A polypeptide of claim 46 that comprises an intron-encoded sequence of amino acids, wherein the intron is from a gene encoding the FGFR-2.

48. A polypeptide of claim 46, wherein the polypeptide comprises at least one domain of the FGFR-2 operatively linked to at least one amino acid encoded by an intron of a gene encoding the FGFR-2.

49. A polypeptide of claim 46, wherein the polypeptide lacks an immunoglobulin domain corresponding to amino acids 41-125 of the FGFR-2.

50. A polypeptide of claim 46 that comprises the sequence of amino acids set forth in SEQ ID NOS: 178, 180, 182 or 184 or an allelic variant thereof.

51. A polypeptide of claim 50, wherein the allelic variant comprises one or more amino acids of the allelic variations as set forth in SEQ ID NO: 301.

52. A polypeptide of claim 46, wherein the polypeptide contains the same number of amino acids as set forth in any of SEQ ID NOS: 178, 180, 182 and 184.

53. An isolated polypeptide, comprising at least one domain of an FGFR-4, wherein the polypeptide comprises an immunoglobulin domain corresponding to amino acids 249-351 of the FGFR-4 set forth in SEQ ID NO: 271 and lacks a transmembrane domain and a protein kinase domain of the FGFR-4, whereby membrane localization and protein kinase activity of the polypeptide is reduced or abolished compared to the FGFR-4.

54. A polypeptide of claim 53 that comprises an intron-encoded sequence of amino acids, wherein the intron is from a gene encoding the FGFR-4.

55. A polypeptide of claim 53, wherein the polypeptide comprises at least one domain of the FGFR-4 operatively linked to at least one amino acid encoded by an intron of a gene encoding the FGFR-4.

56. A polypeptide of claim 53, wherein the polypeptide has at least 80% sequence identity with a sequence of amino acids set forth in SEQ ID NO: 121.

57. A polypeptide of claim 53, that comprises a sequence of amino acids as set forth in SEQ ID NO: 121 or an allelic variant thereof.

58. A polypeptide of claim 57, wherein the allelic variant comprises one or more amino acids of the allelic variations as set forth in SEQ ID NO: 303.

59. A polypeptide of claim 53, wherein the polypeptide contains the same number of amino acids as set forth in SEQ ID NO: 121.

60. An isolated polypeptide, comprising at least one domain of a DDR1 as set forth in SEQ ID NO: 250, wherein the polypeptide lacks a transmembrane domain and a protein kinase domain compared to the DDR1, whereby membrane localization and protein kinase activity of the polypeptide is reduced or abolished compared to the DDR1, and the polypeptide has at least 80% sequence identity with a sequence of amino acids set forth in SEQ ID NOS: 115 or 117.

61. A polypeptide of claim 60 that comprises an intron-encoded sequence of amino acids, wherein the intron is from a gene encoding the DDR1.

62. A polypeptide of claim 61, wherein the polypeptide comprises at least one domain of the DDR1 operatively linked to at least one amino acid encoded by an intron of a gene encoding the DDR1.

63. A polypeptide of claim 60, that comprises the sequence of amino acids set forth in SEQ ID NOS: 115 or 117 or an allelic variant thereof.

64. A polypeptide of claim 63, wherein the allelic variant comprises one or more amino acids of the allelic variations as set forth in SEQ ID NO: 286.

65. A polypeptide of claim 60, wherein the polypeptide contains the same number of amino acids as set forth in SEQ ID NOS: 115 or 117.

66. An isolated polypeptide, comprising at least one domain of a MET receptor, wherein:
the polypeptide lacks a transmembrane domain, a protein kinase domain and at least one additional domain compared to the MET receptor as set forth in SEQ ID NO: 274, whereby membrane localization and protein kinase activity of the polypeptide is reduced or abolished compared to the MET receptor.

67. A polypeptide of claim 66 that comprises an intron-encoded sequence of amino acids, wherein the intron is from a gene encoding a MET receptor.

68. A polypeptide of claim 66, wherein the polypeptide comprises at least one domain of the MET receptor operatively linked to at least one amino acid encoded by an intron of a gene encoding a MET receptor.

69. A polypeptide of claim 66, wherein the additional domain is selected from the group consisting of a Sema domain, a plexin domain and an IPT/TIG domain.

70. A polypeptide of claim 66, wherein the polypeptide has at least 80% sequence identity with a sequence of amino acids as set forth in any of SEQ ID NOS: 186, 188, 190, 192, 194, 196, 198, 200, 202, 204, 206, 208, and 214.

71. A polypeptide of claim 66, that comprises a sequence of amino acids set forth in any of SEQ ID NOS: 186, 188, 190, 192, 194, 196, 198, 200, 202, 204, 206, 208, and 214 or an allelic variant thereof.

72. A polypeptide of claim 71, wherein the allelic variant comprises one or more amino acids of the allelic variations as set forth in SEQ ID NO: 306.

73. A polypeptide of claim 66, wherein the polypeptide contains the same number of amino acids as set forth in any of SEQ ID NOS: 186, 188, 190, 192, 194, 196, 198, 200, 202, 204, 206, 208 and 214.

74. An isolated polypeptide, comprising at least one domain of a RON receptor, wherein:
the polypeptide comprises a plexin domain of the RON receptor as set forth in SEQ ID NO: 277; and
the polypeptide lacks a transmembrane domain of the RON receptor, whereby membrane localization of the polypeptide is reduced or abolished compared to the RON receptor.

75. A polypeptide of claim 74 that comprises an intron-encoded sequence of amino acids, wherein the intron is from a gene encoding the RON receptor.

76. A polypeptide of claim 74, wherein the polypeptide comprises at least one domain of the RON receptor operatively linked to at least one amino acid encoded by an intron of a gene encoding a RON receptor.

77. A polypeptide of claim 74, wherein the polypeptide lacks one or more amino acids of a protein kinase domain compared to the RON receptor as set forth in SEQ ID NO: 277, whereby protein kinase activity of the polypeptide is reduced or abolished compared to the RON receptor.

78. A polypeptide of claim 74, wherein the polypeptide comprises one or more amino acids of at least one IPT/TIG domain of the RON receptor.

79. A polypeptide of claim 74, wherein the polypeptide has at least 80% sequence identity with a sequence of amino acids as set forth in any of SEQ ID NOS: 216, 218 and 220.

80. A polypeptide of claim 74, that comprises a sequence of amino acids set forth in any of SEQ ID NOS: 216, 218 and 220 or an allelic variant thereof.

81. A polypeptide of claim 80, wherein the allelic variant comprises one or more amino acids of the allelic variations as set forth in SEQ ID NO: 308.

82. A polypeptide of claim 74, wherein the polypeptide contains the same number of amino acids as set forth in any of SEQ ID NOS: 216, 218 and 220.

83. An isolated polypeptide, comprising at least one domain of a TEK receptor as set forth in SEQ ID NO: 278, wherein:
the polypeptide lacks a transmembrane domain, and a protein kinase domain whereby membrane localization and protein kinase activity of the polypeptide are reduced or abolished compared to the TEK receptor; and
the polypeptide lacks one or more amino acids of at least one fibronectin domain compared to the TEK receptor.

84. A polypeptide of claim 83 that comprises an intron-encoded sequence of amino acids, wherein the intron is from a gene encoding the TEK receptor.

85. A polypeptide of claim 83, wherein the polypeptide comprises at least one domain of the TEK receptor operatively linked to at least one amino acid encoded by an intron of a gene encoding the TEK receptor.

86. A polypeptide of claim 83, wherein the fibronectin domain lacking in the polypeptide corresponds to amino acids 444-529, 543-626, or 639-724 of SEQ ID NO: 278.

87. A polypeptide of claim 83, wherein the polypeptide lacks one or more amino acids of the three fibronectin domains of the TEK receptor corresponding to amino acids 444-529, 543-626, and 639-724 of SEQ ID NO: 278.

88. A polypeptide of claim 83, wherein the polypeptide has at least 80% sequence identity with a sequence of amino acids as set forth in any of SEQ ID NOS: 131 and 133.

89. A polypeptide of claim 83, that comprises a sequence of amino acids set forth in any of SEQ ID NOS: 131 and 133 or an allelic variant thereof.

90. A polypeptide of claim 89, wherein the allelic variant comprises one or more amino acids of the allelic variations as set forth in SEQ ID NO: 309.

91. A polypeptide of claim 83, wherein the polypeptide contains the same number of amino acids as set forth in any of SEQ ID NOS: 131 and 133.

92. An isolated polypeptide, comprising all or part of at least one domain of a Tie-1 receptor as set forth in SEQ ID NO: 279, wherein:
the polypeptide lacks a transmembrane domain and a protein kinase domain compared to the Tie-1 receptor, whereby membrane localization and protein kinase activity of the polypeptide are reduced or abolished compared to the Tie-1 receptor; and
the polypeptide comprises a sequence of amino acids set forth in any of SEQ ID NOS: 135, 137, 139, 141, 143 and 222 or an allelic variant thereof.

93. A polypeptide of claim 92, wherein the allelic variant comprises one or more amino acids of the allelic variations as set forth in SEQ ID NO: 310.

94. A polypeptide of claim 92, wherein the polypeptide contains the same number of amino acids as set forth in any of SEQ ID NOS: 135, 137, 139, 141, 143 and 222.

95. An isolated polypeptide, wherein:
the polypeptide comprises a sequence of amino acids that has at least 80% sequence identity with a sequence of amino acids as set forth in SEQ ID NO: 123; and
the polypeptide lacks a transmembrane domain and a protein kinase domain compared to a VEGFR-1 receptor set forth in SEQ ID NO: 282.

96. A polypeptide of claim 95, that comprises the sequence of amino acids set forth in SEQ ID NO: 123 or an allelic variant thereof.

97. A polypeptide of claim 95, wherein the polypeptide contains the same number of amino acids as set forth in SEQ ID NO: 123.

98. An isolated polypeptide, comprising at least one domain of a VEGFR set forth in any of SEQ ID NOS: 283 and 284, wherein the polypeptide lacks one or more amino acids of a transmembrane domain of the VEGFR, whereby membrane localization of the polypeptide is reduced or abolished compared to the VEGFR.

99. A polypeptide of claim 98 that comprises an intron-encoded sequence of amino acids, wherein the intron is from a gene encoding the VEGFR.

100. A polypeptide of claim 99, wherein the polypeptide comprises at least one domain of the VEGFR operatively linked to at least one amino acid encoded by an intron of a gene encoding the VEGFR.

101. A polypeptide of claim 98, wherein the polypeptide lacks one or more amino acids of a protein kinase domain, whereby protein kinase activity of the polypeptide is reduced or abolished compared to the VEGFR.

102. A polypeptide of claim 98, wherein the polypeptide lacks one or more amino acids of an immunoglobulin domain compared to the VEGFR.

103. A polypeptide of claim 102, wherein the polypeptide has at least 80% sequence identity with a sequence of amino acids as set forth in any of SEQ ID NOS: 125, 127, 224 and 226.

104. A polypeptide of claim 98, that comprises a sequence of amino acids set forth in any of SEQ ID NOS: 125, 127, 224 and 226 or an allelic variant thereof.

105. A polypeptide of claim 104, wherein the allelic variant comprises one or more amino acids of the allelic variations as set forth in SEQ ID NOS: 313 or 314.

106. A polypeptide of claim 98, wherein the polypeptide contains the same number of amino acids as set forth in any of SEQ ID NOS: 125, 127, 224 and 226.

107. An isolated polypeptide, comprising at least one domain of a PDGFR-B as set forth in SEQ ID NO: 276, wherein the polypeptide lacks one or more amino acids of a transmembrane domain of the PDGFR-B, whereby membrane localization of the polypeptide is reduced or abolished compared to the PDGFR-B.

108. A polypeptide of claim 107, that comprises an intron-encoded sequence of amino acids, wherein the intron is from a gene encoding the PDGFR-B.

109. A polypeptide of claim 107, wherein the polypeptide comprises at least one domain of the PDGFR-B operatively linked to at least one amino acid encoded by an intron of a gene encoding the PDGFR-B.

110. A polypeptide of claim 107, wherein the polypeptide lacks one or more amino acids of a protein kinase domain of the PDGFR-B, whereby protein kinase activity of the polypeptide is reduced or abolished compared to the PDGFR-B.

111. A polypeptide of claim 107, wherein the polypeptide comprises one or more amino acids of an immunoglobulin domain of the PDGFR-B.

112. A polypeptide of claim 107, wherein the polypeptide has at least 80% sequence identity with a sequence of amino acids set forth in SEQ ID NO: 147.

113. A polypeptide of claim 107, that comprises a sequence of amino acids set forth in SEQ ID NO: 147 or an allelic variant thereof.

114. A polypeptide of claim 113, wherein the allelic variant comprises one or more amino acids of the allelic variations as set forth in SEQ ID NO: 307.

115. A polypeptide of claim 107, wherein the polypeptide contains the same number of amino acids as set forth in SEQ ID NO: 147.

116. An isolated polypeptide, comprising at least one domain of a CSF1R as set forth in SEQ ID NO: 249, wherein the polypeptide lacks one or more amino acids of a transmembrane domain of the CSF1R, whereby membrane localization of the polypeptide is reduced or abolished compared to the CSF1R.

117. A polypeptide of claim 116, that comprises an intron-encoded sequence of amino acids, wherein the intron is from a gene encoding the CSF1R.

118. A polypeptide of claim 117, wherein the polypeptide comprises at least one domain of the CSF1R operatively linked to at least one amino acid encoded by an intron of a gene encoding the CSF1R.

119. A polypeptide of claim 116, wherein the polypeptide lacks one or more amino acids of a protein kinase domain of the CSF1R, whereby protein kinase activity of the polypeptide is reduced or abolished compared to the CSF1R.

120. A polypeptide of claim 116, wherein the polypeptide comprises one or more amino acids of an immunoglobulin domain of the CSF1R.

121. A polypeptide of claim 116, wherein the polypeptide has at least 80% sequence identity with a sequence of amino acids set forth in SEQ ID NO: 145.

122. A polypeptide of claim 116, that comprises a sequence of amino acids set forth in SEQ ID NO: 145 or an allelic variant thereof.

123. A polypeptide of claim 122, wherein the allelic variant comprises one or more amino acids of the allelic variations as set forth in SEQ ID NO: 285.

124. A polypeptide of claim 116, wherein the polypeptide contains the same number of amino acids as set forth in SEQ ID NO: 145.

125. An isolated polypeptide, comprising at least one domain of a KIT receptor as set forth in SEQ ID NO:273, wherein the polypeptide lacks one or more amino acids of a transmembrane domain and a protein kinase domain of the KIT receptor, whereby membrane localization and protein kinase activity of the polypeptide are reduced or abolished compared to the KIT receptor.

126. A polypeptide of claim 125, that comprises an intron-encoded sequence of amino acids, wherein the intron is from a gene encoding the KIT receptor.

127. A polypeptide of claim 125, wherein the polypeptide comprises at least one domain of the KIT receptor operatively linked to at least one amino acid encoded by an intron of a gene encoding a KIT receptor.

128. A polypeptide of claim 125, wherein the polypeptide comprises at least one immunoglobulin domain of the KIT receptor.

129. A polypeptide of claim 125, wherein the polypeptide has at least 80% sequence identity with a sequence of amino acids set forth in SEQ ID NO: 93.

130. A polypeptide of claim 125, that comprises a sequence of amino acids set forth in SEQ ID NO: 93 or an allelic variant thereof.

131. A polypeptide of claim 130, wherein the allelic variant comprises one or more amino acids of the allelic variations as set forth in SEQ ID NO: 305.

132. A polypeptide of claim 125, wherein the polypeptide contains the same number of amino acids as set forth in SEQ ID NO: 93.

133. An isolated polypeptide, comprising at least one cysteine rich c6 domain of a TNFR as set forth in SEQ ID NOS: 280 or 281, wherein the polypeptide lacks all of a transmembrane domain of the TNFR, whereby membrane localization of the polypeptide is reduced or abolished compared to the TNFR.

134. A polypeptide of claim 133, that comprises an intron-encoded sequence of amino acids, wherein the intron is from a gene encoding the TNFR.

135. A polypeptide of claim 133, wherein the polypeptide comprises at least one domain of the TNFR operatively linked to at least one amino acid encoded by an intron of a gene encoding the TNFR.

136. A polypeptide of claim 133, wherein the polypeptide comprises at least two cysteine rich c6 domains of the TNFR.

137. A polypeptide of claim 133, wherein the polypeptide has at least 80% sequence identity with a sequence of amino acids set forth in SEQ ID NO: 95.

138. A polypeptide of claim 133, that comprises a sequence of amino acids set forth in SEQ ID NO: 95 or an allelic variant thereof.

139. A polypeptide of claim 138, wherein the allelic variant comprises one or more amino acids of the allelic variations as set forth in SEQ ID NO: 312.

140. A polypeptide of claim 133, wherein the polypeptide contains the same number of amino acids as set forth in SEQ ID NO: 95.

141. A pharmaceutical composition, comprising a polypeptide isoform that lacks a transmembrane domain and at least or part of at least one other domain of a receptor selected from among a EphA, EphB, FGFR-1, FGFR-2, FGFR-4, DDR1, MET, RON, TEK, Tie-1, VEGFR, PDGFR-B, CSF1R, KIT, and TNFR, wherein the polypeptide modulates an activity or function of its cognate receptor.

142. The pharmaceutical composition of claim 141, wherein the polypeptide is selected from among:
(a) an isolated polypeptide, comprising a sequence of amino acids set forth in any one of SEQ ID NOS: 91, 93, 95, 115, 117, 119, 121, 123, 125, 127, 129, 131, 133, 135, 137, 139, 141, 143, 145, 147, 149, 151, 153, 155, 168, 170, 172, 174, 176, 178, 180, 182, 184, 186, 188, 190, 192, 194, 196, 198, 200, 202, 204, 206, 208, 210, 212, 214, 216, 218, 220, 222, 224 and 226;
(b) an isolated polypeptide consisting essentially of a sequence of amino acids set forth in any one of SEQ ID NOS: 91, 93, 95, 115, 117, 119, 121, 123, 125, 127, 129, 131, 133, 135, 137, 139, 141, 143, 145, 147, 149, 151, 153, 155, 168, 170, 172, 174, 176, 178, 180, 182, 184, 186, 188, 190, 192, 194, 196, 198, 200, 202, 204, 206, 208, 210, 212, 214, 216, 218, 220, 222, 224 and 226;
(c) an isolated polypeptide, comprising a sequence of amino acids that has at least 80% sequence identity with a sequence of amino acids set forth in any of SEQ ID NOS: 91, 93, 95, 115, 117, 119, 121, 123, 125, 127, 129, 131, 133, 135, 137, 139, 141, 143, 145, 147, 149, 151, 153 and 155 or an allelic variant thereof, wherein:
sequence identity is compared along the full length of each SEQ ID to the full length sequence of the isolated polypeptide; and
each of SEQ ID NOS: 91, 93, 95, 115, 117, 119, 121, 123, 125, 127, 129, 131, 133, 135, 137, 139, 141, 143, 145, 147, 149, 151, 153 and 155 is a cell surface receptor isoform; and
(d) an isolated polypeptide, comprising a sequence of amino acids set forth in any of SEQ ID NOS: 91, 93, 95, 115, 117, 119, 121, 123, 125, 127, 129, 131, 133, 135, 137, 139, 141, 143, 145, 147, 149, 151, 153 and 155.

143. The composition of claim 141, wherein the activity modulated by the polypeptide is one or more of: dimerization, homodimerization, heterodimerization, trimerization, kinase activity, receptor-associated kinase activity, receptor-associated protease activity, autophosphorylation of the receptor, transphosphorylation of the receptor, phosphorylation of a signal transduction molecule, ligand binding, competition with the receptor for ligand binding, signal transduction, interaction with a signal transduction molecule, induction of apoptosis, membrane association and membrane localization.

144. The composition of claim 143, wherein modulation is an inhibition of activity.

145. An isolated nucleic acid molecule, comprising a sequence of nucleic acids set forth in any of SEQ ID NOS: 90, 92, 94, 114, 116, 118, 120, 122, 124, 126, 128, 130, 132, 134, 136, 138, 140, 142, 144, 146, 148, 150, 152, 154, 167, 169, 171, 173, 175, 177, 179, 181, 183, 185, 187, 189, 191, 193, 195, 197, 199, 201, 203, 205, 207, 209, 211, 213, 215, 217, 219, 221, 223 and 225 or an allelic variant thereof; or
an isolated nucleic acid molecule, comprising a sequence of nucleotides that has at least 90% sequence identity with a sequence of nucleotides set forth in any of SEQ ID NOS: 90, 92, 94, 114, 116, 118, 120, 122, 124, 126, 128, 130, 132, 134, 136, 138, 140, 142, 144, 146, 148, 150, 152 and 154 or an allelic variations variant thereof, wherein:
sequence identity is compared along the full length of each SEQ ID to the full length sequence of the isolated nucleic acid molecule; and
each of SEQ ID NOS: 90, 92, 94, 114, 116, 118, 120, 122, 124, 126, 128, 130, 132, 134, 136, 138, 140, 142, 144, 146, 148, 150, 152 and 154 is a cell surface receptor isoform.

146. A vector, comprising the nucleic acid molecule of claim 145.

147. A cell, comprising the vector of claim 146.

148. A method of treating a disease or condition comprising, administering a pharmaceutical composition of claim 141.

149. An isolated polypeptide, wherein the polypeptide is selected from among:
(a) an isolated polypeptide that comprise comprising a sequence of amino acids set forth in any one of SEQ ID NOS: 91, 93, 95, 115, 117, 119, 121, 123, 125, 127, 129, 131, 133, 135, 137, 139, 141, 143, 145, 147, 149, 151, 153, 155, 168, 170, 172, 174, 176, 178, 180, 182, 184, 186, 188, 190, 192, 194, 196, 198, 200, 202, 204, 206, 208, 210, 212, 214, 216, 218, 220, 222, 224 and 226;
(b) an isolated polypeptide consisting essentially of a sequence of amino acids set forth in any one of SEQ ID NOS: 91, 93, 95, 115, 117, 119, 121, 123, 125, 127, 129, 131, 133, 135, 137, 139, 141, 143, 145, 147, 149, 151, 153, 155, 168, 170, 172, 174, 176, 178, 180, 182, 184, 186, 188, 190, 192, 194, 196, 198, 200, 202, 204, 206, 208, 210, 212, 214, 216, 218, 220, 222, 224 and 226;
(c) an isolated polypeptide comprising a sequence of amino acids that has at least 80% sequence identity with a sequence of amino acids set forth in any of SEQ ID NOS: 91, 93, 95, 115, 117, 119, 121, 123, 125, 127, 129, 131, 133, 135, 137, 139, 141, 143, 145, 147, 149, 151, 153 and 155 or an allelic variant thereof, wherein:
sequence identity is compared along the full length of each SEQ ID to the full length sequence of the isolated polypeptide; and
each of SEQ ID NOS: 91, 93, 95, 115, 117, 119, 121, 123, 125, 127, 129, 131, 133, 135, 137, 139, 141, 143, 145, 147, 149, 151, 153 and 155 is a cell surface receptor isoform; and
(d) an isolated polypeptide, comprising a sequence of amino acids set forth in any of SEQ ID NOS: 91, 93, 95, 115, 117, 119, 121, 123, 125, 127, 129, 131, 133, 135, 137, 139, 141, 143, 145, 147, 149, 151, 153 and 155.

150. An isolated polypeptide of claim 149, wherein the polypeptide occurs in a mammal.

151. An isolated polypeptide of claim 150, wherein the mammal is a rodent, a primate or a human.

152. An isolated polypeptide, comprising at least one domain of a cell surface receptor operatively linked to at least one amino acid encoded by an intron of a gene encoding the cell surface receptor;
wherein the cell surface receptor is selected from the group consisting of a DDR1, KIT, FGFR-1, FGFR-4, TNFR2, VEGFR-1, VEGFR-3, RON, TEK, Tie-1, CSF1R, PDGFR-B, EphA1, and EphB1; or wherein the polypeptide comprises a sequence of amino acids selected from the group consisting of SEQ ID NOS: 91, 93, 95, 115, 117, 119, 121, 123, 125, 127, 129, 131, 133, 135, 137, 139, 141, 143, 145, 147, 149, 151, 153 and 155.

153. An isolated polypeptide, comprising a shortened cell surface receptor lacking at least all or part of a transmembrane domain, wherein:
the polypeptide is not membrane localized;
the polypeptide modulates an activity of the cell surface receptor;
the cell surface receptor is selected from the group consisting of a DDR1, KIT, FGFR-1, FGFR-4, TNFR2, VEGFR-1, VEGFR-3, RON, TEK, Tie-1, CSF1R, PDGFR-B, EphA1, and EphB1, or the isolated polypeptide has at least 80% sequence identity with a sequence of amino acids set forth in any of SEQ ID NOS: 91, 93, 95, 115, 117, 119, 121, 123, 125, 127, 129, 131, 133, 135, 137, 139, 141, 143, 145, 147, 149, 151, 153 and 155; and
sequence identity is compared along the full length of each SEQ ID to the sequence of the full length of the isolated polypeptide.

154. An isolated polypeptide of claim 153, wherein the cell surface receptor further lacks a cell surface receptor cytoplasmic domain.

155. An isolated polypeptide, comprising an intron-encoded sequence of amino acids, wherein:
the intron is from a cell surface receptor gene selected from the group consisting of a KIT, FGFR-4, TNFR, VEGFR-1, RON, TEK, Tie-1 and EphA1; or
the intron-encoded sequence is of any of SEQ ID NOS: 91, 93, 95, 121, 123, 129, 131, 133, 135, 137, 139, 141, 149, 151 and 153;
the polypeptide lacks a cell surface receptor cytoplasmic domain; and
the polypeptide further lacks a transmembrane domain.

156. A combination comprising:
two and one or more different cell surface receptor isoforms and/or a therapeutic drug or a cell surface receptor isoform and a therapeutic drug.

157. An isolated polypeptide of claim 155, wherein the polypeptide comprises a TNFR isoform selected from among a TNFR1, TNFR2, TNFRrp, low-affinity nerve growth factor receptor, Fas antigen, CD40, CD27, CD30, 4-1BB, OX40, DR3, DR4, DR5 and herpesvirus entry mediator (HVEM).

158. A method of regulating development and/or disease states, comprising contacting cells or tissues in vitro or in vivo with a cell surface receptor isoform (CSR) that lacks one or more domains or activities of the CSR, wherein the CSR is involved in angiogenesis or development.

159. The method of claim 158, wherein the CSR is an intron fusion protein.

160. A chimeric polypeptide, comprising a portion of one cell surface receptor (CSR) isoform and a portion of a second, different CSR isoform, wherein:
the chimeric polypeptide modulates an activity of one or more receptor tyrosine kinases; and
each portion contains at least 4, 5, 6, 7, 8, 10, 12, 15, or more amino acid residues.

161. A polypeptide of claim 160, wherein the first portion comprises all or part of an extracellular domain of a cell surface receptor; and the second portion comprises an intron-encoded portion from an intron fusion protein.

162. A polypeptide of claim 161, wherein the intron-encoded portion is a herstatin intron-encoded portion.

163. A polypeptide of claim 162, wherein the intron-encoded portion is set forth in any of SEQ ID NOS: 320-345.

164. An isolated polypeptide, comprising a cell surface receptor isoform, wherein:
the polypeptide is an intron fusion protein that contains at least one amino acid encoded by an intron of a gene encoding a polypeptide receptor isoform selected from among isoforms of FGFR-4, KIT, TNFRs, DDR1, FGFR-1, VEGFR-2, VEGFR-3, RON, TEK, CSF1R, PDGFR-B, EphA, EphB and MET; and the polypeptide does not contain a transmembrane domain or does not contain a sufficient portion of a transmembrane domain to anchor the polypeptide on a cell.

165. An isolated polypeptide of claim 164 that is a receptor antagonist.

166. A conjugate, comprising: a first portion linked directly or via a linker to an intron-encoded portion of an intron fusion polypeptide, wherein the resulting polypeptide modulates an activity of a cell surface receptor.

167. The conjugate of claim 166, wherein:
the first portion comprises all or portion of an extracellular domain of a first cell surface receptor (CSR);
the first portion is sufficient to mediate interaction with a ligand or with a second CSR; and
the first and second CSR are the same or different.

168. The conjugate of claim 167, wherein one or both CSRs is a receptor tyrosine kinase.

169. The conjugate of claim 166, wherein:
the first portion is from a herstatin if the intron-encoded portion is from herstatin.

170. A method of preparing a synthetic intron fusion protein, comprising:
linking an N-terminus of one cell surface receptor (CSR) isoform to an intron from an intron fusion protein, whereby the resulting fusion protein modulates an activity of a cell surface receptor.

171. The method of claim 170, wherein the linkage is covalent.

172. The combination of claim 156, wherein the isoforms and/or drugs are in separate compositions or in a single composition.

173. A method of treatment, comprising administering the components of the combination of claim 156, wherein each component is administered separately, simultaneously, intermittently, in a single composition or combinations thereof.
